SEQUENCE LISTING (14 sequences)

SEQ ID NO: 1 - DNA, 1083 bp, synthetic construct
  source 1..1083: mol_type = other DNA
atggctggccatctcgcaagtgacttcgcattttccccgcccccaggcggcggtggagatggacctggcggcccagaaccaggatgggtggacccccgcacgtggctttcttttcagggtccaccaggcggacccggtattggacccggcgtgggtcctgggtcagaagtctggggtatcccaccctgtcccccaccatacgaattttgcggcggaatggcctattgcggccctcaagtcggggtcggtctggtacctcagggtggtctggaaacatcccaaccagaaggtgaggccggtgtgggagttgaatccaattctgacggagcatctccagaaccttgtactgtgacaccaggagctgttaaattggagaaagaaaagctcgaacagaatccagaagaatcacaagatattaaggcgctccaaaaggagctggaacagttcgcgaaacttcttaaacagaaacgcattactctcgggtacacccaagcggacgttggactgactctcggtgtgctgttcggcaaagtctttagtcagaccacaatatgtcgattcgaagcccttcaactgtcatttaagaatatgtgcaaacttcgacctctgctccagaaatgggtcgaagaggcggataataacgagaacctgcaagaaatctgtaaggcggagactctggttcaagctcgcaaaaggaaacgtacgtctatagaaaatagagtccgtgggaatcttgaaaacctgtttctccaatgtccaaagcctactttgcaacaaatatctcatattgcgcaacaactcggcctggaaaaggacgtagttagagtctggttttgcaatcgcagacagaaagggaaacggtcttccagtgattacgcgcagagggaagacttcgaagcagccggttcaccgttttccggcggcccggtatctttcccattggctcccggtcctcacttcggcacacccgggtacggctcaccacattttaccgccctttattcaagcgttccctttccggaaggcgaggctttcccgccggtgtcagtgactacacttggatccccaatgcacagcaattag

SEQ ID NO: 2 - DNA, 954 bp, synthetic construct
  source 1..954: mol_type = other DNA
atgtataatatgatggaaaccgaattgaaaccacccgggccccaacagacctctggcggcggtggtggtaatagcacagcagcagccgctggtggaaatcaaaagaattctccagatagagtgaaacgacctatgaacgcatttatggtctggtctagaggacaacgaaggaaaatggctcaagaaaatcccaaaatgcataatagcgaaatttccaaacggttgggtgcggaatggaagctcctcagcgaaaccgaaaagaggccatttattgatgaagcgaaaagactcagggcattgcatatgaaagaacatccagactacaagtatagaccacgccgcaagacaaagactctgatgaagaaggacaaatataccctgcctggtggattgttggctcctggcggtaacagtatggcttctggcgtgggcgttggggctggacttggtgccggggtcaatcaacgaatggattcctatgcccatatgaatggatggagtaatggttcctattctatgatgcaagatcaattgggatatcctcaacatcccggtctgaacgctcatggtgctgctcaaatgcaacctatgcatcggtatgatgtaagtgcattgcaatataatagcatgacatccagtcaaacatatatgaatgggtcaccaacatatagtatgagctattcccaacaaggtacaccagggatggccctggggagcatggggagtgtcgttaaaagtgaagcttcaagttcaccacccgtagtgacgagttcttcacattctcgagccccatgtcaagcaggagatcttagggatatgatttcaatgtacttgccaggggctgaagtccccgagccggcagcgccttctaggctgcatatgtctcaacattatcaatccggacccgttccagggaccgctatcaatggtacgttgccattgtcccatatgtaa

SEQ ID NO: 3 - DNA, 1365 bp, synthetic construct
  source 1..1365: mol_type = other DNA
atggacttctttcgagtggtagagaatcaacaacccccggccaccatgccattgaatgtatcatttacaaaccggaactacgacctggactatgattcagttcagccttacttttattgtgacgaagaagaaaatttctatcaacaacaacaacaatcagaattgcaaccacccgctccatcagaagacatttggaagaagtttgaactcctgccaactccaccgctcagcccgagtagacgttctggactgtgttctccttcttatgtggctgtgactccgttttcactgcgtggcgataatgatggcggcggtggctcttttagtacagcagatcaacttgaaatggtcacagaactccttggtggtgatatggttaatcaatcattcatttgtgatcccgatgatgagacatttataaagaacatcatcatacaagactgcatgtggtcagggtttagtgctgctgcgaaactggtgagcgaaaagttggcttcttatcaagccgcccggaaggatagtggatcaccaaatccagcaaggggtcattcagtgtgtagcacaagctctctgtatcttcaagacctctccgcggctgcaagtgaatgtattgatccaagtgtcgttttcccttatcccctgaatgattcttcctctcctaaaagctgtgcgagccaggattcttcagctttctccccaagctccgacagtttgttgagctctactgaaagtagtcctcaagggtcaccggaacctctcgtccttcacgaagaaacaccccctacaactagttccgattccgaagaagaacaggaagacgaagaggagattgacgtggtatcagttgagaaaagacaagcccccgggaagcgaagcgaaagcgggagcccaagcgccggcggacattccaagcccccacattctcctttggtactgaaaagatgtcatgtgagcacccaccaacataattatgctgctcccccatcaaccaggaaagattaccccgccgctaaacgagttaaactggattcagtgagggttcttaggcaaatttcaaataataggaagtgtacttcacctcgcagtagcgatacagaagaaaacgttaaaagacggacgcataatgtgctggaacgacaaagacgaaatgaacttaagaggtccttcttcgcgcttagggatcaaatacctgaactggagaataacgagaaagctccaaaagtggtgattttgaagaaggctactgcgtatatactttctgtacaggccgaagaacagaaactgatatcagaggaagatctgcttcgtaagcgcagagagcaactgaagcataagctcgagcaactccgcaatagctgcgcctag

SEQ ID NO: 4 - DNA, 1542 bp, synthetic construct
  source 1..1542: mol_type = other DNA
atgcggcagccaccaggggaaagtgatatggccgtttccgacgctcttctgccttcattttcaacctttgcttccggacctgccgggcgggaaaagacgcttaggcaggccggagcacccaacaatcgatggagagaagaactgagccatatgaaaagactgccgcctgtactcccggggcggccatacgatctcgccgccgctacagtagcaactgatttggaatccggtggtgctggggcagcctgtggcggatctaatcttgctcctctgccaagacgggaaacggaagaatttaatgacctgctcgatttggatttcatcctttctaactcacttacacacccaccagaaagtgtcgctgctacggtctctagctctgccagcgcaagcagttcatcttctccatcttcctcaggacccgcaagtgccccgagcacttgttcctttacgtacccaatacgagctggcaatgatcctggagttgctcccggtggtacaggcggtggtctgttgtacggaagagaatcagccccaccaccaaccgcaccatttaatttggccgatattaatgatgtttcacctagtggtggttttgttgcggaactgctccgtcccgagctggatcccgtctatatcccaccacaacaaccccaaccacctggcggcggattgatgggtaaatttgttcttaaagcatccctttccgcaccagggtcagaatatggaagtccctcagtgatttccgtatctaagggatcccccgatgggtcccatccagttgtcgttgccccttataatggtggcccgcctagaacctgtccgaaaataaaacaagaagctgtatcatcatgtacgcatctcggggcaggtccacccctgtctaacgggcatcgccctgcagcccatgattttcctttgggccgacaactgccgtcccgtacaactccaacactcggcctcgaagaggtcctcagtagtagagattgccatccagcactccccctgccaccaggttttcaccctcatcccggtcctaactatcccagctttctcccagaccaaatgcaaccccaggttcccccacttcactatcagggccaaagcagaggtttcgtggcgcgagccggagaaccatgcgtctgttggcctcattttggcacccatggaatgatgttgacacccccaagtagtccactcgaactgatgccccctggcagttgtatgcctgaagaacctaaacccaaaagagggcggcggagttggccacgaaagcgaacagcaactcatacctgcgactatgctggatgtgggaagacatataccaaatctagccacctgaaagcgcatctccgcacgcatactggagaaaagccctatcattgcgattgggatgggtgcggctggaagtttgcacgatctgacgagcttactagacattatcgcaagcataccggacatcggccctttcaatgtcagaagtgtgatcgcgccttcagccggagcgatcatctggctctgcatatgaaacgacacttctag

SEQ ID NO: 5 - DNA, 630 bp, synthetic construct
  source 1..630: mol_type = other DNA
atgggctcagtcagcaatcaacaattcgcgggtggatgtgcaaaagctgctgaggaagccccagaagaagctccagaagatgccgctcgcgccgctgatgaaccacaactgctccatggagctgggatttgcaaatggtttaatgtccggatgggctttggtttcttgtctatgacagcaagagctggagtggccctcgatccacccgtcgacgtgttcgtacatcaatctaaacttcatatggaaggatttcgctcactcaaagaaggagaagccgtagaatttacattcaagaaaagtgcgaaagggcttgagagcatacgggtgacagggccaggcggcgtgttttgcatcggatcagaacgaaggcccaagggtaaatccatgcaaaagaggcgatccaaaggggatcggtgttataattgcggtggccttgaccaccacgcgaaagagtgtaaacttcccccgcaacctaagaaatgtcatttctgtcaatccatatcacacatggtggcgagctgcccattgaaagcacaacaaggaccgtccgcccaagggaaacctacttatttcagggaagaggaagaggagattcatagtcccactctccttcctgaagcccaaaactag

SEQ ID NO: 6 - DNA, 918 bp, synthetic construct
  source 1..918: mol_type = other DNA
atgtcagtcgacccggcctgcccacagagtctgccctgtttcgaggcttcagattgcaaggagtcctctccgatgcccgtcatctgcggacccgaggagaattaccccagtctgcagatgtccagtgccgaaatgccacatacagaaacggtttcaccgctcccatcttcaatggaccttttgatccaagattctcccgacagcagcacttccccaaagggaaagcagcctacctcagcggagaaatctgtggccaagaaagaggataaagttcctgttaagaagcaaaagacaaggaccgtattttcctcaacacaactctgcgtccttaacgaccggttccaacgccaaaagtatctgtccttgcaacaaatgcaggagctgagcaatattttgaatctgtcttataagcaagtcaagacttggtttcaaaaccagcgtatgaagagtaaaagatggcagaagaataattggcccaagaactctaacggcgttactcaaaaggcgagtgcccccacttatccatctctgtattcatcatatcatcaaggctgtttggttaatcccacgggcaatctgccgatgtggtccaatcaaacgtggaataacagtacttggtccaatcaaactcaaaatatacaaagttggtcaaatcattcttggaatacccaaacgtggtgtactcagagctggaataaccaagcatggaactccccattttacaattgcggcgaagagtcactccaaagctgtatgcaatttcaacccaacagtcccgcatccgatcttgaagcagcactggaagcggccggtgaggggctgaacgtgattcaacaaacaacaagatacttctccacacctcagactatggaccttttcctgaattattcaatgaatatgcagcccgaggatgtctag

SEQ ID NO: 7 - DNA, 1281 bp, synthetic construct
  source 1..1281: mol_type = other DNA
atggagttcgccatggagctgctgtcgccaccgttgcgtgacgtggacctgacagcccccgacggctctctgtgctcctttgctactaccgacgatttctacgatgacccgtgctttgattctcccgacctgcgctttttcgaagacttagatccgcgcctgatgcatgtaggtgctctgctaaagcccgaggagcacatggctggccacttggcttccgacttcgcgttctccccgccacccggcggcggaggcgatggccctggcggacccgagccaggttgggtcgaccctcggacctggctctccttccagggccccccaggcgggcccggcataggtcccggcgttggacccgggagcgaggtgtggggcatccctccgtgcccacccccgtatgagttctgcgggggtatggcctactgcgggcctcaggtgggcgtcggcctcgtccctcaggggggtctggagacctctcagccggagggagaggctggggtcggcgtggagagcaactccgacggggcgtcgcccgaaccttgcactgtcacgcccggggccgttaagctggagaaggagaaacttgagcagaaccccgaggagagccaggacatcaaggcgctgcagaaggagctggaacagttcgccaagctgctgaagcagaagcgcatcaccctaggttacacccaggcggacgtgggcctgacgcttggtgtgctgttcggaaaggtgttcagccagacgaccatctgccgattcgaggccctccagctgtccttcaagaacatgtgcaagttgcggcccctgctccaaaaatgggtggaggaggctgacaacaacgagaatctccaggagatctgtaaagccgagactctggtgcaggcccgcaaacgcaagcgtacctcgattgaaaacagggtgcgtggcaacctggagaacctgttcctacagtgtcccaagcctacccttcagcagattagccacatcgcacaacagttgggccttgaaaaggatgtggtacgcgtgtggttctgtaaccgccgccagaagggtaagcgcagctccagcgactacgcgcagagagaggactttgaggctgcaggatctcctttttctggcggccctgtgagtttccctctggccccgggaccccactttggtactccgggctacggctccccgcacttcaccgccctgtactctagtgtcccgtttcccgagggcgaggcgttccccccagtgtccgtgaccacactggggtccccaatgcattcaaattga

SEQ ID NO: 8 - DNA, 1056 bp, synthetic construct
  source 1..1056: mol_type = other DNA
atgacaatgaaaatgatggtgcacatctacttcgtctctcttctgctgctgctgtttcacagctacgcaattgacatcgaaaacgagatcaccgagttcttcaacaagatgcgtgacacccttcccgccaaggattctaaatggctcaacccggcctgcatgtttggaggcaccatgaacgacatcgcggcgctgggcgagcccttctccgctaaatgtcccccgatcgaagattctctgctgagccaccgctacaaggactatgtggtcaaatgggagcgcctggagaagaaccgtcggcgccaggtctcaaacaagcgcgtaaagcatggagatctttggatcgccaactacaccagtaaatttagcaaccgccgctacctgtgcactgtcaccaccaagaatggggactgcgtgcagggcatcgtgcggagccacatccgcaagcctccctcttgtattcccaagacctacgagctggggacacatgacaagtacggcattgacctgtattgcgggatcctgtacgcgaagcactacaacaacatcacctggtacaaggacaacaaggagattaacatcgatgacatcaagtactcccagactggcaaggagctcatcatccacaaccctgagctggaggactccggtagatatgattgttacgtgcattacgacgatgtgcgcatcaaaaacgacattgtcgtttccaggtgtaagatactcactgtgatccctagtcaggaccaccgcttcaagctgatcttggacccgaagataaatgtgaccatcggggagccagccaatatcacctgcactgccgtgtccacctcgttgctcattgacgatgtactgattgagtgggagaatccatcgggttggctaatcggcttcgacttcgacgtgtactcggtgctaacctcccgtggtggcatcactgaggctacgctttatttcgagaacgtgacggaggagtacatcggcaacacctacaagtgccgaggccacaactactacttcgaaaaaaccctgacgacgaccgtggttttggagtga

SEQ ID NO: 9 - DNA, 1542 bp, synthetic construct
  source 1..1542: mol_type = other DNA
atgcgtcagccacccggggagagcgacatggccgtgtcggacgcgctgctgccatccttttccaccttcgcctcgggtccggccggccgagagaagactctgcgccaggccggagcccctaacaaccgctggagagaggagctgtcacacatgaaacgcctgccccccgtgctgcctgggcgcccctacgaccttgccgcggccacggtggctaccgacttggagtctggaggtgctggagcagcgtgtggcggaagcaacctggcaccgttgccacgccgggagaccgaggagttcaacgacttgttggatctggactttattctgtccaactcccttacacacccgcccgagagcgtagcagccaccgtgagctccagtgcttccgcttcctcctcatccagcccgtcgtcttctggccctgcctctgcgccgtcgacctgttcgttcacctatcccatccgggccggcaacgatccgggcgtggccccgggcggcaccggtggtggtctcctgtacggcagggagtccgccccccctccaaccgctcccttcaacctcgcggacatcaatgacgtgtccccctctggcggcttcgttgcagaactgttaaggcctgaactggatccagtgtacatcccgccccagcagcctcagccgccgggcggcggtctgatgggcaaatttgtcctgaaggcgtctctgtctgctcctggctccgagtacggcagccccagtgtgattagcgtgtctaaaggcagccccgacgggtcgcaccccgtggtggtcgctccttacaacggtggacccccgcgcacctgcccaaagatcaagcaggaggctgtttcttcatgcactcatctaggcgccggtccacccctttccaatggccaccggcccgccgcgcatgacttccccctgggccgccagctgcccagccggaccacacctaccttaggcctggaggaggtgcttagttcgcgcgactgtcatcctgccctgcctctccctccaggcttccacccccacccgggacccaactacccgtccttcctgcccgaccagatgcaacctcaggtccctcccctgcactaccagggacagagccgcggtttcgtcgcccgtgctggcgagccatgcgtctgttggccgcacttcggcacgcatggaatgatgctaactcccccgagctcccccctggagctgatgcccccgggttcctgtatgccagaggagcccaagcctaagcgcggcagacgtagttggccccgtaagcgcaccgccacacatacgtgcgactacgcggggtgcgggaaaacctacaccaagtcttctcacctcaaggctcacttgcgtactcacactggagagaagccttatcactgcgactgggatgggtgcgggtggaagtttgctcgctccgacgaactcacccgccattaccgcaagcacaccggccaccgccccttccagtgtcagaagtgcgatcgagcgttctcccgctcggaccacctggccctacacatgaagaggcacttttga

SEQ ID NO: 10 - DNA, 630 bp, synthetic construct
  source 1..630: mol_type = other DNA
atgggctccgtgtcgaatcagcagttcgcaggcgggtgcgccaaggcagccgaggaggccccggaggaggctcctgaagacgccgctcgcgcggcggacgagcctcagctactccatggagctggcatctgcaaatggttcaacgtgcgaatgggatttggcttcctgtctatgactgctcgtgccggcgtggcgctggacccgcccgtagacgtgttcgtccaccagagcaagctgcatatggagggtttcagatctctgaaggagggcgaggccgtggagttcacgttcaagaagtcggccaaaggtctggagagcatccgcgtcaccggtcccggcggcgttttttgtattggctccgagcgccggcccaagggcaagtccatgcagaagcgccgctccaagggggacaggtgttacaactgcgggggtttggatcaccacgcgaaggagtgcaaacttcctcctcagccgaagaaatgtcacttttgtcagagcatcagtcacatggtggcttcatgcccactcaaggcccaacagggaccctctgcgcagggcaagcccacctacttccgcgaggaggaagaagagatccactccccaaccctgctgcccgaggctcagaactga

SEQ ID NO: 11 - DNA, 918 bp, synthetic construct
  source 1..918: mol_type = other DNA
atgtctgttgacccggcctgcccacagagccttccctgctttgaggcttccgactgtaaagagagttccccgatgcccgtgatttgtggtcctgaagagaactacccgtccctacagatgtcatcggcggagatgcctcataccgagaccgtgtcccccttaccctcttctatggatctgctgatccaggactcgcccgacagctcgaccagccctaagggcaagcagccaacctccgcagagaagtccgtggctaagaaggaggacaaagtaccggtgaagaaacagaagactcgcaccgtgttctcgtcgacccagctgtgcgtgctcaacgacaggtttcagcgccagaagtacctgtccctgcagcagatgcaagagctcagcaacatcctgaacctgagctacaagcaggtcaagacctggttccagaaccagcgtatgaagtccaagcggtggcaaaaaaacaactggcccaagaacagcaacggtgtcacccagaaggcctccgcccccacctacccgtctctttactccagctatcaccagggctgcctggtgaatccaacaggaaacctgcccatgtggtctaatcagacctggaacaactccacctggtctaaccagacacagaacatccagagttggtctaatcactcttggaacacgcagacctggtgcactcagagttggaataaccaggcctggaattcccccttctacaactgcggggaggagagcttgcagtcatgtatgcagttccagcccaactccccagccagtgacctggaggccgcgctggaggcggctggcgagggcctcaacgtgatccaacagacgacccgctacttctccacgcctcagactatggatttgttcctgaactattcgatgaacatgcagcctgaagatgtgtga

SEQ ID NO: 12 - DNA, 1083 bp, synthetic construct
  source 1..1083: mol_type = other DNA
atggcgggccacttggcttccgacttcgcgttctctcccccgcctggcggaggcggggacggccccggcggccctgagccaggctgggtcgatccacgcacctggctctccttccagggaccccccggggggcccggcatcggtcccggggtaggccccggatcggaggtgtggggcatcccgccgtgcccaccgccctatgagttctgtgggggtatggcctactgcgggcctcaggtcggtgtcggtctggtgcctcagggcggtctcgagaccagccagccggagggcgaggctggcgtgggtgtggagagcaacagcgatggcgcttctcctgaaccatgcactgtcacccccggcgccgtgaagctggagaaagagaaactggagcagaatccagaggagagtcaggacatcaaggccctgcagaaggaactggaacagttcgccaagctgctgaagcagaagcgtatcacccttggatacacccaggcggacgtgggcctcactttaggcgttcttttcggaaaggtgttctcacagaccacaatctgccgattcgaggccctgcaactttctttcaaaaacatgtgcaagttgcggcccctactgcagaaatgggtggaggaagcggacaacaacgagaatctgcaggagatctgtaaggccgagacactggtgcaggctcgcaagcgcaaaagaacgagcattgagaaccgcgtccgcggcaacctggagaacctgttcctgcagtgcccaaagcctaccctccagcaaattagccacatcgctcagcagctgggcctggagaaggatgtggtgagggtgtggttctgtaaccggcgccagaagggcaagcgcagctccagtgactacgcacagcgtgaggactttgaagccgccggctccccgttttcaggaggccccgtgtcctttcctttggctcccggccctcatttcggtactccgggctacggctccccccactttaccgccctttactcctctgtccccttccccgagggagaggcatttcccccggtgtccgtaacgaccctgggttccccaatgcactctaactga

SEQ ID NO: 13 - DNA, 954 bp, synthetic construct
  source 1..954: mol_type = other DNA
atgtataacatgatggaaacagagctgaagcccccggggcctcaacagacctccggcggtgggggcggcaactcgaccgctgccgcagctggtggaaaccagaagaacagtcccgacagagttaagcgcccgatgaacgcgttcatggtgtggtctcgcggccagcgccgcaagatggcgcaggagaatccaaaaatgcacaactcggagatctccaagcggctcggtgccgagtggaagctgctaagcgagaccgagaaacgtccttttattgacgaggccaagcgcctgcgtgcgcttcacatgaaggagcaccccgactacaagtacaggccccgacgcaaaaccaagaccctgatgaaaaaggacaagtacaccctccccggcggcctgctggcccctggtggcaacagcatggcctccggagtcggggtaggcgccggccttggagctggagtcaaccagcgtatggattcttacgcgcacatgaatgggtggtcaaatggctcgtattctatgatgcaggaccagctgggctaccctcaacaccccggcctcaacgcccatggagcggctcagatgcagccaatgcaccgctacgatgtgagcgccctgcagtacaactctatgactagttcacagacttacatgaacggttccccaacctactccatgtcttacagtcagcagggaacgccgggtatggctctgggctccatgggctccgtggtgaagtcggaggcatcctccagccctcccgtggtcacctcctcctctcacagccgcgctccttgccaggccggggacctgcgcgacatgatctctatgtatctgcccggtgcagaggtgcctgaaccggcggccccctctcggttgcatatgtcccagcattaccagagcggcccggtgccaggcactgccatcaacggcaccttgcccctgagccacatgtga

SEQ ID NO: 14 - DNA, 1365 bp, synthetic construct
  source 1..1365: mol_type = other DNA
atggatttcttccgagtggtggagaatcagcagccgcctgccaccatgccccttaacgtgtccttcactaacagaaactacgacctggactacgacagtgtccagccctatttctactgtgatgaggaggagaacttttaccagcagcaacagcagagcgaactgcagcccccagcgccctcggaggacatctggaagaaatttgagctgctgccaaccccccccctatctccatcccgccgttccggtctctgctctcctagctacgtggctgtcactcccttttcgctgcgtggggacaacgatggggggggcggaagtttcagcactgcggaccagctggagatggtgaccgagctgctgggtggtgacatggtcaaccagtcttttatctgcgacccggatgacgagaccttcatcaagaacatcatcatccaggactgtatgtggtctggcttctctgccgctgcaaaactggtctcggagaaacttgctagctaccaggctgctcgcaaggactccggctcgccgaatccagctaggggacatagtgtttgtagtacctcctcgctctacctgcaggacctgtccgcagcggcctctgagtgtattgacccgtccgtggtgttcccctatcctctcaacgactcgtcttcccccaagagctgcgcctcccaggactcatctgcgttctccccctcctccgatagcctgttgagtagcacagagagctcccctcagggctccccggagcccctggtgctacacgaggagaccccgcccaccaccagcagtgactcagaagaggagcaggaggacgaagaggagatcgatgtggtttccgtggagaagcgccaggcccctggcaaacgctccgaatccggctccccttccgccggcggccactccaagcccccgcacagcccgttggtgctgaagagatgtcacgtgtcaacccaccagcataactacgcggcgcctccctctacccgcaaagactaccccgctgccaagcgcgtgaagttggatagcgtccgggtcttacgccagatttctaacaaccgcaagtgcacttctccaaggtcttccgacacggaggagaacgtgaagcggcgcacccacaacgtactggagcgccagcgtcgtaacgagttgaagcgcagcttcttcgcgcttcgggaccagatacctgagctcgagaataacgagaaagcaccaaaggtagtcatcctgaagaaggccacggcctacatcttatcagtgcaggccgaggagcagaagctgatttcagaggaggatctgctgcgcaagcgccgagaacagctgaagcacaagctggaacaactccgcaactcatgcgcctga

(19) United States
(12) Patent Application Publication
(10) Pub. No.: US 2025/0248946 A1
(43) Pub. Date: Aug. 7, 2025

(54) METHODS AND COMPOSITIONS FOR IMMUNE CELL REJUVENATION AND THERAPIES USING REJUVENATED IMMUNE CELLS

(71) Applicant: TURN BIOTECHNOLOGIES, INC., Mountain View, CA (US)

(72) Inventors: Vittorio SEBASTIANO, Mountain View, CA (US); Naveen BOJJIREDDY, Mountain View, CA (US); Mustafa TURKOZ, Mountain View, CA (US)

(21) Appl. No.: 18/856,868

(22) PCT Filed: Apr. 14, 2023
(86) PCT No.: PCT/US2023/018744

§ 371 Date: Oct. 14, 2024

Related U.S. Application Data
(60) Provisional application No. 63/331,623, filed on Apr. 15, 2022.
(60) Provisional application No. 63/412,048, filed on Sep. 30, 2022.
(60) Provisional application No. 63/432,929, filed on Dec. 15, 2022.
(60) Provisional application No. 63/449,026, filed on Feb. 28, 2023.

(51) Int. Cl. A61K 9/51; C07C 229/12; C12N 15/88

(52) U.S. Cl. CPC A61K 9/5123; C07C 229/12; C12N 15/88

(57) ABSTRACT

Methods and compositions for cellular rejuvenation of immune cells, such as T cells, are provided. Cellular rejuvenation can be achieved by exposure, such as transient exposure, of immune cells to mRNAs encoding reprogramming factors. Compositions comprising such rejuvenated immune cells, including rejuvenated T cells, and uses of the rejuvenated immune cells in treating certain diseases and/or disorders, such as cancer and immune disorders, are also provided

### REFERENCE TO SEQUENCE LISTING, TABLE OR COMPUTER PROGRAM

The instant application contains a Sequence Listing which has been filed electronically in XML format and is hereby incorporated by reference in its entirety. Said XML copy, created on Apr. 11, 2023, is named “11277-0065-8004WO00_SEQ” and is 28,299 bytes in size. Nucleotide sequences related to the present disclosure are provided in Table 1.

### TECHNICAL FIELD

The subject matter described herein relates to compositions and methods for cellular rejuvenation of immune cells, such as T cells. Cellular rejuvenation can be achieved by exposure of immune cells to mRNAs encoding reprogramming factors such that the expression of reprogramming factors is transient. Compositions comprising such rejuvenated immune cells, including rejuvenated T cells, and uses of the rejuvenated immune cells in treating certain diseases and/or disorders, such as cancer and immune disorders, are also provided.

The subject matter described herein also relates to compositions and methods for inhibiting, preventing and/or reversing cellular exhaustion of engineered immune cells, such as T cells engineered to express a chimeric antigen receptor (CAR T cells). Inhibition, prevention and/or reversal of cellular exhaustion during CAR T cell manufacture can be achieved by exposure of T cells to mRNAs encoding reprogramming factors, such that the expression of reprogramming factors is transient and cellular identity is retained. Compositions comprising such transiently reprogrammed CAR T cells, and their use in treating certain diseases and/or disorders, such as cancer and immune disorders, are also provided.

### BACKGROUND

T cells exist in a continuation of differentiation states characterized by the gradual acquisition or loss of functional properties and gene expression patterns, the combined effect of which produces different phenotypic traits. At opposite ends of the differentiation spectrum are antigen-inexperienced naïve T cells (Tn or T_{N}) and terminally differentiated effector T cells (Teff, T_{E}, or T_{EFF}). In the middle are memory T cells, which are at an intermediate stage of differentiation, and they can be further divided, along a progressive developmental path, into memory stem cells (Tscm or T_{SCM}), central memory cells (Tcm or T_{CM}), and effector memory T cells (Tem or T_{EM}).

Therapies with functional immune cells, such as T cells, provide potentially effective therapeutic strategies for combating many types of immune cell related diseases and disorders, including cancer and viral infections. Chimeric Antigen Receptor (CAR) T-cell therapy involves genetically engineering a cancer patient's T cells to express a chimeric antigen receptor that targets one or more antigens found on the surface of tumor cells. These genetically engineered T cells, when infused back into the patient, home into tumor sites and exert powerful antitumor effects. CAR T-cell therapy has seen remarkable success in the treatment of specific blood cancers but has not reached its full therapeutic potential in terms of effectiveness or broader application. T-cell dysfunction and exhaustion, which negatively impact proliferation and T-cell mediated cytotoxicity, are major challenges diminishing the efficacy and persistence of CAR T-cell therapies in patients.

T-cell exhaustion is a state of T-cell dysfunction triggered by the continued stimulation and activation of T cells in the presence of antigen. In the case of cancer, continued stimulation of T cells is due to the presence of tumor antigens. T cell exhaustion is characterized by progressive loss of effector function, as well as reduced expression of cytokines such as interferon gamma (IFN-γ) and tumor necrosis factor alpha (TNF-α). It is also characterized by sustained or increased expression of inhibitory receptors such as PD1, Tim3, and LAG3. As T-cell exhaustion progresses, the effectiveness of CAR T-cell therapies is reduced, which leads to ineffective control of cancer.

The process for manufacturing CAR T-cells involves the activation of naïve T cells that have been isolated from a cancer patient. This activation step triggers T-cell proliferation and differentiation. However, prolonged CAR T-cell activation during the manufacturing process results in a higher percentage of the isolated T-cell population having a more differentiated and effector-like phenotype, which is prone to accelerated exhaustion and thus limited in vivo functionality. Despite initially high response and remission rates, 30% to 60% of patients that receive CAR T-cell therapy experience disease relapse, often within one year of completing the treatment (Xu, X. et al. Mechanisms of Relapse After CD19 CAR T-Cell Therapy for Acute Lymphoblastic Leukemia and Its Prevention and Treatment Strategies. Front. Immunol. 10, 2664 (2019); Vercellino, L. et al. Predictive factors of early progression after CAR T-cell therapy in relapsed/refractory diffuse large B-cell lymphoma. Blood Adv. 4, 5607-5615 (2020); Park, J. H. et al. Long-Term Follow-up of CD19 CAR Therapy in Acute Lymphoblastic Leukemia. N. Engl. J. Med. 378, 449-459 (2018); Maude, S. L. et al. Tisagenlecleucel in Children and Young Adults with B-Cell Lymphoblastic Leukemia. N. Engl. J. Med. 378, 439-448 (2018)). In most relapse cases, the target antigen is still expressed on the cancer cells, but the current manufacturing processes produce a single treatment dose, and a separate round of manufacturing is cost prohibitory. Furthermore, approximately 90% of patients experience CAR T-cell related side effects, raising patient safety concerns among clinicians. Collectively, these limitations of CAR T-cell products and their manufacturing preclude many patients from treatment and durable therapeutic responses.

It has been reported that CAR T cells isolated from patients treated for chronic lymphocytic leukemia (CLL) who experienced a robust and long lasting anti-tumor response had a high frequency of memory T cells with a “stem cell-like” phenotype. Less-differentiated memory T cell phenotypes, such as central memory T cells (Tcm) and stem cell memory T cells (Tscm) govern high proliferative capacity, long-term survival, and in vivo durability. In humans, Tscm quickly acquire effector functions after TCR stimulation, and they have a long lifespan. Just as importantly, Tscm resemble traditional stem cells in the sense that they have an enhanced capacity for self-renewal and an enhanced proliferative capacity. They can also replenish more differentiated memory T cells and effector T cells. Unfortunately, Tscm make up only a very small percentage (2% to 3%) of the total number of circulating T cells.

Accordingly, a means for inhibition, prevention and/or reversal of cellular exhaustion during CAR T cell manufacture which mitigates T-cell differentiation, increases proliferative capacity, increases tumor cell killing and promotes T central memory (Tcm) and/or T stem cell memory (Tscm) phenotypes, is highly desired.

In addition, a means for restoring or rejuvenating T cells, without dedifferentiation into stem cells, is also highly desirable. These “rejuvenated” T cells possess activities and properties of younger T cells, (as opposed to exhausted T cells), such as antigen-specific killing activity, self-renewal capability and multipotency. Rejuvenated T cells also demonstrate increased functional persistence and exhibit genetic patterns identical to those of younger T cells. Thus, there is a need for methods of rejuvenating immune cells that avoid dedifferentiation and loss of cell identity and that provide convenient and simple treatment paradigms.

The present disclosure addresses these needs, and provides additional benefits as well.

The foregoing examples of the related art and limitations related therewith are intended to be illustrative and not exclusive. Other limitations of the related art will become apparent to those of skill in the art upon a reading of the specification.

### BRIEF SUMMARY

The following aspects and embodiments thereof described and illustrated below are meant to be exemplary and illustrative, not limiting in scope.

In one aspect, a method for inhibiting, preventing or reversing exhaustion of an immune cell is provided. In some embodiments, the method comprises exposing the immune cell to messenger RNA (mRNA) encoding one or more reprogramming factors, whereby said exposing achieves expression of the one or more reprogramming factors in the immune cell to inhibit or prevent exhaustion of the immune cell with retention of its identity.

In another aspect, a method for increasing the self-renewal capability of an immune cell is provided. In some embodiments, the method comprises exposing the immune cell to mRNA encoding one or more reprogramming factors, whereby said exposing results in the expression of the one or more reprogramming factors in the immune cell to increase its self-renewal capability. In one embodiment, the increase in self-renewal capability is determined by measuring the expression levels—relative to an immune cell of the same type or subtype that is not exposed to the mRNA encoding one or more reprogramming factors—of one or more biomarkers selected from, but not limited to: BACH2, CD45RA, CD45RO, CCR7, CD62L, CD28, CD27, IL-7Rα, CXCR3, CD95, CD11a, IL-2Rβ, CD57, CD58, TCF1 and TCF7. In a further embodiment, the increase in self-renewal capability is demonstrated by a two-fold, three-fold, four-fold or greater increase in expression of one or more biomarkers selected from, but not limited to: BACH2, CD45RA, CD45RO, CCR7, CD62L, CD28, CD27, IL-7Rα, CXCR3, CD95, CD11a, IL-2Rβ, CD58, TCF1 and TCF7. In a further embodiment, the increase in self-renewal capability is demonstrated by an increase of greater than 5%, greater than 10%, greater than 15%, or greater than 20% in the expression of one or more biomarkers selected from, but not limited to: BACH2, CD45RA, CD45RO, CCR7, CD62L, CD28, CD27, IL-7Rα, CXCR3, CD95, CD11a, IL-2Rβ and CD58, TCF1 and TCF7. In an additional embodiment, the increase in self-renewal capability is demonstrated by an increase of greater than 20% in the expression of one or more biomarkers selected from, but not limited to: BACH2, CD45RA, CD45RO, CCR7, CD62L, CD28, CD27, IL-7Rα, CXCR3, CD95, CD11a, IL-2Rβ, CD58, TCF1 and TCF7. In one embodiment, the increase in self-renewal capability is demonstrated when the rejuvenated cell is shown to be capable of self-renewal for a period of time longer than an immune cell of the same type or subtype that is not exposed to the mRNA encoding one or more reprogramming factors.

In another aspect, a method for increasing the multipotency of an immune cell is provided. In some embodiments, the method comprises exposing the immune cell to mRNA encoding one or more reprogramming factors, whereby said exposing results in the expression of the one or more reprogramming factors in the immune cell to increase its multipotency. In one embodiment, the increase in multipotency is determined by measuring the expression levels—relative to an immune cell of the same type or subtype that is not exposed to the mRNA encoding one or more reprogramming factors—of one or more biomarkers selected from, but not limited to: BACH2, CD45RA, CD45RO, CCR7, CD62L, CD28, CD27, IL-7Rα, CXCR3, CD95, CD11a, IL-2Rβ, CD57, CD58, TCF1 and TCF7. In a further embodiment, the increase in multipotency is demonstrated by a two-fold, three-fold, four-fold or greater increase in expression of one or more biomarkers selected from, but not limited to: BACH2, CD45RA, CD45RO, CCR7, CD62L, CD28, CD27, IL-7Rα, CXCR3, CD95, CD11a, IL-2Rβ, CD58, TCF1 and TCF7. In a further embodiment, the increase in multipotency is demonstrated by an increase of greater than 5%, greater than 10%, greater than 15%, or greater than 20% in the expression of one or more biomarkers selected from, but not limited to: BACH2, CD45RA, CD45RO, CCR7, CD62L, CD28, CD27, IL-7Rα, CXCR3, CD95, CD11a, IL-2Rβ and CD58, TCF1 and TCF7. In an additional embodiment, the increase in multipotency is demonstrated by an increase of greater than 20% in the expression of one or more biomarkers selected from, but not limited to: BACH2, CD45RA, CD45RO, CCR7, CD62L, CD28, CD27, IL-7Rα, CXCR3, CD95, CD11a, IL-2Rβ, CD58, TCF1 and TCF7. In one embodiment, the increase in multipotency is demonstrated when the rejuvenated cell is shown to be capable of differentiating into multiple downstream cell types for a period of time longer than an immune cell of the same type or subtype that is not exposed to the mRNA encoding one or more reprogramming factors. In one embodiment, the increase in multipotency is demonstrated when the rejuvenated cell is shown to be capable of differentiating into a larger number of downstream cell types as compared to an immune cell of the same type or subtype that is not exposed to the mRNA encoding one or more reprogramming factors. In one embodiment, the increase in multipotency is determined by measuring the expression levels—relative to an immune cell of the same type or subtype that is not exposed to the mRNA encoding one or more reprogramming factors—of one or more biomarkers selected from, but not limited to: BACH2, CD45RA, CD45RO, CCR7, CD62L, CD28, CD27, IL-7Rα, CXCR3, CD95, CD11a, IL-2Rβ, CD57, CD58, TCF1 and TCF7. In a further embodiment, the increase in multipotency is demonstrated by a two-fold, three-fold, four-fold or greater increase in expression of one or more biomarkers selected from, but not limited to: BACH2, CD45RA, CD45RO, CCR7, CD62L, CD28, CD27, IL-7Rα, CXCR3, CD95, CD11a, IL-2Rβ, CD58, TCF1 and TCF7. In a further embodiment, the increase in multipotency is demonstrated by an increase of greater than 5%, greater than 10%, greater than 15%, or greater than 20% in the expression of one or more biomarkers selected from, but not limited to: BACH2, CD45RA, CD45RO, CCR7, CD62L, CD28, CD27, IL-7Rα, CXCR3, CD95, CD11a, IL-2Rβ and CD58, TCF1 and TCF7. In an additional embodiment, the increase in multipotency is demonstrated by an increase of greater than 20% in the expression of one or more biomarkers selected from, but not limited to: BACH2, CD45RA, CD45RO, CCR7, CD62L, CD28, CD27, IL-7Rα, CXCR3, CD95, CD11a, IL-2Rβ, CD58, TCF1 and TCF7.

In another aspect, a method for increasing the functional persistence of an immune cell is provided. In some embodiments, the method comprises exposing the immune cell to mRNA encoding one or more reprogramming factors, whereby said exposing results in the expression of the one or more reprogramming factors in the immune cell to increase its functional persistence. In one embodiment, the increase in functional persistence is demonstrated when the rejuvenated cell is shown to be capable of killing target cells, including but not limited to tumor cells, for a period of time longer than an immune cell of the same type or subtype that is not exposed to the mRNA encoding one or more reprogramming factors. In one embodiment, the increase in functional persistence is demonstrated when the rejuvenated cell produces an increased concentration of one or more cytokines, including but not limited to: IL-2, IFN-gamma (IFN-γ), GM-CSF and TNF-alpha (TNF-α), for a period of time longer than an immune cell of the same type or subtype that is not exposed to the mRNA encoding one or more reprogramming factors. In a further embodiment, the increase in functional persistence is demonstrated when the rejuvenated cell produces an increased concentration of one or more, two or more, or three or more of the following cytokines: IL-2, IFN-gamma (IFN-γ), GM-CSF and TNF-alpha (TNF-α), for a period of time longer than an immune cell of the same type or subtype that is not exposed to the mRNA encoding one or more reprogramming factors. In a further embodiment, the increase in functional persistence is demonstrated by a two-fold, three-fold, four-fold or greater increase in expression of one or more cytokines selected from, but not limited to: IL-2, IFN-gamma (IFN-γ), GM-CSF and TNF-alpha (TNF-α), for a period of time longer than an immune cell of the same type or subtype that is not exposed to the mRNA encoding one or more reprogramming factors. In a further embodiment, the increase in functional persistence is demonstrated by an increase of greater than 5%, greater than 10%, greater than 15%, or greater than 20% in the expression of one or more, two or more or three or more cytokines selected from, but not limited to IL-2, IFN-gamma (IFN-γ), GM-CSF and TNF-alpha (TNF-α), for a period of time longer than an immune cell of the same type or subtype that is not exposed to the mRNA encoding one or more reprogramming factors. In another embodiment, the increase in functional persistence is demonstrated when the rejuvenated cell is shown to decrease the level of one or more exhaustion markers including, but not limited to: TIGIT, LAG-3, TIM-3, PD-1 and 2B4, for a period of time longer than an immune cell of the same type or subtype that is not exposed to the mRNA encoding one or more reprogramming factors. In a further embodiment, the increase in functional persistence is demonstrated when the rejuvenated cell is shown to decrease the level of one or more exhaustion markers selected from the group consisting of: TIGIT, LAG-3, TIM-3, PD-1 and 2B4, for a period of time longer than an immune cell of the same type or subtype that is not exposed to the mRNA encoding one or more reprogramming factors.

In another aspect, a method for inducing or enhancing proliferation of an immune cell is provided. In some embodiments, the method comprises exposing the immune cell to mRNA encoding one or more reprogramming factors, whereby said exposing achieves expression of the one or more reprogramming factors in the immune cell to enhance the proliferation of the immune cell, with retention of its identity. In some embodiments, the method for inducing proliferation does not induce exhaustion. In some embodiments, the proliferation results from prevention or reversal of exhaustion.

In another aspect, a method for inducing proliferation is performed before, concurrently with, or after a method for inhibiting, preventing, or reversing exhaustion. In some embodiments, a method for inducing proliferation is performed at any time understood by one skilled in the art to provide sufficient proliferation before a method for inhibiting, preventing, or reversing exhaustion. In some embodiments, a method for inducing proliferation is performed at any time understood by one skilled in the art to provide sufficient proliferation after a method for inhibiting, preventing, or reversing exhaustion. In some embodiments, a method for inducing proliferation is performed 1, 2, 3, 4, 5, 6, 7, 8, 9, 10, 11, 12, 13, 14, 15, 16, 17, 18, 19, 20, or 21 days before a method for inhibiting, preventing, or reversing exhaustion. In some embodiments, a method for inducing proliferation is performed 1, 2, 3, 4, 5, 6, 7, 8, 9, 10, 11, 12, 13, 14, 15, 16, 17, 18, 19, 20, or 21 days after a method for inhibiting, preventing, or reversing exhaustion.

In some embodiments, methods of the present technology comprise immune cells that are lymphocytes, granulocytes, monocytes, macrophages, microglia, or dendritic cells. In some embodiments, the lymphocyte is a T-cell, a B-cell or a natural killer (NK) cell. In some embodiments, the lymphocyte is a tumor-infiltrating lymphocyte. In other embodiments, the lymphocyte is a T-cell. In some embodiments, the T cell is a cytotoxic T cell (CD8+), a helper T cell (CD4+), a suppressor or regulatory T cell (Treg), a memory T cell, a natural killer T cell (NKT cell), or a gamma delta T cell. In other embodiments, the helper T cell is a Th1, T2, Th17, Th9, or Tfh T-cell. In other embodiments, the T cell is an antigen-inexperienced naïve T cell (Tn or TN), or a stem cell memory T cell (Tsem or T_{SCM}), a central memory T cell (Tcm or T_{CM}), an effector memory T cell (Tem or T_{EM}), an effector T cell (Teff, T_{EFF} or T_{E}), a precursor to an exhausted T cell (Tpex or T_{PEX}), or an exhausted T cell (Tex or T_{EX}). In further embodiments, the T cells possess the phenotypic markers for their specific sub-type (e.g., Tscm, Tcm, Tem, Teff) as indicated in Table 2. In some embodiments, suppressor or regulatory T cells of the present technology are FOXP3+ (FOXP3-positive) T cells or FOXP3− (FOXP3-negative) T cells. In some embodiments, the NKT cell is a subset of CD1d-restricted T cells.

In some embodiments, a granulocyte of the present technology is a neutrophil, an eosinophil, a basophil, or a mast cell.

In other embodiments, a lymphocyte of the present technology is a B-cell. In some embodiments, a B-cell is a memory B-cell or a plasma cell.

In other embodiments, the immune cell is a monocyte, macrophage, microglial cell, or dendritic cell.

In some embodiments, the methods described herein may be used wherein the immune cell is a natural immune cell or an engineered immune cell. In some embodiments, the methods described herein are performed in parallel or in series with methods of engineering immune cells such that the methods are performed before, during, and/or after the engineering of the immune cells. In some embodiments, such engineering includes engineering so that the immune cells express chimeric antigen receptors. In some embodiments, such chimeric antigen receptors target at least one of CD19, CD20, CD22, CD30, CD33, CD123, FLT3, BCMA, GD2, HER2, MUC1, B7-H3, IL13Ra2, TAG72, MUC16, BCMA, or any other antigen suitable for immunotherapy. In some embodiments, such engineering includes engineering so that the immune cells express other proteins or peptides, such as growth factors and cytokines. In some embodiments, said cytokines include IL-7 and/or IL-15. In some embodiments, such engineering of immune cells is performed ex vivo, e.g., in the manufacturing of a cellular therapy product, such as an autologous or allogenic chimeric antigen receptor (CAR)-T, CAR-NK, CAR-M, or CAR-NKT cells. In some embodiments, the CAR-T cells provided herein are targeted to at least one of CD19, CD20, CD22, HER2, MUC1, CD30, CD33, CD123, FLT3, B7-H3, IL13Ra2, GD2, TAG72, MUC16, or BCMA by the chimeric antigen receptor. In some embodiments, the CAR-T cells provided herein are targeted to at least one of CD19 or BCMA by the chimeric antigen receptor. In some embodiments, the CAR-T cells provided herein are targeted to CD19 by the chimeric antigen receptor. In some embodiments, the CAR-T cells provided herein are targeted to BCMA by the chimeric antigen receptor. In some embodiments, the CAR-NK cells provided herein are targeted to at least one of CD19, FLT3, CD20, CD38, CD138, BCMS, CS1, CD3, CD5, CD7, NKG2D, HER2, EGFR, EpCAM, TF, B7-H6, HLA-G, CD24, CD44, CD133, mesothelin, or alphaFR by the chimeric antigen receptor. In some embodiments, the CAR-M cells provided herein are targeted to at least one of CD19, HER2, CD22, or ALK19 by the chimeric antigen receptor. In some embodiments, the CAR-NK cells provided herein are engineered to express IL-2 and/or IL-15. In some embodiments, the CAR-NKT cells provided herein are targeted to GD2, by the chimeric antigen receptor, and engineered to express IL-15. In such embodiments, the immune cell rejuvenation methods described herein are performed ex vivo during or after the manufacturing of the cell therapy product. In other embodiments, such engineering of immune cells is performed ex vivo, e.g., in so-called “in situ” generation of CAR-engineered cells. In such embodiments, mRNA encoding CARs or growth factors or cytokines is injected in vivo into a subject or patient, for example for CAR engineering of the patient's immune cells, such as T cells, NK cells, macrophages, tumor infiltrating lymphocytes, dendritic cells and/or NKT cells “in situ,” i.e., inside the patient's body without having to remove cells for ex vivo transfection. In such embodiments, the immune cell rejuvenation methods described herein are also performed in vivo, where mRNA encoding the reprogramming factor or factors is injected into the patient before, concurrently with, or after the mRNA encoding CARs or other cell engineering molecules. In some embodiments, the mRNA encoding the reprogramming factor or factors is delivered in vivo using lipid and/or polymer nanoparticles. In some embodiments, the lipid and/or polymer nanoparticles are engineered for targeted delivery to immune cells in vivo, such as T cells, NK cells, macrophages, tumor infiltrating cells, dendritic cells, and/or NKT cells in vivo. In still other embodiments, in vivo treatment is performed in the absence of any other in vivo cell engineering, to enhance or restore the potency of the immune system and treat diseases associated with immune dysfunction or dysregulation, such as improving the effect of the immune system against cancer or infection, or reducing inflammation.

In some embodiments, the methods described herein comprise exposing an immune cell to mRNA encoding one or more reprogramming factors, where the reprogramming factor is any reprogramming factor able to rejuvenate cells while retaining identity or without resulting in a loss of identity or de-differentiation. In some embodiments, the methods described herein comprise exposing an immune cell to mRNA encoding one or more reprogramming factors selected from the group consisting of an Oct, a Sox, a Klf, a Myc, a Lin NANOG, or GLIS1 i.e., any of these factors alone or in any combination. In other embodiments, said exposing comprises exposing to mRNA encoding one or more reprogramming factors selected from the group consisting of Oct4, Sox2, Klf4, cMyc, Lin28, NANOG, and GLIS1. In other embodiments, said exposing comprises exposing to mRNA encoding Oct4, Sox2, Klf4, cMyc, Lin28, and NANOG. In other embodiments, said exposing comprises exposing to mRNA encoding Oct4, Sox2, Klf4, GLIS1, Lin28, and NANOG. In other embodiments, said exposing comprises exposing to mRNA encoding Oct4, Sox2, Klf4, and cMyc. In other embodiments, said exposing comprises exposing to mRNA encoding Oct4, Sox2, Klf4, and GLIS1. In other embodiments, said exposing comprises exposing to mRNA encoding Oct4, Sox2, Lin28, and NANOG. In other embodiments, said exposing comprises exposing to mRNA encoding Oct4, Sox2, and Klf4. In some embodiments, the one or more reprogramming factors are expressed from a single mRNA molecule. In some embodiments, said single mRNA molecule is polycistronic. In other embodiments, two or more reprogramming factors are used, and the reprogramming factors are expressed from more than one mRNA molecule, for example wherein each reprogramming factor is expressed from its own mRNA molecule, or wherein two or more reprogramming factors are expressed from the same mRNA molecule. In some embodiments, at least one reprogramming factor is a fusion protein comprising a reprogramming factor or a domain of a reprogramming factor. In some embodiments, at least one reprogramming factor is a T cell optimized reprogramming factor.

In some embodiments, the mRNA molecule comprises a means to control the expression duration of at least one reprogramming factor, for example an on-off switch for expression, a trans- or self-amplification system, or a mechanism to control the half-life of the mRNA or the transcription factor protein transcribed from the mRNA. In some embodiments, the mRNA contains modifications to increase its half-life. Such means and modifications are known to those skilled in the art. In some embodiments, such control of expression duration, increased mRNA half-life, or increased transcription factor protein half-life allows the mRNA to be administered less frequently than would be required without such control of expression duration. In some embodiments, such control of expression duration, mRNA half-life, or protein transcription factor half-life allows the mRNA to be administered fewer times within a dosing interval, for example once, twice, or three times per dosing interval rather than at least once daily within the dosing interval. In some embodiments, the half-life of linear non replicative mRNA is controlled by altering the 3′ untranslated region, changing the poly (A) tail length, and/or adding an WPRE element to the mRNA. Through this mechanism, the half-life of mRNA is controlled from 6 minutes to 24 hours. Also through this mechanism, the half-life of mRNA is increased up to 72 hours. Use of circular RNA provides a half-life of 1-3 days. Use of trans- or self-amplifying RNA provides a half-life of 2 days or more, for example for up to 10 days or up to 12 days.

In some embodiments, said exposing comprises providing a composition comprising the mRNA, wherein the composition comprises an excipient for transfection. In some embodiments, said composition comprises a lipid and the mRNA are associated with the lipid. In some embodiments, the lipids comprise ionizable lipids that can be used in combination with other lipid components, such as helper lipids, stabilization lipids and structural lipids. In some embodiments, the disclosure also provides lipid-nanoparticle compositions comprising such lipids towards delivery of therapeutic nucleic acids. In other embodiments, said composition comprises a polymer and the mRNA are associated with the polymer. In some embodiments, said polymer is a charge-altering releasable transporter. In some embodiments, the charge-altering releasable transporter contains mixtures of lipophilic blocks in either block (b) or statistical (stat) architectures, such as those described in McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. In some embodiments, said polymer or lipid forms a nanoparticle, complex, or other nanostructure. In other embodiments, said composition comprises both a polymer and lipid and the mRNA are associated with the polymer and/or the lipid. In some embodiments, the use of a lipid or polymer for delivery of the mRNA, such as in a lipid nanoparticle, polymer nanoparticle, or hybrid lipid-polymer nanoparticle, results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA. In some embodiments, the use of a lipid or polymer for delivery of the mRNA results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA due to lower toxicity and/or lower physiological impact on the cell when compared to the different delivery mechanism. In some embodiments, the different delivery mechanism is electroporation such that the use of a lipid or polymer, including lipid or polymer nanoparticles, for delivery of the mRNA results in enhanced rejuvenation, proliferation, recovery from or prevention of exhaustion, anti-pathogenic effects, anti-cancer effects, or anti-inflammatory effects in the exposed immune cell compared to when using electroporation. This improvement compared to electroporation can result from reduced toxicity or reduced physiological impact on the cell compared to electroporation.

In some embodiments, the methods provided herein achieve transfection of the mRNA encoding one or more reprogramming factors into the immune cell. In some embodiments, said exposing achieves transfection of the mRNA encoding one or more reprogramming factors into T-cells or subpopulations of T-cells such as NKT cells or other immune cell types preferentially over other immune cell types or over other cell types.

In other embodiments, the methods described herein include exposing the immune cell to mRNA encoding one or more reprogramming factors cells in vitro, in vivo or ex vivo. In some embodiments, exposing is performed ex vivo using a technique selected from microinjection, electroporation, biolistics, optical transfection, microfluidic squeezing, chemical transfection, or biological transfection. In other embodiments, exposing is ex vivo and the method further comprises, after said exposing, transplanting the immune cell into a subject. In some embodiments of the present technology, exposing is in vivo and said exposing achieves transfection of the mRNA encoding one or more reprogramming factors into the immune cell for expression of the one or more reprogramming factors intracellularly. In other embodiments, exposing is in vivo and wherein said exposing or expression is prior to, concurrent with, or subsequent to administration of a therapeutic antibody, therapeutic protein or peptide, vaccine antigen, or bispecific antibody. In some embodiments, the therapeutic protein or peptide is a cytokine. In some embodiments, the vaccine antigen is a cancer vaccine antigen, including but not limited to a personalized cancer vaccine antigen. In some embodiments, the methods described herein enhance the efficacy and/or safety of an anti-cancer therapy, an anti-inflammatory therapy, or a cancer vaccine. In some embodiments, such methods are performed ex vivo, i.e., in the manufacturing of a cellular therapy product, such as an autologous or allogenic chimeric antigen receptor (CAR)-T, CAR-NK, CAR-M, or CAR-NKT cells. In some embodiments, such chimeric antigen receptors target at least one of CD19, CD20, CD22, CD30, CD33, CD123, FLT3, BCMA, GD2, HER2, MUC1, B7-H3, IL3Ra2, TAG72, MUC16, BCMA, or any other antigen suitable for immunotherapy. In some embodiments, such engineering includes engineering so that the immune cells express other proteins or peptides, such as growth factors and cytokines. In some embodiments, said cytokines include IL-7 and/or IL-15. In some embodiments, such engineering of immune cells is performed ex vivo, e.g., in the manufacturing of a cellular therapy product, such as an autologous or allogenic chimeric antigen receptor (CAR)-T, CAR-NK, CAR-M, or CAR-NKT cells. In some embodiments, the CAR-T cells provided herein are targeted to at least one of CD19, CD20, CD22, HER2, MUC1, CD30, CD33, CD123, FLT3, B7-H3, IL13Ra2, GD2, TAG72, MUC16, or BCMA by the chimeric antigen receptor. In some embodiments, the CAR-T cells provided herein are targeted to at least one of CD19 or BCMA by the chimeric antigen receptor. In some embodiments, the CAR-T cells provided herein are targeted to CD19 by the chimeric antigen receptor. In some embodiments, the CAR-T cells provided herein are targeted to BCMA by the chimeric antigen receptor. In some embodiments, the CAR-NK cells provided herein are targeted to at least one of CD19, FLT3, CD20, CD38, CD138, BCMS, CS1, CD3, CD5, CD7, NKG2D, HER2, EGFR, EpCAM, TF, B7-H6, HLA-G, CD24, CD44, CD133, mesothelin, or alphaFR by the chimeric antigen receptor. In some embodiments, the CAR-M cells provided herein are targeted to at least one of CD19, HER2, CD22, or ALK19 by the chimeric antigen receptor. In some embodiments, the CAR-NK cells provided herein are engineered to express IL-2 and/or IL-15. In some embodiments, the CAR-NKT cells provided herein are targeted to GD2, by the chimeric antigen receptor, and engineered to express IL-15. In such embodiments, the immune cell rejuvenation methods described herein are performed ex vivo during or after the manufacturing of the cell therapy product. In other embodiments, such engineering of immune cells is performed in vivo, e.g., in so-called “in situ” generation of CAR-engineered cells. In such embodiments, mRNA encoding CARs or other cell engineering molecules is injected in vivo into a subject or patient, for example for CAR engineering of the patient's immune cells, such as T cells, NK cells, macrophages, tumor infiltrating lymphocytes, dendritic cells and/or NKT cells “in situ,” i.e., inside the patient's body without having to remove cells for ex vivo transfection. In such embodiments, the immune cell rejuvenation methods described herein are also performed in vivo, where mRNA encoding the reprogramming factor or factors is injected into the patient before, concurrently with, or after the mRNA encoding CARs or other cell engineering molecules. In some embodiments, the mRNA encoding the reprogramming factor or factors is delivered in vivo using lipid and/or polymer nanoparticles. In some embodiments, the lipid and/or polymer nanoparticles are engineered for targeted delivery to immune cells in vivo, such as T cells, NK cells, macrophages, tumor infiltrating cells, dendritic cells, and/or NKT cells in vivo. In still other embodiments, in vivo treatment is performed in the absence of any other in vivo cell engineering, to enhance or restore the potency of the immune system and treat diseases associated with immune dysfunction or dysregulation, such as improving the effect of the immune system against cancer or infection, or reducing inflammation.

In some embodiments, the methods of the present technology comprise exposing an immune cell to mRNA or to at least one reprogramming factor expressed from transfected mRNA, or expressing at least one reprogramming factor from transfected mRNA, for a dosing interval understood by one of ordinary skill in the art to rejuvenate the immune cell without resulting in a loss of identity or differentiation. In some embodiments, the methods of the present technology comprise exposing an immune cell to mRNA or to at least one reprogramming factor expressed from transfected mRNA, or expressing at least one reprogramming factor from transfected mRNA, for a dosing interval of not more than 25, 24, 23, 22, 21, 20, 19, 18, 17, 16, 15, 14, 13, 12, 11, 10, 9, 8, 7, 6, 5, 4, or 3 consecutive days. In some embodiments, the mRNA dosing is performed at least once daily during the dosing interval. In some embodiments, the mRNA dosing is performed once daily during the dosing interval. In some embodiments, the mRNA dosing is performed twice daily during the dosing interval. In some embodiments, the mRNA dosing is performed at least twice daily during the dosing interval. In some embodiments, the mRNA dosing is performed less frequently than once per day during the dosing interval, for example once every two days, once every three days, once every four days, once every x days, where x is a number from 4 to 25. Thus, in such embodiments, for example, dosing mRNA once every 5 days in a 5 day dosing interval means that the mRNA is dosed once in the interval, i.e., once in the total treatment period of 5 days, whereas dosing mRNA twice daily in a 5 day dosing interval means that the mRNA is dosed 10 times in the interval, i.e., 10 times in the 5 days. In some embodiments, the methods of the present technology comprise exposing an immune cell to mRNA or to at least one reprogramming factor expressed from transfected mRNA, or expressing at least one reprogramming factor from transfected mRNA, for not more than 21, 18, 14, 10, 7, or 5 consecutive days.

In some embodiments, the methods of the present technology comprise exposing an immune cell to mRNA or to at least one reprogramming factor expressed from transfected mRNA, or expressing at least one reprogramming factor from transfected mRNA, for not more than 18 consecutive days. In some embodiments, the methods of the present technology comprise exposing an immune cell to mRNA or to at least one reprogramming factor expressed from transfected mRNA, or expressing at least one reprogramming factor from transfected mRNA, for not more than 14 consecutive days. In some embodiments, the methods of the present technology comprise exposing an immune cell to mRNA or to at least one reprogramming factor expressed from transfected mRNA, or expressing at least one reprogramming factor from transfected mRNA, for not more than 10 consecutive days. In embodiments, the methods of the present technology comprise exposing an immune cell to mRNA or to at least one reprogramming factor expressed from transfected mRNA, or expressing at least one reprogramming factor from transfected mRNA, for not more than 7 consecutive days. In embodiments, the methods of the present technology comprise exposing an immune cell to mRNA or to at least one reprogramming factor expressed from transfected mRNA, or expressing at least one reprogramming factor from transfected mRNA, for not more than 5 consecutive days. In some embodiments, the methods of the present technology comprise exposing an immune cell to mRNA or to at least one reprogramming factor expressed from transfected mRNA, or expressing at least one reprogramming factor from transfected mRNA, for not more than 5 consecutive days, and in some embodiments, the methods further comprise interrupting said exposing and repeating said exposing after said interrupting. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time.

In some embodiments, the methods of the present technology comprise exposing an immune cell to mRNA or to at least one reprogramming factor expressed from transfected mRNA, or expressing at least one reprogramming factor from transfected mRNA, for not more than 4 consecutive days, and in some embodiments, the methods further comprise interrupting said exposing and repeating said exposing after said interrupting. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time.

In some embodiments, the methods of the present technology comprise exposing an immune cell to mRNA or to at least one reprogramming factor expressed from transfected mRNA, or expressing at least one reprogramming factor from transfected mRNA, for not more than 3 consecutive days, and in some embodiments, the methods further comprise interrupting said exposing and repeating said exposing after said interrupting. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time.

In some embodiments, the methods of the present technology comprise exposing an immune cell to mRNA or to at least one reprogramming factor expressed from transfected mRNA, or expressing at least one reprogramming factor from transfected mRNA, for not more than 2 consecutive days, and in some embodiments, the methods further comprise interrupting said exposing and repeating said exposing after said interrupting. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time.

In some embodiments, the methods of the present technology comprise exposing an immune cell to mRNA or to at least one reprogramming factor expressed from transfected mRNA, or expressing at least one reprogramming factor from transfected mRNA, for only one day, and in some embodiments, further comprising interrupting said exposing and repeating said exposing after said interrupting. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time.

In other embodiments, said exposing comprises interrupting said exposing and repeating said exposing after said interrupting. In some embodiments, said exposing comprises exposing the immune cell to mRNA or to at least one reprogramming factor expressed from transfected mRNA, or expressing at least one reprogramming factor from transfected mRNA, for between about 2-5 consecutive days, between about 5-7 consecutive days, between about 7-10 consecutive days, between about 10-12 consecutive days, between about 12-14 consecutive days, between about 14-17 consecutive days, between about 17-19 consecutive, or between about 19-21 consecutive days and in some embodiments, further comprising interrupting said exposing and repeating said exposing after said interrupting. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time, for example until a disease is successfully treated or cured, or throughout the life of a subject or patient. In some embodiments, said repeating is performed any time after said interrupting, for example 0, 1, 2, 3, 4, 5, 6, 7, 8, 9, or 10 days, up to 20 days, up to 30 days, up to 3 months, up to 6 months, or up to 1 year after said interrupting. One exposure period is considered to be a dosing interval, such that, for example, a sequence of exposure-interruption-repeat exposure contains two dosing intervals. In some embodiments, the exposing to mRNA is performed once, twice, three times, four times, five times, or six times in a six day interval. In some embodiments, the exposing to mRNA is performed on the first or second day in a six day interval. In some embodiments, the exposing to mRNA is performed on the first or second and then on the fourth or fifth day in a six day interval. In some embodiments, the exposing to mRNA is performed on the first or second and then on the third or fourth and then on the fifth or sixth day in a six day interval. In some embodiments, the exposing to mRNA is performed every day in a six day interval. In some embodiments, the exposure interval occurs immediately after activation of the immune cell. In some embodiments, the exposure interval occurs one, two, three, four, five, six, seven, eight, nine, or ten days after activation of the immune cell. In some embodiments, the immune cell is activated with CD3 and/or CD28. In some embodiments, the activation is performed for one, two, three, four, five, six, or seven days.

In some embodiments, the methods of the present technology comprise exposing an immune cell to mRNA or to at least one reprogramming factor expressed from transfected mRNA, or expressing at least one reprogramming factor from transfected mRNA, twice daily for not more than 5 consecutive days. In some embodiments, the methods of the present technology comprise exposing an immune cell to mRNA or to at least one reprogramming factor expressed from transfected mRNA, or expressing at least one reprogramming factor from transfected mRNA, twice daily for not more than 4 consecutive days. In some embodiments, the methods of the present technology comprise exposing an immune cell to mRNA or to at least one reprogramming factor expressed from transfected mRNA, or expressing at least one reprogramming factor from transfected mRNA, twice daily for not more than 3 consecutive days. In some embodiments, the methods of the present technology comprise exposing an immune cell to mRNA or to at least one reprogramming factor expressed from transfected mRNA, or expressing at least one reprogramming factor from transfected mRNA, twice daily for not more than 2 consecutive days. In some embodiments, the methods of the present technology comprise exposing an immune cell to mRNA or to at least one reprogramming factor expressed from transfected mRNA, or expressing at least one reprogramming factor from transfected mRNA, twice daily for not more than 2 consecutive days. In some embodiments, the methods of the present technology comprise exposing an immune cell to mRNA or to at least one reprogramming factor expressed from transfected mRNA, or expressing at least one reprogramming factor from transfected mRNA, twice daily for not more than 1 day.

In another aspect, the present technology provides a method for preparing a composition for cell therapy. In some embodiments, the method comprises obtaining or providing a sample comprising one or more of the same or different types of immune cells; treating the one or more immune cells with mRNA encoding one or more reprogramming factors, whereby said treating does not cause loss of identity or loss of differentiation, and whereby said treating rejuvenates or reinvigorates the immune cells as evidenced by increased immune cell activity. In some embodiments, said treating is performed during the manufacturing process of a cell therapy product. In other embodiments, said treating is performed on a finished cell therapy product, after the manufacturing process is complete. In other embodiments, said treating is performed both during the manufacturing process and on the finished cell therapy product after the manufacturing process. In other embodiments, said treating is performed at the point of care before administration of the cells to a patient.

The rejuvenation of immune cells, such as T cells, can allow fewer cells to be used per administration when administered as a cell therapy product compared to the same immune cells when not rejuvenated, thus reducing cost per dose and overall cost, allowing for more doses to be produced, and allowing for banking of autologous cells. The rejuvenation of immune cells, such as T cells, can allow for fewer infusions required when compared to the same immune cells when not rejuvenated. In embodiments, immune cells rejuvenated as described herein, such as rejuvenated T cells, described herein are administered at a dose that is about 2-100, 3-100, 4-100, 5-100, 6-100, 7-100, 8-100, 9-100, or 10-100 fold lower than a dose clinically used for the same immune cells when not rejuvenated. In embodiments, immune cells rejuvenated as described herein, such as rejuvenated T cells, are administered at a dose that is at least 3-fold lower than a dose clinically used for the same immune cells when not rejuvenated. In embodiments, immune cells rejuvenated as described herein, such as rejuvenated T cells, are administered at a dose that is about 2-100, 3-100, 4-100, 5-100, 6-100, 7-100, 8-100, 9-100, or 10-100 fold lower than a dose clinically used for the same immune cells when not rejuvenated. In embodiments, immune cells rejuvenated as described herein, such as rejuvenated T cells, are administered at a dose of 0.01-1.5×10⁸ cells per infusion. In embodiments, CAR-T cells rejuvenated as described herein are administered at a dose of 0.01-1.5×10⁸ cells per infusion. In embodiments, immune cells rejuvenated as described herein, such as rejuvenated T cells, are administered at a dose of 0.02-1.2×10⁸ cells per infusion. In embodiments, CAR-T cells rejuvenated as described herein are administered at a dose of 0.02-1.2×10⁸ cells per infusion. In another aspect, the methods for preparing a composition for cell therapy utilizes immune cells such as lymphocytes, granulocytes, monocytes, macrophages, microglia, or dendritic cells. In some embodiments, the lymphocytes are T-cells, B-cells or natural killer (NK) cells. In some embodiments, the lymphocyte is a tumor-infiltrating lymphocyte.

In some embodiments, the immune cell is a T-cell that is a cytotoxic T cell (CD8+), a helper T cell (CD4+), a suppressor or regulatory T cell (Treg), memory T cell, a natural killer T cell (NKT cell), or a gamma delta T cell. In some embodiments, the helper T cell is a T1, Th2, Th17, Th9, or Tfh T-cell. In some embodiments, the T cell is a naïve T cell, (Tn or T_{N}), a stem cell memory T cell (Tscm or T_{SCM}), a central memory T cell (Tcm or T_{CM}), an effector memory T cell (Tem or T_{EM}), an effector T cell (Teff, T_{E}, or T_{EFF}), a precursor to an exhausted T cell (Tpex or T_{PEX}), or an exhausted T cell (Tex or T_{EX}). In some embodiments, the method uses T cells that possess the phenotypic markers for their specific sub-type (e.g., Tscm, Tcm, Tem, Teff) as indicated in Table 2. In other embodiments, the T cell is a memory T cell that is a central memory T cell, an effector memory T cell, a tissue resident memory T cell, or a virtual memory T cell. In other embodiments, the suppressor or regulatory T cell is a FOXP3+ T cell or a FOXP3− T cell. In some embodiments, the NKT cell is part of a subset of CD1d-restricted T cells.

In other embodiments, the immune cell is a granulocyte such as a neutrophil, an eosinophil, a basophil, or a mast cell.

In other embodiments, the immune cell is a monocyte, macrophage, microglial cell, or dendritic cell.

In some embodiments, the immune cell is a lymphocyte such as a B-cell including memory B-cells or plasma cells.

In other embodiments, the method for preparing a composition for cell therapy uses an immune cell that is an engineered immune cell. In some embodiments, the method is performed in parallel or in series with engineering of the immune cell, i.e., the method is performed before, during, and/or after the engineering of the immune cell. In some embodiments, the immune cell is engineered to express a chimeric antigen receptor. In some embodiments, the method for preparing a composition for cell therapy comprises treating with mRNA encoding one or more reprogramming factors selected from the group consisting of an Oct, an Sox, a Klf, a Myc, a Lin, NANOG, or GLIS1 including but not limited to, Oct4, Sox2, Klf4, cMyc, Lin28, NANOG, and GLIS1. In other embodiments, the method for preparing a composition for cell therapy comprises treating with mRNA encoding Oct4, Sox2, Klf4, cMyc, Lin28, and NANOG. In other embodiments, the method for preparing a composition for cell therapy comprises treating with mRNA encoding Oct4, Sox2, Klf4, GLIS1, Lin28, and NANOG. In other embodiments, the method for preparing a composition for cell therapy comprises treating with mRNA encoding Oct4, Sox2, Klf4, and cMyc. In other embodiments, the method for preparing a composition for cell therapy comprises treating with mRNA encoding Oct4, Sox2, Klf4, and GLIS1. In other embodiments, the method for preparing a composition for cell therapy comprises treating with mRNA encoding Oct4, Sox2, Lin28, and NANOG. In other embodiments, method for preparing a composition for cell therapy comprises treating with mRNA encoding Oct4, Sox2, and Klf4. In some embodiments, the one or more reprogramming factors are expressed from a single mRNA molecule. In some embodiments, said single mRNA molecule is polycistronic. In other embodiments, two or more reprogramming factors are used, and the reprogramming factors are expressed from more than one mRNA molecule, for example wherein each reprogramming factor is expressed from its own mRNA molecule, or wherein two or more reprogramming factors are expressed from the same mRNA molecule.

In some embodiments, the mRNA molecule used in the method for preparing a composition for cell therapy comprises a means to control the expression duration of at least one reprogramming factor, for example an on-off switch for expression, a self-amplification system, or a mechanism to control the half-life of the mRNA or the protein transcribed from the mRNA. In some embodiments, such control of expression duration allows the mRNA to be administered less frequently than would be required without such control of expression duration. In some embodiments, such control of expression duration allows the mRNA to be administered once per dosing interval rather than multiple times per dosing interval, for example once per dosing interval rather than at least once daily within the dosing interval.

In some embodiments, the method for preparing a composition for cell therapy comprises providing a composition comprising the mRNA, wherein the composition comprises an excipient for transfection. In some embodiments, said composition comprises a lipid and wherein the mRNA is associated with the lipid. In some embodiments, the lipids are selected from those described in FIGS. 1-18. In some embodiments, the lipids are those in Billingsley et al., Nano Let. 2020 (20, 3, 1578-1589), incorporated herein by reference. In other embodiments, said composition comprises a polymer and the mRNA are associated with the polymer. In some embodiments, said polymer is a charge-altering releasable transporter. The charge-altering releasable transporter may contain mixtures of lipophilic blocks in either block (b) or statistical (stat) architectures, such as those described in McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. In some embodiments, said polymer or lipid forms a nanoparticle, complex, or other nanostructure. In other embodiments, said composition comprises both a polymer and lipid and the mRNA are associated with the polymer and/or the lipid.

In some embodiments, the method for preparing a composition for cell therapy comprises treating that achieves transfection of the mRNA encoding one or more reprogramming factors into the immune cell. In some embodiments, said treating achieves transfection of the mRNA encoding one or more reprogramming factors into T-cells preferentially over other immune cells. In some embodiments, said treating is in vitro, in vivo or ex vivo. In some embodiments, said treating is ex vivo using a technique selected from microinjection, electroporation, biolistics, optical transfection, microfluidic squeezing, chemical transfection, or biological transfection. In other embodiments, said treating is ex vivo and the method further comprises, after said treating, transplanting the immune cell into a subject. In some embodiments, treating is in vivo and said treating achieves transfection of the mRNA encoding one or more reprogramming factors into the immune cell for expression of the one or more reprogramming factors intracellularly. In other embodiments, treating is in vivo and wherein said treating is prior to, concurrent with, or subsequent to administration of a bispecific antibody. In some embodiments, treating comprises treating the immune cell with the mRNA or with at least one reprogramming factor expressed from transfected mRNA, or with expression of at least one reprogramming factor from transfected mRNA, for a dosing interval of not more than 25, 24, 23, 22, 21, 20, 19, 18, 17, 16, 15, 14, 13, 12, 11, 10, 9, 8, 7, 6, 5, 4, or 3 consecutive days. In some embodiments, mRNA dosing is performed at least once daily during the dosing interval. In some embodiments, mRNA dosing is performed less frequently than once per day during the dosing interval, for example once every two days, once every three days, once every four days, once every x days, where x is a number from 4 to 25. Thus, in such embodiments, for example, dosing mRNA once every 5 days in a 5 day dosing interval means that the mRNA is dosed once in the interval, i.e., once in the total treatment period of 5 days, whereas dosing mRNA twice daily in a 5 day dosing interval means that the mRNA is dosed 10 times in the interval, i.e., 10 times in the 5 days. In some embodiments, the methods of the present technology comprise treating an immune cell with mRNA or with at least one reprogramming factor expressed from transfected mRNA, or with expression of at least one reprogramming factor from transfected mRNA, for not more than 21, 18, 14, 10, 7, or 5 consecutive days. In some embodiments, the methods of the present technology comprise treating an immune cell with mRNA or with at least one reprogramming factor expressed from transfected mRNA, or with expression of at least one reprogramming factor from transfected mRNA, for not more than 18 consecutive days. In some embodiments, the methods of the present technology comprise treating an immune cell with mRNA or with at least one reprogramming factor expressed from transfected mRNA, or with expression of at least one reprogramming factor from transfected mRNA, for not more than 14 consecutive days. In some embodiments, the methods of the present technology comprise treating an immune cell with mRNA or with at least one reprogramming factor expressed from transfected mRNA, or with expression of at least one reprogramming factor from transfected mRNA, for not more than 10 consecutive days. In embodiments, the methods of the present technology comprise treating an immune cell with mRNA or with at least one reprogramming factor expressed from transfected mRNA, or with expression of at least one reprogramming factor from transfected mRNA, for not more than 7 consecutive days. In embodiments, the methods of the present technology comprise treating an immune cell with mRNA or with at least one reprogramming factor expressed from transfected mRNA, or with expression of at least one reprogramming factor from transfected mRNA, for not more than 5 consecutive days. In other embodiments, said treating comprises interrupting said treating and repeating said treating after said interrupting. In some embodiments, said treating comprises treating the immune cell with mRNA or with at least one reprogramming factor expressed from transfected mRNA, or with expression of at least one reprogramming factor from transfected mRNA, for between about 2-5 consecutive days, between about 5-7 consecutive days, between about 7-10 consecutive days, between about 10-12 consecutive days, between about 12-14 consecutive days, between about 14-17 consecutive days, between about 17-19 consecutive days, or between about 19-21 consecutive days and in some embodiments, further comprising interrupting said treating and repeating said treating after said interrupting. In some embodiments, said repeating is performed any number of times, for example 1, 2, 3, 4, 5, 6, 7, 8, 9, or 10 times, or up to 20 times, or up to 30 times. In some embodiments, said repeating is performed any time after said interrupting, for example 0, 1, 2, 3, 4, 5, 6, 7, 8, 9, or 10 days, up to 20 days, up to 30 days, up to 3 months, up to 6 months, or up to 1 year after said interrupting. One treatment period is considered to be a dosing interval, such that, for example, a sequence of treatment-interruption-repeat treatment contains two dosing intervals. In some embodiments, the treating with mRNA is performed once, twice, three times, four times, five times, or six times in a six day interval. In some embodiments, the treating with mRNA is performed on the first or second day in a six day interval. In some embodiments, the treating with mRNA is performed on the first or second and then on the fourth or fifth day in a six day interval. In some embodiments, the treatment with mRNA is performed on the first or second and then on the third or fourth and then on the fifth or sixth day in a six day interval. In some embodiments, the treatment with mRNA is performed every day in a six day interval. In some embodiments, the treatment interval occurs immediately after activation of the immune cell. In some embodiments, the treatment interval occurs one, two, three, four, five, six, seven, eight, nine, or ten days after activation of the immune cell. In some embodiments, the immune cell is activated with CD3 and/or CD28. In some embodiments, the activation is performed for one, two, three, four, five, six, or seven days.

In another aspect, the present technology is related to a population of immune cells prepared according to the methods described herein.

In another aspect, the present technology is related to the use of a population of immune cells prepared according to the methods described herein for treating a disease or disorder. In some embodiments, the disease or disorder is cancer. In some embodiments, the cancer is a solid tumor. In some embodiments, the disease or disorder is an autoimmune disease, including but not limited to multiple sclerosis, rheumatoid arthritis, lupus, celiac disease, Sjögren's syndrome, ankylosing spondylitis, polymyalgia rheumatica, alopecia areata, vasculitis, or temporal arteritis, or T-cell immunodeficiency diseases that include, but are not limited to, severe combined immunodeficiencies (SCIDs), Wiskott-Aldrich syndrome, ataxia telangiectasia, DiGeorge syndrome (22q11.2 deletion syndrome), immuno-osseous dysplasias, dyskeratosis congenita, and chronic mucocutaneous candidiasis. In some embodiments, the disease or disorder is fibrosis, including but not limited to fibrosis of the heart, liver, lung, kidney, pancreas, bladder, uterus, or gastrointestinal tract. In some embodiments, the disease or disorder is associated with inflammation, such as neuroinflammation or inflammation of the heart, liver, lung, kidney, pancreas, bladder, uterus, or gastrointestinal tract. In some embodiments, the disease or disorder is dementia, Alzheimer's disease, Parkinson's disease, or spinal cord injury. In some embodiments, the disease is related to viral or bacterial infection, for example, sepsis, hepatitis, or COVID-19. In some embodiments, the disease or disorder is related to aging and/or chronic tissue damage, such as diabetes, fibrosis, cancer, neurodegeneration, arthritis, sarcopenia. In some embodiments, the population of immune cells prepared according to the methods described herein is used to destroy or inhibit senescent cells, e.g., for senotherapy, senolysis, senomorphic effects, or senoblocking.

In another aspect, the present technology is related to method for rejuvenating an immune cell, comprising: introducing mRNA encoding one or more reprogramming factors into the immune cell for expression of the one or more reprogramming factors, thereby generating an immune cell that expresses the one or more reprogramming factor to obtain a rejuvenated immune cell. In some embodiments, the immune cell is a lymphocyte or a granulocyte. In other embodiments, the lymphocyte is a T-cell, a B-cell or a natural killer (NK) cell. In some embodiments, the lymphocyte is a tumor-infiltrating lymphocyte. In other embodiments, the lymphocyte is a T-cell, such as a cytotoxic T cell (CD8+), a helper T cell (CD4+), a suppressor or regulatory T cell (Treg), a memory T cell, or a natural killer T cell (NKT cell). In some embodiments, the helper T cell is a Th1, T2, Th17, Th9, or Tfh T-cell. In other embodiments, the helper T cell is a Th1, Th2, Th17, Th9, or Tfh T-cell. In other embodiments, the T cell is an antigen-inexperienced naïve T cell (Tn or T_{N}), or a stem cell memory T cell (Tscm or T_{SCM}), a central memory T cell (Tcm or T_{CM}), an effector memory T cell (Tem or T_{EM}), an effector T cell (Teff, T_{EFF} or T_{E}), a precursor to an exhausted T cell (Tpex or T_{PEX}), or an exhausted T cell (Tex or T_{EX}). In further embodiments, the T cells possess the phenotypic markers for their specific sub-type (e.g., Tscm, Tcm, Tem, Teff) as indicated in Table 2. In other embodiments, the T cell is a memory T cell such as a central memory T cell, an effector memory T cell, a tissue resident memory T cell, or a virtual memory T cell. In other embodiments, the T cell is a suppressor or regulatory T cell that is a FOXP3+ T cell or a FOXP3− T cell. In other embodiments, the immune cell is a granulocyte such as a neutrophil, an eosinophil, a basophil, or mast cell. In some embodiments, the immune cells is a monocyte, a macrophage, microglial cell, or dendritic cell. In some embodiments, the immune cell is a lymphocyte such as a B-cell, including memory B-cells or plasma cells. In some embodiments, the immune cell is a natural immune cell. In other embodiments, the immune cell is an engineered immune cell. In some embodiments, the engineered immune cell is engineered in parallel or in series with the method of rejuvenation such that the rejuvenation occurs before, at the same time, or after the engineering. In some embodiments, the immune cells is engineered to express a chimeric antigen receptor.

In some embodiments, the method for rejuvenating an immune cell, comprises introducing mRNA encoding one or more reprogramming factors that are able to rejuvenate cells without resulting in a loss of identity or de-differentiation. In some embodiments, the method for rejuvenating an immune cell, comprises introducing mRNA encoding one or more reprogramming factors selected from the group consisting of an Oct, an Sox, a Klf, a Myc, a Lin, NANOG, or GLIS1, including, but not limited to, Oct4, Sox2, Klf4, cMyc, Lin28, NANOG, and GLIS1. In other embodiments, the method for rejuvenating an immune cell, comprises introducing mRNA encoding Oct4, Sox2, Klf4, cMyc, Lin28, and NANOG. In other embodiments, the method for rejuvenating an immune cell, comprises introducing mRNA encoding Oct4, Sox2, Klf4, GLIS1, Lin28, and NANOG. In other embodiments, the method for rejuvenating an immune cell, comprises introducing mRNA encoding Oct4, Sox2, Klf4, and cMyc. In other embodiments, the method for rejuvenating an immune cell, comprises introducing mRNA encoding Oct4, Sox2, Klf4, and GLIS1. In other embodiments, the method for rejuvenating an immune cell, comprises introducing mRNA encoding Oct4, Sox2, Lin28, and NANOG. In other embodiments, method for rejuvenating an immune cell, comprises introducing mRNA encoding Oct4, Sox2, and Klf4. In some embodiments, the one or more reprogramming factors are expressed from a single mRNA molecule. In some embodiments, said single mRNA molecule is polycistronic. In other embodiments, two or more reprogramming factors are used, and the reprogramming factors are expressed from more than one mRNA molecule, for example wherein each reprogramming factor is expressed from its own mRNA molecule, or wherein two or more reprogramming factors are expressed from the same mRNA molecule.

In some embodiments, the introduced mRNA molecule comprises a means to control the expression duration of at least one reprogramming factor, for example an on-off switch for expression, a self-amplification system, or a mechanism to control the half-life of the mRNA or the protein transcribed from the mRNA. In some embodiments, such control of expression duration allows the mRNA to be administered less frequently than would be required without such control of expression duration. In some embodiments, such control of expression duration allows the mRNA to be administered once per dosing interval rather than multiple times per dosing interval, for example once per dosing interval rather than at least once daily within the dosing interval.

In some embodiments, introducing the mRNA comprises providing a composition comprising the mRNA, wherein the composition comprises an excipient for transfection. In some embodiments, said composition comprises a lipid and the mRNA is associated with the lipid. In some embodiments, the lipids are those described in FIGS. 1-18. In some embodiments, the lipids are those in Billingsley et al., Nano Lett. 2020 (20, 3, 1578-1589), incorporated herein by reference. In other embodiments, said composition comprises a polymer and the mRNA are associated with the polymer. In some embodiments, said polymer is a charge-altering releasable transporter. In some embodiments, the charge-altering releasable transporter contains mixtures of lipophilic blocks in either block (b) or statistical (stat) architectures, i.e., such as those described in McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161). In some embodiments, said polymer or lipid forms a nanoparticle, complex, or other nanostructure. In other embodiments, said composition comprises both a polymer and lipid and the mRNA are associated with the polymer and/or the lipid. In some embodiments, the use of a lipid or polymer for delivery of the mRNA, such as in a lipid nanoparticle, polymer nanoparticle, or hybrid lipid-polymer nanoparticle, results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA. In some embodiments, the use of a lipid or polymer for delivery of the mRNA results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA due to lower toxicity and/or lower physiological impact on the cell when compared to the different delivery mechanism. In some embodiments, the different delivery mechanism is electroporation, i.e., the use of a lipid or polymer, such as lipid or polymer nanoparticles, for delivery of the mRNA results in enhanced rejuvenation, proliferation, recovery from or prevention of exhaustion, anti-pathogenic effects, anti-cancer effects, or anti-inflammatory effects in the exposed immune cell compared to using electroporation. This improvement compared to electroporation can result from reduced toxicity or reduced physiological impact on the cell compared to electroporation.

In some embodiments, introducing the mRNA achieves transfection of the mRNA encoding one or more reprogramming factors into the immune cell. In some embodiments, introducing the mRNA achieves transfection of the mRNA encoding one or more reprogramming factors into T-cells preferentially over other immune cells or over other cell types. In some embodiments, the mRNA is introduced in vitro, in vivo or ex vivo. In some embodiments, mRNA is introduced using an ex vivo technique selected from microinjection, electroporation, biolistics, optical transfection, microfluidic squeezing, chemical transfection, or biological transfection. In other embodiments, mRNA is introduced ex vivo and the method further comprises, after said introducing, transplanting the immune cell into a subject. In some embodiments, mRNA is introduced in vivo and said introducing achieves transfection of the mRNA encoding one or more reprogramming factors into the immune cell for expression of the one or more reprogramming factors intracellularly. In some embodiments, introducing is in vivo and is prior to, concurrent with, or subsequent to administration of a bispecific antibody. In some embodiments, such engineering of immune cells is performed ex vivo, i.e., in the manufacturing of a cellular therapy product, such as an autologous or allogenic chimeric antigen receptor (CAR)-T, CAR-NK, CAR-M, or CAR-NKT cells. In some embodiments, such chimeric antigen receptors target at least one of CD19, CD20, CD22, CD30, CD33, CD123, FLT3, BCMA, GD2, HER2, MUC1, B7-H3, IL13Ra2, TAG72, MUC16, BCMA, or any other antigen suitable for immunotherapy. In some embodiments, such engineering includes engineering so that the immune cells express other proteins or peptides, such as growth factors and cytokines. In some embodiments, said cytokines include IL-7 and/or IL-15. In some embodiments, such engineering of immune cells is performed ex vivo, e.g., in the manufacturing of a cellular therapy product, such as an autologous or allogenic chimeric antigen receptor (CAR)-T, CAR-NK, CAR-M, or CAR-NKT cells. In some embodiments, the CAR-T cells provided herein are targeted to at least one of CD19, CD20, CD22, HER2, MUC1, CD30, CD33, CD123, FLT3, B7-H3, IL13Ra2, GD2, TAG72, MUC16, or BCMA by the chimeric antigen receptor. In some embodiments, the CAR-T cells provided herein are targeted to at least one of CD19 or BCMA by the chimeric antigen receptor. In some embodiments, the CAR-T cells provided herein are targeted to CD19 by the chimeric antigen receptor. In some embodiments, the CAR-T cells provided herein are targeted to BCMA by the chimeric antigen receptor. In some embodiments, the CAR-NK cells provided herein are targeted to at least one of CD19, FLT3, CD20, CD38, CD138, BCMS, CS1, CD3, CD5, CD7, NKG2D, HER2, EGFR, EpCAM, TF, B7-H6, HLA-G, CD24, CD44, CD133, mesothelin, or alphaFR by the chimeric antigen receptor. In some embodiments, the CAR-M cells provided herein are targeted to at least one of CD19, HER2, CD22, or ALK19 by the chimeric antigen receptor. In some embodiments, the CAR-NK cells provided herein are engineered to express IL-2 and/or IL-15. In some embodiments, the CAR-NKT cells provided herein are targeted to GD2, by the chimeric antigen receptor, and engineered to express IL-15. In such embodiments, the immune cell rejuvenation methods described herein are performed ex vivo during or after the manufacturing of the cell therapy product. In other embodiments, such engineering of immune cells is performed ex vivo, e.g., in so-called “in situ” generation of CAR-engineered cells. In such embodiments, mRNA encoding CARs or other cell engineering molecules is injected in vivo into a subject or patient, for example for CAR engineering of the patient's immune cells, such as T cells, NK cells, macrophages, tumor infiltrating lymphocytes, dendritic cells and/or NKT cells “in situ,” i.e., inside the patient's body without having to remove cells for ex vivo transfection. In such embodiments, the immune cell rejuvenation methods described herein are also performed in vivo, where mRNA encoding the reprogramming factor or factors is injected into the patient before, concurrently with, or after the mRNA encoding CARs or other cell engineering molecules. In some embodiments, the mRNA encoding the reprogramming factor or factors is delivered in vivo using lipid and/or polymer nanoparticles. In some embodiments, the lipid and/or polymer nanoparticles are engineered for targeted delivery to immune cells in vivo, such as T cells, NK cells, macrophages, tumor infiltrating cells, dendritic cells, and/or NKT cells in vivo. In still other embodiments, in vivo treatment is performed in the absence of any other in vivo cell engineering, to enhance or restore the potency of the immune system and treat diseases associated with immune dysfunction or dysregulation, such as improving the effect of the immune system against cancer or infection, or reducing inflammation.

In some embodiments, the mRNA or at least one reprogramming factor expressed from transfected mRNA is introduced to the immune cell or expressed in the immune cell for a dosing interval of not more than of not more than 25, 24, 23, 22, 21, 20, 19, 18, 17, 16, 15, 14, 13, 12, 11, 10, 9, 8, 7, 6, 5, 4, or 3 consecutive days. In some embodiments, the mRNA dosing is performed at least once daily during the dosing interval. In some embodiments, the mRNA dosing is performed less frequently than once per day during the dosing interval, for example once every two days, once every three days, once every four days, once every x days, where x is a number from 4 to 25. Thus, in such embodiments, for example, dosing mRNA once every 5 days in a 5 day dosing interval means that the mRNA is dosed once in the interval, i.e., once in the total treatment period of 5 days, whereas dosing mRNA twice daily in a 5 day dosing interval means that the mRNA is dosed 10 times in the interval, i.e., 10 times in the 5 days. In some embodiments, the methods of the present technology comprise introducing an immune cell to mRNA or at least one reprogramming factor expressed from transfected mRNA, or to or expression of at least one reprogramming factor from transfected mRNA, for not more than 21, 18, 14, 10, 7, or 5 consecutive days. In some embodiments, the methods of the present technology comprise introducing an immune cell to mRNA or at least one reprogramming factor expressed from transfected mRNA, or to or expression of at least one reprogramming factor from transfected mRNA, for not more than 18 consecutive days. In some embodiments, the methods of the present technology comprise introducing an immune cell to mRNA or at least one reprograming factor expressed from transfected mRNA, or to or expression of at least one reprogramming factor from transfected mRNA, for not more than 14 consecutive days. In some embodiments, the methods of the present technology comprise introducing an immune cell to mRNA or at least one reprograming factor expressed from transfected mRNA, or to or expression of at least one reprogramming factor from transfected mRNA, for not more than 10 consecutive days. In embodiments, the methods of the present technology comprise introducing an immune cell to mRNA or at least one reprograming factor expressed from transfected mRNA, or to or expression of at least one reprogramming factor from transfected mRNA, for not more than 7 consecutive days. In embodiments, the methods of the present technology comprise introducing an immune cell to mRNA or at least one reprograming factor expressed from transfected mRNA, or to or expression of at least one reprogramming factor from transfected mRNA, for not more than 5 consecutive days. In other embodiments, said exposing comprises interrupting said introducing and repeating said introducing after said interrupting. In some embodiments, said introducing comprises introducing the immune cell to mRNA or at least one reprograming factor expressed from transfected mRNA, or to or expression of at least one reprogramming factor from transfected mRNA, for between about 2-5 consecutive days, between about 5-7 consecutive days, between about 7-10 consecutive days, between about 10-12 consecutive days, between about 12-14 consecutive days, between about 14-17 consecutive days, between about 17-19 consecutive days, or between about 19-21 consecutive days and in some embodiments, further comprising interrupting said introducing and repeating said introducing after said interrupting. In some embodiments, said repeating is performed any number of times, for example 1, 2, 3, 4, 5, 6, 7, 8, 9, or 10 times, or up to 20 times, or up to 30 times. In some embodiments, said repeating is performed any time after said interrupting, for example 0, 1, 2, 3, 4, 5, 6, 7, 8, 9, or 10 days, up to 20 days, up to 30 days, up to 3 months, up to 6 months, or up to 1 year after said interrupting. Said repeating may continue for any duration of time, for example until a disease is successfully treated or cured, or throughout the life of a subject or patient. One exposure period is considered to be a dosing interval, such that, for example, a sequence of introduction-interruption-repeat introduction contains two dosing intervals. In some embodiments, the exposing to mRNA is performed once, twice, three times, four times, five times, or six times in a six day interval. In some embodiments, the exposing to mRNA is performed on the first or second day in a six day interval. In some embodiments, the exposing to mRNA is performed on the first or second and then on the fourth or fifth day in a six day interval. In some embodiments, the exposing to mRNA is performed on the first or second and then on the third or fourth and then on the fifth or sixth day in a six day interval. In some embodiments, the exposing to mRNA is performed every day in a six day interval. In some embodiments, the exposure interval occurs immediately after activation of the immune cell. In some embodiments, the exposure interval occurs one, two, three, four, five, six, seven, eight, nine, or ten days after activation of the immune cell. In some embodiments, the immune cell is activated with CD3 and/or CD28. In some embodiments, the activation is performed for one, two, three, four, five, six, or seven days.

In embodiments, at least one reprogramming factor is expressed from the transfected mRNA within cells, cells are exposed to at least one reprogramming factor expressed from the transfected mRNA, for not more than about 20, 19, 18, 17, 16, 15, 14, 13, 12, 11, 10, 9, 8, 7, 6, 5, 4, 3, 2, or 1 days, or for less than 1 day. In embodiments, the at least one reprogramming factor is expressed from the transfected mRNA within cells, or the cells are exposed to the at least one reprogramming factor expressed from the transfected mRNA, for not more than about 14, 13, 12, 11, 10, 9, 8, 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 14, 13, 12, 11, 10, 9, 8, 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 10, 9, 8, 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 10, 9, 8, 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 5, 4, 3, or 2 days. In embodiments, the rejuvenated cells have a phenotype or activity profile similar to a young cell. The phenotype or activity profile includes one or more of the transcriptomic profile, gene expression of one or more nuclear and/or epigenetic markers, proteolytic activity, mitochondrial health and function, SASP cytokine expression, and methylation landscape.

As described herein, cells may be rejuvenated by transient reprogramming with mRNAs encoding one or more cellular reprogramming factors transfected into the cells. Transient reprogramming is accomplished, in some embodiments, by transfecting cells with non-integrative mRNAs for not more than about 20, 19, 18, 17, 16, 15, 14, 13, 12, 11, 10, 9, 8, 7, 6, 5, 4, 3, 2, or 1 days, or less than 1 day. Transient reprogramming is accomplished, in some embodiments, by transfecting cells with non-integrative mRNAs for not more than about 14, 13, 12, 11, 10, 9, 8, 7, 6, 5, 4, 3, 2, or 1 days, or less than 1 day. Transient reprogramming is accomplished, in some embodiments, by transfecting cells with non-integrative mRNAs for not more than about 9, 8, 7, 6, 5, 4, 3, 2, or 1 days, or less than 1 day. Transient reprogramming is accomplished, in embodiments, by transfecting cells with non-integrative mRNAs for not more than about 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 20, 19, 18, 17, 16, 15, 14, 13, 12, 11, 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 20, 19, 18, 17, 16, 15, 14, 13, 12, 11, 10, 9, 8, 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 14, 13, 12, 11, 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 14, 13, 12, 11, 10, 9, 8, 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 10, 9, 8, 7, 6, 5, 4, 3, or 2 days. Transient reprogramming is accomplished, in embodiments, by expressing at least one reprogramming factor from non-integrative mRNAs transfected in cells, or exposing cells to at least one reprogramming factor expressed from non-integrative mRNAs transfected in cells, for not more than about 7, 6, 5, 4, 3, 2, or 1 days, or less than 1 day. Transient reprogramming is accomplished, in embodiments, by expressing at least one reprogramming factor from non-integrative mRNAs transfected in cells, or exposing cells to at least one reprogramming factor expressed from non-integrative mRNAs transfected in cells, for at least 2 days and not more than about 7, 6, 5, 4, 3, or 2 days. Transient reprogramming is accomplished, in embodiments, by expressing at least one reprogramming factor from non-integrative mRNAs transfected in cells, or exposing cells to at least one reprogramming factor expressed from non-integrative mRNAs transfected in cells, for not more than about 5, 4, 3, 2, or 1 days, or less than 1 day. Transient reprogramming is accomplished, in embodiments, by expressing at least one reprogramming factor from non-integrative mRNAs transfected in cells, or exposing cells to at least one reprogramming factor expressed from non-integrative mRNAs transfected in cells, for at least 2 days and not more than about 5, 4, 3, or 2 days. In embodiments, transient reprogramming of cells eliminates various hallmarks of aging while avoiding complete dedifferentiation of the cells into stem cells.

In some embodiments, exposing comprises providing a composition comprising the mRNA and administering the mRNA to the immune cell 1, 2, 3, 4, 5, or 6 times over a period of 1, 2, 3, 4, 5, or 6 days. For example, the mRNA could be administered once on the first or second day of a five- or six-day period, or it could be administered once on the first day and once on the third day of a five- or six-day period, or it could be administered once in a one-day period. In some embodiments, the mRNA is administered after an immune cell activation step. In some embodiments, the immune cell activation step comprises activating the immune cells for 1, 2, or 3 days. In some embodiments, the immune cell activation step comprises activating the immune cells using at least one of CD3, CD28, and IL-2. In some embodiments, the immune cells are activated with CD3 and CD28. In some embodiments, the mRNA administration period occurs immediately after the immune cell activation step. In some embodiments, the mRNA administration period occurs 1, 2, 3, 4, 5, 6, 7, 8, 9, 10, 11, 12, 13, or 14 days after the immune cell activation step. In some embodiments, the administration of the mRNA encoding reprogramming factors reverses immune cell exhaustion caused by the immune cell activation step. In some embodiments, the administration of the mRNA encoding reprogramming factors reverses immune cell exhaustion in immune cells from an aged patient or donor. In some embodiments, the administration of the mRNA is performed during a manufacturing process to make immune cells for transplantation, for example CAR-T, CAR-M, or CAR-NK cells.

In some embodiments, the methods and compositions related to rejuvenating immune cells, such as T cells, comprise exposing the immune cell to messenger RNA (mRNA) encoding one or more reprogramming factors wherein the reprogramming factor encoding mRNA encodes a polypeptide encoded by a polynucleotide having at least about 70%, 75%, 80%, 85%, 90%, 95%, 96%, 97%, 98% or 99% sequence identity to any one of SEQ ID Nos: 1-14 (Table 1). In some embodiments, the methods and compositions related to rejuvenating immune cells, such as T cells, comprise exposing the immune cell to messenger RNA (mRNA) encoding one or more reprogramming factors wherein the reprogramming factor encoding mRNA encodes a polypeptide encoded by a polynucleotide having about 70%, 75%, 80%, 85%, 90%, 95%, 96%, 97%, 98% or 99% sequence identity to any one of SEQ ID Nos: 1-14 (Table 1).

In some embodiments, the methods and compositions related to rejuvenating immune cells, such as T cells, comprise exposing the immune cell to messenger RNA (mRNA) encoding one or more reprogramming factors wherein the reprogramming factor encoding mRNA encodes a polypeptide encoded by a polynucleotide having at least about 95%, 96%, 97%, 98% or 99% sequence identity to any one of SEQ ID Nos: 1-14 (Table 1). In some embodiments, the methods and compositions related to rejuvenating immune cells, such as T cells, comprise exposing the immune cell to messenger RNA (mRNA) encoding one or more reprogramming factors wherein the reprogramming factor encoding mRNA encodes a polypeptide encoded by a polynucleotide having at about 95%, 96%, 97%, 98% or 99% sequence identity to any one of SEQ ID Nos: 1-14 (Table 1).

TABLE 1

| | | SEQ |
|---|---|---|
| | | ID |
| NAME | SEQUENCE | NO |
| OCT4 | ATGGCTGGCCATCTCGCAAGTGACTTCG | 1 |
| | CATTTTCCCCGCCCCCAGGCGGCGGTGG | |
| | AGATGGACCTGGCGGCCCAGAACCAGG | |
| | ATGGGTGGACCCCCGCACGTGGCTTTCT | |
| | TTTCAGGGTCCACCAGGCGGACCCGGTA | |
| | TTGGACCCGGCGTGGGTCCTGGGTCAGA | |
| | AGTCTGGGGTATCCCACCCTGTCCCCCA | |
| | CCATACGAATTTTGCGGCGGAATGGCCT | |
| | ATTGCGGCCCTCAAGTCGGGGTCGGTCT | |
| | GGTACCTCAGGGTGGTCTGGAAACATCC | |
| | CAACCAGAAGGTGAGGCCGGTGTGGGA | |
| | GTTGAATCCAATTCTGACGGAGCATCTC | |
| | CAGAACCTTGTACTGTGACACCAGGAGC | |
| | TGTTAAATTGGAGAAAGAAAAGCTCGA | |
| | ACAGAATCCAGAAGAATCACAAGATATT | |
| | AAGGCGCTCCAAAAGGAGCTGGAACAG | |
| | TTCGCGAAACTTCTTAAACAGAAACGCA | |
| | TTACTCTCGGGTACACCCAAGCGGACGT | |
| | TGGACTGACTCTCGGTGTGCTGTTCGGC | |
| | AAAGTCTTTAGTCAGACCACAATATGTC | |
| | GATTCGAAGCCCTTCAACTGTCATTTAA | |
| | GAATATGTGCAAACTTCGACCTCTGCTC | |
| | CAGAAATGGGTCGAAGAGGCGGATAAT | |
| | AACGAGAACCTGCAAGAAATCTGTAAG | |
| | GCGGAGACTCTGGTTCAAGCTCGCAAAA | |
| | GGAAACGTACGTCTATAGAAAATAGAGT | |
| | CCGTGGGAATCTTGAAAACCTGTTTCTC | |
| | CAATGTCCAAAGCCTACTTTGCAACAAA | |
| | TATCTCATATTGCGCAACAACTCGGCCT | |
| | GGAAAAGGACGTAGTTAGAGTCTGGTTT | |
| | TGCAATCGCAGACAGAAAGGGAAACGG | |
| | TCTTCCAGTGATTACGCGCAGAGGGAAG | |
| | ACTTCGAAGCAGCCGGTTCACCGTTTTC | |
| | CGGCGGCCCGGTATCTTTCCCATTGGCT | |
| | CCCGGTCCTCACTTCGGCACACCCGGGT | |
| | ACGGCTCACCACATTTTACCGCCCTTTAT | |
| | TCAAGCGTTCCCTTTCCGGAAGGCGAGG | |
| | CTTTCCCGCCGGTGTCAGTGACTACACTT | |
| | GGATCCCCAATGCACAGCAATTAG | |
| SOX2 | ATGTATAATATGATGGAAACCGAATTGA | 2 |
| | AACCACCCGGGCCCCAACAGACCTCTGG | |
| | CGGCGGTGGTGGTAATAGCACAGCAGC | |
| | AGCCGCTGGTGGAAATCAAAAGAATTCT | |
| | CCAGATAGAGTGAAACGACCTATGAAC | |
| | GCATTTATGGTCTGGTCTAGAGGACAAC | |
| | GAAGGAAAATGGCTCAAGAAAATCCCA | |
| | AAATGCATAATAGCGAAATTTCCAAACG | |
| | GTTGGGTGCGGAATGGAAGCTCCTCAGC | |
| | GAAACCGAAAAGAGGCCATTTATTGATG | |
| | AAGCGAAAAGACTCAGGGCATTGCATAT | |
| | GAAAGAACATCCAGACTACAAGTATAG | |
| | ACCACGCCGCAAGACAAAGACTCTGATG | |
| | AAGAAGGACAAATATACCCTGCCTGGTG | |
| | GATTGTTGGCTCCTGGCGGTAACAGTAT | |
| | GGCTTCTGGCGTGGGCGTTGGGGCTGGA | |
| | CTTGGTGCCGGGGTCAATCAACGAATGG | |
| | ATTCCTATGCCCATATGAATGGATGGAG | |
| | TAATGGTTCCTATTCTATGATGCAAGAT | |
| | CAATTGGGATATCCTCAACATCCCGGTC | |
| | TGAACGCTCATGGTGCTGCTCAAATGCA | |
| | ACCTATGCATCGGTATGATGTAAGTGCA | |
| | TTGCAATATAATAGCATGACATCCAGTC | |
| | AAACATATATGAATGGGTCACCAACATA | |
| | TAGTATGAGCTATTCCCAACAAGGTACA | |
| | CCAGGGATGGCCCTGGGGAGCATGGGG | |
| | AGTGTCGTTAAAAGTGAAGCTTCAAGTT | |
| | CACCACCCGTAGTGACGAGTTCTTCACA | |
| | TTCTCGAGCCCCATGTCAAGCAGGAGAT | |
| | CTTAGGGATATGATTTCAATGTACTTGC | |
| | CAGGGGCTGAAGTCCCCGAGCCGGCAG | |
| | CGCCTTCTAGGCTGCATATGTCTCAACA | |
| | TTATCAATCCGGACCCGTTCCAGGGACC | |
| | GCTATCAATGGTACGTTGCCATTGTCCC | |
| | ATATGTAA | |
| C-Myc | ATGGACTTCTTTCGAGTGGTAGAGAATC | 3 |
| | AACAACCCCCGGCCACCATGCCATTGAA | |
| | TGTATCATTTACAAACCGGAACTACGAC | |
| | CTGGACTATGATTCAGTTCAGCCTTACTT | |
| | TTATTGTGACGAAGAAGAAAATTTCTAT | |
| | CAACAACAACAACAATCAGAATTGCAA | |
| | CCACCCGCTCCATCAGAAGACATTTGGA | |
| | AGAAGTTTGAACTCCTGCCAACTCCACC | |
| | GCTCAGCCCGAGTAGACGTTCTGGACTG | |
| | TGTTCTCCTTCTTATGTGGCTGTGACTCC | |
| | GTTTTCACTGCGTGGCGATAATGATGGC | |
| | GGCGGTGGCTCTTTTAGTACAGCAGATC | |
| | AACTTGAAATGGTCACAGAACTCCTTGG | |
| | TGGTGATATGGTTAATCAATCATTCATTT | |
| | GTGATCCCGATGATGAGACATTTATAAA | |
| | GAACATCATCATACAAGACTGCATGTGG | |
| | TCAGGGTTTAGTGCTGCTGCGAAACTGG | |
| | TGAGCGAAAAGTTGGCTTCTTATCAAGC | |
| | CGCCCGGAAGGATAGTGGATCACCAAAT | |
| | CCAGCAAGGGGTCATTCAGTGTGTAGCA | |
| | CAAGCTCTCTGTATCTTCAAGACCTCTCC | |
| | GCGGCTGCAAGTGAATGTATTGATCCAA | |
| | GTGTCGTTTTCCCTTATCCCCTGAATGAT | |
| | TCTTCCTCTCCTAAAAGCTGTGCGAGCC | |
| | AGGATTCTTCAGCTTTCTCCCCAAGCTCC | |
| | GACAGTTTGTTGAGCTCTACTGAAAGTA | |
| | GTCCTCAAGGGTCACCGGAACCTCTCGT | |
| | CCTTCACGAAGAAACACCCCCTACAACT | |
| | AGTTCCGATTCCGAAGAAGAACAGGAA | |
| | GACGAAGAGGAGATTGACGTGGTATCA | |
| | GTTGAGAAAAGACAAGCCCCCGGGAAG | |
| | CGAAGCGAAAGCGGGAGCCCAAGCGCC | |
| | GGCGGACATTCCAAGCCCCCACATTCTC | |
| | CTTTGGTACTGAAAAGATGTCATGTGAG | |
| | CACCCACCAACATAATTATGCTGCTCCC | |
| | CCATCAACCAGGAAAGATTACCCCGCCG | |
| | CTAAACGAGTTAAACTGGATTCAGTGAG | |
| | GGTTCTTAGGCAAATTTCAAATAATAGG | |
| | AAGTGTACTTCACCTCGCAGTAGCGATA | |
| | CAGAAGAAAACGTTAAAAGACGGACGC | |
| | ATAATGTGCTGGAACGACAAAGACGAA | |
| | ATGAACTTAAGAGGTCCTTCTTCGCGCT | |
| | TAGGGATCAAATACCTGAACTGGAGAAT | |
| | AACGAGAAAGCTCCAAAAGTGGTGATTT | |
| | TGAAGAAGGCTACTGCGTATATACTTTC | |
| | TGTACAGGCCGAAGAACAGAAACTGAT | |
| | ATCAGAGGAAGATCTGCTTCGTAAGCGC | |
| | AGAGAGCAACTGAAGCATAAGCTCGAG | |
| | CAACTCCGCAATAGCTGCGCCTAG | |
| KLF4 | ATGCGGCAGCCACCAGGGGAAAGTGAT | 4 |
| | ATGGCCGTTTCCGACGCTCTTCTGCCTTC | |
| | ATTTTCAACCTTTGCTTCCGGACCTGCCG | |
| | GGCGGGAAAAGACGCTTAGGCAGGCCG | |
| | GAGCACCCAACAATCGATGGAGAGAAG | |
| | AACTGAGCCATATGAAAAGACTGCCGCC | |
| | TGTACTCCCGGGGCGGCCATACGATCTC | |
| | GCCGCCGCTACAGTAGCAACTGATTTGG | |
| | AATCCGGTGGTGCTGGGGCAGCCTGTGG | |
| | CGGATCTAATCTTGCTCCTCTGCCAAGA | |
| | CGGGAAACGGAAGAATTTAATGACCTGC | |
| | TCGATTTGGATTTCATCCTTTCTAACTCA | |
| | CTTACACACCCACCAGAAAGTGTCGCTG | |
| | CTACGGTCTCTAGCTCTGCCAGCGCAAG | |
| | CAGTTCATCTTCTCCATCTTCCTCAGGAC | |
| | CCGCAAGTGCCCCGAGCACTTGTTCCTT | |
| | TACGTACCCAATACGAGCTGGCAATGAT | |
| | CCTGGAGTTGCTCCCGGTGGTACAGGCG | |
| | GTGGTCTGTTGTACGGAAGAGAATCAGC | |
| | CCCACCACCAACCGCACCATTTAATTTG | |
| | GCCGATATTAATGATGTTTCACCTAGTG | |
| | GTGGTTTTGTTGCGGAACTGCTCCGTCCC | |
| | GAGCTGGATCCCGTCTATATCCCACCAC | |
| | AACAACCCCAACCACCTGGCGGCGGATT | |
| | GATGGGTAAATTTGTTCTTAAAGCATCC | |
| | CTTTCCGCACCAGGGTCAGAATATGGAA | |
| | GTCCCTCAGTGATTTCCGTATCTAAGGG | |
| | ATCCCCCGATGGGTCCCATCCAGTTGTC | |
| | GTTGCCCCTTATAATGGTGGCCCGCCTA | |
| | GAACCTGTCCGAAAATAAAACAAGAAG | |
| | CTGTATCATCATGTACGCATCTCGGGGC | |
| | AGGTCCACCCCTGTCTAACGGGCATCGC | |
| | CCTGCAGCCCATGATTTTCCTTTGGGCCG | |
| | ACAACTGCCGTCCCGTACAACTCCAACA | |
| | CTCGGCCTCGAAGAGGTCCTCAGTAGTA | |
| | GAGATTGCCATCCAGCACTCCCCCTGCC | |
| | ACCAGGTTTTCACCCTCATCCCGGTCCTA | |
| | ACTATCCCAGCTTTCTCCCAGACCAAAT | |
| | GCAACCCCAGGTTCCCCCACTTCACTAT | |
| | CAGGGCCAAAGCAGAGGTTTCGTGGCGC | |
| | GAGCCGGAGAACCATGCGTCTGTTGGCC | |
| | TCATTTTGGCACCCATGGAATGATGTTG | |
| | ACACCCCCAAGTAGTCCACTCGAACTGA | |
| | TGCCCCCTGGCAGTTGTATGCCTGAAGA | |
| | ACCTAAACCCAAAAGAGGGCGGCGGAG | |
| | TTGGCCACGAAAGCGAACAGCAACTCAT | |
| | ACCTGCGACTATGCTGGATGTGGGAAGA | |
| | CATATACCAAATCTAGCCACCTGAAAGC | |
| | GCATCTCCGCACGCATACTGGAGAAAAG | |
| | CCCTATCATTGCGATTGGGATGGGTGCG | |
| | GCTGGAAGTTTGCACGATCTGACGAGCT | |
| | TACTAGACATTATCGCAAGCATACCGGA | |
| | CATCGGCCCTTTCAATGTCAGAAGTGTG | |
| | ATCGCGCCTTCAGCCGGAGCGATCATCT | |
| | GGCTCTGCATATGAAACGACACTTCTAG | |
| LIN28 | ATGGGCTCAGTCAGCAATCAACAATTCG | 5 |
| | CGGGTGGATGTGCAAAAGCTGCTGAGG | |
| | AAGCCCCAGAAGAAGCTCCAGAAGATG | |
| | CCGCTCGCGCCGCTGATGAACCACAACT | |
| | GCTCCATGGAGCTGGGATTTGCAAATGG | |
| | TTTAATGTCCGGATGGGCTTTGGTTTCTT | |
| | GTCTATGACAGCAAGAGCTGGAGTGGCC | |
| | CTCGATCCACCCGTCGACGTGTTCGTAC | |
| | ATCAATCTAAACTTCATATGGAAGGATT | |
| | TCGCTCACTCAAAGAAGGAGAAGCCGTA | |
| | GAATTTACATTCAAGAAAAGTGCGAAAG | |
| | GGCTTGAGAGCATACGGGTGACAGGGC | |
| | CAGGCGGCGTGTTTTGCATCGGATCAGA | |
| | ACGAAGGCCCAAGGGTAAATCCATGCA | |
| | AAAGAGGCGATCCAAAGGGGATCGGTG | |
| | TTATAATTGCGGTGGCCTTGACCACCAC | |
| | GCGAAAGAGTGTAAACTTCCCCCGCAAC | |
| | CTAAGAAATGTCATTTCTGTCAATCCAT | |
| | ATCACACATGGTGGCGAGCTGCCCATTG | |
| | AAAGCACAACAAGGACCGTCCGCCCAA | |
| | GGGAAACCTACTTATTTCAGGGAAGAGG | |
| | AAGAGGAGATTCATAGTCCCACTCTCCT | |
| | TCCTGAAGCCCAAAACTAG | |
| NANOG | ATGTCAGTCGACCCGGCCTGCCCACAGA | 6 |
| | GTCTGCCCTGTTTCGAGGCTTCAGATTGC | |
| | AAGGAGTCCTCTCCGATGCCCGTCATCT | |
| | GCGGACCCGAGGAGAATTACCCCAGTCT | |
| | GCAGATGTCCAGTGCCGAAATGCCACAT | |
| | ACAGAAACGGTTTCACCGCTCCCATCTT | |
| | CAATGGACCTTTTGATCCAAGATTCTCC | |
| | CGACAGCAGCACTTCCCCAAAGGGAAA | |
| | GCAGCCTACCTCAGCGGAGAAATCTGTG | |
| | GCCAAGAAAGAGGATAAAGTTCCTGTTA | |
| | AGAAGCAAAAGACAAGGACCGTATTTTC | |
| | CTCAACACAACTCTGCGTCCTTAACGAC | |
| | CGGTTCCAACGCCAAAAGTATCTGTCCT | |
| | TGCAACAAATGCAGGAGCTGAGCAATAT | |
| | TTTGAATCTGTCTTATAAGCAAGTCAAG | |
| | ACTTGGTTTCAAAACCAGCGTATGAAGA | |
| | GTAAAAGATGGCAGAAGAATAATTGGC | |
| | CCAAGAACTCTAACGGCGTTACTCAAAA | |
| | GGCGAGTGCCCCCACTTATCCATCTCTG | |
| | TATTCATCATATCATCAAGGCTGTTTGGT | |
| | TAATCCCACGGGCAATCTGCCGATGTGG | |
| | TCCAATCAAACGTGGAATAACAGTACTT | |
| | GGTCCAATCAAACTCAAAATATACAAAG | |
| | TTGGTCAAATCATTCTTGGAATACCCAA | |
| | ACGTGGTGTACTCAGAGCTGGAATAACC | |
| | AAGCATGGAACTCCCCATTTTACAATTG | |
| | CGGCGAAGAGTCACTCCAAAGCTGTATG | |
| | CAATTTCAACCCAACAGTCCCGCATCCG | |
| | ATCTTGAAGCAGCACTGGAAGCGGCCGG | |
| | TGAGGGGCTGAACGTGATTCAACAAACA | |
| | ACAAGATACTTCTCCACACCTCAGACTA | |
| | TGGACCTTTTCCTGAATTATTCAATGAAT | |
| | ATGCAGCCCGAGGATGTCTAG | |
| OCT4MyoD for | ATGGAGTTCGCCATGGAGCTGCTGTCGC | 7 |
| T-cells (T- | CACCGTTGCGTGACGTGGACCTGACAGC | |
| OCT4MyoD) | CCCCGACGGCTCTCTGTGCTCCTTTGCTA | |
| | CTACCGACGATTTCTACGATGACCCGTG | |
| | CTTTGATTCTCCCGACCTGCGCTTTTTCG | |
| | AAGACTTAGATCCGCGCCTGATGCATGT | |
| | AGGTGCTCTGCTAAAGCCCGAGGAGCAC | |
| | ATGGCTGGCCACTTGGCTTCCGACTTCG | |
| | CGTTCTCCCCGCCACCCGGCGGCGGAGG | |
| | CGATGGCCCTGGCGGACCCGAGCCAGGT | |
| | TGGGTCGACCCTCGGACCTGGCTCTCCT | |
| | TCCAGGGCCCCCCAGGCGGGCCCGGCAT | |
| | AGGTCCCGGCGTTGGACCCGGGAGCGA | |
| | GGTGTGGGGCATCCCTCCGTGCCCACCC | |
| | CCGTATGAGTTCTGCGGGGGTATGGCCT | |
| | ACTGCGGGCCTCAGGTGGGCGTCGGCCT | |
| | CGTCCCTCAGGGGGGTCTGGAGACCTCT | |
| | CAGCCGGAGGGAGAGGCTGGGGTCGGC | |
| | GTGGAGAGCAACTCCGACGGGGCGTCG | |
| | CCCGAACCTTGCACTGTCACGCCCGGGG | |
| | CCGTTAAGCTGGAGAAGGAGAAACTTG | |
| | AGCAGAACCCCGAGGAGAGCCAGGACA | |
| | TCAAGGCGCTGCAGAAGGAGCTGGAAC | |
| | AGTTCGCCAAGCTGCTGAAGCAGAAGCG | |
| | CATCACCCTAGGTTACACCCAGGCGGAC | |
| | GTGGGCCTGACGCTTGGTGTGCTGTTCG | |
| | GAAAGGTGTTCAGCCAGACGACCATCTG | |
| | CCGATTCGAGGCCCTCCAGCTGTCCTTC | |
| | AAGAACATGTGCAAGTTGCGGCCCCTGC | |
| | TCCAAAAATGGGTGGAGGAGGCTGACA | |
| | ACAACGAGAATCTCCAGGAGATCTGTAA | |
| | AGCCGAGACTCTGGTGCAGGCCCGCAAA | |
| | CGCAAGCGTACCTCGATTGAAAACAGGG | |
| | TGCGTGGCAACCTGGAGAACCTGTTCCT | |
| | ACAGTGTCCCAAGCCTACCCTTCAGCAG | |
| | ATTAGCCACATCGCACAACAGTTGGGCC | |
| | TTGAAAAGGATGTGGTACGCGTGTGGTT | |
| | CTGTAACCGCCGCCAGAAGGGTAAGCGC | |
| | AGCTCCAGCGACTACGCGCAGAGAGAG | |
| | GACTTTGAGGCTGCAGGATCTCCTTTTTC | |
| | TGGCGGCCCTGTGAGTTTCCCTCTGGCC | |
| | CCGGGACCCCACTTTGGTACTCCGGGCT | |
| | ACGGCTCCCCGCACTTCACCGCCCTGTA | |
| | CTCTAGTGTCCCGTTTCCCGAGGGCGAG | |
| | GCGTTCCCCCCAGTGTCCGTGACCACAC | |
| | TGGGGTCCCCAATGCATTCAAATTGA | |
| B18R for T | ATGACAATGAAAATGATGGTGCACATCT | 8 |
| cells | ACTTCGTCTCTCTTCTGCTGCTGCTGTTT | |
| (T-B18R) | CACAGCTACGCAATTGACATCGAAAACG | |
| | AGATCACCGAGTTCTTCAACAAGATGCG | |
| | TGACACCCTTCCCGCCAAGGATTCTAAA | |
| | TGGCTCAACCCGGCCTGCATGTTTGGAG | |
| | GCACCATGAACGACATCGCGGCGCTGGG | |
| | CGAGCCCTTCTCCGCTAAATGTCCCCCG | |
| | ATCGAAGATTCTCTGCTGAGCCACCGCT | |
| | ACAAGGACTATGTGGTCAAATGGGAGC | |
| | GCCTGGAGAAGAACCGTCGGCGCCAGG | |
| | TCTCAAACAAGCGCGTAAAGCATGGAG | |
| | ATCTTTGGATCGCCAACTACACCAGTAA | |
| | ATTTAGCAACCGCCGCTACCTGTGCACT | |
| | GTCACCACCAAGAATGGGGACTGCGTGC | |
| | AGGGCATCGTGCGGAGCCACATCCGCAA | |
| | GCCTCCCTCTTGTATTCCCAAGACCTACG | |
| | AGCTGGGGACACATGACAAGTACGGCA | |
| | TTGACCTGTATTGCGGGATCCTGTACGC | |
| | GAAGCACTACAACAACATCACCTGGTAC | |
| | AAGGACAACAAGGAGATTAACATCGAT | |
| | GACATCAAGTACTCCCAGACTGGCAAGG | |
| | AGCTCATCATCCACAACCCTGAGCTGGA | |
| | GGACTCCGGTAGATATGATTGTTACGTG | |
| | CATTACGACGATGTGCGCATCAAAAACG | |
| | ACATTGTCGTTTCCAGGTGTAAGATACT | |
| | CACTGTGATCCCTAGTCAGGACCACCGC | |
| | TTCAAGCTGATCTTGGACCCGAAGATAA | |
| | ATGTGACCATCGGGGAGCCAGCCAATAT | |
| | CACCTGCACTGCCGTGTCCACCTCGTTG | |
| | CTCATTGACGATGTACTGATTGAGTGGG | |
| | AGAATCCATCGGGTTGGCTAATCGGCTT | |
| | CGACTTCGACGTGTACTCGGTGCTAACC | |
| | TCCCGTGGTGGCATCACTGAGGCTACGC | |
| | TTTATTTCGAGAACGTGACGGAGGAGTA | |
| | CATCGGCAACACCTACAAGTGCCGAGGC | |
| | CACAACTACTACTTCGAAAAAACCCTGA | |
| | CGACGACCGTGGTTTTGGAGTGA | |
| KLF4 for T | ATGCGTCAGCCACCCGGGGAGAGCGAC | 9 |
| cells | ATGGCCGTGTCGGACGCGCTGCTGCCAT | |
| (T-KLF4) | CCTTTTCCACCTTCGCCTCGGGTCCGGCC | |
| | GGCCGAGAGAAGACTCTGCGCCAGGCC | |
| | GGAGCCCCTAACAACCGCTGGAGAGAG | |
| | GAGCTGTCACACATGAAACGCCTGCCCC | |
| | CCGTGCTGCCTGGGCGCCCCTACGACCT | |
| | TGCCGCGGCCACGGTGGCTACCGACTTG | |
| | GAGTCTGGAGGTGCTGGAGCAGCGTGTG | |
| | GCGGAAGCAACCTGGCACCGTTGCCACG | |
| | CCGGGAGACCGAGGAGTTCAACGACTTG | |
| | TTGGATCTGGACTTTATTCTGTCCAACTC | |
| | CCTTACACACCCGCCCGAGAGCGTAGCA | |
| | GCCACCGTGAGCTCCAGTGCTTCCGCTT | |
| | CCTCCTCATCCAGCCCGTCGTCTTCTGGC | |
| | CCTGCCTCTGCGCCGTCGACCTGTTCGTT | |
| | CACCTATCCCATCCGGGCCGGCAACGAT | |
| | CCGGGCGTGGCCCCGGGCGGCACCGGTG | |
| | GTGGTCTCCTGTACGGCAGGGAGTCCGC | |
| | CCCCCCTCCAACCGCTCCCTTCAACCTCG | |
| | CGGACATCAATGACGTGTCCCCCTCTGG | |
| | CGGCTTCGTTGCAGAACTGTTAAGGCCT | |
| | GAACTGGATCCAGTGTACATCCCGCCCC | |
| | AGCAGCCTCAGCCGCCGGGCGGCGGTCT | |
| | GATGGGCAAATTTGTCCTGAAGGCGTCT | |
| | CTGTCTGCTCCTGGCTCCGAGTACGGCA | |
| | GCCCCAGTGTGATTAGCGTGTCTAAAGG | |
| | CAGCCCCGACGGGTCGCACCCCGTGGTG | |
| | GTCGCTCCTTACAACGGTGGACCCCCGC | |
| | GCACCTGCCCAAAGATCAAGCAGGAGG | |
| | CTGTTTCTTCATGCACTCATCTAGGCGCC | |
| | GGTCCACCCCTTTCCAATGGCCACCGGC | |
| | CCGCCGCGCATGACTTCCCCCTGGGCCG | |
| | CCAGCTGCCCAGCCGGACCACACCTACC | |
| | TTAGGCCTGGAGGAGGTGCTTAGTTCGC | |
| | GCGACTGTCATCCTGCCCTGCCTCTCCCT | |
| | CCAGGCTTCCACCCCCACCCGGGACCCA | |
| | ACTACCCGTCCTTCCTGCCCGACCAGAT | |
| | GCAACCTCAGGTCCCTCCCCTGCACTAC | |
| | CAGGGACAGAGCCGCGGTTTCGTCGCCC | |
| | GTGCTGGCGAGCCATGCGTCTGTTGGCC | |
| | GCACTTCGGCACGCATGGAATGATGCTA | |
| | ACTCCCCCGAGCTCCCCCCTGGAGCTGA | |
| | TGCCCCCGGGTTCCTGTATGCCAGAGGA | |
| | GCCCAAGCCTAAGCGCGGCAGACGTAGT | |
| | TGGCCCCGTAAGCGCACCGCCACACATA | |
| | CGTGCGACTACGCGGGGTGCGGGAAAA | |
| | CCTACACCAAGTCTTCTCACCTCAAGGC | |
| | TCACTTGCGTACTCACACTGGAGAGAAG | |
| | CCTTATCACTGCGACTGGGATGGGTGCG | |
| | GGTGGAAGTTTGCTCGCTCCGACGAACT | |
| | CACCCGCCATTACCGCAAGCACACCGGC | |
| | CACCGCCCCTTCCAGTGTCAGAAGTGCG | |
| | ATCGAGCGTTCTCCCGCTCGGACCACCT | |
| | GGCCCTACACATGAAGAGGCACTTTTGA | |
| LIN28 for T | ATGGGCTCCGTGTCGAATCAGCAGTTCG | 10 |
| cells | CAGGCGGGTGCGCCAAGGCAGCCGAGG | |
| (T-LIN28) | AGGCCCCGGAGGAGGCTCCTGAAGACG | |
| | CCGCTCGCGCGGCGGACGAGCCTCAGCT | |
| | ACTCCATGGAGCTGGCATCTGCAAATGG | |
| | TTCAACGTGCGAATGGGATTTGGCTTCC | |
| | TGTCTATGACTGCTCGTGCCGGCGTGGC | |
| | GCTGGACCCGCCCGTAGACGTGTTCGTC | |
| | CACCAGAGCAAGCTGCATATGGAGGGTT | |
| | TCAGATCTCTGAAGGAGGGCGAGGCCGT | |
| | GGAGTTCACGTTCAAGAAGTCGGCCAAA | |
| | GGTCTGGAGAGCATCCGCGTCACCGGTC | |
| | CCGGCGGCGTTTTTTGTATTGGCTCCGA | |
| | GCGCCGGCCCAAGGGCAAGTCCATGCA | |
| | GAAGCGCCGCTCCAAGGGGGACAGGTG | |
| | TTACAACTGCGGGGGTTTGGATCACCAC | |
| | GCGAAGGAGTGCAAACTTCCTCCTCAGC | |
| | CGAAGAAATGTCACTTTTGTCAGAGCAT | |
| | CAGTCACATGGTGGCTTCATGCCCACTC | |
| | AAGGCCCAACAGGGACCCTCTGCGCAG | |
| | GGCAAGCCCACCTACTTCCGCGAGGAGG | |
| | AAGAAGAGATCCACTCCCCAACCCTGCT | |
| | GCCCGAGGCTCAGAACTGA | |
| NANOG for T | ATGTCTGTTGACCCGGCCTGCCCACAGA | 11 |
| cells | GCCTTCCCTGCTTTGAGGCTTCCGACTGT | |
| (T-NANOG) | AAAGAGAGTTCCCCGATGCCCGTGATTT | |
| | GTGGTCCTGAAGAGAACTACCCGTCCCT | |
| | ACAGATGTCATCGGCGGAGATGCCTCAT | |
| | ACCGAGACCGTGTCCCCCTTACCCTCTTC | |
| | TATGGATCTGCTGATCCAGGACTCGCCC | |
| | GACAGCTCGACCAGCCCTAAGGGCAAG | |
| | CAGCCAACCTCCGCAGAGAAGTCCGTGG | |
| | CTAAGAAGGAGGACAAAGTACCGGTGA | |
| | AGAAACAGAAGACTCGCACCGTGTTCTC | |
| | GTCGACCCAGCTGTGCGTGCTCAACGAC | |
| | AGGTTTCAGCGCCAGAAGTACCTGTCCC | |
| | TGCAGCAGATGCAAGAGCTCAGCAACAT | |
| | CCTGAACCTGAGCTACAAGCAGGTCAAG | |
| | ACCTGGTTCCAGAACCAGCGTATGAAGT | |
| | CCAAGCGGTGGCAAAAAAACAACTGGC | |
| | CCAAGAACAGCAACGGTGTCACCCAGA | |
| | AGGCCTCCGCCCCCACCTACCCGTCTCTT | |
| | TACTCCAGCTATCACCAGGGCTGCCTGG | |
| | TGAATCCAACAGGAAACCTGCCCATGTG | |
| | GTCTAATCAGACCTGGAACAACTCCACC | |
| | TGGTCTAACCAGACACAGAACATCCAGA | |
| | GTTGGTCTAATCACTCTTGGAACACGCA | |
| | GACCTGGTGCACTCAGAGTTGGAATAAC | |
| | CAGGCCTGGAATTCCCCCTTCTACAACT | |
| | GCGGGGAGGAGAGCTTGCAGTCATGTAT | |
| | GCAGTTCCAGCCCAACTCCCCAGCCAGT | |
| | GACCTGGAGGCCGCGCTGGAGGCGGCT | |
| | GGCGAGGGCCTCAACGTGATCCAACAG | |
| | ACGACCCGCTACTTCTCCACGCCTCAGA | |
| | CTATGGATTTGTTCCTGAACTATTCGATG | |
| | AACATGCAGCCTGAAGATGTGTGA | |
| OCT4 for T | ATGGCGGGCCACTTGGCTTCCGACTTCG | 12 |
| cells | CGTTCTCTCCCCCGCCTGGCGGAGGCGG | |
| (T-OCT4) | GGACGGCCCCGGCGGCCCTGAGCCAGG | |
| | CTGGGTCGATCCACGCACCTGGCTCTCC | |
| | TTCCAGGGACCCCCCGGGGGGCCCGGCA | |
| | TCGGTCCCGGGGTAGGCCCCGGATCGGA | |
| | GGTGTGGGGCATCCCGCCGTGCCCACCG | |
| | CCCTATGAGTTCTGTGGGGGTATGGCCT | |
| | ACTGCGGGCCTCAGGTCGGTGTCGGTCT | |
| | GGTGCCTCAGGGCGGTCTCGAGACCAGC | |
| | CAGCCGGAGGGCGAGGCTGGCGTGGGT | |
| | GTGGAGAGCAACAGCGATGGCGCTTCTC | |
| | CTGAACCATGCACTGTCACCCCCGGCGC | |
| | CGTGAAGCTGGAGAAAGAGAAACTGGA | |
| | GCAGAATCCAGAGGAGAGTCAGGACAT | |
| | CAAGGCCCTGCAGAAGGAACTGGAACA | |
| | GTTCGCCAAGCTGCTGAAGCAGAAGCGT | |
| | ATCACCCTTGGATACACCCAGGCGGACG | |
| | TGGGCCTCACTTTAGGCGTTCTTTTCGGA | |
| | AAGGTGTTCTCACAGACCACAATCTGCC | |
| | GATTCGAGGCCCTGCAACTTTCTTTCAA | |
| | AAACATGTGCAAGTTGCGGCCCCTACTG | |
| | CAGAAATGGGTGGAGGAAGCGGACAAC | |
| | AACGAGAATCTGCAGGAGATCTGTAAG | |
| | GCCGAGACACTGGTGCAGGCTCGCAAGC | |
| | GCAAAAGAACGAGCATTGAGAACCGCG | |
| | TCCGCGGCAACCTGGAGAACCTGTTCCT | |
| | GCAGTGCCCAAAGCCTACCCTCCAGCAA | |
| | ATTAGCCACATCGCTCAGCAGCTGGGCC | |
| | TGGAGAAGGATGTGGTGAGGGTGTGGTT | |
| | CTGTAACCGGCGCCAGAAGGGCAAGCG | |
| | CAGCTCCAGTGACTACGCACAGCGTGAG | |
| | GACTTTGAAGCCGCCGGCTCCCCGTTTT | |
| | CAGGAGGCCCCGTGTCCTTTCCTTTGGCT | |
| | CCCGGCCCTCATTTCGGTACTCCGGGCT | |
| | ACGGCTCCCCCCACTTTACCGCCCTTTAC | |
| | TCCTCTGTCCCCTTCCCCGAGGGAGAGG | |
| | CATTTCCCCCGGTGTCCGTAACGACCCT | |
| | GGGTTCCCCAATGCACTCTAACTGA | |
| SOX2 for T | ATGTATAACATGATGGAAACAGAGCTGA | 13 |
| cells | AGCCCCCGGGGCCTCAACAGACCTCCGG | |
| (T-SOX2) | CGGTGGGGGCGGCAACTCGACCGCTGCC | |
| | GCAGCTGGTGGAAACCAGAAGAACAGT | |
| | CCCGACAGAGTTAAGCGCCCGATGAACG | |
| | CGTTCATGGTGTGGTCTCGCGGCCAGCG | |
| | CCGCAAGATGGCGCAGGAGAATCCAAA | |
| | AATGCACAACTCGGAGATCTCCAAGCGG | |
| | CTCGGTGCCGAGTGGAAGCTGCTAAGCG | |
| | AGACCGAGAAACGTCCTTTTATTGACGA | |
| | GGCCAAGCGCCTGCGTGCGCTTCACATG | |
| | AAGGAGCACCCCGACTACAAGTACAGG | |
| | CCCCGACGCAAAACCAAGACCCTGATGA | |
| | AAAAGGACAAGTACACCCTCCCCGGCG | |
| | GCCTGCTGGCCCCTGGTGGCAACAGCAT | |
| | GGCCTCCGGAGTCGGGGTAGGCGCCGGC | |
| | CTTGGAGCTGGAGTCAACCAGCGTATGG | |
| | ATTCTTACGCGCACATGAATGGGTGGTC | |
| | AAATGGCTCGTATTCTATGATGCAGGAC | |
| | CAGCTGGGCTACCCTCAACACCCCGGCC | |
| | TCAACGCCCATGGAGCGGCTCAGATGCA | |
| | GCCAATGCACCGCTACGATGTGAGCGCC | |
| | CTGCAGTACAACTCTATGACTAGTTCAC | |
| | AGACTTACATGAACGGTTCCCCAACCTA | |
| | CTCCATGTCTTACAGTCAGCAGGGAACG | |
| | CCGGGTATGGCTCTGGGCTCCATGGGCT | |
| | CCGTGGTGAAGTCGGAGGCATCCTCCAG | |
| | CCCTCCCGTGGTCACCTCCTCCTCTCACA | |
| | GCCGCGCTCCTTGCCAGGCCGGGGACCT | |
| | GCGCGACATGATCTCTATGTATCTGCCC | |
| | GGTGCAGAGGTGCCTGAACCGGCGGCCC | |
| | CCTCTCGGTTGCATATGTCCCAGCATTAC | |
| | CAGAGCGGCCCGGTGCCAGGCACTGCCA | |
| | TCAACGGCACCTTGCCCCTGAGCCACAT | |
| | GTGA | |
| cMYC for T- | ATGGATTTCTTCCGAGTGGTGGAGAATC | 14 |
| cells | AGCAGCCGCCTGCCACCATGCCCCTTAA | |
| (T-cMyc) | CGTGTCCTTCACTAACAGAAACTACGAC | |
| | CTGGACTACGACAGTGTCCAGCCCTATT | |
| | TCTACTGTGATGAGGAGGAGAACTTTTA | |
| | CCAGCAGCAACAGCAGAGCGAACTGCA | |
| | GCCCCCAGCGCCCTCGGAGGACATCTGG | |
| | AAGAAATTTGAGCTGCTGCCAACCCCCC | |
| | CCCTATCTCCATCCCGCCGTTCCGGTCTC | |
| | TGCTCTCCTAGCTACGTGGCTGTCACTCC | |
| | CTTTTCGCTGCGTGGGGACAACGATGGG | |
| | GGGGGCGGAAGTTTCAGCACTGCGGACC | |
| | AGCTGGAGATGGTGACCGAGCTGCTGGG | |
| | TGGTGACATGGTCAACCAGTCTTTTATCT | |
| | GCGACCCGGATGACGAGACCTTCATCAA | |
| | GAACATCATCATCCAGGACTGTATGTGG | |
| | TCTGGCTTCTCTGCCGCTGCAAAACTGG | |
| | TCTCGGAGAAACTTGCTAGCTACCAGGC | |
| | TGCTCGCAAGGACTCCGGCTCGCCGAAT | |
| | CCAGCTAGGGGACATAGTGTTTGTAGTA | |
| | CCTCCTCGCTCTACCTGCAGGACCTGTCC | |
| | GCAGCGGCCTCTGAGTGTATTGACCCGT | |
| | CCGTGGTGTTCCCCTATCCTCTCAACGAC | |
| | TCGTCTTCCCCCAAGAGCTGCGCCTCCC | |
| | AGGACTCATCTGCGTTCTCCCCCTCCTCC | |
| | GATAGCCTGTTGAGTAGCACAGAGAGCT | |
| | CCCCTCAGGGCTCCCCGGAGCCCCTGGT | |
| | GCTACACGAGGAGACCCCGCCCACCACC | |
| | AGCAGTGACTCAGAAGAGGAGCAGGAG | |
| | GACGAAGAGGAGATCGATGTGGTTTCCG | |
| | TGGAGAAGCGCCAGGCCCCTGGCAAAC | |
| | GCTCCGAATCCGGCTCCCCTTCCGCCGG | |
| | CGGCCACTCCAAGCCCCCGCACAGCCCG | |
| | TTGGTGCTGAAGAGATGTCACGTGTCAA | |
| | CCCACCAGCATAACTACGCGGCGCCTCC | |
| | CTCTACCCGCAAAGACTACCCCGCTGCC | |
| | AAGCGCGTGAAGTTGGATAGCGTCCGGG | |
| | TCTTACGCCAGATTTCTAACAACCGCAA | |
| | GTGCACTTCTCCAAGGTCTTCCGACACG | |
| | GAGGAGAACGTGAAGCGGCGCACCCAC | |
| | AACGTACTGGAGCGCCAGCGTCGTAACG | |
| | AGTTGAAGCGCAGCTTCTTCGCGCTTCG | |
| | GGACCAGATACCTGAGCTCGAGAATAAC | |
| | GAGAAAGCACCAAAGGTAGTCATCCTG | |
| | AAGAAGGCCACGGCCTACATCTTATCAG | |
| | TGCAGGCCGAGGAGCAGAAGCTGATTTC | |
| | AGAGGAGGATCTGCTGCGCAAGCGCCG | |
| | AGAACAGCTGAAGCACAAGCTGGAACA | |
| | ACTCCGCAACTCATGCGCCTGA | |

In another aspect, a method for inhibiting, preventing, and/or reversing exhaustion of an engineered immune cell is provided. In some embodiments, the method comprises exposing the immune cell to mRNA encoding one or more reprogramming factors, whereby said exposing achieves expression of the one or more reprogramming factors in the immune cell to inhibit, prevent and/or reverse exhaustion of the immune cell with retention of its identity.

In another aspect, a method for reducing T cell exhaustion that occurs during the manufacture of CAR T cells is provided. In some embodiments, the method comprises activating T cells by exposing them to CD3 and/or CD28; transfecting said activated T cells with mRNA encoding one or more reprogramming factors; wherein 1 ng to 500 ug of mRNA per million Tcells is used per transfection, wherein said transfection achieves expression of the one or more reprogramming factors in said T cells to increase their cytotoxicity or proliferation capacity as compared to untreated or control T cells. In certain embodiments, the method uses 1 ng to 400 ug of mRNA per million T cells per transfection. In certain embodiments, the method uses 1 ng to 300 ug of mRNA per million T cells per transfection. In certain embodiments, the method uses 1 ng to 250 ug of mRNA per million T cells per transfection. In certain embodiments, the method uses 1 ng to 200 ug of mRNA per million T cells per transfection. In certain embodiments, the method uses 1 ng to 150 ug of mRNA per million T cells per transfection. In certain embodiments, the method uses 1 ng to 100 ug of mRNA per million T cells per transfection. In certain embodiments, the method uses 1 ng to 50 ug of mRNA per million T cells per transfection. In certain embodiments, the method uses 1 ng to 25 ug of mRNA per million T cells per transfection. In certain embodiments, the method uses 1 ng to 20 ug of mRNA per million T cells per transfection. In certain embodiments, the method uses 1 ng to 15 ug of mRNA per million T cells per transfection. In certain embodiments, the method uses 1 ng to 10 ug of mRNA per million T cells per transfection. In certain embodiments, the method uses 1 ng to 9 ug of mRNA per million T cells per transfection. In certain embodiments, the method uses 1 ng to 8 ug of mRNA per million T cells per transfection. In certain embodiments, the method uses 1 ng to 7 ug of mRNA per million T cells per transfection. In certain embodiments, the method uses 1 ng to 6 ug of mRNA per million T cells per transfection. In certain embodiments, the method uses 1 ng to 5 ug of mRNA per million T cells per transfection. In certain embodiments, the method uses 1 ng to 4 ug of mRNA per million T cells per transfection. In certain embodiments, the method uses 1 ng to 3 ug of mRNA per million T cells per transfection. In certain embodiments, the method uses 1 ng to 2 ug of mRNA per million T cells per transfection. In certain embodiments, the method uses 1 ng to 1 ug of mRNA per million T cells per transfection. In certain embodiments, the method uses 10 ng to 10 ug of mRNA per million T cells per transfection. In certain embodiments, the method uses 50 ng to 10 ug of mRNA per million T cells per transfection. In certain embodiments, the method uses 100 ng to 10 ug of mRNA per million T cells per transfection. In certain embodiments, the method uses 150 ng to 10 ug of mRNA per million T cells per transfection. In certain embodiments, the method uses 200 ng to 10 ug of mRNA per million T cells per transfection. In certain embodiments, the method uses 300 ng to 10 ug of mRNA per million T cells per transfection. In certain embodiments, the method uses 350 ng to 10 ug of mRNA per million T cells per transfection. In certain embodiments, the method uses 400 ng to 10 ug of mRNA per million T cells per transfection. In certain embodiments, the method uses 500 ng to 10 ug of mRNA per million T cells per transfection. In certain embodiments, the method uses 600 ng to 10 ug of mRNA per million T cells per transfection. In certain embodiments, the method uses 750 ng to 10 ug of mRNA per million T cells per transfection. In certain embodiments, the method uses 900 ng to 10 ug of mRNA per million T cells per transfection. In certain embodiments, the method uses 1 ug to 10 ug of mRNA per million T cells per transfection. In certain embodiments, the method uses 2 ug to 10 ug of mRNA per million T cells per transfection. In certain embodiments, the method uses 2 ug to 6 ug of mRNA per million T cells per transfection. In certain embodiments, the method uses 1 ug to 9 ug of mRNA per million T cells per transfection. In certain embodiments, the method uses 1 ug to 8 ug of mRNA per million T cells per transfection. In certain embodiments, the method uses 1 ug to 7 ug of mRNA per million T cells per transfection. In certain embodiments, the method uses 1 ug to 6 ug of mRNA per million T cells per transfection. In certain embodiments, the method uses 1 ug to 5 ug of mRNA per million T cells per transfection. In certain embodiments, the method uses 1 ug to 4 ug of mRNA per million T cells per transfection. In certain embodiments, the method uses 1 ug to 3 ug of mRNA per million T cells per transfection. In certain embodiments, the method uses 1 ug to 2 ug of mRNA per million T cells per transfection. In certain embodiments, the mRNA encoding the one or more reprogramming factors is transfected into the T cells at least once daily for between about 2-5 consecutive days. In certain embodiments, the mRNA encoding the one or more reprogramming factors is transfected into the T cells at least once daily for 5 consecutive days. In certain embodiments, the mRNA encoding the one or more reprogramming factors is transfected into the T cells at least once daily for 4 consecutive days. In certain embodiments, the mRNA encoding the one or more reprogramming factors is transfected into the T cells at least once daily for 3 consecutive days. In certain embodiments, the mRNA encoding the one or more reprogramming factors is transfected into the T cells at least once daily for 2 consecutive days. In certain embodiments, the mRNA encoding the one or more reprogramming factors is transfected into the T cells at least once daily for 1 day. In certain embodiments, the mRNA encoding the one or more reprogramming factors is transfected into the T cells once daily for 5 consecutive days. In certain embodiments, the mRNA encoding the one or more reprogramming factors is transfected into the T cells once daily for 4 consecutive days. In certain embodiments, the mRNA encoding the one or more reprogramming factors is transfected into the T cells once daily for 3 consecutive days. In certain embodiments, the mRNA encoding the one or more reprogramming factors is transfected into the T cells once daily for 2 consecutive days.

In another aspect, a method for reducing T cell exhaustion that occurs during the manufacture of CAR T cells is provided. In some embodiments, the method comprises activating T cells by exposing them to CD3 and/or CD28; transfecting said activated T cells with mRNA encoding one or more reprogramming factors; wherein 1 ng to 500 ug of mRNA per million T cells is used per transfection, wherein said transfection achieves expression of the one or more reprogramming factors in said T cells to increase their cytotoxicity or proliferation capacity as compared to untreated or control T cells. In certain embodiments, the mRNA encoding the one or more reprogramming factors is transfected into the T cells once daily for 1 day. In certain further embodiments, the amount of mRNA encoding the one or more reprogramming factors that is used per million T cells is about 1 ng to 100 ug, and the mRNA encodes the reprogramming factors OCT4, SOX2, KLF4, c-MYC, LIN28, and NANOG. In certain other further embodiments, the amount of mRNA encoding the one or more reprogramming factors that is used per million T cells is about 50 ug, and the mRNA encodes the reprogramming factors OCT4, SOX2, KLF4, c-MYC, LIN28, and NANOG. In additional further embodiments, the amount of mRNA encoding the one or more reprogramming factors that is used per million T cells is about 25 ug, and the mRNA encodes the reprogramming factors OCT4, SOX2, KLF4, c-MYC, LIN28 and NANOG. In some further embodiments, the mRNA encodes the reprogramming factors OCT4, SOX2, KLF4, c-MYC, LIN28, NANOG and Gls1

In another aspect, a method for reducing T cell exhaustion that occurs during the manufacture of CAR T cells is provided. In some embodiments, the method comprises activating T cells by exposing them to CD3 and/or CD28; transfecting said activated T cells with mRNA encoding one or more reprogramming factors; wherein 1 ng to 500 ug of mRNA per million T cells is used per transfection, wherein said transfection achieves expression of the one or more reprogramming factors in said T cells to increase their cytotoxicity or proliferation capacity as compared to untreated or control T cells. In certain embodiments, the mRNA encoding the one or more reprogramming factors is transfected into the T cells twice daily for between about 2-5 consecutive days. In certain further embodiments, the amount of mRNA encoding the one or more reprogramming factors that is used per million T cells is (a) about 1 ng to 100 ug mRNA per million T cells for the first of two daily transfections; and (b) about 1 ng to 100 ug mRNA per million T cells for the second of two daily transfections. In certain other further embodiments, the amount of mRNA encoding the one or more reprogramming factors that is used per million T cells is (a) about 100 ng to 10 ug mRNA per million T cells for the first of two daily transfections; and (b) about 100 ng to 10 ug mRNA per million T cells for the second of two daily transfections. In certain other further embodiments, the amount of mRNA encoding the one or more reprogramming factors that is used per million T cells is (a) about 10 ng to 10 ug mRNA per million T cells for the first of two daily transfections; (b) about 10 ng to 8 ug mRNA per million T cells for the second of two daily transfections; and the mRNA used for the fust of two daily transfections encodes OCT4, SOX2, KLF4, c-MYC, LIN28 and NANOG; and the mRNA used for the second of two daily transfections encodes OCT4, c-MYC and NANOG.

In another aspect, a method for reducing T cell exhaustion that occurs during the manufacture of CAR T cells is provided. In some embodiments, the method comprises activating T cells by exposing them to CD3 and/or CD28; transfecting said activated T cells with mRNA encoding one or more reprogramming factors; wherein 1 ng to 500 ug of mRNA per million T cells is used per transfection, wherein said transfection achieves expression of the one or more reprogramming factors in said T cells to increase their cytotoxicity or proliferation capacity as compared to untreated or control T cells. In certain embodiments, the mRNA encoding the one or more reprogramming factors is transfected into the T cells twice daily for 5 consecutive days. In certain further embodiments, the amount of mRNA encoding the one or more reprogramming factors that is used per million T cells is (a) about 1 ng to 100 ug mRNA per million T cells for the first of two daily transfections; and (b) about 1 ng to 100 ug mRNA per million T cells for the second of two daily transfections. In certain other further embodiments, the amount of mRNA encoding the one or more reprogramming factors that is used per million T cells is (a) about 100 ng to 10 ug mRNA per million T cells for the first of two daily transfections; and (b) about 100 ng to 10 ug mRNA per million T cells for the second of two daily transfections. In certain other further embodiments, the amount of mRNA encoding the one or more reprogramming factors that is used per million T cells is (a) about 10 ng to 10 ug mRNA per million T cells for the first of two daily transfections; (b) about 10 ng to 8 ug mRNA per million T cells for the second of two daily transfections; and the mRNA used for the first of two daily transfections encodes OCT4, SOX2, KLF4, c-MYC, LIN28 and NANOG; and the mRNA used for the second of two daily transfections encodes OCT4, c-MYC and NANOG.

In another aspect, a method for reducing T cell exhaustion that occurs during the manufacture of CAR T cells is provided. In some embodiments, the method comprises activating T cells by exposing them to CD3 and/or CD28; transfecting said activated T cells with mRNA encoding one or more reprogramming factors; wherein 1 ng to 500 ug of mRNA per million T cells is used per transfection, wherein said transfection achieves expression of the one or more reprogramming factors in said T cells to increase their cytotoxicity or proliferation capacity as compared to untreated or control T cells. In certain embodiments, the mRNA encoding the one or more reprogramming factors is transfected into the T cells twice daily for 4 consecutive days. In certain further embodiments, the amount of mRNA encoding the one or more reprogramming factors that is used per million T cells is (a) about 1 ng to 100 ug mRNA per million T cells for the first of two daily transfections; and (b) about 1 ng to 100 ug mRNA per million T cells for the second of two daily transfections. In certain other further embodiments, the amount of mRNA encoding the one or more reprogramming factors that is used per million T cells is (a) about 100 ng to 10 ug mRNA per million T cells for the first of two daily transfections; and (b) about 100 ng to 10 ug mRNA per million T cells for the second of two daily transfections. In certain other further embodiments, the amount of mRNA encoding the one or more reprogramming factors that is used per million T cells is (a) about 10 ng to 10 ug mRNA per million T cells for the first of two daily transfections; (b) about 10 ng to 8 ug mRNA per million T cells for the second of two daily transfections; and the mRNA used for the first of two daily transfections encodes OCT4, SOX2, KLF4, c-MYC, LIN28 and NANOG; and the mRNA used for the second of two daily transfections encodes OCI4, c-MYC and NANOG.

In another aspect, a method for reducing T cell exhaustion that occurs during the manufacture of CAR T cells is provided. In some embodiments, the method comprises activating T cells by exposing them to CD3 and/or CD28; transfecting said activated T cells with mRNA encoding one or more reprogramming factors; wherein 1 ng to 500 ug of mRNA per million T cells is used per transfection, wherein said transfection achieves expression of the one or more reprogramming factors in said T cells to increase their cytotoxicity or proliferation capacity as compared to untreated or control T cells. In certain embodiments, the mRNA encoding the one or more reprogramming factors is transfected into the T cells twice daily for 3 consecutive days. In certain further embodiments, the amount of mRNA encoding the one or more reprogramming factors that is used per million T cells is (a) about 1 ng to 100 ug mRNA per million T cells for the first of two daily transfections; and (b) about 1 ng to 100 ug mRNA per million T cells for the second of two daily transfections. In certain other further embodiments, the amount of mRNA encoding the one or more reprogramming factors that is used per million T cells is (a) about 100 ng to 10 ug mRNA per million T cells for the first of two daily transfections; and (b) about 100 ng to 10 ug mRNA per million T cells for the second of two daily transfections. In certain other further embodiments, the amount of mRNA encoding the one or more reprogramming factors that is used per million T cells is (a) about 10 ng to 10 ug mRNA per million T cells for the first of two daily transfections; (b) about 10 ng to 8 ug mRNA per million T cells for the second of two daily transfections; and the mRNA used for the first of two daily transfections encodes OCT4, SOX2, KLF4, c-MYC, LIN28 and NANOG; and the mRNA used for the second of two daily transfections encodes OCT4, c-MYC and NANOG. In certain other further embodiments, the amount of mRNA encoding the one or more reprogramming factors that is used per million T cells is: (a) about 10 ng to 10 ug mRNA per million T cells for the first of two daily transfections; and (b) about 10 ng to 8 ug mRNA per million T cells for the second of two daily transfections; and the mRNA used for the first of two daily transfections encodes OCT4, SOX2, KLF4, c-MYC, LIN28 and NANOG; and the mRNA used for the second of two daily transfections encodes OCT4, c-MYC and NANOG.

In another aspect, a method for reducing T cell exhaustion that occurs during the manufacture of CAR T cells is provided. In some embodiments, the method comprises activating T cells by exposing them to CD3 and/or CD28; transfecting said activated T cells with mRNA encoding one or more reprogramming factors; wherein 1 ng to 500 ug of mRNA per million T cells is used per transfection, wherein said transfection achieves expression of the one or more reprogramming factors in said T cells to increase their cytotoxicity or proliferation capacity as compared to untreated or control T cells. In certain embodiments, the mRNA encoding the one or more reprogramming factors is transfected into the T cells twice daily for 2 consecutive days. In certain further embodiments, the amount of mRNA encoding the one or more reprogramming factors that is used per million T cells is (a) about 1 ng to 100 ug mRNA per million T cells for the first of two daily transfections; and (b) about 1 ng to 100 ug mRNA per million T cells for the second of two daily transfections. In certain other further embodiments, the amount of mRNA encoding the one or more reprogramming factors that is used per million T cells is (a) about 100 ng to 10 ug mRNA per million T cells for the first of two daily transfections; and (b) about 100 ng to 10 ug mRNA per million T cells for the second of two daily transfections. In certain other further embodiments, the amount of mRNA encoding the one or more reprogramming factors that is used per million T cells is (a) about 10 ng to 10 ug mRNA per million T cells for the first of two daily transfections; (b) about 10 ng to 8 ug mRNA per million T cells for the second of two daily transfections; and the mRNA used for the first of two daily transfections encodes OCT4, SOX2, KLF4, c-MYC, LIN28 and NANOG; and the mRNA used for the second of two daily transfections encodes OCT4, c-MYC and NANOG. In certain other further embodiments, the amount of mRNA encoding the one or more reprogramming factors that is used per million T cells is: (a) about 1 ng to 100 ug mRNA per million T cells for the first of two daily transfections; and (b) about 1 ng to 100 ug mRNA per million T cells for the second of two daily transfections; and the mRNA used for the first of two daily transfections encodes OCT4, SOX2, KLF4, c-MYC, LIN28 and NANOG; and the mRNA used for the second of two daily transfections encodes OCT4, c-MYC and NANOG.

In another aspect, a method for reducing T cell exhaustion that occurs during the manufacture of CAR T cells is provided. In some embodiments, the method comprises activating T cells by exposing them to CD3 and/or CD28; transfecting said activated T cells with mRNA encoding one or more reprogramming factors; wherein 1 ng to 500 ug of mRNA per million T cells is used per transfection, wherein said transfection achieves expression of the one or more reprogramming factors in said T cells to increase their cytotoxicity or proliferation capacity as compared to untreated or control T cells. In certain embodiments, the mRNA encoding the one or more reprogramming factors is transfected into the T cells twice daily for 1 day. In certain further embodiments, the amount of mRNA encoding the one or more reprogramming factors that is used per million T cells is (a) about 1 ng to 100 ug mRNA per million T cells for the first of two daily transfections; and (b) about 1 ng to 100 ug mRNA per million T cells for the second of two daily transfections. In certain other further embodiments, the amount of mRNA encoding the one or more reprogramming factors that is used per million T cells is (a) about 100 ng to 10 ug mRNA per million T cells for the first of two daily transfections; and (b) about 100 ng to 10 ug mRNA per million T cells for the second of two daily transfections. In certain other further embodiments, the amount of mRNA encoding the one or more reprogramming factors that is used per million T cells is (a) about 10 ng to 10 ug mRNA per million T cells for the first of two daily transfections; (b) about 10 ng to 8 ug mRNA per million T cells for the second of two daily transfections; and the mRNA used for the first of two daily transfections encodes OCT4, SOX2, KLF4, c-MYC, LIN28 and NANOG; and the mRNA used for the second of two daily transfections encodes OCT4, c-MYC and NANOG. In certain other further embodiments, the amount of mRNA encoding the one or more reprogramming factors that is used per million T cells is: (a) about 1 ng to 100 ug mRNA per million T cells for the first of two daily transfections; and (b) about 1 ng to 100 ug mRNA per million T cells for the second of two daily transfections; and the mRNA used for the first of two daily transfections encodes OCT4, SOX2, KLF4, c-MYC, LIN28 and NANOG; and the mRNA used for the second of two daily transfections encodes OCT4, c-MYC and NANOG.

In another aspect, a method for reducing T cell exhaustion that occurs during the manufacture of CAR T cells is provided. In certain embodiments the method comprises activating T cells by exposing them to CD3 and/or CD28; transfecting said activated T cells with mRNA encoding one or more reprogramming factors; wherein 1 ng to 500 ug of mRNA per million T cells is used per transfection, wherein said transfection achieves expression of the one or more reprogramming factors in said T cells to increase the percentage of Tcm or Tscm cells as compared to untreated or control T cells. In certain embodiments, the increase in the percentage of Tcm or Tscm cells is less than 200%. In certain embodiments, the increase in the percentage of Tcm or Tscm cells is less than 150%. In certain embodiments, the increase in the percentage of Tcm or Tscm cells is less than 125%. In certain embodiments, the increase in the percentage of Tcm or Tscm cells is less than 100%. In certain embodiments, the increase in the percentage of Tcm or Tscm cells is less than 80%. In certain embodiments, the increase in the percentage of Tcm or Tscm cells is less than 60%. In certain embodiments, the increase in the percentage of Tcm or Tscm cells is less than 40%. In certain embodiments, the increase in the percentage of Tcm or Tscm cells is less than 30%. In certain embodiments, the increase in the percentage of Tcm or Tscm cells is less than 20%. In certain embodiments, the increase in the percentage of Tcm or Tscm cells is less than 10%. In certain embodiments, the increase in the percentage of Tcm or Tscm cells is between about 10% and 40%. In certain embodiments, the increase in the percentage of Tcm or Tscm cells is between about 10% and 35%. In certain embodiments, the increase in the percentage of Tcm or Tscm cells is between about 5% and 40%. In certain embodiments, the increase in the percentage of Tcm or Tscm cells is between about 5% and 35%. In certain embodiments, the increase in the percentage of Tcm or Tscm cells is between about 5% and 30%. In certain embodiments, the increase in the percentage of Tcm or Tscm cells is between about 5% and 25%. In certain embodiments, the increase in the percentage of Tcm or Tscm cells is between about 10% and 40%.

In certain embodiments, the increase in the percentage of Tcm or Tscn cells is between about 10% and 50%. In certain embodiments, the increase in the percentage of Tcm or Tscm cells is between about 5% and 50%. In certain embodiments, the increase in the percentage of Tcm or Tscm cells is between about 5% and 65%. In certain embodiments, the increase in the percentage of Tcm or Tscm cells is between about 5% and 75%. In certain embodiments, the increase in the percentage of Tcm or Tscm cells is between 1% and 50%. In certain embodiments, the increase in the percentage of Tcm or Tscm cells is between 1% and 40%. In certain embodiments, the increase in the percentage of Tcm or Tscm cells is between 1% and 25%. In certain embodiments, the increase in the percentage of Tcm or Tscm cells is between 20% and 80%. In certain embodiments, the increase in the percentage of Tcm or Tscm cells is between about 2-fold and 10-fold. In certain embodiments, the increase in the percentage of Tcm or Tscm cells is between about 2-fold and 8-fold. In certain embodiments, the increase in the percentage of Tcm or Tscm cells is between about 2-fold and 6-fold. In certain embodiments, the increase in the percentage of Tcm or Tscm cells is between about 1-fold and 4-fold. In certain further embodiments, the mRNA molecule comprises a self-amplifying RNA that exhibits a half-life of 2 days or more. In certain other further embodiments, the mRNA molecule is a self-amplifying RNA that exhibits a half-life of 2 days or more. In certain other further embodiments, the mRNA molecule comprises a trans-amplifying RNA that exhibits a half-life of 2 days or more. In certain other further embodiments, the mRNA molecule is a trans-amplifying RNA, that exhibits a half-life of 2 days or more. In certain other further embodiments, the mRNA molecule comprises a trans- or self-amplifying RNA that exhibits a half-life of up to 10 days.

In addition to the exemplary aspects and embodiments described above, further aspects and embodiments will become apparent by study of the following descriptions.

Additional embodiments of the present methods and compositions, and the like, will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts ionizable lipids of Formula (I) (FIG. 1A) and exemplary lipid structures of the same (FIG. 1B and FIG. 1C) that may be used in some embodiments of the present technology, for example, in transfecting immune cells.

FIG. 2 depicts ionizable lipids of Formula (I-A) (FIG. 2A) and an exemplary lipid structure of the same (FIG. 2B) that may be used in some embodiments of the present technology, for example, in transfecting immune cells.

FIG. 3 depicts ionizable lipids of Formula (I-B) (FIG. 3A) and an exemplary lipid structure of the same (FIG. 3B) that may be used in some embodiments of the present technology, for example, in transfecting immune cells.

FIG. 4 depicts ionizable lipids of Formula (II) (FIG. 4A) and exemplary lipid structures of the same (FIG. 4B and FIG. 4C) that may be used in some embodiments of the present technology, for example, in transfecting immune cells.

FIG. 5 depicts ionizable lipids of Formula (III)(FIG. 5A) and exemplary lipid structures of the same (FIG. 5B and FIG. 5C) that may be used in some embodiments of the present technology, for example, in transfecting immune cells.

FIG. 6 depicts ionizable lipids of Formula (VI) (FIG. 6A) and exemplary lipid structures of the same (FIG. 6B and FIG. 6C) that may be used in some embodiments of the present technology, for example, in transfecting immune cells.

FIG. 7 depicts ionizable lipids of Formula (V) (FIG. 7A) and an exemplary lipid structure of the same (FIG. 7B) that may be used in some embodiments of the present technology, for example, in transfecting immune cells.

FIG. 8 depicts ionizable lipids of Formula (VI)(FIG. 8A) and exemplary lipid structures of the same (FIG. 8B and FIG. 8C) that may be used in some embodiments of the present technology, for example, in transfecting immune cells.

FIG. 9 depicts ionizable lipids of Formula (VII) (FIG. 9A) and exemplary lipid structures of the same (FIG. 9B and FIG. 9C) that may be used in some embodiments of the present technology, for example, in transfecting immune cells.

FIG. 10 depicts ionizable lipids of Formula (VIII) (FIG. 10A) and an exemplary lipid structure of the same (FIG. 10B) that may be used in some embodiments of the present technology, for example, in transfecting immune cells.

FIG. 11 depicts ionizable lipids of Formula (IX) (FIG. 11A) and exemplary lipid structures of the same (FIG. 11B and FIG. 11C) that may be used in some embodiments of the present technology, for example, in transfecting immune cells.

FIG. 12 depicts ionizable lipids of Formula (X) (FIG. 12A) and exemplary lipid structures of the same (FIG. 12B and FIG. 12C) that may be used in some embodiments of the present technology, for example, in transfecting immune cells.

FIG. 13 depicts ionizable lipids of Formula (XI) (FIG. 13A) and an exemplary lipid structure of the same (FIG. 13B) that may be used in some embodiments of the present technology, for example, in transfecting immune cells.

FIG. 14 depicts ionizable lipids of Formula (XII) (FIG. 14A) and an exemplary lipid structure of the same (FIG. 14B) that may be used in some embodiments of the present technology, for example, in transfecting immune cells.

FIG. 15 depicts ionizable lipids of Formula (XIII) (FIG. 15A) and an exemplary lipid structure of the same (FIG. 15B) that may be used in some embodiments of the present technology, for example, in transfecting immune cells.

FIG. 16 depicts ionizable lipids of Formula (XIV) (FIG. 16A) and an exemplary lipid structure of the same (FIG. 16B) that may be used in some embodiments of the present technology, for example, in transfecting immune cells.

FIG. 17 depicts ionizable lipids of Formula (XV) (FIG. 17A) and exemplary lipid structures of the same (FIG. 17B and FIG. 17C) that may be used in some embodiments of the present technology, for example, in transfecting immune cells.

FIG. 18 depicts ionizable lipids of Formula (XVI) (FIG. 18A) and exemplary lipid structures of the same (FIG. 18B and FIG. 18C) that may be used in some embodiments of the present technology, for example, in transfecting immune cells.

FIGS. 19(A) to 19(F) provide marker expression data of rejuvenated T cells expressing OSKMLN reprogramming factors as described herein, at day 15 of manufacture.

FIGS. 20(A)-20(F) provide T cell mediated cytotoxicity data of rejuvenated T cells expressing OSKLMN reprogramming factors as described herein compared with un-rejuvenated control T cells. Starting at the third addition of tumor cells (also referred to as the “3^{rd} target engagement” or the “third engagement”), T-cells expressing reprogramming factors exhibited an increased efficiency in killing tumor cells as compared to non-treated T-cell controls. Using the cytotoxicity of ERA-treated T cells as a 100% baseline value, the cytotoxicity of non-treated cells was only 20% to 30% that of the ERA-treated T cells, as indicated by the double arrows in FIG. 20(C) through FIG. 20(F). In addition, this increased efficiency in killing tumor cells was a durable benefit, as it remained high from the third to the sixth addition of tumor cells.

FIGS. 21(A)-21(B) provide proliferation data of rejuvenated T cells expressing OSKMLN reprogramming factors as described herein compared with un-rejuvenated control T cells. As emphasized in the shaded portion of FIG. 21B, T cell expressing OSKLMN reprogramming factors have a three-to-five fold higher proliferation rate than untreated control T cells. For example, at the 2^{nd} target engagement, the T cells expressing OSKLMN reprogramming factors had a 20-fold increase in proliferation as compared to the untreated control T cells (FIG. 21(B); p<0.01 for ERA-treated T cells versus the “No Treatment” control T cells). The T cells used in this proliferation experiment were obtained from a 63-year old donor. Treatment with the OSKLMN reprogramming factors significantly increased T-cell proliferation in this older patient that would otherwise be ineligible for CAR-T therapies.

FIG. 21C provides T cell mediated cytotoxicity data of rejuvenated T cells obtained from the same 63-year-old donor as in FIGS. 21(A) and (B). Their cytotoxicity was compared with un-rejuvenated control T cells at the third engagement/third addition of tumor cells. The T cells expressing OSKLMN reprogramming factors killed tumor cells 4-5 times more efficiently (FIG. 21(C); p<0.05 for ERA-treated T cells versus “No Treatment” control T cells). Treatment with the OSKLMN reprogramming factors significantly increased tumor cell killing in this older patient that would otherwise be ineligible for CAR-T therapies.

FIGS. 22(A) and 22(B) provide marker expression data of rejuvenated T cells expressing OSKLMN reprogramming factors as described herein, at the end of manufacture and upon Daudi target cell engagement. The expression of the Tscm marker CD28 was 20% higher in the reprogrammed T cells versus the untreated control T cells, and FIG. 22(C) shows that expression of Tscm marker CD95 was 20% higher than in the reprogrammed T cells versus the untreated control T cells. These increased levels of Tscm markers support increased proliferation and increased killing capacity.

FIGS. 22(C) and 22(D) provide exhaustion marker expression data for the same rejuvenated T cells expressing OSKLMN reprogramming factors as described in FIGS. 22(A) and 22(B). After the second round of adding tumor cells, the levels of exhaustion markers TIGIT and LAG-3 were decreased by 25% and 80%, respectively.

FIGS. 22(E) and 22(F) also provide exhaustion marker expression data for the same rejuvenated T cells expressing OSKLMN reprogramming factors as described in FIGS. 22(A) and 22(B). After the third round of adding tumor cells, the expression levels of exhaustion markers TIGIT and LAG-3 were decreased by 50% and 70%, respectively.

FIG. 23(A) provides proliferation data of rejuvenated T cells obtained from a 34-year-old donor. The T cells expressing OSKMLN reprogramming factors as described herein were compared with un-rejuvenated control T cells. As emphasized in the shaded portion of FIG. 23A, T cells expressing OSKLMN reprogramming factors have a 4 to 6-fold increase in proliferation as compared to untreated control T cells and with T cells expressing GFP instead of reprogramming factors. For example, at the 4^{th} target engagement, the “ERA_2” T cells expressing OSKLMN reprogramming factors had an approximately 30-fold increase in proliferation as compared to the untreated control T cells as well as compared to the GFP control T cells (FIG. 21(B); p<0.001). Treatment with the OSKLMN reprogramming factors significantly increased T-cell proliferation.

FIG. 23(B) provides T cell mediated cytotoxicity data of rejuvenated T cells obtained from the same 34-year-old donor as in FIG. 23(A). Their cytotoxicity during the 5^{th} tumor cell addition/target engagement was compared with un-rejuvenated control T cells (for “ERA_1”) or with T cells expressing GFP instead of reprogramming factors (for “ERA_2”). The T cells expressing OSKLMN reprogramming factors killed tumor cells 4-5 times more efficiently (FIG. 23(B); p<0.01 for “ERA_2” T cells versus GFP, and also for “ERA_1” T cells versus the no treatment control T cells). Treatment with the OSKLMN reprogramming factors significantly also increased tumor cell killing in this young donor patient that would be eligible for CAR-T therapies.

FIG. 24 depicts a method of incorporating ERA-treated T cells into the manufacturing process of any cell-based immunotherapy in order to reduce or eliminate manufacturing-induced differentiation and/or exhaustion. Primary T cells are activated with CD3 and/or CD28, and the addition of mRNA encoding one or more reprogramming factors as described herein results in an increased number of T cells with increases in at least one or more of the following properties: proliferation, tumor cell killing, self-renewal, multipotency and/or functional persistence. In contrast, activated T cells that are not treated with mRNA encoding one or more reprogramming factors as described herein are more differentiated, have limited proliferative capacity, and are prone to exhaustion.

FIG. 25 depicts a three week-long, 2-arm comparative study that was conducted to demonstrate that treatment with mRNA encoding one or more reprogramming factors enhanced T-cell proliferation and T cell mediated cytotoxicity. The study employed a long-term killing assay using CD19-CD3 bispecific antibody to simulate the repeated engagement between T cells and tumor cells in vivo.

FIGS. 26(A) and 26(B) provide CCR7 and CD27 marker expression data of rejuvenated T cells obtained from a 32-year-old donor that expressed OSKLMN reprogramming factors as described herein. The expression of CCR7 was approximately 1.3-fold higher in the reprogrammed T cells versus the untreated control T cells (FIG. 26(A)).

FIG. 27 provides CCR7 and CD27 marker expression data of rejuvenated T cells obtained from a 73-year-old donor that expressed OSKLMN reprogramming factors as described herein. The expression of CCR7 was over 2-fold higher in the reprogrammed T cells versus the untreated control T cells (FIG. 27(A)).

FIG. 28 provides cytokine concentrations for IL-2, GM-CSF, IFNγ and TNF-α, as determined using an Luminex assay on the rejuvenated T cells obtained from a 57-year old donor.

FIG. 29 provides CCR7 and CD45RA expression data after an mRNA cocktail encoding OSKLMN reprogramming factors was transfected by electroporation into CD3/CD28-activated T cells. Increased CCR7 expression indicates expression of reprogramming factors enhanced central memory (Tcm) and stem memory T cell (Tscm)-like phenotype. The increased % of Tcm and Tscm population will lead to higher proliferation and longer persistence in patients. 1× ERA and 2× ERA indicated different doses of ERA treatment. EoM=End of Manufacturing. T_{N-SCM} (T Naïve-Stem Cell Memory), T_{CM} (T central memory), T_{EM} (T Effector Memory), T_{E} (T Effector).

### BRIEF DESCRIPTION OF THE SEQUENCES

In some embodiments, the methods and compositions related to rejuvenating immune cells, such as T cells, comprise exposing the immune cell to messenger RNA (mRNA) encoding one or more reprogramming factors wherein the reprogramming factor encoding mRNA encodes a polypeptide encoded by a polynucleotide having at least 95% sequence identity to any one of SEQ Id NOs: 1-14 (Table 1).

In some aspects, a reprogramming factor encoding polynucleotide having at least 95% sequence identity to any one of SEQ Id NOs: 1-14 is contemplated for use in conjunction with the methods and compositions comprising rejuvenated immune cells provided herein.

In another aspect, a reprogramming factor protein or polypeptide encoded by a polynucleotide having at least 95% sequence identity to any one of SEQ Id NOs: 1-14 is contemplated for use in conjunction with the methods and compositions comprising rejuvenated immune cells provided herein.

In another aspect, an RNA vector, comprising one or more reprogramming factor polynucleotide sequences, wherein the one or more polynucleotide sequences comprises at least 95% sequence identity to any one of SEQ Id NOs: 1-14 is contemplated for use in conjunction with the methods and compositions comprising rejuvenated immune cells provided herein.

In some embodiments, the present technology is related to mRNA encoding one or more reprogramming factors wherein the reprogramming factor encoding mRNA encodes a polypeptide encoded by a polynucleotide having at least about 70%, about 75%, about 80%, about 85%, about 90%, about 95%, about 96%, about 97%, about 98%, about 99%, about 100% sequence identity to any one of SEQ Id NOs: 1-14 (Table 1).

In some embodiments, the present technology is related to mRNA encoding one or more reprogramming factors wherein the reprogramming factor encoding mRNA encodes a polypeptide encoded by a polynucleotide having about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% sequence identity to any one of SEQ Id NOs: 1-14 (Table 1).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μM.

The singular forms “a”, “an” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent; when used in reference to a given range or set of values, it encompasses deviations of plus or minus five percent and applies to each stated value within the given range or set of values.

The compositions of the present disclosure can comprise, consist essentially of, or consist of, the components disclosed.

All percentages, parts and ratios are based upon the total weight of the topical compositions and all measurements made are at about 25′C, unless otherwise specified.

As used herein, the term “cell” refers to an intact live cell, naturally occurring or modified. The cell may be isolated from other cells, mixed with other cells in a culture, or within a tissue (partial or intact), or an organism. The methods described herein can be performed, for example, on a sample comprising a single cell, a population of cells, or a tissue or organ comprising cells.

As used herein, the term “mRNA” refers to a riboxynucleic acid (RNA) encoding at least one amino acid, for example a peptide or protein. Said riboxynucleic acid is synthesized according to methods known in the art. Said riboxynucleic acid can mimic or recapitulate natural messenger RNA encoding reprogramming factors, or it can have a different structure or format, for example polycistronic RNA, circular RNA, self-amplifying RNA, or chemically modified RNA. Herein, all such RNA is referred to as “mRNA.”

As used herein, the term “immune cell” refers to a cell that is part of the immune system and helps the body fight infections and other diseases. In some instances, immune cells develop from stem cells in the bone marrow and become different types of white blood cells. Immune cells provided herein include, but are not limited to, lymphocytes, granulocytes (neutrophils, eosinophils, basophils, mast cells), monocytes, macrophages, microglia, dendritic cells, T-cells (cytotoxic T cells (CD8+), helper T cells (CD4+), suppressor or regulatory T cells (Tregs), memory T cells, Th1 T cells, Th2 T cells, Th17 T cells, Th9 T cells, Tfh T cells, antigen-inexperienced naïve T cells (Tn or TN), stem cell memory T cells (Tscm or T_{SCM}), central memory T cells (Tcm or T_{CM}), effector memory T cells (Tem or T_{EM}), effector T cells (Teff, T_{EFF} or T_{E}), precursors to an exhausted T cell (Tpex or T_{PEX}), or exhausted T cells (Tex or T_{EX}), central memory T cells, effector memory T cells, tissue resident memory T cells, virtual memory T cells, natural killer T cells (NKT cells), FOXP3+ T cells, FOXP3− T cells), B-cells (memory B-cells or plasma cells), natural killer (NK) cells, tumor-infiltrating lymphocytes, and the like, including natural immune cells or engineered immune cells of all the preceding immune cell types.

In some embodiments, the cell is a non-adherent immune cell. In some embodiments, non-adherent immune cells, are treated, transiently reprogrammed, rejuvenated, or manufactured in a manner wherein the cells remain non-adherent, without adhering to a tissue culture substrate or forming or giving rise to cells or colonies of cells that adhere to a tissue culture substrate. In some embodiments, the reprogramming interval and factors are selected such that cells are rejuvenated with retention of cellular identity, wherein the cells remain non-adherent, without adhering to a tissue culture substrate or forming or giving rise to cells or colonies of cells that adhere to a tissue culture substrate. Accordingly, in some embodiments, the present technology provides a reprogramming method wherein cells, including any non-adherent cells and/or non-adherent immune cells (e.g., non-adherent T cells, NK cells, macrophages, tumor infiltrating cells, dendritic cells, and/or NKT cells), are reprogrammed in a manner wherein the cells are rejuvenated with retention of cellular identity, and wherein the cells stay in suspension and are not adherent, nor do they become or give rise to cells that are adherent, become adherent, or form adherent colonies.

As used herein, the term “engineered cell” refers to a cell that has been altered to contain exogenous DNA, RNA, proteins or polypeptides, as compared to an unmodified cell of the same type. Mere isolation or purification of a cell from an organism in which it is found in the wild does not convert it into an engineered cell.

As used herein, the term “transfection” refers to the uptake of exogenous DNA or RNA by a cell, such as an immune cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3.sup.rd edition, Cold Spring Harbor Laboratories, New York, Davis et al. (1995) Basic Methods in Molecular Biology, 2.sup.nd edition, McGraw-Hill, and Chu et al. (1981) Gene 13:197. Such techniques can be used to introduce one or more exogenous DNA or RNA molecules into cells. The term refers to both stable and transient uptake of the DNA or RNA molecules. For example, transfection can be used for transient uptake of mRNAs encoding cellular reprogramming factors into cells in need of rejuvenation.

As used herein, the term “transient reprogramming” refers to exposure of cells, such as immune cells including T cells, to cellular reprogramming factors for a period of time sufficient to rejuvenate cells (i.e., eliminate all or some hallmarks of aging), but not long enough to cause loss of identity or dedifferentiation. Such transient reprogramming results in rejuvenated cells that retain their identity (i.e., differentiated cell-type).

As used herein, the term “rejuvenated immune cells” refers to aged, exhausted, live immune cells that have been treated or transiently reprogrammed with one or more cellular reprogramming factors such that the immune cells have a transcriptomic profile, epigenetic profile or “clock”, or functionality of a younger cell or non-exhausted cell while still retaining one or more cell identity markers.

As used herein, the term “mammalian cell” refers to any cell derived from a mammalian subject suitable for transplantation into the same or a different subject or cells present in a mammalian subject. The cell suitable for transplantation may be xenogeneic, autologous, or allogeneic. The cell can be a primary cell obtained directly from a mammalian subject. The cell may also be a cell derived from the culture and expansion of a cell obtained from a subject. In some embodiments, the cell has been genetically engineered to express a recombinant protein and/or nucleic acid.

As used herein, the term “stem cell” refers to a cell that retains the ability to renew itself through mitotic cell division and that can differentiate into a diverse range of specialized cell types. Mammalian stem cells can be divided into four broad categories: embryonic stem cells, which are derived from blastocysts; induced pluripotent stem cells, which are generated by de-differentiation of somatic cells, adult stem cells, or cord blood stem cells; adult stem cells, which are found in adult tissues; and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells or by secreting trophic factors, cytokines, and/or signaling molecules. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as skin or blood cells.

The phrase “pharmaceutically acceptable” is employed herein to refer to those compounds, salts, compositions, dosage forms, etc., which are, within the scope of sound medical judgment, suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

As used herein, the term “cellular reprogramming factors” or “reprogramming factors” refers to any factor able to rejuvenate cells without resulting in a loss of identity or de-differentiation. In embodiments, cellular reprogramming factors or reprogramming factors are transcription factors and sets and combinations thereof that can convert adult or differentiated cells into pluripotent stem cells. Exemplary reprogramming factors include OCT4, SOX2, KLF4, c-MYC, LIN28, NANOG, and/or GLIS1. Other exemplary reprogramming factors include CMYC, DPPA2, DPPA4, ESRRB, GDF3, GLIS1, KLF2, KLF4, KLF5, LIN28, LMYC, NANOG, NMYC, NR5A1, NR5A2, OCT4, RCOR2, SALL1, SALL4, SOX1, SOX2, SOX3, TDRD12, TET1, TH2A, TH2B, UTF1, ZFP42, MDM2, CyclinD1, SV40 large T antigen, SIRT6, TCL1A, and RARy.

In some instances, the “cellular reprogramming factors” or “reprogramming factors” are selected from the group consisting of an Oct, a Sox, a Klf, a Myc, a Lin or NANOG, i.e., any of these factors alone or in any combination, including in combination with any additional factor able to rejuvenate cells without resulting in a loss of identity or de-differentiation. In some instances, the “cellular reprogramming factors” or “reprogramming factors” are selected from the group consisting of Oct4, Sox2, Klf4, cMyc, Lin28, and NANOG.

The “cellular reprogramming factors” or “reprogramming factors” described herein may comprise proteins, for example transcription factors, which play a role in changing adult or differentiated cells, such as immune cells, into pluripotent stem cells. The terms “cellular reprogramming factors” and “reprogramming factors” further include any analogue molecule that mimics the function of the factor. In embodiments, the reprogramming factor is a factor from the Oct family, the Sox family, the Klf family, the Myc family, Nanog family, or Lin family.

The compositions provided herein comprise reprogramming factor polynucleotides, proteins, polypeptides, and RNA and DNA vectors containing said reprogramming factor polynucleotides for production of said reprogramming factor proteins and polypeptides; for example for expression of such reprogramming factors in cells such as mammalian cells.

The term “treating” is used herein, for instance, in reference to methods of treating a cell, a tissue or a subject, and generally includes the administration of a compound or composition which reduces the frequency or magnitude of, or delays the onset of, symptoms or markers of aging or of a medical condition in the cell, tissue, or subject relative to a cell, tissue, or subject not receiving the compound or composition. This can include reversing, reducing, or arresting the symptoms, clinical signs, and underlying pathology of a condition in a manner to improve or stabilize a subject's condition. Treating also includes the administration of a compound or composition which reduces transcriptomic, proteomic, and/or epigenetic markers of aging and/or exhaustion of immune cells, and/or enhances functions of immune cells including but not limited to proliferation, anti-pathogen activity, anti-tumor activity, and anti-inflammation activity.

As used herein, the term “rejuvenated cell(s)” refers to cells that have been treated or transiently reprogrammed with one or more cellular reprogramming factors such that the cells have a transcriptomic profile of a younger cell while still retaining one or more cell identity markers. In some embodiments, treated cells are rejuvenated and reprogrammed to express markers and a transcriptomic profile of a younger cell while still retaining cell identity markers, such as rejuvenated cells being reprogrammed to express at least 1%, 2%, 3%, 4%, 5%, 10%, 15%, 20%, 25%, 30%, 40%, 50%, 60%, 70%, 75% or more than 75% increase/improvement in expression of at least one rejuvenation marker compared to untreated cells.

The terms “inhibiting” or “reducing” are non-limiting expressions used in reference to methods that affect cancer and/or tumor development or advancement, slow tumor growth, and/or cause stagnation or decrease in tumor size, such as, but not limited to, inhibiting and/or to reducing tumor growth (e.g., decrease the size of a tumor) in a population as compared to an untreated control population. These terms are also used in reference to methods that affect other diseases of cell proliferation, for example fibrosis, such as, but not limited to inhibiting and/or reducing fibrosis (e.g., decreasing the amount of fibrotic tissue) in a population as compared to an untreated control population. These terms are also used in reference to methods that affect other diseases of cell proliferation, for example fibrosis, such as, but not limited to inhibiting and/or reducing fibrosis (e.g., decreasing the amount of fibrotic tissue) in a population as compared to an untreated control population. These terms are also used in reference to methods that affect autoimmune or inflammatory diseases, such as, but not limited to inhibiting and/or reducing autoimmunity or inflammation (e.g., decreasing the amount of autoimmunity or inflammation) in a population as compared to an untreated control population. These terms are also used in reference to methods that affect senescent cells, such as, but not limited to inhibiting and/or reducing senescence (e.g., through senolytic destruction of senescent cells, senomorphic modulation of the phenotype of senescent cells, or senoblocking inhibition of senescence) in a population as compared to an untreated control population.

By reserving the right to proviso out or exclude any individual members of any such group, including any sub-ranges or combinations of sub-ranges within the group, which can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Immune Cell Rejuvenation

The cellular aging process has been postulated to be caused by the loss of both genetic and epigenetic information. Loss of genetic information that contributes to cellular aging is typically in the form of genetic mutations such as substitutions, and deletions in an organism's genome. Loss of or changes in epigenetic information associated with cellular aging can take the form of covalent modifications to DNA, such as 5-methylcytosine (5mC), hydroxymethylcytosine (5hmeC), 5-formylcytosine (fC), and 5-carboxylcytosine (caC) and adenine methylation, and to certain proteins, such as lysine acetylation, lysine and arginine methylation, serine and threonine phosphorylation, and lysine ubiquitination and sumoylation of histone proteins. Loss of and changes in the epigenetic information can result in dysregulation of cellular processes, including processes that maintain cell identity, causing immune cells to exhibit traits that are associated with aging such as senescence.

Immune cell exhaustion is a broad term that has been used to describe the response of immune cells, such as T cells, to chronic antigen stimulation, for example in chronic viral infections and in response to tumors, or during aging. Many features and pathways are implicated as having crucial implications in immune cell exhaustion, including checkpoint blockades, and as having important effects on immune cell therapies, such as adoptive T cell transfer therapies. In some instances, immune cell exhaustion may indicate complete lack of effector function, while in other instances altered/decreased immune cell functionality may be exhibited by immune cells demonstrating or beginning to exhibit exhaustion.

The methods, compositions, and kits of the present disclosure rejuvenate immune cells by preventing and reversing the cellular causes of aging, and/or by preventing and/or reversing cellular exhaustion. The methods, compositions and kits of the present disclosure rejuvenate immune cells by restoring epigenetic information, including epigenetic information that has been lost due to the aging process, cell therapy manufacturing process, injury, or disease, as described, for example, in WO20191782%, incorporated by reference herein in its entirety. The methods, compositions and kits of the present disclosure rejuvenate immune cells by preventing and reversing their exhaustion. The methods, compositions and kits of the present disclosure enhance the function of immune cells by increasing their proliferation and their anti-pathogen activity and anti-tumor activity, or in the case of suppressor or regulatory T cells, their anti-inflammation activity.

The methods for rejuvenating immune cells provided herein include transfecting immune cells with one or more mRNA, such as mRNA encoding one or more cellular reprogramming factors, thereby producing rejuvenated immune cells. In embodiments, the immune cells are contacted with, exposed to, or transfected with the mRNA for not more than about 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, or 2 days. In embodiments, the immune cells are contacted with, exposed to, or transfected with the mRNA for not more than about 14 days, 10 days, 7 days, 6 days, 5 days, or 4 days. In embodiments, the immune cells are contacted with, exposed to, or transfected with the mRNA for not more than about 14 days, 10 days, 7 days, 6 days, 5 days, 4 days, 3 days, 2 days, 1 day, or less than 1 day. In embodiments, the immune cells are contacted with, exposed to, or transfected with the mRNA for not more than about 7 days, 6 days, 5 days, or 4 days. In embodiments, the immune cells are contacted with, exposed to, or transfected with the mRNA for not more than about 3 days, 2 days, 1 day, or less than 1 day. In embodiments, the immune cells are contacted with, exposed to, or transfected with the mRNA for not more than 6 days. In embodiments, the immune cells are contacted with, exposed to, or transfected with the mRNA for 1, 2, 3, 4, 5, 6 days. In embodiments, the immune cells are contacted with, exposed to, or transfected with the mRNA for less than one day. In embodiments, the rejuvenated immune cells have a phenotype or activity profile similar to a young immune cell or non-exhausted immune cell. The phenotype or activity profile includes one or more of the transcriptomic profile, proteomic profile, secretomic profile, gene expression of one or more nuclear and/or epigenetic markers, proteolytic activity, mitochondrial health, cellular function, SASP cytokine expression, and methylation landscape (e.g., methylation markers or methylation clock such as the Horvath methylation clock).

In some embodiments, the rejuvenated immune cells have a transcriptomic profile, proteomic, and/or secretomic profile that is more similar to such profiles of young immune cells. For example, with respect to T cells, exhausted T cells exhibit sequential phenotypic and functional changes. Exhausted T cells express arrays of inhibitory molecules and distinctive patterns of cytokine receptors, transcription factors and effector molecules, which distinguish these cells from conventional effector, memory and anergic T cells. For example, changes in expression patterns of GranB, granzyme B; IFN-γ, interferon-γ; IL-2, interleukin-2; TNF-α, tumor necrosis factor-α may be indicative of T cell exhaustion and/or rejuvenation. Exhausted T (T_{EX}) cells express high levels of inhibitory receptors PD-1, TIM-3, LAG-3 and TIGIT.

On the other hand, changes in expression of CD62L, CCR7 and/or TCF7, particularly increases in expression, may be associated with “sternness” characteristics in T cells and thus associated with rejuvenation, and higher and more prolonged anti-pathogen, anti-cancer, or anti-inflammatory activity. In some embodiments, interleukin-2 (IL-2) production is one of the first effector activities to be extinguished, followed by tumor necrosis factor-α (TNF-α) production. However, these gene expression pattern changes represent only a small subset of the overall alterations that manifest as the exhausted state develops.

In embodiments, the rejuvenated cells have a transcriptomic profile that is more similar to the transcriptomic profile of young cells. In embodiments, the transcriptomic profile of the rejuvenated cells includes an increase in gene expression of one or more genes selected from RPL37, RHOA, SRSF3, EPHB4, ARHGAP18, RPL31, FKBP2, MAP1LC3B2, Elf1, Phf8, Pol2s2, Taf1 and Sin3a. In embodiments, the transcriptomic profile of the rejuvenated cells includes an increase in gene expression of RPL37. In embodiments, the transcriptomic profile of the rejuvenated cells includes an increase in gene expression of RHOA. In embodiments, the transcriptomic profile of the rejuvenated cells includes an increase in gene expression of SRSF3. In embodiments, the transcriptomic profile of the rejuvenated cells includes an increase in gene expression of EPHB4. In embodiments, the transcriptomic profile of the rejuvenated cells includes an increase in gene expression of ARHGAP18. In embodiments, the transcriptomic profile of the rejuvenated cells includes an increase in gene expression of RPL31. In embodiments, the transcriptomic profile of the rejuvenated cells includes an increase in gene expression of FKBP2. In embodiments, the transcriptomic profile of the rejuvenated cells includes an increase in gene expression of MAP1LC3B2. In embodiments, the transcriptomic profile of the rejuvenated cells includes an increase in gene expression of Elf1. In embodiments, the transcriptomic profile of the rejuvenated cells includes an increase in gene expression of Phf8. In embodiments, the transcriptomic profile of the rejuvenated cells includes an increase in gene expression of Pol2s2. In embodiments, the transcriptomic profile of the rejuvenated cells includes an increase in gene expression of Taf1. In embodiments, the transcriptomic profile of the rejuvenated cells includes an increase in gene expression of Sin3a. In embodiments, the transcriptomic profile of the rejuvenated cells includes an increase in gene expression of RPL37, RHOA, SRSF3, EPHB4, ARHGAP18, RPL31, FKBP2, MAP1LC3B2, Elf1, Phf8, Pol2s2, Taf1 and Sin3a.

In embodiments, the rejuvenated cells exhibit increased gene expression of one or more nuclear and/or epigenetic markers compared to a reference value. In embodiments, the one or more nuclear and/or epigenetic markers is selected from HP1 gamma, H3K9me3, lamina support protein LAP2alpha, and SIRT1 protein. In embodiments, the rejuvenated cells exhibit increased gene expression of HP1 gamma. In embodiments, the rejuvenated cells exhibit increased gene expression of H3K9me3. In embodiments, the rejuvenated cells exhibit increased gene expression of lamina support protein LAP2alpha. In embodiments, the rejuvenated cells exhibit increased gene expression of SIRT1 protein. In embodiments, the rejuvenated cells exhibit increased gene expression of HP1 gamma, H3K9me3, lamina support protein LAP2alpha, and SIRT1 protein.

In embodiments, immune cells are rejuvenated by transient reprogramming with mRNAs encoding one or more cellular reprogramming factors. Transient reprogramming is accomplished, in some embodiments, by transfecting immune cells with non-integrative mRNAs for at least about two days and not more than about 21, 20, 19, 18, 17, 16, 15, 14, 13, 12, 1, 10, 9, 8, 7, 6, 5 or 4 days, or not more than about 10, 9, 8, 7, 6, 5 or 4 days, or not more than about 5 or 4 days. In embodiments, such transfection is performed at least once a day or less than once a day, for example once every two days, three days, four days, five days, six days, seven days, or more. In embodiments, transient reprogramming of immune cells eliminates various hallmarks of aging or exhaustion, or enhances immune cell function, while avoiding cell identity loss or de-differentiation.

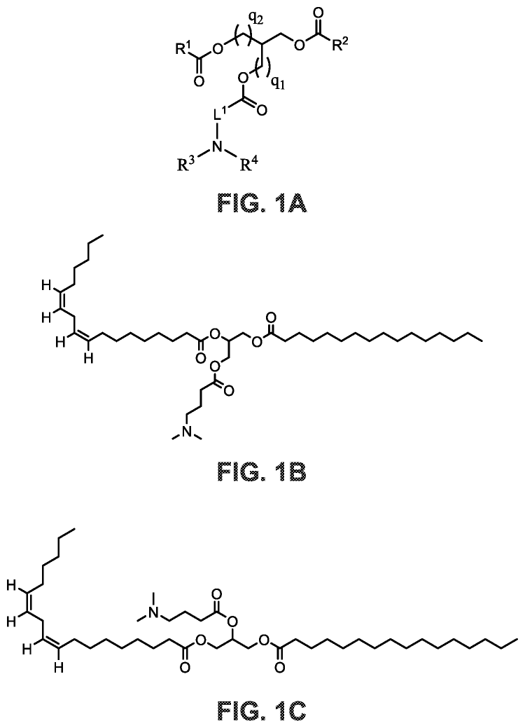

In embodiments, transfecting immune cells with messenger RNAs may be accomplished by a transfection method, including but not limited to non-viral techniques such as Lipofectamine transfection reagent, Polyplus transfection reagent, Fugene transfection reagent, LT-1 mediated transfection, dextran-mediated transfection, calcium phosphate precipitation, polybrene-mediated transfection, electroporation, microfluidic squeezing of cells, encapsulation of the mRNAs in liposomes, lipid-nanoparticle compositions, and/or direct microinjection. Transfection may be achieved using lipid nanoparticles, polymer nanoparticles, polymer polyplexes, or polymer anioplexes. In some embodiments, the lipid nanoparticles include combinations of ionizable or cationic lipids, uncharged lipids including but not limited to phospholipids and cholesterol, lipids conjugated or modified with moieties such as polyethylene glycol for stabilization, and/or biodegradable lipids. In some embodiments, the lipids are those described in FIGS. 1-18. In some embodiments, the lipids are those in Billingsley et al., Nano Lett. 2020 (20, 3, 1578-1589), incorporated herein by reference. In some embodiments, the polymers include cationic polymers; biodegradable polymers; natural polymers such as peptides, proteins, or polysaccharides; and/or charge-altering releasable transporters. In some embodiments, the polymers are charge-altering releasable transporters. The charge-altering releasable transporter may contain mixtures of lipophilic blocks in either block (b) or statistical (stat) architectures, i.e., it may be at least one “block CART” or “stat CART” such as those described in McKinlay et al. 2017 (PNAS Jan. 24, 2017114(4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. In some embodiments, lipids are combined with polymers to generate hybrid nanoparticles or micelles. In some embodiments, the use of a lipid or polymer for delivery of the mRNA, such as in a lipid nanoparticle, polymer nanoparticle, or hybrid lipid-polymer nanoparticle, results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA. In some embodiments, the use of a lipid or polymer for delivery of the mRNA results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA due to lower toxicity and/or lower physiological impact on the cell when compared to the different delivery mechanism. In some embodiments, the different delivery mechanism is electroporation, i.e., the use of a lipid or polymer for delivery of the mRNA results in enhanced rejuvenation, proliferation, recovery from or prevention of exhaustion, anti-pathogenic effects, anti-cancer effects, or anti-inflammatory effects in the exposed immune cell compared to using electroporation. This improvement compared to electroporation can result from reduced toxicity or reduced physiological impact on the cell compared to electroporation.

The compositions, methods and kits described herein relate to delivery of nucleic acids and other therapeutic molecules to target cells and/or tissues. For example, compositions, methods and kits described herein may relate to delivery of DNA and/or mRNA to a cell by various gene delivery systems. “Gene delivery” refers to any method or system for introducing exogenous nucleic acid sequences into a target, such as a target cell or tissue, including diseased and/or aging cells or tissues. In some embodiments, gene delivery systems may be categorized as: viral-based, non-viral-based and combined hybrid systems. In some embodiments, non-viral gene delivery systems provide an alternative to viral-based systems. One of the most important advantages of these systems is improved safety through enhanced control of transgene expression and dosing as well as lack of chromosomal integration. Such systems include systems for delivering mRNA.

Some non-viral gene delivery systems may be referred to as physical or chemical methods. In some embodiments, physical methods include, but are not limited to, microinjection, jet injection, electroporation, ultrasound, gene gun, and hydrodynamic systems. In general terms, physical methods refer to delivery of the gene via the application of physical force to increase permeability of the cell membrane. In contrast, chemical methods utilize natural or synthetic carriers to deliver genes into cells. In some embodiments, polymers, cationic polymers, liposomes, lipids, cationic lipids, helper lipids, spacer lipids, ionizable lipids, dendrimers, or nanoparticle compositions formed from any of the foregoing or any combination of the foregoing may be used as gene delivery systems in conjunction with the compositions, methods and kits described herein. Such gene delivery systems may be used to deliver the mRNA described herein. Such nanoparticle compositions may also be sued to deliver non-gene therapeutic molecules such a proteins, peptides, or small molecules.

In some embodiments, lipid-based delivery system such as, but not limited to lipid nanoparticles (LNP) may provide an approach to stabilize and deliver nucleic acids and other therapeutic molecules such as proteins, peptides, and/or small molecules. Design features, such as optimal particle size, high encapsulation efficiency, robust manufacturing process, and optimal lipophilicity and surface charge, are included to provide efficient lipid-based delivery systems for nucleic acids and other therapeutic molecules.

In some embodiments, the lipids for transfecting immune cells, such as T cells, provided herein relate to an ionizable lipid of Formula (I)

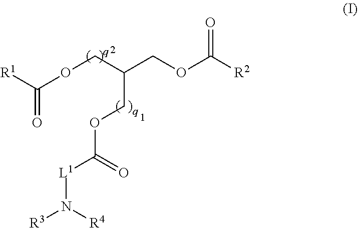

or a pharmaceutically acceptable salt or a stereoisomer thereof, wherein:
L¹ is C₁-C₆ alkylene; R¹ is C₆-C₆₀ alkenyl; R² is C₆-C₂₀ alkyl; R³ and R⁴ are each independently H or C₁-C₃ alkyl; q₁ is absent or 1; and q₂ is absent or 1 (FIG. 1A).

In other embodiments, the ionizable lipid of Formula (I) have one of the following structures (FIG. 1B and FIG. 1C):

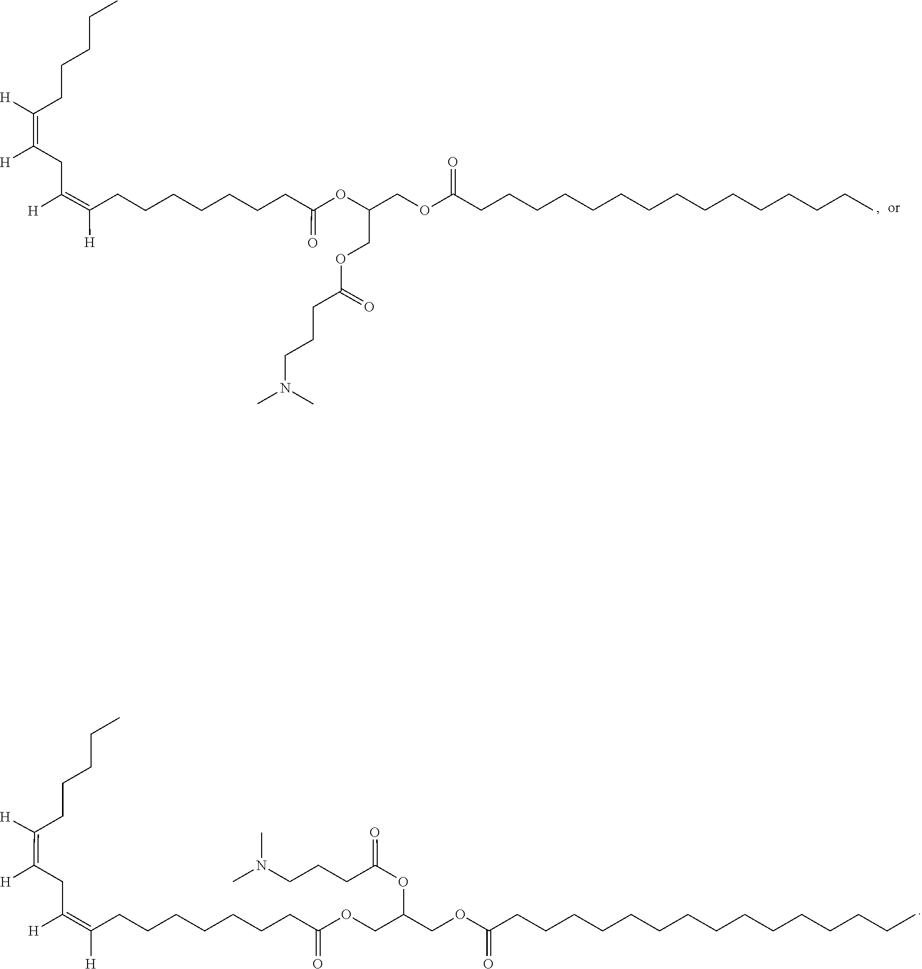 [SMILES: CCCCC/C=C\C/C=C\CCCCCCCC(=O)OC(COC(=O)CCCCCCCCCCCCCCC)COC(=O)CCCN(C)C ; CCCCC/C=C\C/C=C\CCCCCCCC(=O)OCC(COC(=O)CCCCCCCCCCCCCCC)OC(=O)CCCN(C)C]

In other embodiments, the lipids for transfecting immune cells provided herein relate to an ionizable lipid of Formula (I-A)

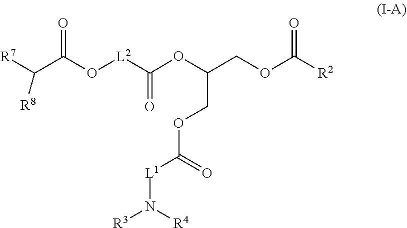

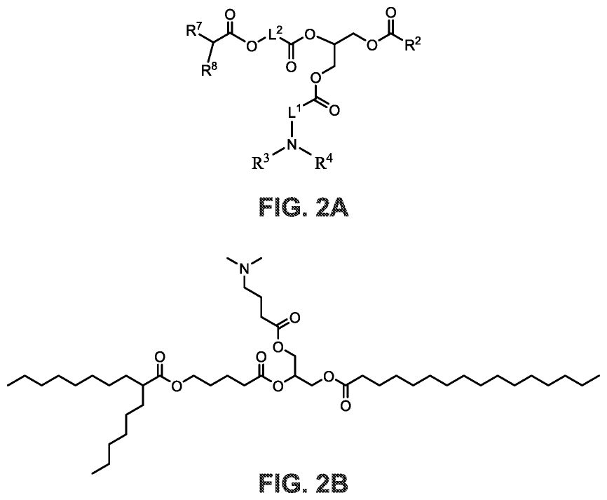

or a pharmaceutically acceptable salt or a stereoisomer thereof, wherein:
L¹ is C₁-C₆ alkylene; L² is C₁-C₈ alkylene; R² is C₆-C₂₀ alkyl; R³ and R⁴ are each independently H or C₁-C₃ alkyl; R⁷ is C₄-C₂₀alkyl; and R⁸ is C₄-C₂₀ alkyl (FIG. 2A).

In other embodiments, the ionizable lipids of Formula (I-A) have the following structure (FIG. 2B):

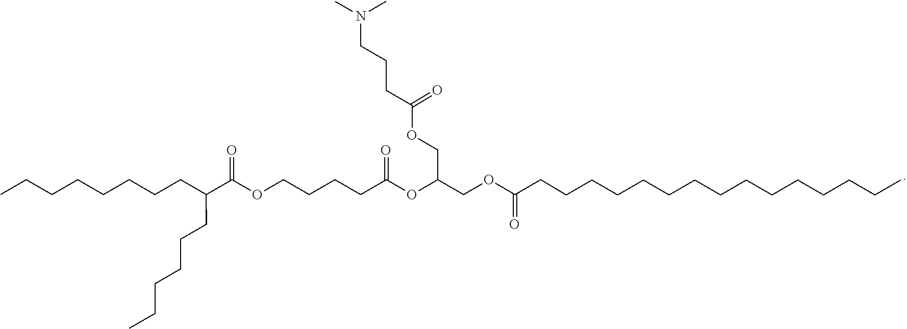 [SMILES: CCCCCCCCCCCCCCCC(=O)OCC(COC(=O)CCCN(C)C)OC(=O)CCCCOC(=O)C(CCCCCC)CCCCCCCC]

In one embodiment, the lipids relate to an ionizable lipid of Formula (I-B)

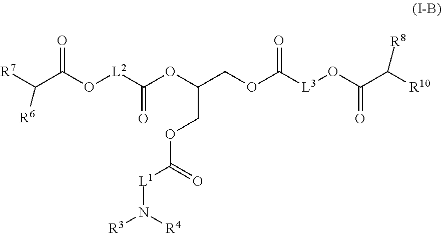

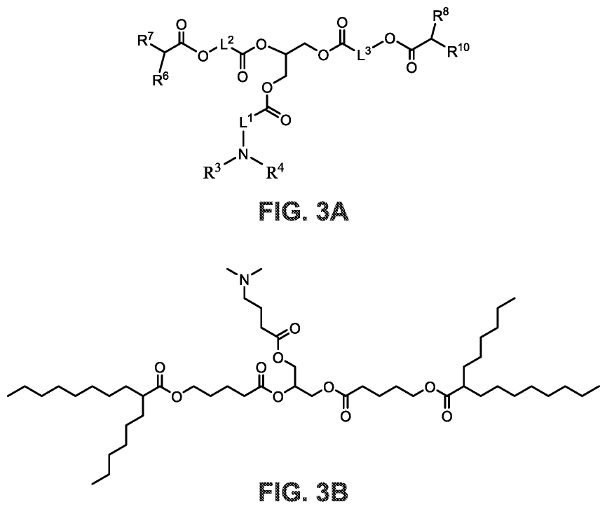

or a pharmaceutically acceptable salt or astereoisomer thereof, wherein:
   L¹ is C₁-C₆ alkylene; L² is C₁-C₈ alkylene; L³ is C₁-C₈ alkylene; R³ and R⁴ are each independently H or C₁-C₃ alkyl; R⁶ is C₄-C₂₀alkyl; R⁷is C₄-C₂₀alkyl; R⁸ is C₄-C₂₀ alkyl; and R¹⁰ is C₄-C₂₀ alkyl (FIG. 3A).

In another embodiment, the ionizable lipids of Formula (I-B) have the following structure (FIG. 3B):

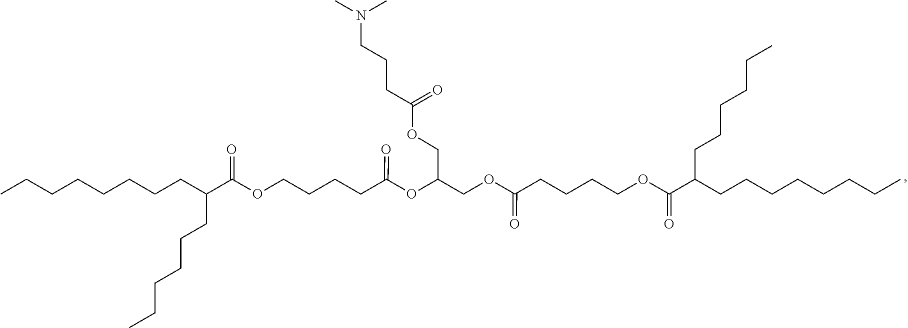 [SMILES: CCCCCCCCC(CCCCCC)C(=O)OCCCCC(=O)OCC(COC(=O)CCCN(C)C)OC(=O)CCCCOC(=O)C(CCCCCC)CCCCCCCC]

In another embodiment, the lipids relate to an ionizable lipid of Formula (II)

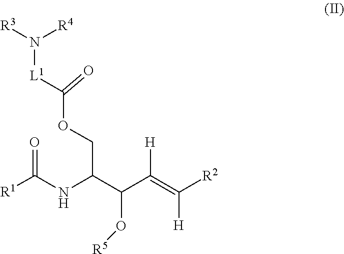

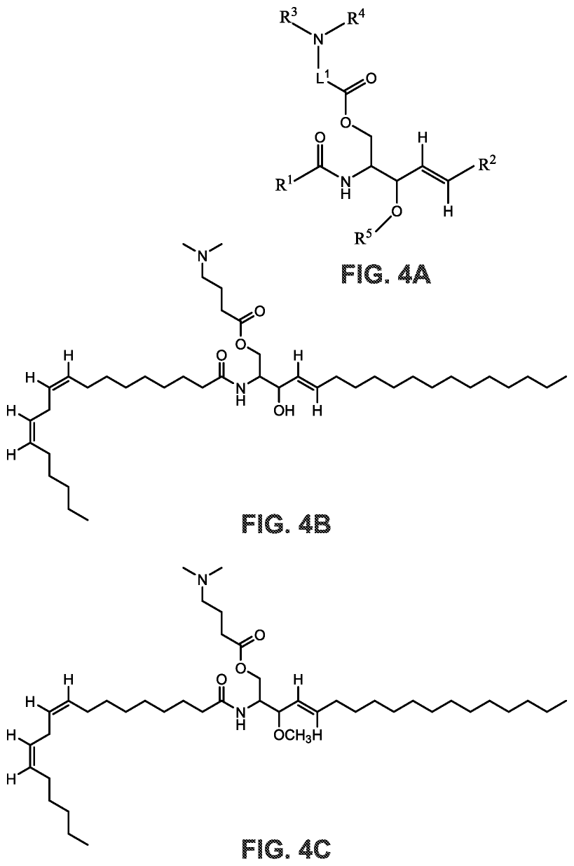

or a pharmaceutically acceptable salt or a stereoisomer thereof, wherein:
L¹ is C₁-C₆ alkylene; R¹ is C₆-C₂₀ alkenyl; R² is C₆-C₂₀ alkyl; R³ and R⁴ are each independently H or C₁-C₃ alkyl; and R⁵ is H or C₁-C₃ alkyl (FIG. 4A).

In other embodiment, the ionizable lipid of Formula (II) has one of the following structures (FIG. 4B and FIG. 4C):

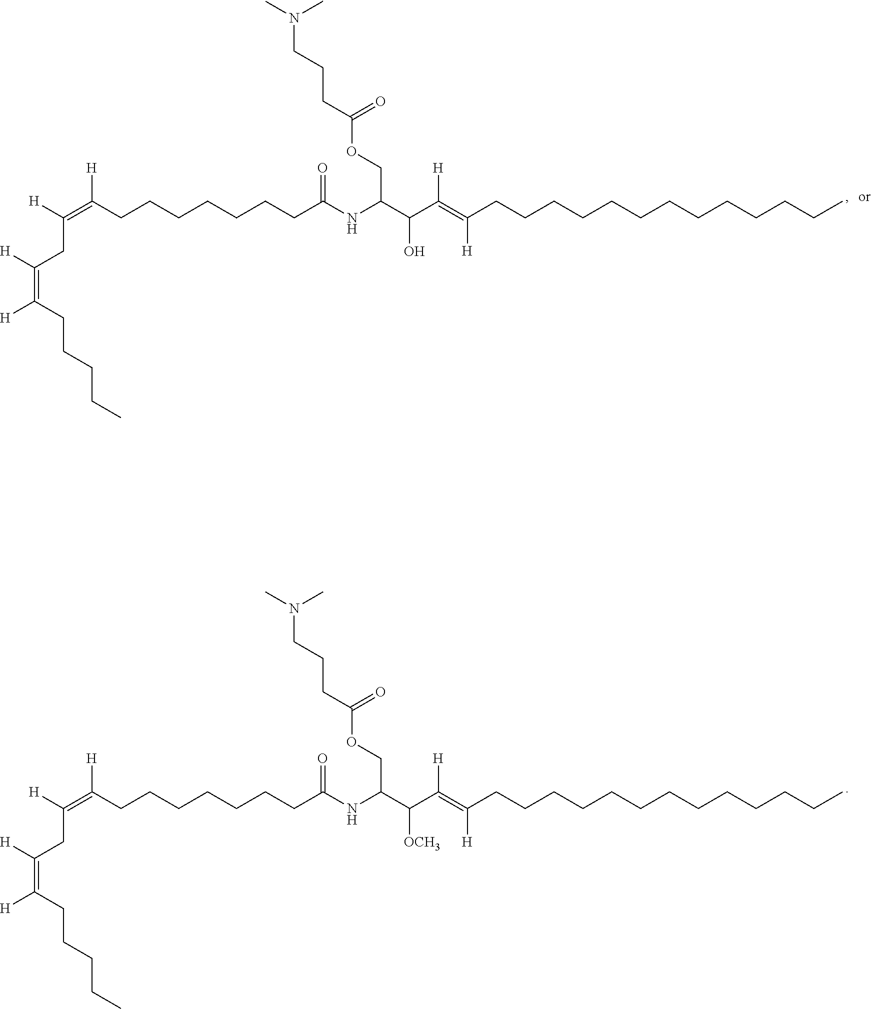 [SMILES: CCCCC/C=C\C/C=C\CCCCCCCC(=O)NC(COC(=O)CCCN(C)C)C(O)/C=C/CCCCCCCCCCCCC ; CCCCC/C=C\C/C=C\CCCCCCCC(=O)NC(COC(=O)CCCN(C)C)C(/C=C/CCCCCCCCCCCCC)OC]

In another embodiment, the lipids relate to an ionizable lipid of Formula (III)

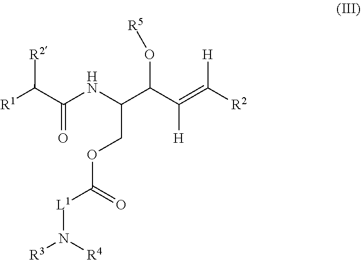

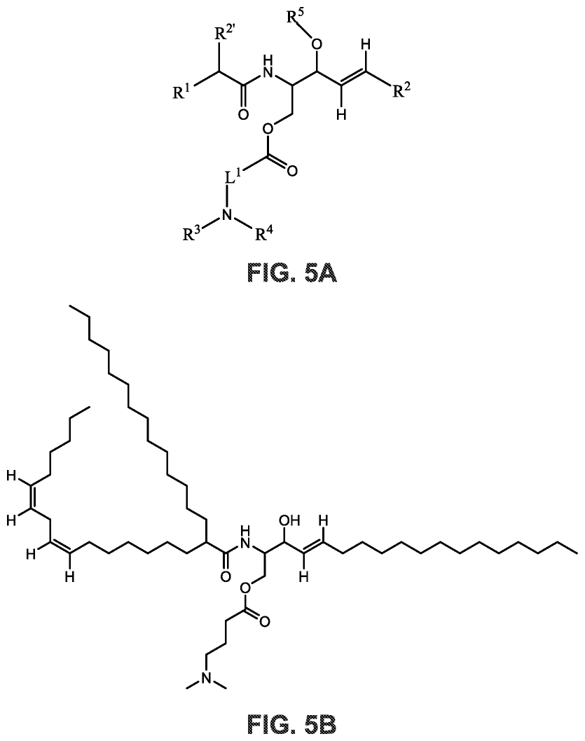

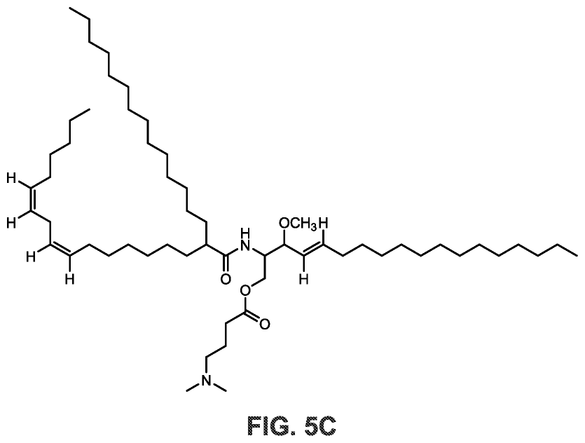

or a pharmaceutically acceptable salt or a stereoisomer thereof, wherein:
L¹ is C₁-C₆ alkylene; R¹ is C₆-C₂₀ alkenyl; R² is C₆-C₂₀ alkyl; R^{2′} is C₆-C₂₀ alkyl; R³ and R⁴ are each independently H or C₁-C₃ alkyl; and R⁵ is H or C₁-C₃ alkyl (FIG. 5A).

In other embodiment, the ionizable lipids of Formula (III) have one of the following structures (FIG. 5B and FIG. 5C):

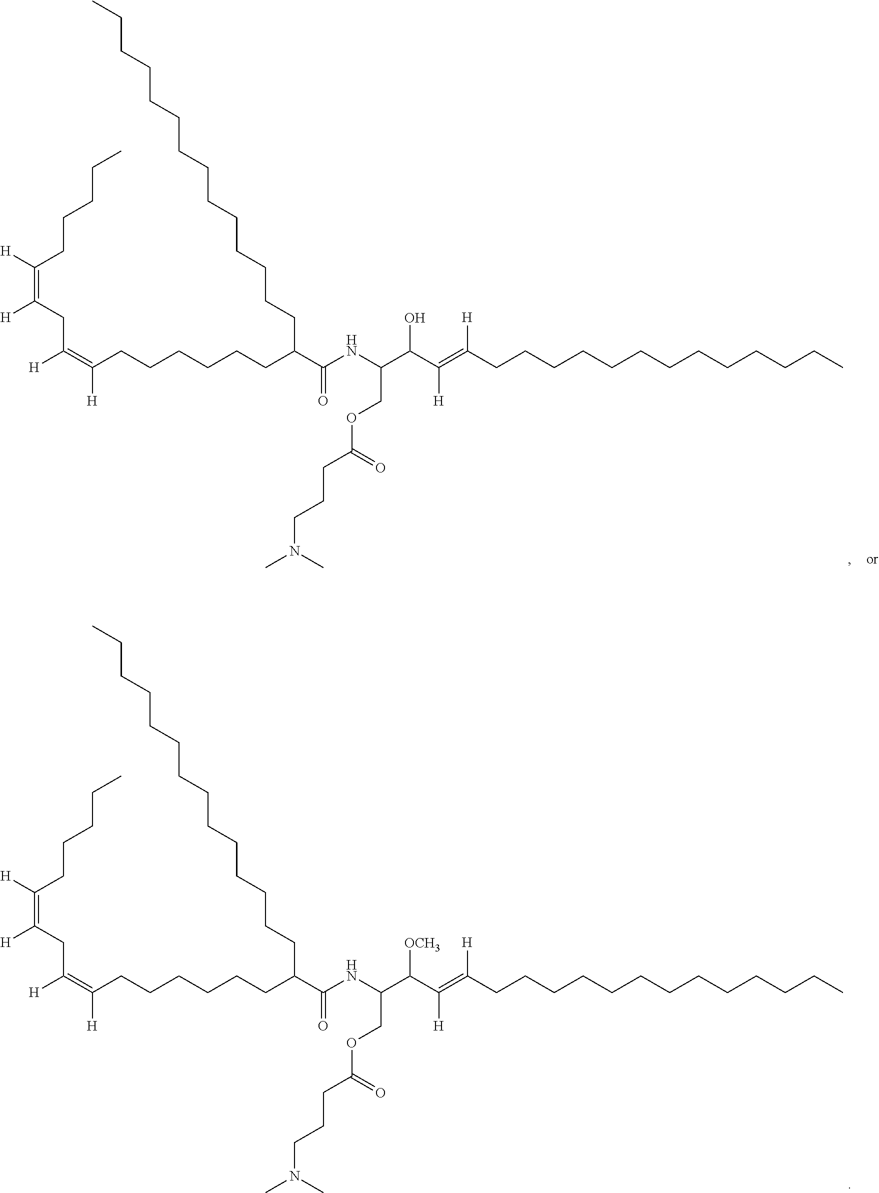 [SMILES: CCCCC/C=C\C/C=C\CCCCCCC(CCCCCCCCCCCCCC)C(=O)NC(COC(=O)CCCN(C)C)C(O)/C=C/CCCCCCCCCCCCC ; CCCCC/C=C\C/C=C\CCCCCCC(CCCCCCCCCCCCCC)C(=O)NC(COC(=O)CCCN(C)C)C(/C=C/CCCCCCCCCCCCC)OC]

In another embodiment, the lipids relate to an ionizable lipid of Formula (IV)

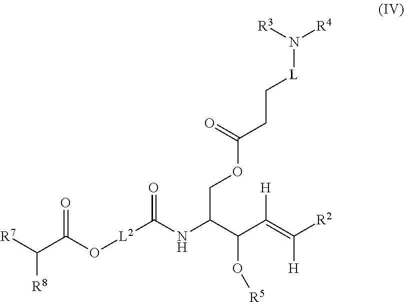

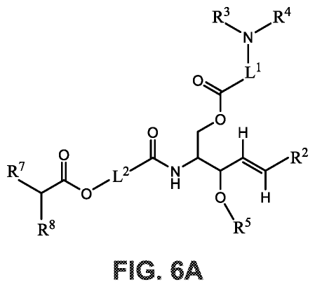

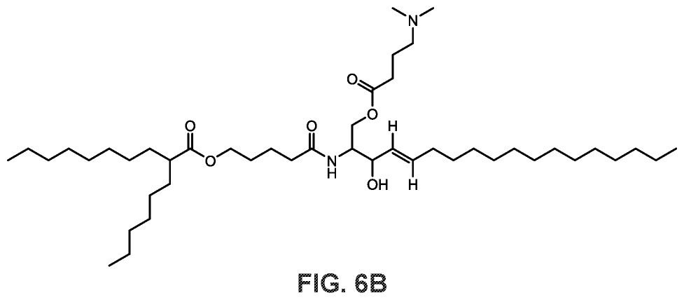

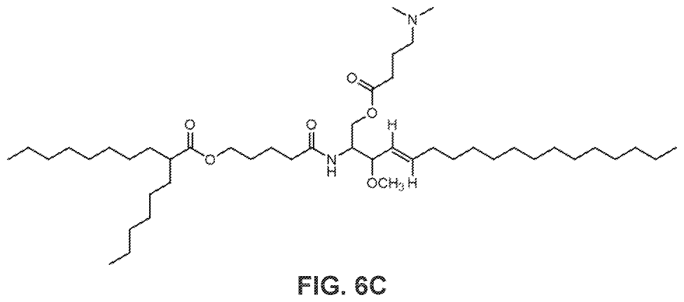

or a pharmaceutically acceptable salt or a stereoisomer thereof, wherein:
L¹ is C₁-C₆ alkylene; L² is C₁-C₈ alkylene; R² is C₆-C₂₀ alkyl; R³ and R⁴ are each independently H or C₁-C₃ alkyl; W⁵ is H or C₁-C₃ alkyl; R⁷ is C₄-C₂₀ alkyl; and R⁸ is C₄-C₃₀ alkyl (FIG. 6A).

In other embodiment, the ionizable lipid of Formula (IV) has one of the following structures (FIG. 6B and FIG. 6C):

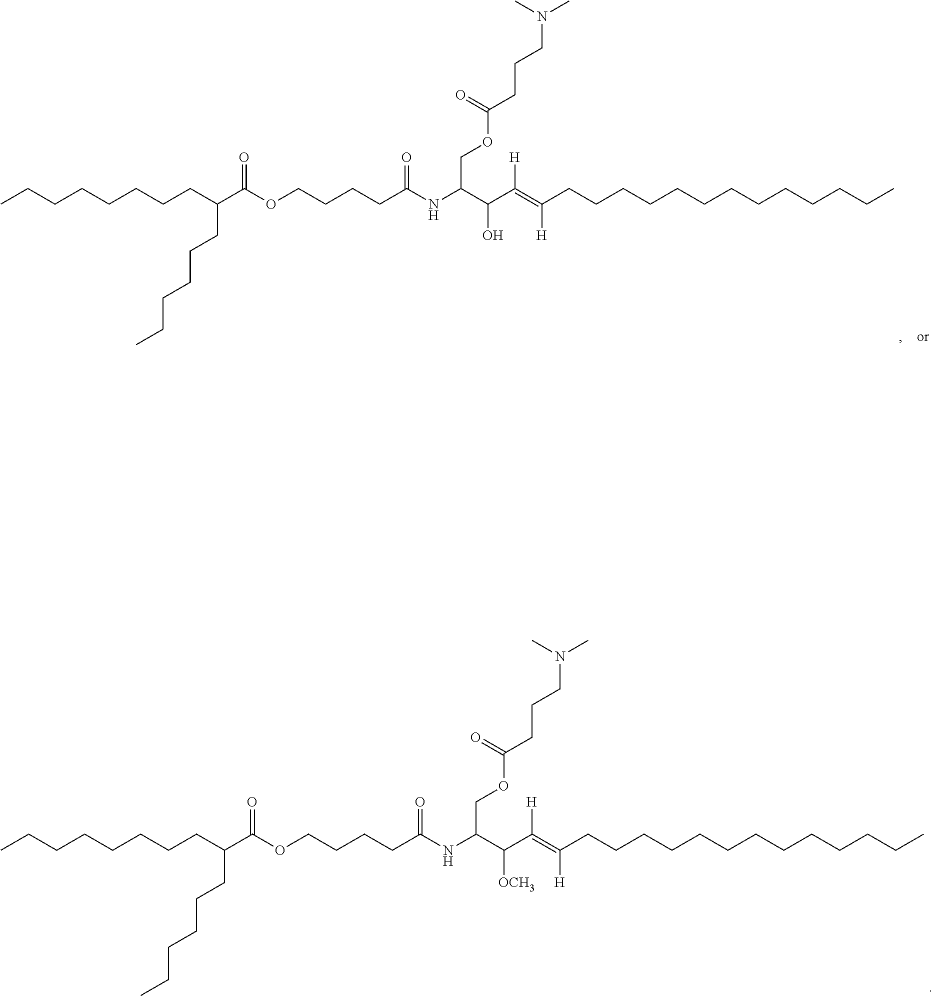

In another embodiment, the lipids relate to an ionizable lipid of Formula (V)

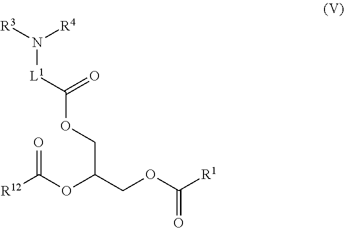

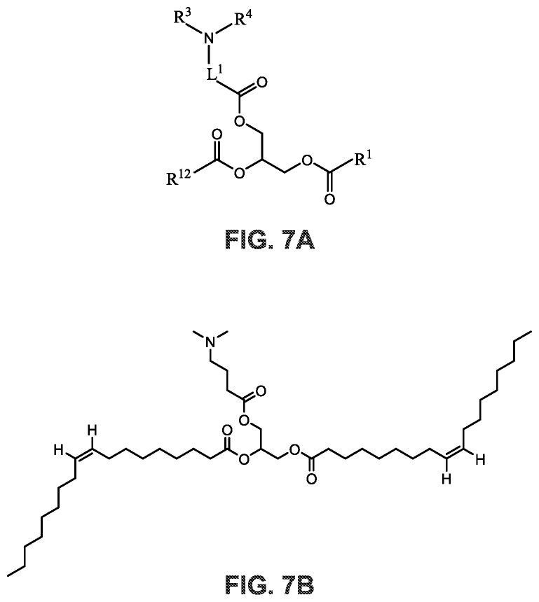

or a pharmaceutically acceptable salt or a stereoisomer thereof, wherein:
L¹ is C₁-C₆ alkylene; R¹ is C₆-C₂₀ alkenyl; R³ and R⁴ are each independently H or C₁-C₃ alkyl; and R¹² is C₆-C₂₀ alkenyl (FIG. 7A).

In other embodiment, the ionizable lipids of Formula (V) have the following structure (FIG. 7B):

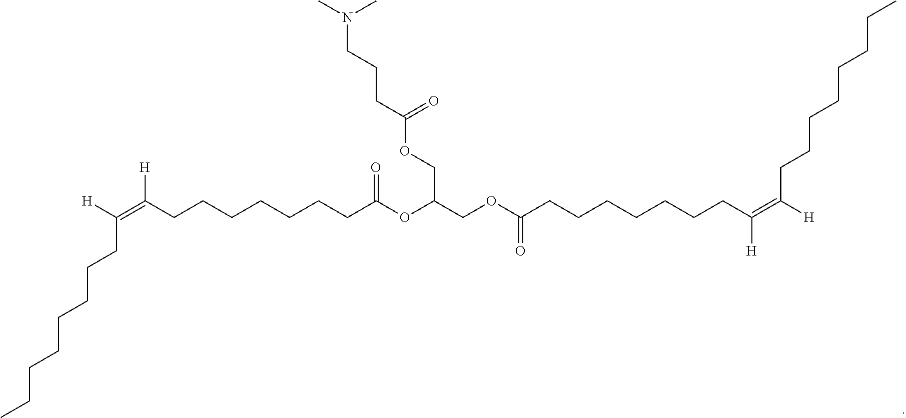 [SMILES: CCCCCCCC/C=C\CCCCCCCC(=O)OCC(COC(=O)CCCN(C)C)OC(=O)CCCCCCC/C=C\CCCCCCCC]

In another embodiment, the lipids relate to an ionizable lipid of Formula (VI)

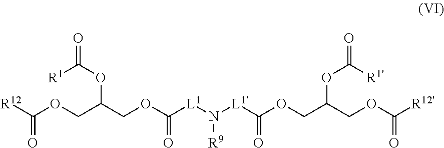

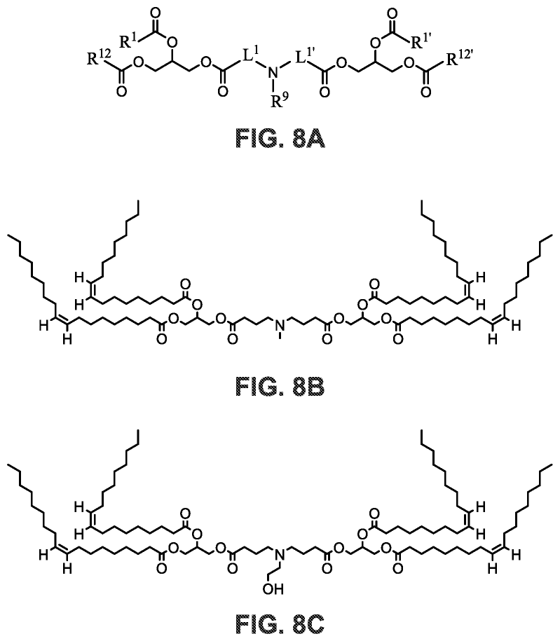

or a pharmaceutically acceptable salt or a stereoisomer thereof, wherein:
L¹ is C₁-C₆ alkylene; L^{1′} is C₁-C₆ alkylene; R¹ is C₆-C₂₀ alkenyl; R^{1′} is C₆-C₂₀ alkenyl; R⁹ is H, C₁-C₆ alkyl or —(CH₂)ₙOH; R¹² is C₆-C₂₀ alkenyl; R^{12′} is C₆-C₂₀ alkenyl; and n is 2, 3 or 4 (FIG. 8A).

In other embodiments, the ionizable lipids of Formula (VI) have one of the following structures (FIG. 8B and FIG. 8C):

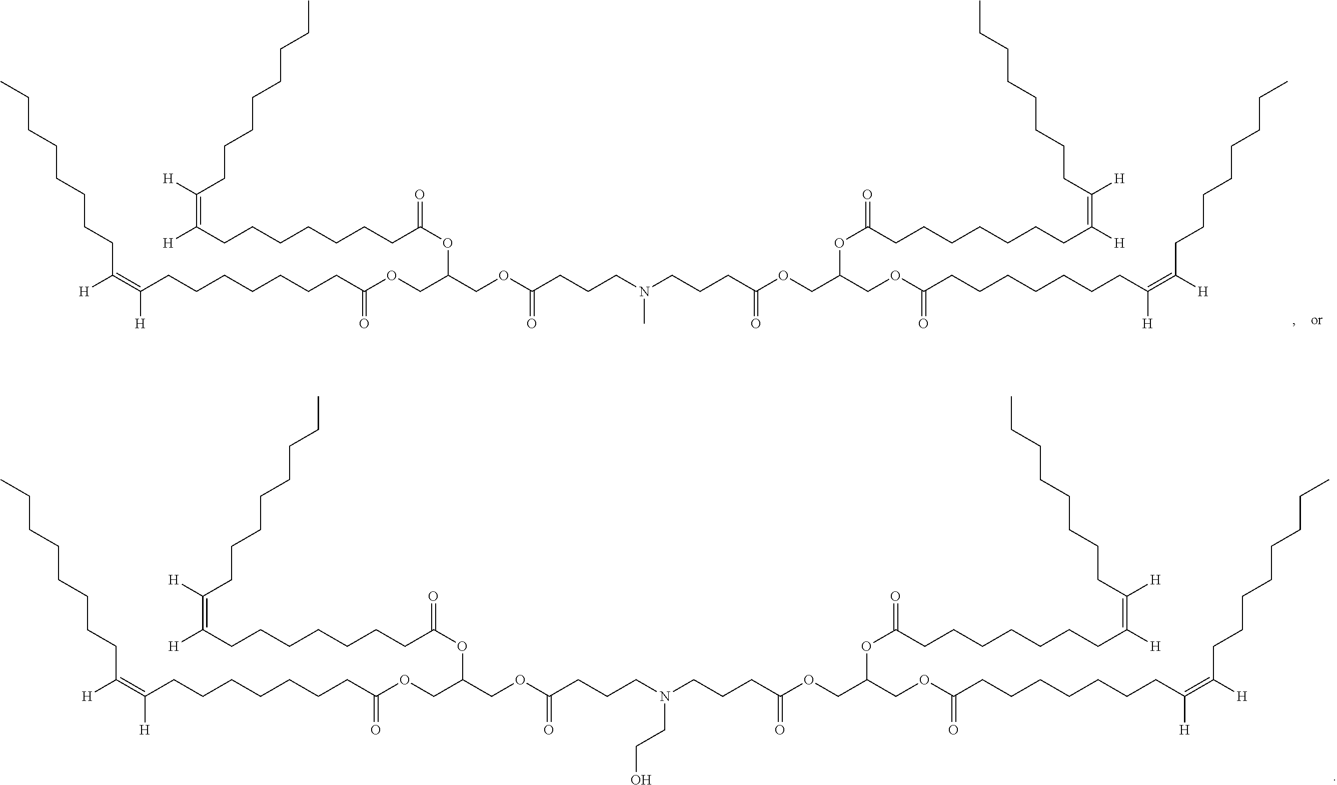

In another embodiment, the lipids relate to an ionizable lipid of Formula (VII)

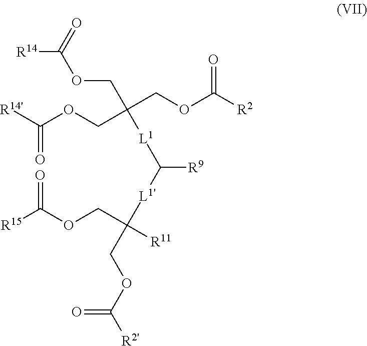

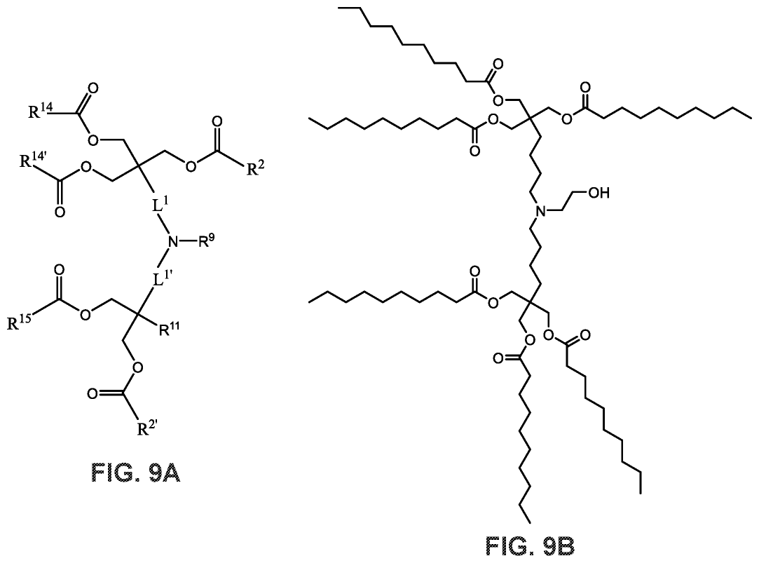

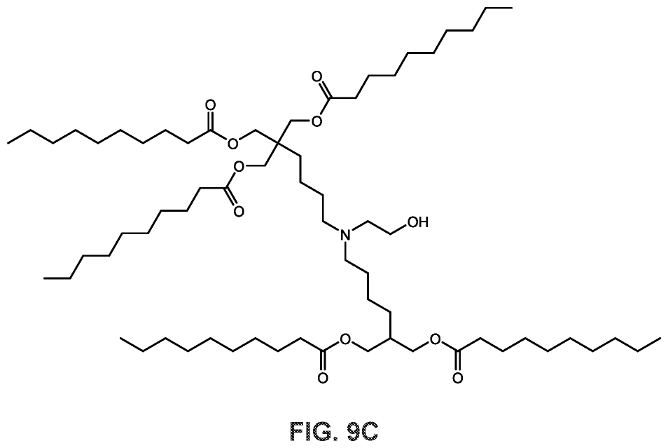

or a pharmaceutically acceptable salt or a stereoisomer thereof, wherein:
L¹ is C₁-C₆ alkylene; L^{1′} is C₁-C₆ alkylene; R² is C₆-C₂₀ alkyl; R^{2′} is C₆-C₂₀ alkyl; R⁹ is H, C₁-C₆ alkyl or —(CH₂)ₙOH; R¹ is H or —CH₂)OC(═O)R¹⁶; R¹⁴ is C₆-C₂₀ alkyl; R^{14′} is C₆-C₂₀ alkyl; R¹⁵ is C₆-C₂₀ alkyl; R¹⁶ is C₆-C₂₀ alkyl; and n is 2, 3 or 4 (FIG. 9A).

In another embodiment, the ionizable lipids of Formula (VII) have one of the following structures (FIG. 9B and FIG. 9C):

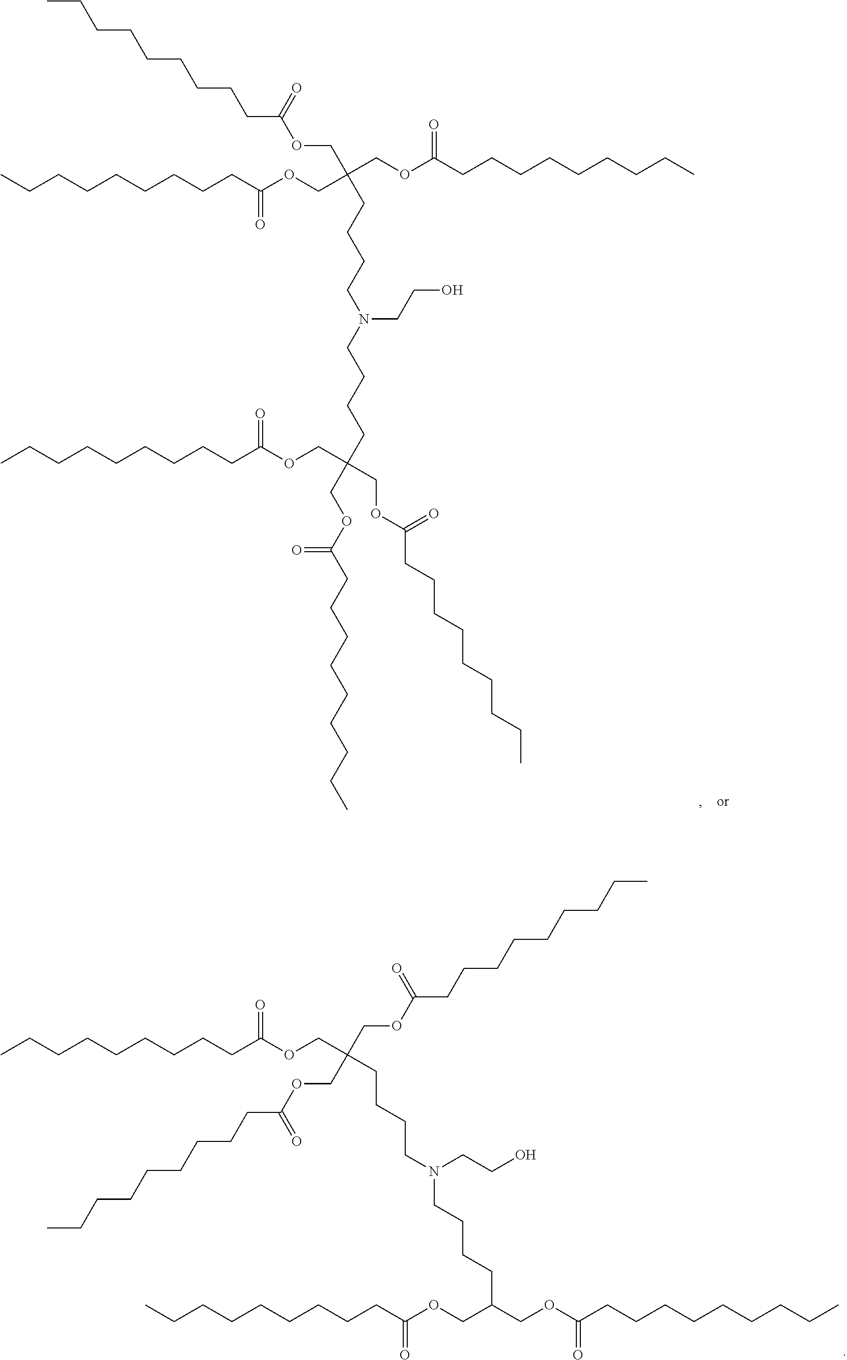

In another embodiment, the lipids relate to ionizable lipids of Formula (VIII)

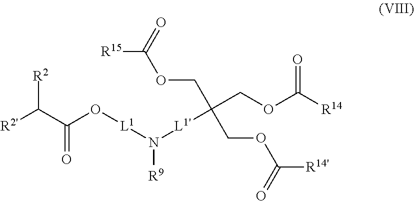

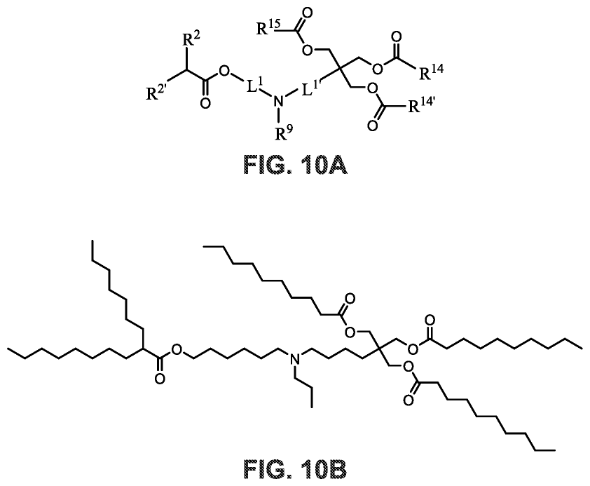

or a pharmaceutically acceptable salt or a stereoisomer thereof, wherein:
L¹ is C₁-C₆ alkylene; L^{1′} is C₁-C₆ alkylene; R² is C₆-C₂₀ alkyl; R^{2′} is C₆-C₂₀ alkyl; R⁹ is H, C₁-C₆ alkyl or —(CH₂)ₙOH; R¹⁴ is C₆-C_{3D} alkyl; R^{14′} is C₆-C₂₀ alkyl; R¹⁵ is C₆-C₂₀ alkyl; and n is 2, 3 or 4 (FIG. 10A).

In some embodiments, the ionizable lipids of Formula (VIII) have the following structure (FIG. 10B):

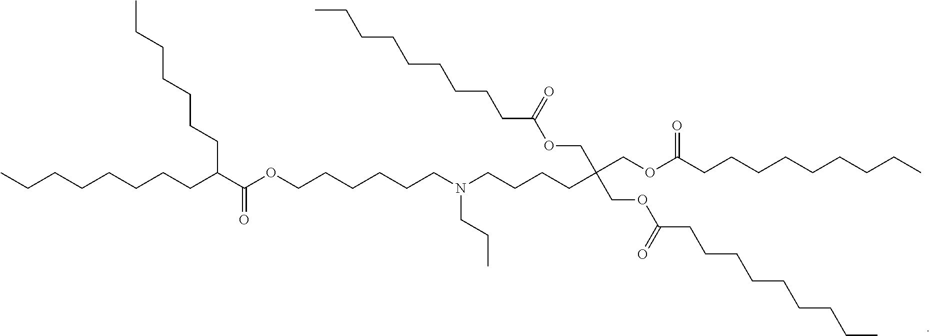 [SMILES: CCCCCCCCCC(=O)OCC(CCCCN(CCC)CCCCCCOC(=O)C(CCCCCCC)CCCCCCCC)(COC(=O)CCCCCCCCC)COC(=O)CCCCCCCCC]

In another embodiment, the lipids relate to an ionizable lipid of Formula (IX)

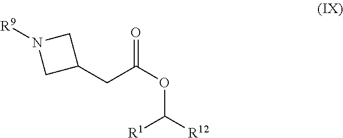

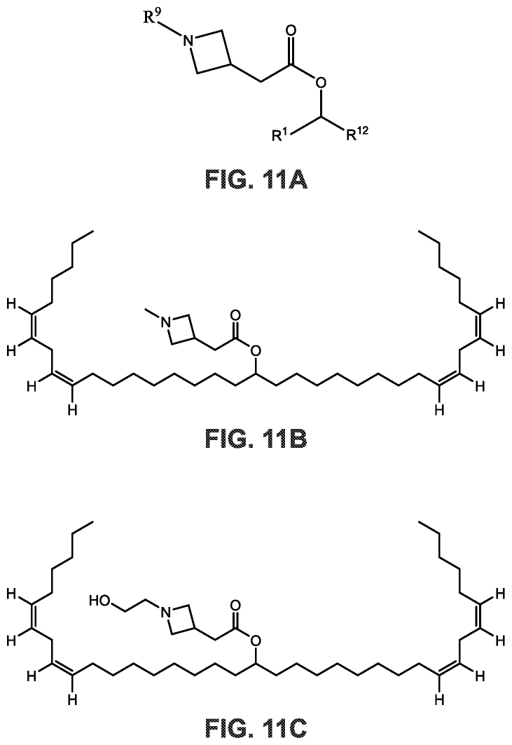

or a pharmaceutically acceptable salt or a stereoisomer thereof, wherein:
R¹ is C₆-C₂₀ alkenyl; R⁹ is H, C₁-C₆ alkyl or —(CH₂)ₙOH; R¹² is C₆-C₂₀ alkenyl; and n is 2, 3 or 4 (FIG. 11A).

In some embodiments, the ionizable lipids of Formula (IX) have one of the following structures (FIG. 11B and FIG. 11C):

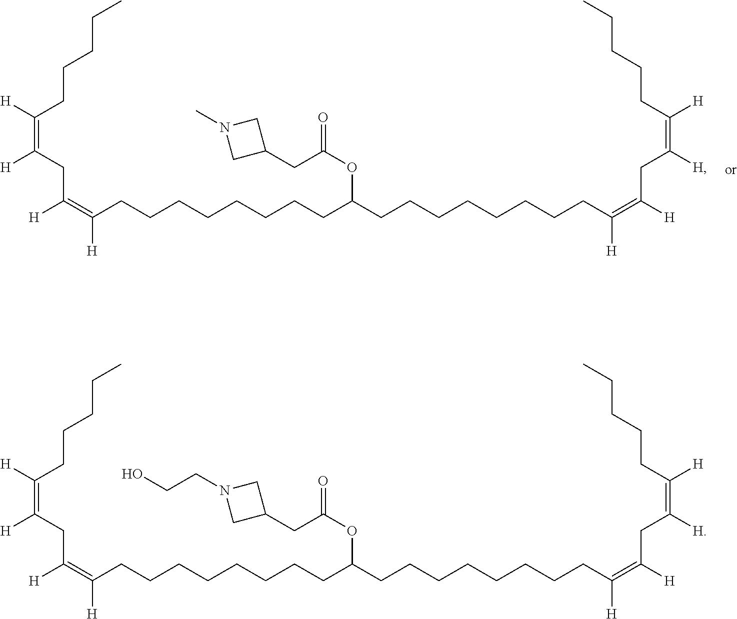

In another embodiment, the lipids relate to an ionizable lipid of Formula (X)

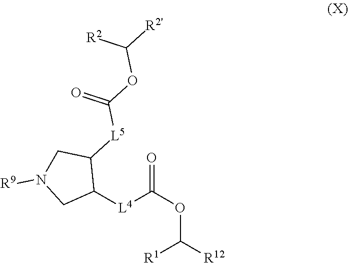

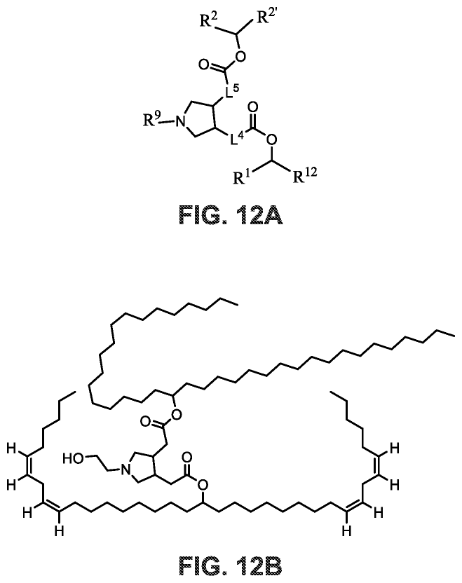

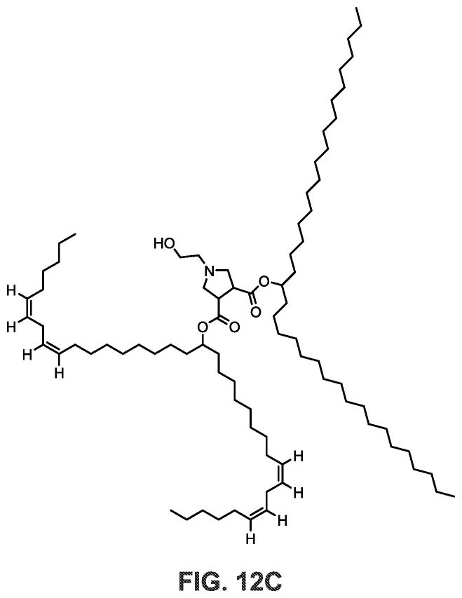

or a pharmaceutically acceptable salt or a stereoisomer thereof, wherein:
L⁴ is absent or C₁-C₆ alkylene; L⁵ is absent or C₁-C₆ alkylene; R¹ is C₆-C₂₀ alkenyl; R² is C₆-C₂₀ alkyl; R^{2′} is C₆-C₂₀ alkyl; R⁹ is H, C₁-C₆ alkyl or —(CH₂)ₙOH; R¹² is C₆-C₂₀ alkenyl; and n is 2, 3 or 4 (FIG. 12A).

In some embodiments, the ionizable lipids of Formula (X) have one of the following structures (FIG. 12B and FIG. 12C):

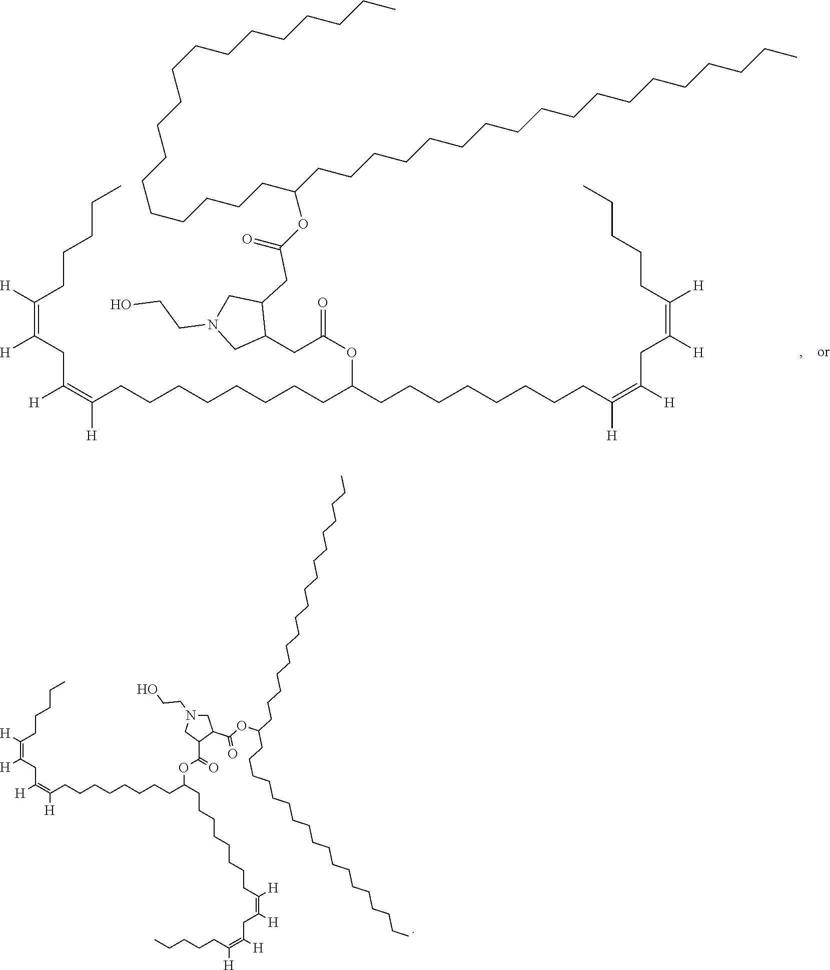 [SMILES: CCCCC/C=C\C/C=C\CCCCCCCCC(CCCCCCCC/C=C\C/C=C\CCCCC)OC(=O)CC1CN(CCO)CC1CC(=O)OC(CCCCCCCCCCCCCCCCCC)CCCCCCCCCCCCCCCCCC ; CCCCC/C=C\C/C=C\CCCCCCCCC(CCCCCCCC/C=C\C/C=C\CCCCC)OC(=O)C1CN(CCO)CC1C(=O)OC(CCCCCCCCCCCCCCCCCC)CCCCCCCCCCCCCCCCCC]

In another embodiment, the lipids relate to an ionizable lipid of Formula (XI)

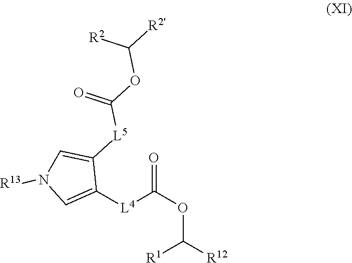

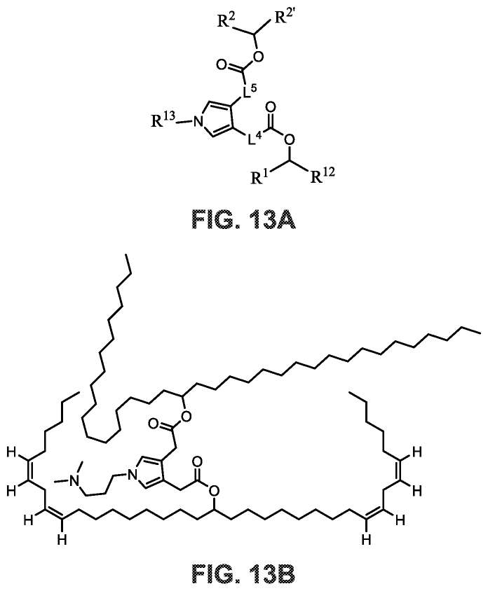

or a pharmaceutically acceptable salt or a stereoisomer thereof, wherein:
L⁴ is absent or C₁-C₆ alkylene; L⁵ is absent or C₁-C₆ alkylene; R¹ is C₆-C₂₀ alkenyl; R² is C₆-C₂₀ alkyl; R^{2′} is C₆-C₂₀ alkyl; R¹² is C₆-C₂₀ alkenyl; R¹³ is H, C₁-C₆ alkyl, —(CH₂)ₙOH or —(CH₂)_{q}N(CH₃)₂; n is 2, 3 or 4; and q is 2, 3 or 4 (FIG. 13A).

In some embodiments, the ionizable lipids of Formula (XI) have the following structure (FIG. 13B):

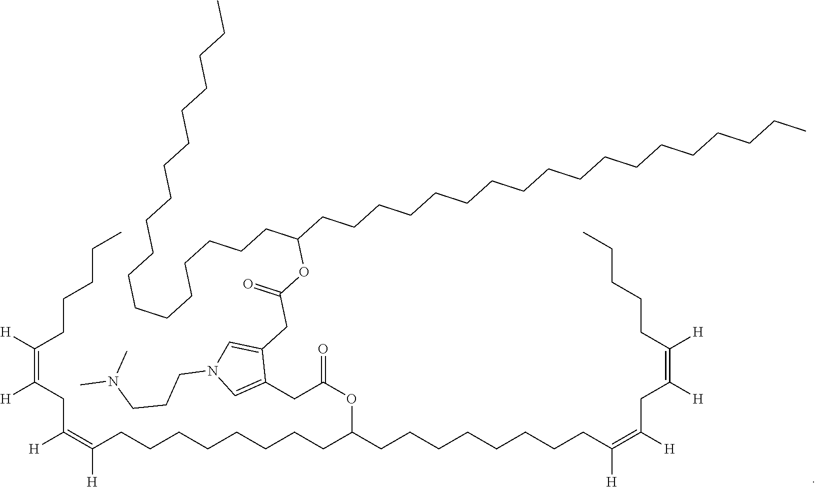 [SMILES: CCCCC/C=C\C/C=C\CCCCCCCCC(CCCCCCCC/C=C\C/C=C\CCCCC)OC(=O)Cc1cn(CCCN(C)C)cc1CC(=O)OC(CCCCCCCCCCCCCCCCCC)CCCCCCCCCCCCCCCCCC]

In another embodiment, the lipids relate to an ionizable lipid of Formula (XII)

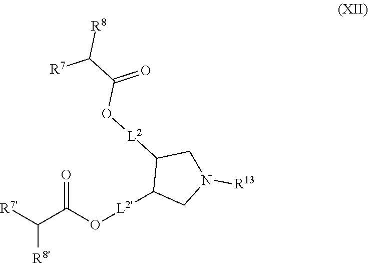

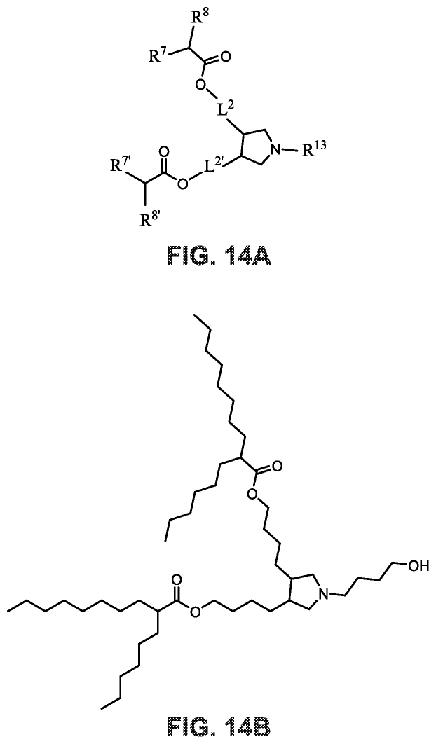

or a pharmaceutically acceptable salt or a stereoisomer thereof, wherein:
L² is C₁-C₈ alkylene; L^{2′} is C₁-C₈ alkylene; R⁷ is C₄-C₂₀ alkyl; R^{7′} is C₄-C₂₀ alkyl; R⁸ is C₄-C₂₀ alkyl; R^{8′} is C₄-C₂₀ alkyl; R¹³ is H, C₁-C₆ alkyl, —(CH₂)ₙOH, or —(CH₂)_{q}N(CH₃)₂; n is 2, 3 or 4; and q is 2, 3 or 4 (FIG. 14A).

In some embodiments, the ionizable lipids of Formula (XII) have the following structure (FIG. 14B):

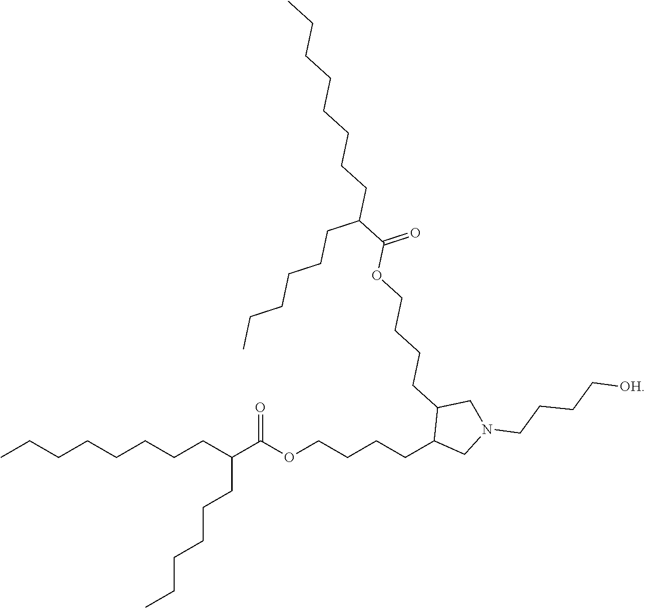 [SMILES: CCCCCCCCC(CCCCCC)C(=O)OCCCCC1CN(CCCCO)CC1CCCCOC(=O)C(CCCCCC)CCCCCCCC]

In another embodiment, the lipids relate to an ionizable lipid of Formula (XIII)

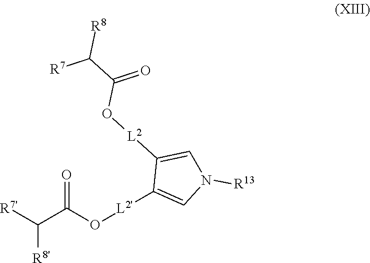

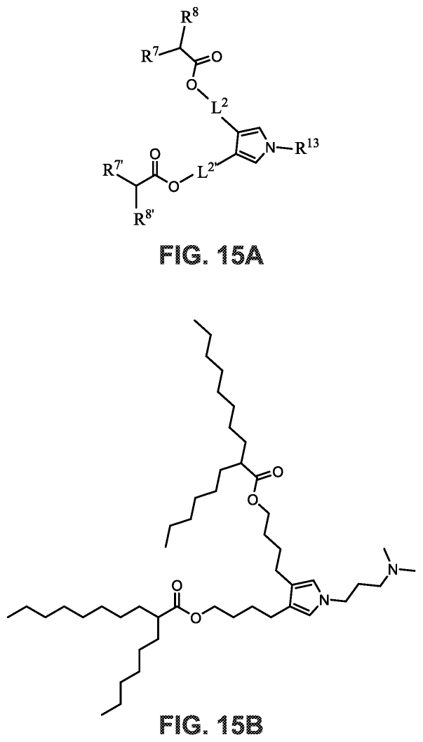

or a pharmaceutically acceptable salt or a stereoisomer thereof, wherein:
L² is C₁-C₈ alkylene; L^{2′} is C₁-C₈ alkylene; R⁷ is C₄-C₂₀ alkyl; R^{7′} is C₄-C₂₀ alkyl; R⁸ is C₄-C₂₀ alkyl; R^{8′} is C₄-C₂₀ alkyl; R¹³ is H, C₁-C₆ alkyl, —(CH₂)ₙOH, or —(CH₂)_{q}N(CH₃)₂; n is 2, 3 or 4; and q is 2, 3 or 4 (FIG. 15A).

In some embodiments, the ionizable lipids of Formula (XIII) have the following structure (FIG. 15B):

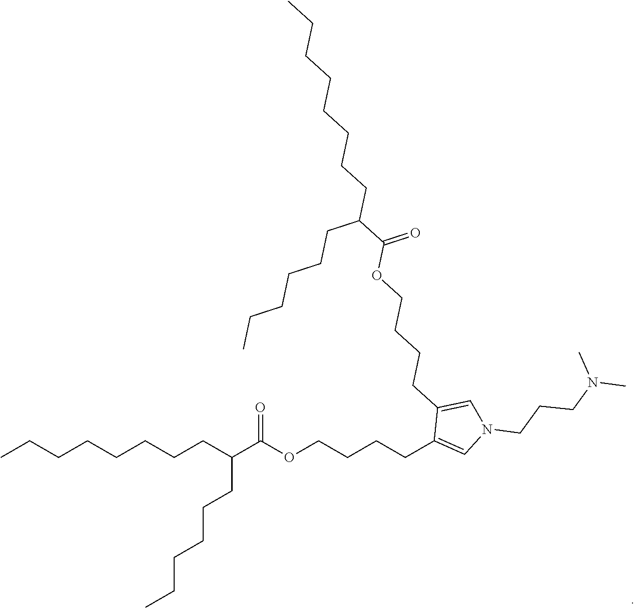 [SMILES: CCCCCCCCC(CCCCCC)C(=O)OCCCCc1cn(CCCN(C)C)cc1CCCCOC(=O)C(CCCCCC)CCCCCCCC]

In another embodiment, the lipids relate to an ionizable lipid of Formula (XIV)

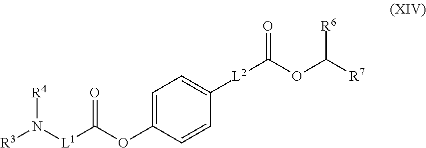

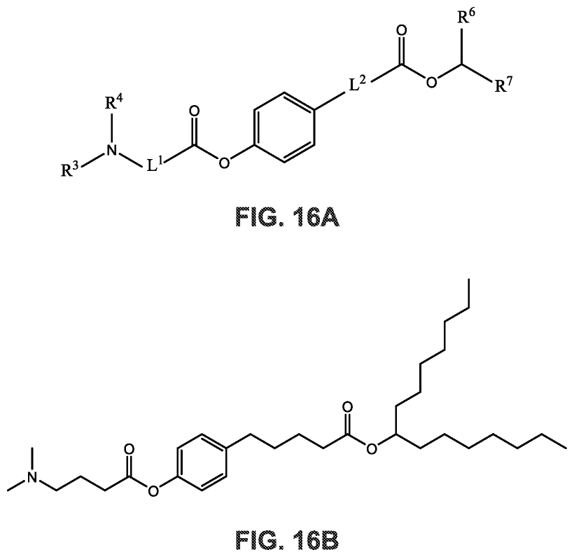

or a pharmaceutically acceptable salt or a stereoisomer thereof, wherein:
L¹ is C₁-C₆ alkylene; L² is C₁-C₈ alkylene; R³ and R⁴ are each independently H or C₁-C₃ alkyl; R⁶ is C₄-C₂₀ alkyl; and R⁷ is C₄-C₂₀ alkyl (FIG. 16A).

In some embodiments, the ionizable lipids of Formula (XIV) have the following structure (FIG. 16B):

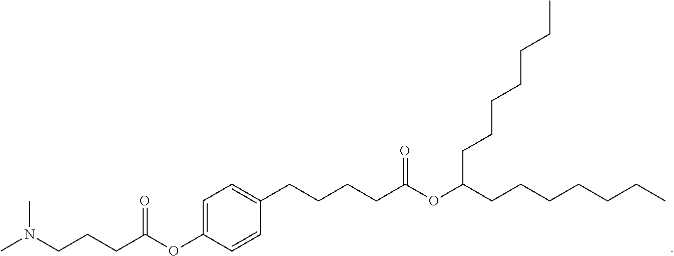 [SMILES: CCCCCCCC(CCCCCCC)OC(=O)CCCCc1ccc(OC(=O)CCCN(C)C)cc1]

In another embodiment, the lipids relate to ionizable lipids of Formula (XV)

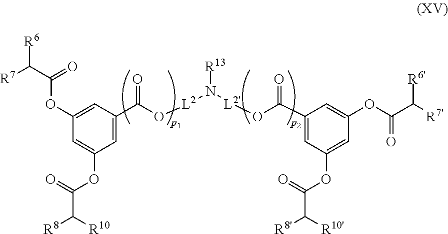

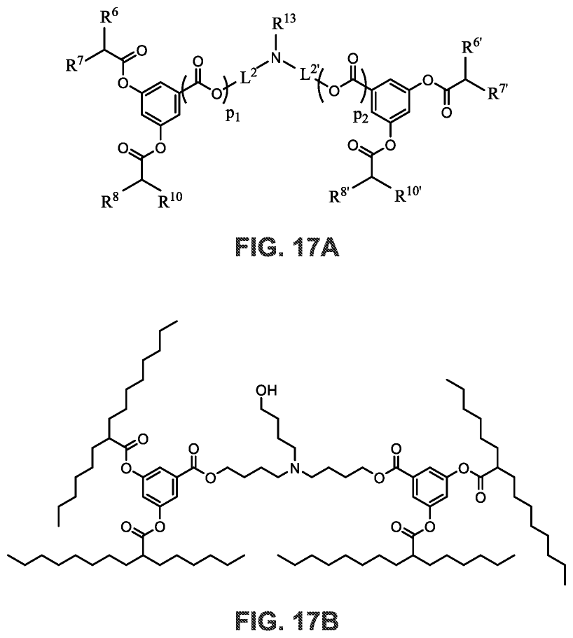

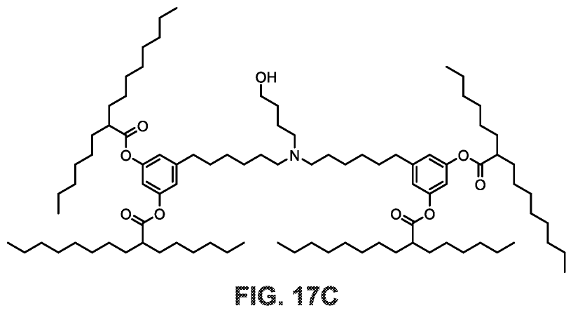

or a pharmaceutically acceptable salt or a stereoisomer thereof, wherein:
L² is C₁-C₈ alkylene; L^{2′} is C₁-C₈ alkylene; R⁶ is C₄-C₂₀alkyl; R^{6′} is C₄-C₂₀ alkyl; R⁷ is C₄-C₂₀ alkyl; R^{7′} is C₄-C_{2D} alkyl; R⁸ is C₄-C₂₀ alkyl; R^{8′} is C₄-C₂₀ alkyl; R¹⁰ is C₄-C₂₀ alkyl; R^{10′} is C₄-C₂₀ alkyl; R¹³ is H, C₁-C₆ alkyl, —(CH₂)ₙOH, or —(CH₂)_{q}N(CH₃)₂; n is 2, 3 or 4; p₁ is absent or 1; p₂ is absent or 1; and q is 2, 3, or 4 (FIG. 17A).

In some embodiments, the ionizable lipids of Formula (XV) have one of the following structures (FIG. 17B and FIG. 17C):

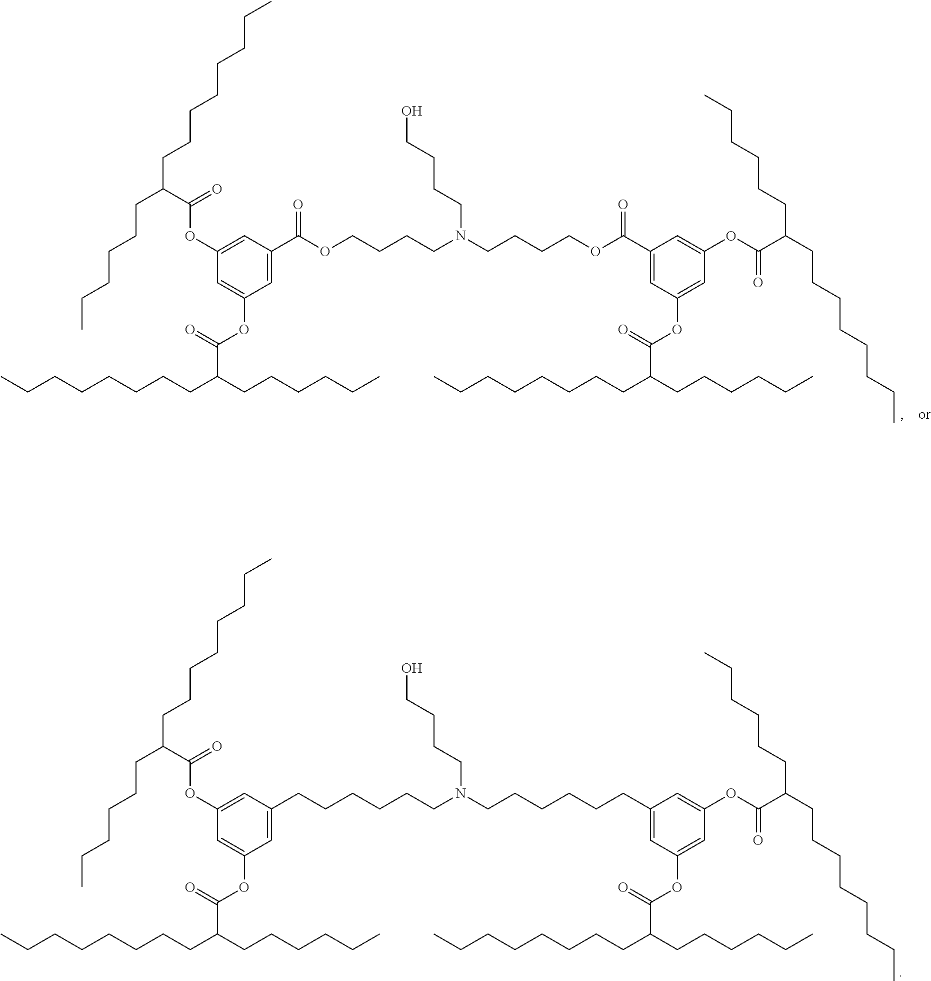

In another embodiment, the lipids relate to ionizable lipids of Formula (XVI)

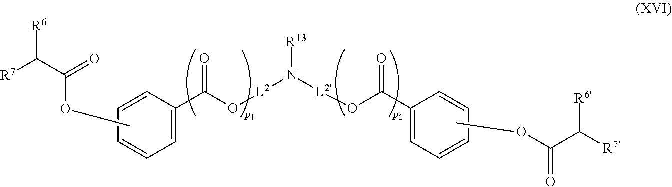

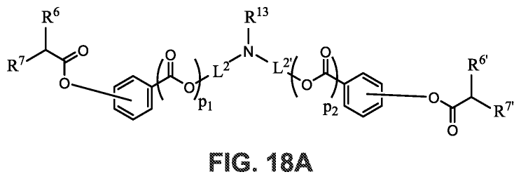

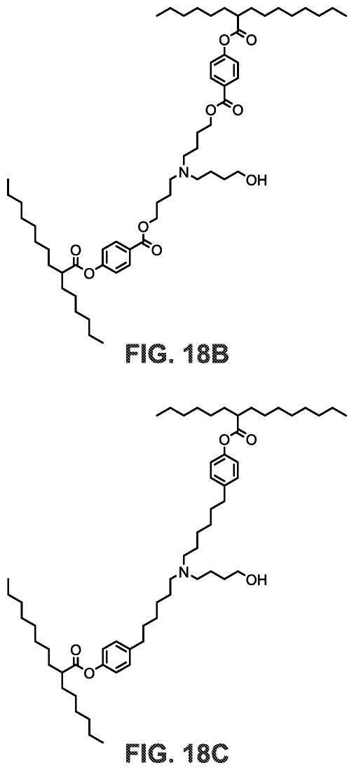

or a pharmaceutically acceptable salt or a stereoisomer thereof, wherein:
L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R¹³ is H, C₁-C₆ alkyl, —(CH₂)ₙOH, or —(CH₂)_{q}N(CH₃)₂; n is 2, 3 or 4; p₁ is absent or 1; p₂ is absent or 1; and q is 2, 3 or 4 (FIG. 18A).

In some embodiments, the ionizable lipids of Formula (XVI) have one of the following structures (FIG. 18B and FIG. 18C):

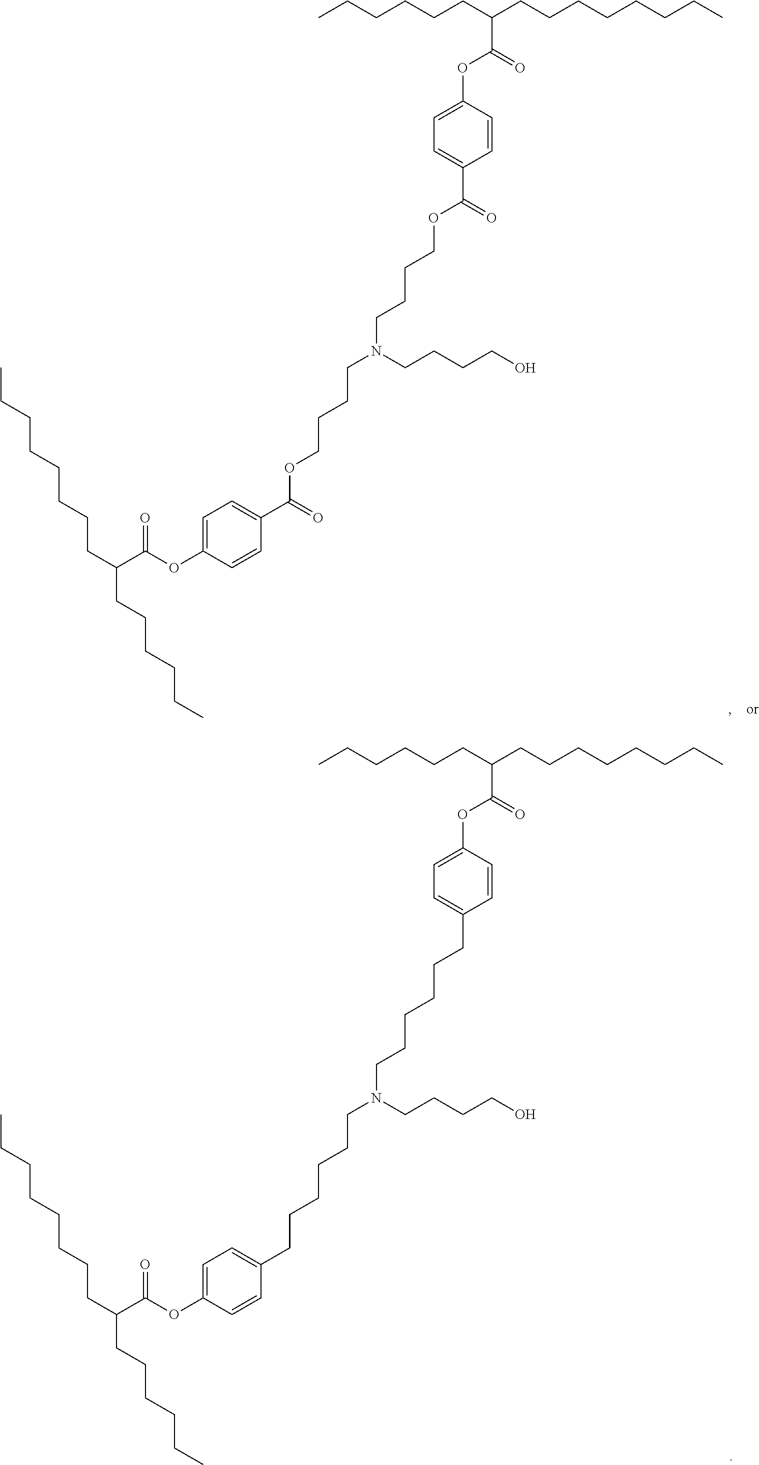

In one aspect, the lipids, such as transfection lipids, described herein relate to a lipid-nanoparticle composition comprising an ionizable lipid of any one of Formula (I) to Formula (XVI). The lipid-nanoparticle composition can further comprise a helper lipid, a stabilization lipid, a structural lipid, and a nucleic acid.

In some embodiments, the helper lipid in the lipid-nanoparticle composition is selected from the group consisting of 1,2-dilinoleoyl-sn-glycero-3-phosphocholine (DLPC), 1,2-dimyristoyl-sn-glycero-phosphocholine (DMPC), 1,2dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

In some embodiments, the stabilization lipid in the lipid-nanoparticle composition is 1-(monomethoxy-polyethyleneglycol)-2,3-dimyristoylglycerol (PEG-DMG), with an average PEG molecular weight of 2000.

In some embodiments, the structural lipid in the lipid-nanoparticle composition is selected from the group consisting of cholesterol, cholesterol derivatives, fecosterol, sitosterol, ergosterol, campesterol, stigmasterol, brassicasterol, tomatidine, ursolic acid, alpha tocopherol, and mixtures thereof.

In some embodiments, the nucleic acid, such as nucleic acid for transfection into an immune cell, is selected from a group consisting of small interfering RNA (siRNA), an asymmetrical interfering RNA (aiRNA), a microRNA (miRNA), a Dicer-substrate RNA (dsRNA), a small hairpin RNA (shRNA), mRNA, and mixtures thereof.

In other embodiments, viral-mediated gene delivery systems consist of viruses that may be modified to be replication-deficient, but which can deliver nucleic acids for exogenous expression. In some embodiments, adenoviruses, retroviruses, RNA viruses, such as Sendai viruses, and lentiviruses may be used as viral systems for delivery of exogenous nucleic acid sequences. For example, in some embodiments, Sendai virus and/or related paramyxoviruses may be used as vectors for nucleic acid delivery. Such viruses may be used to deliver mRNA either through the RNA in their genomes in the case of RNA viruses, or through mRNA expressed from the DNA in their genomes in the case of DNA viruses.

In some embodiments, the disclosure relates to a transfection composition comprising a nanoparticle composition and a nucleic acid for transfection into an immune cell, such as a T cell.

Cellular age-reversal, or rejuvenating, is achieved by transient overexpression of one or more mRNAs encoding cellular reprogramming factors. Such cellular reprogramming factors may include transcription factors, epigenetic remodelers, or small molecules affecting mitochondrial function, proteolytic activity, heterochromatin levels, histone methylation, nuclear lamina polypeptides, cytokine secretion, or senescence. In embodiments, the cellular reprogramming factors include one or more of OCT4, SOX2, KLF4, c-MYC, LIN28, NANOG and GLIS1. In embodiments, the cellular reprogramming factors are applied in different mass or molar ratios, for example OCT4, SOX2, KLF4, c-MYC, LIN28, and NANOG at mass or molar ratios of a:b:c:d:e:f, wherein a, b, c, d, e, and f can all be the same number (for example, 1:1:1:1:1:1), some the same number and some different numbers (for example, 3:1:1:1:1:1, 2:1:1:1:1:1, 2:2:1:1:1:1, 2:2:2:1:1:1, 2:2:2:2:1:1, 2:2:2:2:2:1, 3:3:3:3:2:2), or all different numbers (for example 6:4:5:3:2:1), and wherein a, b, c, d, e, and f are each 1-7, i.e., 1-7:1-7:1-7:1-7:1-7:1-7 (or 1-7:1-7:1-7:1-7:1-7, 1-7:1-7:1-7:1-7, 1-7:1-7:1-7, 1-7:1-7, or 1-7:1 in the case of combinations with fewer than 6 factors).

In some embodiments, mRNA encoding OCT4, SOX2, KLF4, c-MYC, LIN28 and NANOG is applied in mass ratios of (1-5:1-5:1-5:1-5:1-5:1-5), and mRNA encoding OCT4, c-MYC and NANOG is applied in mass ratios of (1-5:1-5:1-5); for example, mRNA encoding OSKMLN is applied in a mass ratio of 5:1:1:1:1:1 and mRNA encoding OMN is applied in a mass ratio of 5:1:1. In some further embodiments, mRNA encoding OCT4, SOX2, KLF4, c-MYC, LIN28 and NANOG is applied in mass ratios of (1-4:1-4:1-4:1-4:1-4:1-4), and mRNA encoding OCT4, c-MYC and NANOG is applied in mass ratios of (1-4:1-4:1-4); for example mRNA encoding OSKMLN is applied at a mass ratio of 4:1:1:1:1:1, and mRNA encoding OMN is applied at a mass ratio of 4:2:1. In some further embodiments, mRNA encoding OCT4, SOX2, KLF4, c-MYC, LIN28 and NANOG is applied in mass ratios of (1-3:1-3:1-3:1-3:1-3:1-3), and mRNA encoding OCT4, c-MYC and NANOG is applied in mass ratios of (1-3:1-3:1-3); for example mRNA encoding OSKMLN is applied at a mass ratio of 3:1:1:1:1:1, and mRNA encoding OMN is applied at a mass ratio of 1:1:1.

In some embodiments, mRNA encoding OCT4, SOX2, KLF4, c-MYC, LIN28 and NANOG is applied in molar ratios of (1-5:1-5:1-5:1-5:1-5:1-5), and mRNA encoding OCT4, c-MYC and NANOG is applied in molar ratios of (1-5:1-5:1-5); for example, mRNA encoding OSKMLN is applied in a molar ratio of 5:1:1:1:1:1 and mRNA encoding OMN is applied in a molar ratio of 5:1:1. In some further embodiments, mRNA encoding OCT4, SOX2, KLF4, c-MYC, LIN28 and NANOG is applied in molar ratios of (1-4:1-4:1-4:1-4:1-4:1-4), and mRNA encoding OCT4, c-MYC and NANOG is applied in molar ratios of (1-4:1-4:1-4); for example mRNA encoding OSKMLN is applied at a molar ratio of 4:1:1:1:1:1, and mRNA encoding OMN is applied at a molar ratio of 4:2:1. In some further embodiments, mRNA encoding OCT4, SOX2, KLF4, c-MYC, LIN28 and NANOG is applied in molar ratios of (1-3:1-3:1-3:1-3:1-3:1-3), and mRNA encoding OCT4, c-MYC and NANOG is applied in molar ratios of (1-3:1-3:1-3); for example mRNA encoding OSKMLN is applied at a molar ratio of 3:3:2:1:1:1, and mRNA encoding OMN is applied at a molar ratio of 2:1:1.

In embodiments, a cytokine or mRNA expressing a cytokine is administered to the immune cell before, concurrently with, or after the reprogramming factors. In some embodiments, the cytokine is IL-2. In some embodiments, the cytokine is the combination of IL-7 and IL-15. In some embodiments, the combination of IL-7 and IL-15 enhances T-cell responses or activity or used in methods of enhancing T-cell responses or activity.

In embodiments, the methods provided herein may be applied to any type of immune cell. The methods of the disclosure can be used to rejuvenate immune cells in culture (e.g., ex vivo or in vitro) to improve function and potency for use in immune cell therapy. The immune cells used in treatment of a patient may be autologous or allogeneic. The immune cells may be derived from the patient or a matched donor. For example, in ex vivo therapy, immune cells are obtained directly from the patient to be treated, transfected with mRNAs encoding cellular reprogramming factors, as described herein, and reimplanted in the patient. Such immune cells can be obtained, for example, from a biopsy or surgical procedure performed on the patient. Alternatively, immune cells in need of rejuvenation can be transfected directly in vivo with mRNAs encoding cellular reprogramming factors. In some embodiments, such engineering of immune cells is performed ex vivo, i.e., in the manufacturing of a cellular therapy product, such as an autologous or allogenic chimeric antigen receptor (CAR)-T, CAR-NK, CAR-M, or CAR-NKT cells. In some embodiments, such chimeric antigen receptors target at least one of CD19, CD20, CD22, CD30, CD33, CD123, FLT3, BCMA, GD2, HER2, MUC1, B7-H3, IL13Ra2, TAG72, MUC16, BCMA, or any other antigen suitable for immunotherapy. In some embodiments, such engineering includes engineering so that the immune cells express other proteins or peptides, such as growth factors and cytokines. In some embodiments, said cytokines include IL-7 and/or IL-15. In some embodiments, such engineering of immune cells is performed ex vivo, e.g., in the manufacturing of a cellular therapy product, such as an autologous or allogenic chimeric antigen receptor (CAR)-T, CAR-NK, CAR-M, or CAR-NKT cells. In some embodiments, the CAR-T cells provided herein are targeted to at least one of CD19, CD20, CD22, HER2, MUC1, CD30, CD33, CD123, FLT3, B7-H3, IL13Ra2, GD2, TAG72, MUC16, or BCMA by the chimeric antigen receptor. In some embodiments, the CAR-T cells provided herein are targeted to at least one of CD19 or BCMA by the chimeric antigen receptor. In some embodiments, the CAR-T cells provided herein are targeted to CD19 by the chimeric antigen receptor. In some embodiments, the CAR-T cells provided herein are targeted to BCMA by the chimeric antigen receptor. In some embodiments, the CAR-NK cells provided herein are targeted to at least one of CD19, FLT3, CD20, CD38, CD138, BCMS, CS1, CD3, CD5, CD7, NKG2D, HER2, EGFR, EpCAM, TF, B7-H6, HLA-G, CD24, CD44, CD133, mesothelin, or alphaFR by the chimeric antigen receptor. In some embodiments, the CAR-M cells provided herein are targeted to at least one of CD19, HER2, CD22, or ALK19 by the chimeric antigen receptor. In some embodiments, the CAR-NK cells provided herein are engineered to express IL-2 and/or IL-15. In some embodiments, the CAR-NKT cells provided herein are targeted to GD2, by the chimeric antigen receptor, and engineered to express IL-15. In such embodiments, the immune cell rejuvenation methods described herein are performed ex vivo during or after the manufacturing of the cell therapy product. In other embodiments, such engineering of immune cells is performed ex vivo, e.g., in so-called “in situ” generation of CAR-engineered cells. In such embodiments, mRNA encoding CARs or other cell engineering molecules is injected in vivo into a subject or patient, for example for CAR engineering of the patient's immune cells, such as T cells, NK cells, macrophages, tumor infiltrating lymphocytes, dendritic cells and/or NKT cells “in situ,” i.e., inside the patient's body without having to remove cells for ex vivo transfection. In such embodiments, the immune cell rejuvenation methods described herein are also performed in vivo, where mRNA encoding the reprogramming factor or factors is injected into the patient before, concurrently with, or after the mRNA encoding CARs or other cell engineering molecules. In some embodiments, the mRNA encoding the reprogramming factor or factors is delivered in vivo using lipid and/or polymer nanoparticles. In some embodiments, the lipid and/or polymer nanoparticles are engineered for targeted delivery to immune cells in vivo, such as T cells, NK cells, macrophages, tumor infiltrating cells, dendritic cells, and/or NKT cells in vivo. In still other embodiments, in vivo treatment is performed in the absence of any other in vivo cell engineering, to enhance or restore the potency of the immune system and treat diseases associated with immune dysfunction or dysregulation, such as improving the effect of the immune system against cancer or infection, or reducing inflammation.

### Compositions

In an aspect, provided herein are pharmaceutical compositions including rejuvenated immune cells obtained by transfecting immune cells with one or more messenger RNAs encoding one or more cellular reprogramming factors, or by expressing at least one reprogramming factor from transfected mRNA in immune cells, for not more than 3, 4, 5, 6, 7, 8, 9, 10, 11, 12, 13, 14, 15, 16, 17, 18, 19, 20, or 21 continuous days, to transiently reprogram the immune cells for rejuvenation. In an aspect, provided herein are pharmaceutical compositions including rejuvenated immune cells obtained by transfecting immune cells with one or more messenger RNAs encoding one or more cellular reprogramming factors, or by expressing at least one reprogramming factor from transfected mRNA in immune cells, for not more than 3, 4, 5, or 6 continuous days, to transiently reprogram the immune cells for rejuvenation.

The compositions provided herein comprise reprogramming factor polynucleotides, proteins, polypeptides, and RNA and DNA vectors containing said reprogramming factor polynucleotides for production of said reprogramming factor proteins and polypeptides. In some embodiments, the RNA vectors are mRNA and transfected into immune cells using lipid or polymer nanoparticles. In some embodiments, the RNA vectors are RNA viral vectors. In some embodiments, the DNA vectors are DNA viral vectors, such as adenovirus, lentivirus, or adeno-associated virus, where the DNA viral vectors express mRNA encoding the reprogramming factors. In such embodiments, methods may involve treating, introducing, or exposing immune cells to mRNA, where the mRNA is provided by a DNA viral vector. RNA vectors such as mRNA are preferable to viral vectors because they are non-integrative and provide better control over dosing and expression timing. Additionally, they can be less toxic to the cell, permitting greater efficacy of rejuvenation. Thus, use of mRNA to express reprogramming factors can result in enhanced rejuvenation, proliferation, recovery from or prevention of exhaustion, anti-pathogenic effects, anti-cancer effects, or anti-inflammatory effects in the exposed immune cell compared to when using a viral vector. In some embodiments, the reprogramming factors provided herein provide more robust cellular rejuvenation because the reprogramming factors have been engineered to decrease any triggered immune response to the protein/polypeptide/RNA/DNA, increase stability of the protein/polypeptide/RNA/DNA, or alter the protein/polypeptide activity or activity of the protein/polypeptide expressed from the RNA/DNA, such as increased activity when compared to wild-type reprogramming factors.

In some embodiments, the compositions also comprise B18R polynucleotides, proteins, polypeptides, and RNA and DNA vectors containing said reprogramming factor polynucleotides for production of said B18R proteins and polypeptides. B18R is a vaccinia virus-encoded B18R protein that functions as a soluble receptor for IFNα and IFNβ. This protein can exist as a soluble extracellular as well as a cell surface-bound protein, and it has a high affinity for type I IFNs. Thus, the binding of B18R protein can block the autocrine and paracrine function of type I IFNs. Furthermore, it can also bind to the cell surface of uninfected and infected cells, and thereby reduce the inflammatory signal. Because synthetic mRNA induces a cellular immune response, recombinant B18R protein can be applied to avoid the immune activation of cells and to block the activity of type I IFNs. In previous studies, addition of recombinant B18R protein during long-term cell reprogramming experiments with synthetic mRNAs led to an increased cell viability and a successful reprogramming of cells into induced pluripotent stem cells. In particular, a B18R polynucleotide sequence that has been codon-optimized for increased RNA expression in T-cells may be used.

In some embodiments, the polynucleotides encoding reprogramming factors from the Oct, Sox, Klf, Lin, Nanog, GLIS1, or Myc families may be codon-optimized to increase the levels of mRNA expression in any immune cell of interest, without altering the wild-type amino acid sequences of those reprogramming factors. In further embodiments, the polynucleotide sequences listed in Table 1 and/or provided with any sequence listing filed with this application are codon-optimized.

In other embodiments, the polynucleotide sequences encoding the reprogramming factors from the Oct, Sox, Klf, Lin, Nanog, GLIS1, or Myc families have been altered to modulate the function of the encoded reprogramming factors. In some embodiments, a polynucleotide sequence has been altered so that the reprogramming factor it encodes triggers a reduced immune response, is more stable, and/or elicits a more desirable activity, when compared to its corresponding wild-type or unaltered reprogramming factor. For example, Oct4MyoD contains a transcriptional activation domain from the myogenic determination gene MyoD that enhances its transcriptional activity.

In other embodiments, the polynucleotide sequences encoding the reprogramming factors from the Oct, Sox, Klf, Lin, Nanog, GLIS1, or Myc families are at least 95%, 96%, 97%, 98% or 99% identical to the polynucleotide sequences in Table 1. In other embodiments, the polynucleotide sequences encoding the reprogramming factors from the Oct, Sox, Klf, Lin, Nanog, GLIS1, or Myc families are at least 98% or 99% identical to the polynucleotide sequences in Table 1. In still other embodiments, the polynucleotide sequences encoding the reprogramming factors from the Oct, Sox, Klf, Lin, Nanog, GLIS1, or Myc families are at least 99% identical to the polynucleotide sequences in Table 1.

In other embodiments, the polynucleotide sequences encoding the reprogramming factors from the Oct, Sox, Klf, Lin, Nanog, GLIS1, or Myc families are about 90%, 85%, 80%, 75% or 70% identical to the polynucleotide sequences in Table 1. In other embodiments, the polynucleotide sequences encoding the reprogramming factors from the Oct, Sox, Klf, Lin, Nanog, GLIS1, or Myc families are about 80% or 85% identical to the polynucleotide sequences in Table 1. In still other embodiments, the polynucleotide sequences encoding the reprogramming factors from the Oct, Sox, Klf, Lin, Nanog, GLIS1, or Myc families are about 75% identical to the polynucleotide sequences in Table 1. In other embodiments, the polynucleotide sequences encoding the reprogramming factors from the Oct, Sox, Klf, Lin, Nanog, GLIS1, or Myc families are about 70% identical to the polynucleotide sequences in Table 1.

In embodiments, the reprogramming factor is a protein, for example a transcription factor, that plays a role in changing adult or differentiated immune cells into pluripotent stem cells. The term “reprogramming factor” further includes any analogue molecule that mimics the function of the factor. In embodiments, the reprogramming factor is a factor from the Oct family, the Sox family, the Klf family, the Myc family, the Nanog family, or the Lin family.

“Oct family” refers to the family of octamer (“Oct”) transcription factors which play a crucial role in maintaining pluripotency. POU5F1 (POU domain, class 5, transcription factor 1) also known as Oct3/4 is one representative of Oct family. The absence of Oct3/4 in Oct-3/4+ cells, such as blastomeres and embryonic stem cells, leads to spontaneous trophoblast differentiation, and presence of Oct-3/4 thus gives rise to the pluripotency and differentiation potential of embryonic stem cells. Exemplary Oct3/4 proteins are the proteins encoded by the murine Oct3/4 gene (Genbank accession number NM_013633) and the human Oct3/4 gene (Genbank accession number NM_002701). The terms “Oct3/4”, “Oct4,” “OCT4,” “Oct4 protein,” “OCT4 protein” and the like thus refer to any of the naturally-occurring forms of the Octomer 4 transcription factor, or variants thereof that maintain Oct4 transcription factor activity (e.g. within at least 50%, 80%, 90% or 100% activity compared to wild-type Oct4 as measured by methods known in the art). In some embodiments, variants have at least 90% amino acid sequence identity across their whole sequence compared to the naturally occurring Oct4 polypeptide. In other embodiments, the Oct4 protein is the protein as identified by the Genbank reference ADW77327.1. In other embodiments, the Oct4 protein is Oct4MyoD containing the transactivation domain of MyoD for enhanced transcription factor activity. In other embodiments, the Oct4 polynucleotide sequence is codon-optimized for immune cell expression, such as T cell expression.

An Oct reprogramming factor refers to any of the naturally-occurring members of octamer family of transcription factors, or variants thereof that maintain transcription factor activity, similar (within at least 50%, 80%, or 90% activity) compared to the closest related naturally-occurring Oct family member, or polypeptides comprising at least the DNA-binding domain of the naturally occurring family member, and can further comprise a transcriptional activation domain. Exemplary Oct polypeptides include Oct-1, Oct-2, Oct-3/4, Oct-6, Oct-7, Oct-8, Oct-9, and Oct-11. e.g. Oct3/4 (referred to herein as “Oct4”) contains the POU domain, a 150 amino acid sequence conserved among Pit-1, Oct-1, Oct-2, and uric-86. See, Ryan, A. K. & Rosenfeld, M. G. Genes Dev. 11, 1207-1225 (1997). In some embodiments, variants have at least 85%, 90%, or 95% amino acid sequence identity across their whole sequence compared to a naturally occurring Oct polypeptide family member such as to those listed above or such as listed in Genbank accession number NP002692.2 (human Oct4) or NP038661.1 (mouse Oct4). Oct polypeptides (e.g., Oct3/4) can be from human, mouse, rat, bovine, porcine, or other animals.

In some embodiments, the OCT4 reprogramming factor protein/polypeptide provided herein is encoded by the codon-optimized polynucleotide sequence of SEQ ID NO: 1. Accordingly, the polynucleotide of SEQ ID NO: 1 differs from the polynucleotide sequence of wild-type OCT4, but the amino acid sequence it encodes does not. In other embodiments, the codon-optimized nucleotide sequences, such as SEQ ID NO: 4, may also be modified to encode, for example, a more robust OCT4 reprogramming factor that elicits a smaller triggered immune response, is more stable and/or provides a more desirable activity level when compared to proteins or polypeptides corresponding to wild-type nucleotide sequences. In some embodiments, the OCT4 reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 1. In some embodiments, the OCT4 reprogramming factor protein/polypeptide is encoded by a polynucleotide sequence comprising SEQ ID NO: 1. In some embodiments, the OCT4 reprogramming factor protein/polypeptide is encoded by a polynucleotide sequence consisting essentially of SEQ ID NO: 1. In some embodiments, the OCT4 reprogramming factor protein/polypeptide is encoded by a polynucleotide sequence consisting of SEQ ID NO: 1.

“Sox family” refers to genes that encode for SRY (sex determining region Y)-box 2, also known as SOX2, associated with maintaining pluripotency. Exemplary Sox2 proteins are the proteins encoded by the murine Sox2 gene (Genbank accession number NM_011443) and the human Sox2 gene (Genbank accession number NM_003106). The terms “Sox2,” “SOX2,” “Sox2 protein,” “SOX2 protein” and the like as referred to herein thus includes any of the naturally-occurring forms of the Sox2 transcription factor, or variants thereof that maintain Sox2 transcription factor activity (e.g. within at least 50%, 80%, 90% or 100% activity compared to wild-type Sox2 as measured by methods known in the art). In some embodiments, variants have at least 90% amino acid sequence identity across their whole sequence compared to the naturally occurring Sox2 polypeptide. In other embodiments, the Sox2 protein is the protein as identified by the NCBI reference NP_003097.1. In other embodiments, the Sox2 protein and/or polynucleotide sequence is optimized for immune cell expression and/or activity, such as T cell expression and/or activity.

A Sox reprogramming factor refers to any of the naturally-occurring members of the SRY-related HMG-box (Sox) transcription factors, characterized by the presence of the high-mobility group (HMG) domain, or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, e.g., Dang, D. T., et al., Int. J. Biochem. Cell Biol. 32:1103-1121 (2000). Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. In some embodiments, variants have at least 85%, 90%, or 95% amino acid sequence identity across their whole sequence compared to a naturally occurring Sox polypeptide family member such as to those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals.

In some embodiments, the SOX2 reprogramming factor protein/polypeptide provided herein is encoded by the codon-optimized polynucleotide sequence of SEQ ID NO: 2. Accordingly, the polynucleotide of SEQ ID NO: 2 differs from the polynucleotide sequence of wild-type SOX2, but the amino acid sequence it encodes does not. In other embodiments, the codon-optimized nucleotide sequences, such as SEQ ID NO: 2, may also be modified to encode, a more robust SOX2 reprogramming factor that elicits a smaller triggered immune response, is more stable and/or provides a more desirable activity level when compared to proteins or polypeptides corresponding to wild-type nucleotide sequences. In some embodiments, the SOX2 reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 2. In some embodiments, the SOX2 reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 2. In some embodiments, the SOX2 reprogramming factor protein/polypeptide is encoded by a polynucleotide sequence comprising SEQ ID NO: 2. In some embodiments, the SOX2reprogramming factor protein/polypeptide is encoded by a polynucleotide sequence consisting essentially of SEQ ID NO: 2. In some embodiments, the SOX2 reprogramming factor protein/polypeptide is encoded by a polynucleotide sequence consisting of SEQ ID NO: 2.

“Klf family” refers to Kruppel-like factor 4 or “Klf” genes that encode for Klf4 proteins are the proteins encoded by the murine klf4 gene (Genbank accession number NM_010637) and the human klf4 gene (Genbank accession number NM_004235). The terms “KLF4,” “KLF4 protein” and the like as referred to herein thus includes any of the naturally-occurring forms of the KLF4 transcription factor, or variants thereof that maintain KLF4 transcription factor activity (e.g. within at least 50%, 80%, 90% or 100% activity compared to wild-type KLF4 as measured by methods known in the art). In some embodiments, variants have at least 90% amino acid sequence identity across their whole sequence compared to the naturally occurring KLF4 polypeptide. In other embodiments, the KLF4 protein is the protein as identified by the NCBI reference NP_004226.3. In other embodiments, the Klf4 protein and/or polynucleotide sequence is optimized for immune cell expression and/or activity, such as T cell expression and/or activity.

In other embodiments, the Klf reprogramming factor refers to any of the naturally-occurring members of the family of Kruppel-like factors (Klfs), zinc-finger proteins that contain amino acid sequences similar to those of the Drosophila embryonic pattern regulator Kruppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, Dang, D. T., Pevsner, J.& Yang, V. W., Cell Biol. 32, 1103-1121 (2000). Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. In some embodiments, variants have at least 85%, 90%, or 95% amino acid sequence identity across their whole sequence compared to a naturally occurring Klf polypeptide family member such as to those listed above or such as listed in Genbank accession number CAX16088 (mouse Klf4) or CAX14962 (human Klf4). Klf polypeptides (e.g., Klf1, Klf4, and Klf5) can be from human, mouse, rat, bovine, porcine, or other animals.

In some embodiments, the KLF4 reprogramming factor protein/polypeptide provided herein is encoded by the codon-optimized polynucleotide sequence of SEQ ID NO: 4. Accordingly, the polynucleotide of SEQ ID NO: 4 differs from the polynucleotide sequence of wild-type KLF4, but the amino acid sequence it encodes does not. In other embodiments, the codon-optimized nucleotide sequences, such as SEQ ID NO:4, may also be modified to encode, for example, a more robust KLF4 reprogramming factor that elicits a smaller triggered immune response, is more stable and/or provides a more desirable activity level when compared to proteins or polypeptides corresponding to wild-type nucleotide sequences. In some embodiments, the KLF4 reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 4. In some embodiments, the KLF4 reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 4. In some embodiments, the KLF4 reprogramming factor protein/polypeptide is encoded by a polynucleotide sequence comprising SEQ ID NO: 4. In some embodiments, the KLF4 reprogramming factor protein/polypeptide is encoded by a polynucleotide sequence consisting essentially of SEQ ID NO: 4. In some embodiments, the KLF4 reprogramming factor protein/polypeptide is encoded by a polynucleotide sequence consisting of SEQ ID NO: 4.

Factors of the Myc family refers to factors encoded by myc proto-oncogenes implicated in cancer. Exemplary c-Myc proteins are the proteins encoded by the murine c-myc gene (Genbank accession number NM_010849) and the human c-myc gene (Genbank accession number NM_002467). N-Myc or L-myc was also used as possible reprogramming factor replacing c-Myc. The terms “c-Myc,” “C-MYC,” “c-Myc protein”, “C-MYC protein” and the like as referred to herein thus includes any of the naturally-occurring forms of the cMyc transcription factor, or variants thereof that maintain cMyc transcription factor activity (e.g. within at least 50%, 80%, 90% or 100% activity compared to wild-type cMyc as measured by methods known in the art). In some embodiments, variants have at least 90% amino acid sequence identity across their whole sequence compared to the naturally occurring c-Myc polypeptide. In other embodiments, the c-Myc protein is the protein as identified by the NCBI reference NP_002458.2.

The Myc family of cellular genes is comprised of c-myc, N-myc, and L-myc, and reference to Myc refers any of the naturally-occurring members of the Myc family (see, e.g., Adhikary, S. & Eilers, M. Nat. Rev. Mol. Cell Biol. 6:635-645 (2005)), or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in Genbank accession number CAA25015 (human Myc). Myc polypeptides (e.g., c-Myc) can be from human, mouse, rat, bovine, porcine, or other animals.

In some embodiments, the c-Myc reprogramming factor protein/polypeptide provided herein is encoded by the codon-optimized polynucleotide sequence of SEQ ID NO: 3. Accordingly, the polynucleotide of SEQ ID NO: 3 differs from the polynucleotide sequence of wild-type c-Myc, but the amino acid sequence it encodes does not. In other embodiments, the codon-optimized nucleotide sequences, such as SEQ ID NO:3, may also be modified to encode, for example, a more robust c-Myc reprogramming factor that elicits a smaller triggered immune response, is more stable and/or provides a more desirable activity level when compared to proteins or polypeptides corresponding to wild-type nucleotide sequences. In some embodiments, the c-Myc reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 3. In some embodiments, the c-Myc reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 3. In some embodiments, the c-Myc reprogramming factor protein/polypeptide is encoded by a polynucleotide sequence comprising SEQ ID NO: 3. In some embodiments, the c-Myc reprogramming factor protein/polypeptide is encoded by a polynucleotide sequence consisting essentially of SEQ ID NO: 3. In some embodiments, the c-Myc reprogramming factor protein/polypeptide is encoded by a polynucleotide sequence consisting of SEQ ID NO: 3. In other embodiments, the c-Myc protein and/or polynucleotide sequence is optimized for immune cell expression and/or activity, such as T cell expression and/or activity.

The term “Nanog” or “nanog” refers to a transcription factor critically involved with self-renewal of undifferentiated embryonic stem cells. In humans, this protein is encoded by the NANOG gene. Exemplary nanog is the protein encoded by murine gene (Genbank accession number XM.sub.13 132755) and human Nanog gene (Genbank accession number NM_024865). The term “Nanog” or “nanog” and the like includes any of the naturally-occurring forms of the Nanog transcription factor, or variants thereof that maintain Nanog transcription factor activity (e.g., within at least 50%, 80%, 90% or 100% activity compared to wild-type Nanog as measured by methods known in the art). In some embodiments, variants have at least 90% amino acid sequence identity across their whole sequence compared to the naturally occurring Nanog polypeptide. In other embodiments, the Nanog protein is the protein as identified by the NCBI reference NP_079141. In other embodiments, the Nanog protein and/or polynucleotide sequence is optimized for immune cell expression and/or activity, such as T cell expression and/or activity.

In some embodiments, the Nanog reprogramming factor protein/polypeptide provided herein is encoded by the codon-optimized polynucleotide sequence of SEQ ID NO: 6. Accordingly, the polynucleotide of SEQ ID NO: 6 differs from the polynucleotide sequence of wild-type Nanog, but the amino acid sequence it encodes does not. In other embodiments, the codon-optimized nucleotide sequences, such as SEQ ID NO:6, may also be modified to encode, for example, a more robust Nanog reprogramming factor that elicits a smaller triggered immune response, is more stable and/or provides a more desirable activity level when compared to proteins or polypeptides corresponding to wild-type nucleotide sequences. In some embodiments, the Nanog reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 6. In some embodiments, the Nanog reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 6. In some embodiments, the Nanog reprogramming factor protein/polypeptide is encoded by a polynucleotide sequence comprising SEQ ID NO: 6. In some embodiments, the Nanog reprogramming factor protein/polypeptide is encoded by a polynucleotide sequence consisting essentially of SEQ ID NO: 6. In some embodiments, the Nanog reprogramming factor protein/polypeptide is encoded by a polynucleotide sequence consisting of SEQ ID NO: 6.

The term “Lin28” or “Lin-28 homolog A” is a protein that is encoded by the LIN28 gene in humans. Exemplary Lin28 is the protein encoded by murine gene (Genbank accession number NM_145833) and human Lin28 gene (Genbank accession number NM_024674). The term “Lin28” or “Lin28 homolog A” and the like as referred to herein thus includes any of the naturally-occurring forms of the Lin28 transcription factor, or variants thereof that maintain Lin28 transcription factor activity (e.g. within at least 50%, 80%, 90% or 100% activity compared to wild-type Lin28 as measured by methods known in the art). In some embodiments, variants have at least 90% amino acid sequence identity across their whole sequence compared to the naturally occurring Lin28 polypeptide. In other embodiments, the Lin28 protein is the protein as identified by the NCBI reference NP_078950. In other embodiments, the Lin28 protein and/or polynucleotide sequence is optimized for immune cell expression and/or activity, such as T cell expression and/or activity.

In some embodiments, the Lin28 reprogramming factor protein/polypeptide provided herein is encoded by the codon-optimized polynucleotide sequence of SEQ ID NO: 5. Accordingly, the polynucleotide sequence of SEQ ID NO: 5 differs from the polynucleotide sequence of wild-type Lin28, but the amino acid sequence it encodes does not. In other embodiments, the codon-optimized nucleotide sequences, such as SEQ ID NO: 5, may also be modified to encode, for example, a more robust Lin28 reprogramming factor that elicits a smaller triggered immune response, is more stable and/or provides a more desirable activity level when compared to proteins or polypeptides corresponding to wild-type nucleotide sequences. In some embodiments, the Lin28 reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 5. In some embodiments, the Lin28 reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 5. In some embodiments, the Lin28 reprogramming factor protein/polypeptide is encoded by a polynucleotide sequence comprising SEQ ID NO: 5. In some embodiments, the Lin28 reprogramming factor protein/polypeptide is encoded by a polynucleotide sequence consisting essentially of SEQ ID NO: 5. In some embodiments, the Lin28 reprogramming factor protein/polypeptide is encoded by a polynucleotide sequence consisting of SEQ ID NO: 5.

In some embodiments, the reprogramming factor protein/polypeptides provided herein in SEQ ID NOs. 7-14 are encoded by nucleotide sequences that have been codon-optimized for expression in T cells. Accordingly, in some embodiments, the polynucleotides of SEQ ID NOs: 7-14 differ from the polynucleotide sequences of the corresponding wild-type reprogramming factors, although the amino acid sequences they encode do not. In other embodiments, the codon-optimized nucleotide sequences of the reprogramming factors described herein, such as SEQ ID NOs: 7-14, may also be modified to encode, for example, a more robust T cell reprogramming factor that elicits a smaller triggered immune response, is more stable and/or provides a more desirable activity level when compared to proteins or polypeptides corresponding to wild-type nucleotide sequences. In some embodiments, the T cell optimized reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to any one of the sequences of SEQ ID NOs: 7-14. In some embodiments, the T cell optimized reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to any one of the sequences of SEQ ID NOs: 7-14. In some embodiments, the T cell optimized reprogramming factor protein/polypeptide is encoded by a polynucleotide sequence comprising any one of the sequences of SEQ ID NOs: 7-14. In some embodiments, the T cell optimized reprogramming factor protein/polypeptide is encoded by a polynucleotide sequence consisting essentially of any one of the sequences of SEQ ID NOs: 7-14. In some embodiments, the T cell optimized reprogramming factor protein/polypeptide is encoded by a polynucleotide sequence consisting of any one of the sequences of SEQ ID NOs: 7-14.

In some embodiments, the T cell optimized reprogramming factor comprises OCT4MyoD for T-cells (T-OCT4MyoD, SEQ ID NO: 7) or reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 7. In some embodiments, the T cell optimized reprogramming factor is encoded by a polynucleotide sequence having about 96% sequence identity to SEQ ID NO: 7. In some embodiments, the T cell optimized reprogramming factor is encoded by a polynucleotide sequence having about 97% sequence identity to SEQ ID NO: 7. In some embodiments, the T cell optimized reprogramming factor is encoded by a polynucleotide sequence having about 98% sequence identity to SEQ ID NO: 7. In some embodiments, the T cell optimized reprogramming factor is encoded by a polynucleotide sequence with about 99% sequence identity to SEQ ID NO: 7. In some embodiments, the T cell optimized reprogramming factor is encoded by a polynucleotide sequence comprising SEQ ID NO: 7. In some embodiments, the T cell optimized reprogramming factor is encoded by a polynucleotide sequence consisting of SEQ ID NO: 7.

In some embodiments, the T cell optimized reprogramming factor comprises OCT4MyoD for T-cells (T-OCT4MyoD, SEQ ID NO: 7) or reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 7. In some embodiments, the T cell optimized reprogramming factor comprises B18R for T cells (T-B18R, SEQ ID NO: 8) or a reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 8. In some embodiments, the T cell optimized reprogramming factor comprises B18R for T cells (T-B18R, SEQ ID NO: 8) or a reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 8. In some embodiments, the T cell optimized reprogramming factor comprises KLF4 for T cells (T-KLF4, SEQ ID NO: 9) or a reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 9. In some embodiments, the T cell optimized reprogramming factor comprises KLF4 for T cells (T-KLF4, SEQ ID NO: 9) or a reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 9. In some embodiments, the T cell optimized reprogramming factor comprises LIN28 for T cells (T-LIN28, SEQ ID NO: 10) or a reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 10. In some embodiments, the T cell optimized reprogramming factor comprises LIN28 for T cells (T-LIN28, SEQ ID NO: 10) or a reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 10. In some embodiments, the T cell optimized reprogramming factor comprises NANOG for T cells (T-NANOG, SEQ ID NO: 11) or a reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 11. In some embodiments, the T cell optimized reprogramming factor comprises NANOG for T cells (T-NANOG, SEQ ID NO: 11) or a reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 11. In some embodiments, the T cell optimized reprogramming factor comprises OCT4 for T cells (T-OCT4, SEQ ID NO: 12) or a reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 12. In some embodiments, the T cell optimized reprogramming factor comprises OCT4 for T cells (T-OCT4, SEQ ID NO: 12) or a reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 12. In some embodiments, the T cell optimized reprogramming factor comprises SOX2 for T cells (T-SOX2, SEQ ID NO: 13) or a reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 13. In some embodiments, the T cell optimized reprogramming factor comprises SOX2 for T cells (T-SOX2, SEQ ID NO: 13) or a reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 13. In some embodiments, the T cell optimized reprogramming factor comprises cMYC for T-cells (T-cMyc, SEQ ID NO: 14) or a reprogramming factor protein/polypeptide is encoded by a polynucleotide sequence having at least about 70%, about 75%, about 80%, about 85%, about 90%, about 95%, about 96%, about 97%, about 98%, about 99%, or about 100% sequence identity to SEQ ID NO: 14. In some embodiments, the T cell optimized reprogramming factor comprises cMYC for T-cells (T-cMyc, SEQ ID NO: 14) or a reprogramming factor protein/polypeptide is encoded by a polynucleotide sequence having about 70%, about 75%, about 80%, about 85%, about 90%, about 95%, about 96%, about 97%, about 98%, about 99%, or about 100% sequence identity to SEQ ID NO: 14.

In an embodiment, the RNA vectors provided herein encode for expression of a combination of one, two, three, four, five, six, or more, reprogramming factors. In an embodiment, the reprogramming factors are selected from Oct4, Klf4, Sox2, c-Myc (or L-myc), Lin28 and Nanog. In an embodiment, the reprogramming factors are Oct4, Klf4, Sox2, c-Myc (or L-myc), Lin28 and Nanog. In an embodiment, the reprogramming factors are Oct4, Klf4, Sox2, c-Myc (or L-myc). In an embodiment, the reprogramming factors are Oct4, Klf4, Sox2. In an embodiment, the reprogramming factors are Oct4, Sox2, Lin28 and Nanog.

In certain embodiments, compositions comprising rejuvenated immune cells for use in cell therapy may further comprise one or more additional factors, such as nutrients, cytokines, growth factors, vaccine antigens including cancer vaccine antigens, extracellular matrix (ECM) components, antibiotics, anti-oxidants, or immunosuppressive agents to improve immune cell function or viability. The composition may also further comprise a pharmaceutically acceptable carrier.

Examples of growth factors include, but are not limited to, fibroblast growth factor (FGF), insulin-like growth factor (IGF), transforming growth factor beta (TGF-B), epiregulin, epidermal growth factor (“EGF”), endothelial cell growth factor (“ECGF”), nerve growth factor (“NGF”), leukemia inhibitory factor (“LIF”), bone morphogenetic protein-4 (“BMP-4”), hepatocyte growth factor (“HGF”), vascular endothelial growth factor-A (“VEGF-A”), and cholecystokinin octapeptide.

Examples of ECM components include, but are not limited to, proteoglycans (e.g., chondroitin sulfate, heparan sulfate, and keratan sulfate), non-proteoglycan polysaccharides (e.g., hyaluronic acid), fibers (e.g., collagen and elastin), and other ECM components (e.g., fibronectin and laminin).

Examples of immunosuppressive agents include, but are not limited to, steroidal (e.g., prednisone) or non-steroidal (e.g., sirolimus (Rapamune, Wyeth-Ayerst Canada), tacrolimus (Prograf, Fujisawa Canada), and anti-IL2R daclizumab (Zenapax, Roche Canada). Other immunosuppressant agents include 15-deoxyspergualin, cyclosporin, methotrexate, rapamycin, Rapamune (sirolimus/rapamycin), FK506, or Lisofylline (LSF).

One or more pharmaceutically acceptable excipients may also be included. Examples include, but are not limited to, carbohydrates, inorganic salts, antimicrobial agents, antioxidants, surfactants, buffers, acids, bases, and combinations thereof.

For example, an antimicrobial agent for preventing or deterring microbial growth may be included. Non-limiting examples of antimicrobial agents suitable for the present disclosure include benzalkonium chloride, benzethonium chloride, benzyl alcohol, cetylpyridinium chloride, chlorobutanol, phenol, phenylethyl alcohol, phenylmercuric nitrate, thimersol, and combinations thereof. Antimicrobial agents also include antibiotics that can also be used to prevent bacterial infection. Examples antibiotics include amoxicillin, penicillin, sulfa drugs, cephalosporins, erythromycin, streptomycin, gentamicin, tetracycline, chlarithromycin, ciproflozacin, azithromycin, and the like. Also included are antifungal agents such as myconazole and terconazole.

Various antioxidants can also be included, such as molecules having thiol groups such as reduced glutathione (GSH) or its precursors, glutathione or glutathione analogs, glutathione monoester, and N-acetylcysteine. Other suitable anti-oxidants include superoxide dismutase, catalase, vitamin E, Trolox, lipoic acid, lazaroids, butylated hydroxyanisole (BHA), vitamin K, and the like.

Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate di basic, and combinations thereof.

Acids or bases can also be present as an excipient. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting ofsodiumhydroxide,sodiumacetate,ammoniumhydroxide,potassiumhydroxide,ammonium acetate, potassium acetate, sodium phosphate, potassium phosphate, sodium citrate, sodium formate, sodium sulfate, potassium sulfate, potassium fumerate, and combinations thereof.

Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight being included in some embodiments. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A.H., Handbook of Pharmaceutical Excipients, 3rd Edition, American Pharmaceutical Association, Washington, D.C., 2000.

### Methods of Use

The methods and compositions provided herein may be used to treat or reduce any condition, disease, or disorder associated with immune cell function such as, but not limited to, neurodegenerative diseases (e.g., Alzheimer's disease, Parkinson's disease, Huntington's disease, amyotrophic lateral sclerosis, dementia, spinal cord injury, and stroke), cardiovascular and peripheral vascular diseases (e.g., atherosclerosis, peripheral arterial disease (PAD), hematomas, calcification, thrombosis, embolisms, and aneurysms), eye diseases (e.g., age-related macular degeneration, glaucoma, cataracts, dry eye, diabetic retinopathy, vision loss), dermatologic diseases (dermal atrophy and thinning, elastolysis and skin wrinkling, sebaceous gland hyperplasia or hypoplasia, senile lentigo and other pigmentation abnormalities, graying hair, hair loss or thinning, and chronic skin ulcers), autoimmune diseases (e.g., polymyalgia rheumatica (PMR), giant cell arteritis (GCA), rheumatoid arthritis (RA), crystal arthropathies, and spondyloarthropathy (SPA)), endocrine and metabolic dysfunction (e.g., adult hypopituitarism, hypothyroidism, apathetic thyrotoxicosis, osteoporosis, diabetes mellitus, adrenal insufficiency, various forms of hypogonadism, and endocrine malignancies), musculoskeletal disorders (e.g., arthritis, osteoporosis, myeloma, gout, Paget's disease, bone fractures, bone marrow failure syndrome, ankylosis, diffuse idiopathic skeletal hyperostosis, hematogenous osteomyelitis, muscle atrophy, peripheral neuropathy, multiple sclerosis, amyotrophic lateral sclerosis (ALS), Duchenne muscular dystrophy, primary lateral sclerosis, and myasthenia gravis), diseases of the digestive system (e.g., liver cirrhosis, liver fibrosis, Barrett's esophagus), respiratory diseases (e.g., pulmonary fibrosis, chronic obstructive pulmonary disease, asthma, chronic bronchitis, pulmonary embolism (PE), lung cancer, and infections), conditions associated with cellular proliferation, and any other diseases and disorders associated with aging. The methods and compositions provided herein may be used to treat fibrosis, including but not limited to fibrosis of the heart, liver, lung, kidney, pancreas, bladder, uterus, or gastrointestinal tract. The methods and compositions provided herein may be used to treat any disease or disorder associated with inflammation, such as neuroinflammation or inflammation of the heart, liver, lung, kidney, pancreas, bladder, uterus, or gastrointestinal tract. The methods and compositions provided herein may be used to treat diseases or disorders related to aging and/or chronic tissue damage, such as diabetes, fibrosis, cancer (including solid tumors), neurodegeneration, arthritis, sarcopenia. The methods and compositions provided herein may be used to destroy, inhibit, or rejuvenate senescent cells, e.g., for senotherapy, senolysis, senomorphic effects, or senoblocking.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

“Cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer, anal cancer; angiosarcoma (e.g., lymphangio sarcoma, lymphangioendotheliosarcoma, hemangiosarcoma); appendix cancer, benign monoclonal gammopathy; biliary cancer (e.g., cholangiocarcinoma); bladder cancer, breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer, carcinoid tumor, cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer, gastric cancer (e.g., stomach adenocarcinoma); gastrointestinal stromal tumor (GIST); germ cell cancer, head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macro globulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL)(e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer, and vulvar cancer (e.g., Paget's disease of the vulva).

By “therapeutically effective dose or amount” is intended an amount of rejuvenated immune cells that brings about a positive therapeutic response in a subject in need of rejuvenated immune cells, such as an amount that restores normal anatomy and/or physiology at a treatment site. The rejuvenated cells may be produced by transfection in vitro, ex vivo, or in vivo with the RNA, or RNA vector for expression of the one or more reprogramming nucleotide sequences encoding one or more cellular reprogramming factors, as described herein (including SEQ ID NOs: 1-14). Thus, for example, a “positive therapeutic response” would be an improvement in the immune cell related disease or condition in association with the rejuvenated immune cell therapy, and/or an improvement in one or more symptoms of the immune cell related disease or condition in association with the therapy, such as restored functionality, reduced pain, improved stamina, increased strength, increased mobility, and/or improved cognitive function. The exact amount (of immune cells or mRNA) required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

As used herein, the terms “subject,” “individual,” and “patient,” are used interchangeably herein and refer to any vertebrate subject, including, without limitation, humans and other primates, including non-human primates such as chimpanzees and other apes and monkey species; farm animals such as cattle, sheep, pigs, goats and horses; domestic mammals such as dogs and cats; rodents such as mice, rats, rabbits, hamsters, and guinea pigs; birds, including domestic, wild and game birds such as chickens, turkeys and other gallinaceous birds, ducks, geese, and the like. In some cases, the methods of the disclosure find use in experimental animals, in veterinary application, and in the development of animal models for disease. The term does not denote a particular age. Thus, both adult and newborn individuals are intended to be covered.

### Kits

The disclosure also provides kits comprising one or more containers holding compositions comprising one or more mRNAs encoding one or more cellular reprogramming factors for transient reprogramming of immune cells. Kits may further comprise transfection agents, media for culturing cells, and optionally one or more other factors, such as growth factors, ECM components, antibiotics, and the like. The mRNAs encoding cellular reprogramming factors and/or other compositions can be in liquid form or lyophilized. Such kits may also include components that preserve or maintain the mRNAs that protect against their degradation. Such components may be RNAse-free or protect against RNAses. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating immune cell related disease or conditions. The package insert can be an unapproved draft package insert or can be a package insert approved by the Food and Drug Administration (FDA) or other regulatory body.

In certain embodiments, the kit comprises T cells and mRNAs encoding one or more cellular reprogramming factors selected from the group consisting of OCT4, SOX2, KLF4, c-MYC, LIN28, NANOG, and GLIS1. In certain embodiments, the kit comprises T cells and mRNAs encoding one or more cellular reprogramming factors selected from the group consisting of OCT4, SOX2, KLF4, c-MYC, LIN28, NANOG, and GLIS1. In certain embodiments, the kit comprises T cells and mRNAs encoding two or more cellular reprogramming factors selected from the group consisting of OCT4, SOX2, KLF4, c-MYC, LIN28, NANOG, and GLIS1. In certain embodiments, the kit comprises T cells and mRNAs encoding three or more cellular reprogramming factors selected from the group consisting of OCT4, SOX2, KLF4, c-MYC, LIN28, NANOG, and GLIS1. In certain embodiments, the kit comprises T cells and mRNAs encoding four or more cellular reprogramming factors selected from the group consisting of OCT4, SOX2, KLF4, c-MYC, LIN28, NANOG, and GLIS1. In certain embodiments, the kit comprises T cells and mRNAs encoding five or more cellular reprogramming factors selected from the group consisting of OCT4, SOX2, KLF4, c-MYC, LIN28, NANOG, and GLIS1. In certain embodiments, the kit comprises T cells and mRNAs encoding six cellular reprogramming factors selected from the group consisting of OCT4, SOX2, KLF4, c-MYC, LIN28, NANOG, and GLIS1. In certain embodiments, the kit comprises T cells and mRNAs encoding the cellular reprogramming factors OCT4, SOX2, KLF4, c-MYC, LIN28, NANOG, and GLIS1. In certain embodiments, the kit comprises T cells and mRNAs encoding the cellular reprogramming factors OCT4, SOX2, KLF4, c-MYC, LIN28, and NANOG.

### III. Examples

The following examples are illustrative in nature and are in no way intended to be limiting.

### Example 1

### Effect of Dosing Duration and Factor Combination on Rejuvenation, Exhaustion, Proliferation, and Efficacy Oft Cells

mRNA molecules encoding OCT4 (0), SOX2 (S), KLF4 (K), c-MYC (M), LIN28 (L), and NANOG (N)(i.e., one factor per molecule) are synthesized in vitro from plasmid DNA and purified. Pan T-cells are isolated by using MACSxpress® Buffy Coat Pan T Cell Isolation Kit, human (Cat: 130-120-001) from Buffy Coat obtained from blood samples of young (age 20-39 years), middle-aged (age 40-59 years), and aged (age >60 years) individuals, or from commercially available human buffy coat, human buffy coat leukocytes, or human blood samples. Pan T-cells are transduced with lentivirus containing Anti-CD19-ScFv-CD28 (no CD3ζ, Anti-CD19-ScFv-CD28-CD3ζ, and Anti-CD19-ScFv-4-1BB-CD3ζ obtained from GenTarget Inc. and activated by Dynabeads™ Human T-Activator CD3/CD28 for T Cell Expansion and Activation kit (ThermoFisher Cat: 11161D) in X-VIVO-15 (Lonza Cat:BEBP02-054Q) medium, or used in a naïve, non-transduced, non-activated state. In additional conditions, multiple activations are performed, for example 2, 3, or 4 sequential activations.

mRNA molecules in the combinations OSKMLN, OSKM, and OSK are prepared as naked mRNA in nuclease free H₂O and applied to T cells using electroporation with the following parameters and conditions: 3-days post activation with Dynabeads, Pan T-cells are electroporated with Neon Transfection System at 1350V, 10 ms, 3 pulse or 1600V, 10 ms, 3 pulse. In each replicate in each condition, 50 nanograms to 10 micrograms of mRNA are used per 1 million cells. An electroporation-only and non-treated control are also included. After electroporation, cells are returned to culture conditions. Cells are cultured for a total of 3, 5, 7, 9, 12, 14, 17, 15, and 21 days, with electroporation delivery of mRNA performed once per day, every other day, or every three days. At 3-18 days after the last electroporation, cells are collected for analysis. In additional conditions, mRNA molecules O, S, K, M, L, and N are applied in all single-factor, two-factor, three-factor, four-factor, and five-factor combinations, for example OSKMLN, OSKML, SKMLN, OSKM, SKML, KMLN, OSLN, KOMN, OMN, MLN, OS, etc. In additional conditions, transfection is performed at the same time intervals but using lipid nanoparticles and polymer nanoparticles for transfection rather than electroporation; such nanoparticles are as in Example 3. In some experiments, delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of T-cell identity, T-cell markers CD8 and CD3 are assessed using flow cytometry. Other markers of T-cell identity or lineage known in the art may also be used.

For analysis of rejuvenation, stemness markers CCR7, CD62L, TCF7 are assessed using flow cytometry. The Horvath epigenetic clock is also used. Other markers of stemness or rejuvenation known in the art may also be used.

For analysis of prevention and reversal of exhaustion and senescence, surface expression of CD39, CD57, PD-1, 2B4, LAG-3 (CD223), KLRG-1 (MAFA), TIGIT, CTLA-4, CD56, CD27, CD28, and TIM-3 (CD366) proteins is assessed using flow cytometry. Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, GzmB, Perforin, IL12p70, and IL-8) assay using ELISA or Luminex are performed. Additionally, short-term and long-term killing assays are performed according to methods well known to those skilled in the art, for example as described in Sommer et al. (Molecular Therapy, 2019 Jun. 5; 27(6):1126-38), in Davenport et al. (Cancer Immunology Research, 2015 May 1; 3(5):483-94), Zolov et al. (Cytotherapy, 2018 Oct. 1; 20(10):1259-66), Lee et al. (Molecular Therapy, 2022 Feb. 2; 30(2):579-92), Wei et al. (Journal for Immunotherapy of Cancer. 2019 December; 7(1):1-5), or Konduri et al. (Science Translational Medicine. 2021 May 5; 13(592):eabc3196).

Data from Example 1 demonstrate reprogramming factors and/or reprogramming durations which provide advantageous T cell features and characteristics. Reprogramming factors, and combinations and conditions such as reprogramming duration thereof, which decrease or reverse T cell exhaustion, increase T cell rejuvenation, increase or extend self-renewal, increase or extend multipotency, increase or extend functional persistence, provide retention of T cell identity, enhance T cell proliferation and provide increased efficacy, such as increased tumor killing, may provide attractive candidates for immune cell rejuvenation compositions, methods, kits and associated therapies.

### Example 2

### Effect of Dosing Interval and Factor Combination on Rejuvenation, Exhaustion, Proliferation, and Efficacy of T Cells

mRNA molecules encoding OCT4 (0), SOX2 (S), KLF4 (K), c-MYC (M), LIN28 (L), and NANOG (N)(i.e., one factor per molecule) are synthesized in vitro from plasmid DNA and purified. Pan T-cells isolated by using MACSxpress® Buffy Coat Pan T Cell Isolation Kit, human (Cat: 130-120-001) from Buffy Coat from blood samples of young (age 20-25 years), middle-aged (age 40-50 years), and aged (age >65 years) individuals or from commercially available human buffy coat, human buffy coat leukocytes, or human blood samples. Pan T-cells are transduced with lentivirus containing Anti-CD19-ScFv-CD28 (no CD3ζ), Anti-CD19-ScFv-CD28-CD3ζ, and Anti-CD19-ScFv-4-1BB-CD3ζ obtained from GenTarget Inc. and activated by Dynabeads™ Human T-Activator CD3/CD28 for T Cell Expansion and Activation kit (ThermoFisher Cat: 11161D) in X-VIVO-15 (Lonza Cat:BEBP02-054Q) medium, or used in a naïve, non-transduced, non-activated state. In additional conditions, multiple activations are performed, for example 2, 3, or 4 sequential activations.

mRNA molecules in the combinations OSKMLN, OSKM, and OSK are prepared as naked mRNA in nuclease free H₂O and applied to T cells using electroporation with the following parameters and conditions: 3-days post activation with Dynabeads, Pan T-cells are electroporated with Neon Transfection System at 1350V, 10 ms, 3 pulse or 1600V, 10 ms, 3 pulse. In each replicate in each condition, 50 nanograms to 10 micrograms of mRNA are used and per 1 million cells. Three dosing intervals of electroporation performed: (1) prevention of exhaustion on days 4-9, (2) reversal of exhaustion on days 10-15, and (3) reversal of exhaustion for the cell therapy product on days 16-21. An electroporation-only and non-treated control are also included. After electroporation, cells are returned to culture conditions. Cells are cultured for total of 15 or 21 days, with electroporation delivery of mRNA performed once per day, every other day, or every three days for each interval separately (i.e., days 4-9 every day, every other day or every three days; days 10-15 every day, every other day or every three days; days 16-21 every day, every other day or every three days). At the end of the culture period (15 days or 21 days), cells are collected for analysis. In additional conditions, transfection is performed at the same time intervals but using lipid nanoparticles and polymer nanoparticles for transfection rather than electroporation; such nanoparticles are as in Example 3. In some experiments, delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of T-cell identity, T-cell markers CD8 and CD3 are assessed using flow cytometry. Other markers of T-cell identity or lineage known in the art may also be used.

For analysis of rejuvenation, stemness markers CCR7, CD62L, TCF7 are assessed using flow cytometry. The Horvath epigenetic clock is also used. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, 2B4, and TIM-3 (CD366) proteins is assessed using flow cytometry.

Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, GzmB, Perforin, and IL-8) assay using ELISA or Luminex are performed. Short-term (20 hours to 3 days) cytotoxicity assays are performed using a ONE-Glo™ Luciferase Assay System Kit (Promega, Cat: E6110) following manufacturer's protocol. Briefly, eGFP-FLuc expressing CD19+ Daudi target cells are incubated with anti-CD19 CAR-T cells for 20 hours-72 hours at various effector-target ratios and placed into 96-well plates. The amount of oxyfluoroluciferin that is converted into 5′-fluoroluciferin by live Daudi target cells expressing FLuc is measured on a luminometer. Measurements in the long-term killing assay are identical to those in the short-term killing assay. However, the starting effector:target ratio is 1:2, 1:1, 2:1, or 4:1. Every 3 days, half of the culture is transferred to a new plate and analyzed for percent killing, and the rest of the culture is re-challenged with fresh Daudi target cells.

Additionally, short-term and long-term killing assays are performed according to methods well known to those skilled in the art, for example as described in Sommer et al. (Molecular Therapy, 2019 Jun. 5; 27(6):1126-38), in Davenport et al. (Cancer Immunology Research, 2015 May 1; 3(5):483-94), Zolov et al. (Cytotherapy, 2018 Oct. 1; 20(10):1259-66), Lee et al. (Molecular Therapy, 2022 Feb. 2; 30(2):579-92), Wei et al. (Journal for Immunotherapy of Cancer. 2019 December; 7(1):1-5), or Konduri et al. (Science Translational Medicine. 2021 May 5; 13(592):eabc31%).

Data from Example 2 experiments demonstrate reprogramming factors and/or dosing intervals which provide advantageous T cell features and characteristics. Reprogramming factors, and combinations and conditions such dosing intervals thereof, which inhibit, reduce or reverse T cell exhaustion, increase T cell rejuvenation, provide retention of T cell identity, enhance T cell proliferation and provide increased efficacy, such as increased tumor killing, may provide attractive candidates for immune cell rejuvenation compositions, methods, kits and associated therapies.

### Example 3

### Comparison of Nanoparticle mRNA Transfection to Electroporation: Effects on Rejuvenation, Exhaustion, and Proliferation, and Efficacy of T Cells

mRNA molecules encoding OCT4 (0), SOX2 (S), KLF4 (K), c-MYC (M), LIN28 (L), and NANOG (N)(i.e., one factor per molecule) are synthesized in vitro from plasmid DNA and purified. Pan T-cells isolated by using MACSxpress® Buffy Coat Pan T Cell Isolation Kit, human (Cat: 130-120-001) from Buffy Coat of from blood samples of young (age 20-39 years), middle-aged (age 40-59 years), and aged (age >60 years) individuals or from commercially available human buffy coat, human buffy coat leukocytes, or human blood samples. Pan T-cells are transduced with lentivirus containing Anti-CD19-ScFv-CD28 (no CD3ζ, Anti-CD19-ScFv-CD28-CD3∂, and Anti-CD19-ScFv4-1BB-CD3ζ obtained from GenTarget Inc. and activated by Dynabeads™ Human T-Activator CD3/CD28 for T Cell Expansion and Activation kit (ThermoFisher Cat: 11161D) in X-VIVO-15 (Lonza Cat:BEBP02-054Q) medium, or used in a naïve, non-transduced, non-activated state. In additional conditions, multiple activations are performed, for example 2, 3, or 4 sequential activations.

mRNA molecules in the combination OSKMLN are prepared as naked mRNA in nuclease free H₂O or incorporated into nanoparticles as described in manufacturer's protocols for Lipofectamine, Fugene, or Polyplus, or in Billingsley et al., Nano Lett. 2020 (20, 3, 1578-1589), McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. Nanoparticles incorporating lipids selected from those described in FIGS. 1-18 are also used. Naked mRNA are transfected into T cells using electroporation with the following parameters and conditions: 3-days post activation with Dynabeads, Pan T-cells are electroporated with Neon Transfection System at 1350V, 10 ms, 3 pulse or 1600V, 10 ms, 3 pulse. Nanoparticle-incorporated mRNA are transfected into T cells under conditions described in manufacturer's protocols for Lipofectamine, Fugene, or Polyplus, or in Billingsley et al., Nano Lett. 2020 (20, 3, 1578-1589), McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. In each replicate in each condition, 50 nanograms to 10 micrograms of mRNA are used per 1 million cells. After electroporation or nanoparticle transfection, cells are returned to culture conditions. Via transfection, cells are exposed to mRNA for 0-10 days and then cultured for 0-18 days. Cells are then collected for analysis.

For analysis of T-cell identity, T-cell markers CD8 and CD3 are assessed using flow cytometry. Other markers of T-cell identity or lineage known in the art may also be used.

For analysis of rejuvenation, sternness markers CCR7, CD62L, TCF7 are assessed using flow cytometry. The Horvath epigenetic clock is also used. Other markers of sternness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, 2B4, and TIM-3 (CD366) proteins is assessed using flow cytometry.

Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, GzmB, Perforin, and IL-8) assay using ELISA or Luminex is performed. Short-term (20 h-3 days) cytotoxicity assays are performed using a ONE-Glo™ Luciferase Assay System Kit (Promega, Cat: E6110) following manufacturer's protocol. Briefly, eGFP-FLuc expressing CD19+ Daudi target cells are incubated with anti-CD19 CAR-T cells for 20 h-72 h at various effector:target ratios and placed into 96-well plates. The amount of oxyfluoroluciferin that is converted into 5′-fluoroluciferin by live Daudi target cells expressing FLuc is measured on a luminometer. Measurements in the long-term killing assay are identical to those in the short-term killing assay. However, the starting effector:target ratio is 1:2, 1:1, 2:1, or 4:1. Every 3 days, half of the culture is transferred to a new plate and analyzed for percent killing, and the rest of the culture is re-challenged with fresh Daudi target cells. Additionally, short-term and long-term killing assays are performed according to methods well known to those skilled in the art, for example as described in Sommer et al. (Molecular Therapy, 2019 Jun. 5; 27(6):1126-38), in Davenport et al. (Cancer Immunology Research, 2015 May 1; 3(5):483-94), Zolov et al. (Cytotherapy, 2018 Oct. 1; 20(10):1259-66), Lee et al. (Molecular Therapy, 2022 Feb. 2; 30(2):579-92), Wei et al. (Journal for Immunotherapy of Cancer. 2019 December; 7(1):1-5), or Konduri et al. (Science Translational Medicine. 2021 May 5; 13(592):eabc3l %).

Data from Example 3 is analyzed to demonstrate that delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to delivery of mRNA molecules using electroporation. In some experiments, delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

Data from Example 3 experiments also demonstrate reprogramming factors which provide advantageous T cell features and characteristics. Reprogramming factors, and combinations thereof, that prevent, decrease or reverse T cell exhaustion, increase T cell rejuvenation, provide retention of T cell identity, enhance T cell proliferation and provide increased efficacy, such as increased tumor killing, may provide attractive candidates for immune cell rejuvenation compositions, methods, kits and associated therapies.

### Example 4

### Effect of Reprogramming Factor Ratio on Rejuvenation, Exhaustion, Proliferation, AND EFFICACY OF T CELLS

mRNA molecules encoding OCT4 (0), SOX2 (S), KLF4 (K), c-MYC (M), LIN28 (L), and NANOG (N)(i.e., one factor per molecule) are synthesized in vitro from plasmid DNA and purified. Pan T-cells isolated by using MACSxpress® Buffy Coat Pan T Cell Isolation Kit, human (Cat: 130-120-001) from Buffy Coat from blood samples of young (age 20-39 years), middle-aged (age 40-60 years), and aged (age >60 years) individuals, or from commercially available human buffy coat, human buffy coat leukocytes, or human blood samples. Pan T-cells are transduced with lentivirus containing Anti-CD19-ScFv-CD28 (no CD3ζ, Anti-CD19-ScFv-CD28-CD3ζ, and Anti-CD19-ScFv4-1BB-CD3ζ obtained from GenTarget Inc. and activated by Dynabeads™ Human T-Activator CD3/CD28 for T Cell Expansion and Activation kit (Thermofisher Cat: 11161D) in X-VIVO-15 (Lonza Cat: BEBPO2-054Q) medium, or used in a naïve, non-transduced, non-activated state. In additional conditions, multiple activations are performed, for example 2, 3, or 4 sequential activations.

mRNA molecules are prepared as naked mRNA in nuclease free H₂O, and mixed into cocktails with OSKLMN mass ratios of 1-5:1-5:1-5:1-5:1-5:1-5. Naked mRNA are transfected into T cells using electroporation with the following parameters and conditions: 3-days post activation with Dynabeads, Pan T-cells are electroporated with Neon Transfection System at 1350V, 10 ms, 3 pulse or 1600V, 10 ms, 3 pulse. In each replicate in each condition, 50 nanograms to 10 micrograms of mRNA are used per 1 million cells. An electroporation-only and non-treated control are also included. After electroporation, cells are returned to culture conditions. Via electroporation, cells are exposed to mRNA for 0-10 days and then cultured for 0-18 days. In additional conditions, transfection is performed at the same time intervals but using lipid nanoparticles and polymer nanoparticles for transfection rather than electroporation; such nanoparticles are as in Example 3. In some experiments, delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of T-cell identity, T-cell markers CD8 and CD3 are assessed using flow cytometry. Other markers of T-cell identity or lineage known in the art may also be used.

For analysis of rejuvenation, stemness markers CCR7, CD62L, TCF7 are assessed using flow cytometry.

Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, 2B4, and TIM-3 (CD366) proteins is assessed using flow cytometry. Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, GzmB, Perforin. and IL-8) assay using ELISA or Luminex as well as short-term and long-term killing assays is performed. Short-term (20 h-3 days) cytotoxicity assays are performed using a ONE-Glo™ Luciferase Assay System Kit (Promega, Cat: E6110) following manufacturer's protocol. Briefly, eGFP-FLuc expressing CD19+ Daudi target cells are incubated with anti-CD19 CAR-T cells for 20 h-72 h at various effector:target ratios and placed into 96-well plates. The amount of oxyfluoroluciferin that is converted into 5′-fluoroluciferin by live Daudi target cells expressing FLuc is measured on a luminometer. Measurements in the long-term killing assay are identical to those in the short-term killing assay. However, the starting effector target ratio is 1:2, 1:1, 2:1, or 4:1. Every 3 days, half of the culture is transferred to a new plate and analyzed for percent killing, and the rest of the culture is re-challenged with fresh Daudi target cells. Additionally, short-term and long-term killing assays are performed according to methods well known to those skilled in the art, for example as described in Sommer et al. (Molecular Therapy, 2019 Jun. 5; 27(6):1126-38), in Davenport et al. (Cancer Immunology Research, 2015 May 1; 3(5):483-94), Zolov et al. (Cytotherapy, 2018 Oct. 1; 20(10):1259-66), Lee et al. (Molecular Therapy, 2022 Feb. 2; 30(2):579-92), Wei et al. (Journal for Immunotherapy of Cancer. 2019 December; 7(1):1-5), or Konduri et al. (Science Translational Medicine. 2021 May 5; 13(592):eabc3196).

Data from Example 4 experiments demonstrate reprogramming factors and/or reprogramming factor ratios which provide advantageous T cell features and characteristics. Reprogramming factors, and combinations including ratios thereof, that decrease or reverse T cell exhaustion, increase T cell rejuvenation, provide retention of T cell identity, enhance T cell proliferation and provide increased efficacy, such as increased tumor killing, may provide attractive candidates for immune cell rejuvenation compositions, methods, kits and associated therapies.

### Example 5

### Effect of Persistent Reprogramming Factor Exposure Within a Dosing Interval on Rejuvenation, Exhaustion, Proliferation, and Efficacy of T Cells

mRNA molecules encoding OCT4 (O), SOX2 (S), KLF4 (K), c-MYC (M), LIN28 (L), and NANOG (N) (i.e., one factor per molecule) are synthesized in vitro from plasmid DNA and purified. To control the persistence of reprogramming factor exposure, linear non replicative mRNA molecules are used with the 3′ untranslated region altered, poly (A) tail length optimized, and WPRE element included such that the half-life of the mRNA is 6 minutes to 24 hours. Circular RNA is used with a half-life of 1-3 days. Trans- or self-amplifying RNA is used with a half-life of up to 10-14 days. Pan T-cells isolated by using MACSxpress® Buffy Coat Pan T Cell Isolation Kit, human (Cat: 130-120-001) from Buffy Coat from blood samples of young (age 20-39 years), middle-aged (age 40-59 years), and aged (age >60 years) individuals, or from commercially available human buffy coat, human buffy coat leukocytes, or human blood samples. Pan T-cells are transduced with lentivirus containing Anti-CD19-ScFv-CD28 (no CD3ζ), Anti-CD19-ScFv-CD28-CD3ζ, and Anti-CD19-ScFv-4-1BB-CD3ζ obtained from GenTarget Inc. and activated by Dynabeads™ Human T-Activator CD3/CD28 for T Cell Expansion and Activation kit (Thermofisher Cat: 11161D) in X-VIVO-15 (Lonza Cat:BEBP02-054Q) medium, or used in a naïve, non-transduced, non-activated state. In additional conditions, multiple activations are performed, for example 2, 3, or 4 sequential activations.

mRNA molecules in the combination OSKMLN are prepared as naked mRNA in nuclease free H₂O and transfected into T cells using electroporation with the following parameters and conditions: 3-days post activation with Dynabeads, Pan T-cells are electroporated with Neon Transfection System at 1350V, 10 ms, 3 pulse or 1600V, 10 ms, 3 pulse. In each replicate in each condition, 50 nanograms to 10 micrograms of mRNA are used per 1 million cells. An electroporation-only and non-treated control are also included. Cells are cultured for 3-10 days, with electroporation delivery of mRNA performed on day 1, day 2, day 3, day 4, day 5, day 6, day 7, day 8, day 9, and/or day 10, depending on the mRNA construct used (i.e., transfection only performed on day 1 for mRNA with the longest expression, longest half-life, or expressing the transcription factors with the longest half-lives, and every three days, every two days, or every day for mRNA with the shortest expression). At each time point, cells are collected for analysis. In additional conditions, transfection is performed at the same time intervals but using lipid nanoparticles and polymer nanoparticles for transfection rather than electroporation; such nanoparticles are as in Example 3. In some experiments, delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of T-cell identity, T-cell markers CD8 and CD3 are assessed using flow cytometry. Other markers of T-cell identity or lineage known in the art may also be used.

For analysis of rejuvenation, sternness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, 2B4, and TIM-3 (CD366) proteins is assessed using flow cytometry. Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, GzmB, Perforin, and IL-8) assay using ELISA or Luminex is performed. Short-term (20 h-3 days) cytotoxicity assays are performed using a ONE-Glo™ Luciferase Assay System Kit (Promega, Cat: E6110) following manufacturer's protocol. Briefly, eGFP-FLuc expressing CD19+ Daudi target cells are incubated with anti-CD19 CAR-T cells for 20 h-72 h at various effector:target ratios and placed into 96-well plates. The amount of oxyfluoroluciferin that is converted into 5′-fluoroluciferin by live Daudi target cells expressing FLuc is measured on a luminometer. Measurements in the long-term killing assay are identical to those in the short-term killing assay. However, the starting effector:target ratio is 1:2, 1:1, 2:1, or 4:1. Every 3 days, half of the culture is transferred to a new plate and analyzed for percent killing, and the rest of the culture is re-challenged with fresh Daudi target cells. Additionally, short-term and long-term killing assays are performed according to methods well known to those skilled in the art, for example as described in Sommer et al. (Molecular Therapy, 2019 Jun. 5; 27(6):1126-38), in Davenport et al. (Cancer Immunology Research, 2015 May 1; 3(5):483-94), Zolov et al. (Cytotherapy, 2018 Oct. 1; 20(10):1259-66), Lee et al. (Molecular Therapy, 2022 Feb. 2; 30(2):579-92), Wei et al. (Journal for Immunotherapy of Cancer. 2019 December; 7(1):1-5), or Konduri et al. (Science Translational Medicine. 2021 May 5; 13(592):eabc3196). Each of these references is incorporated herein.

Data from Example 5 experiments demonstrate reprogramming factors and/or mechanisms for persistent reprogramming factor exposure which provide advantageous T cell features and characteristics. Reprogramming factors, and combinations and expression regulatory mechanisms such as persistent expression thereof, that decrease or reverse T cell exhaustion, increase T cell rejuvenation, provide retention of T cell identity, enhance T cell proliferation and provide increased efficacy, such as increased tumor killing, may provide attractive candidates for immune cell rejuvenation compositions, methods, kits and associated therapies.

### Example 6

### Comparison of mRNA Transfection to Viral Transduction: Effects on Rejuvenation, Exhaustion, and Proliferation, and Efficacy of T Cells

mRNA molecules encoding OCT4 (0), SOX2 (S), KLF4 (K), c-MYC (M), LIN28 (L), and NANOG (N)(i.e., one factor per molecule) are synthesized in vitro from plasmid DNA and purified. Lentivirus, retrovirus, or sendaivirus vectors encoding O, S, K, M, L, and N are generated according to methods known in the art, for example as described in Egusa et al. (PLoS One. 2010 Sep. 14; 5(9):e12743). Pan T-cells isolated by using MACSxpress® Buffy Coat Pan T Cell Isolation Kit, human (Cat: 130-120-001) from Buffy Coat of from blood samples of young (age 20-39 years), middle-aged (age 40-59 years), and aged (age >60 years) individuals or from commercially available human buffy coat, human buffy coat leukocytes, or human blood samples. Pan T-cells are transduced with lentivirus containing Anti-CD19-ScFv-CD28 (no CD3ζ), Anti-CD19-ScFv-CD28-CD3ζ, and Anti-CD19-ScFv-4-1BB-CD3ζ obtained from GenTarget Inc. and activated by Dynabeads™ Human T-Activator CD3/CD28 for T Cell Expansion and Activation kit (Thermofisher Cat: 11161D) in X-VIVO-15 (Lonza Cat:BEBP02-054Q) medium, or used in a naïve, non-transduced, non-activated state. In additional conditions, multiple activations are performed, for example 2, 3, or 4 sequential activations.

mRNA molecules in the combination OSKMLN are incorporated into nanoparticles as described in manufacturer's protocols for Lipofectamine, Fugene, or Polyplus, or in Billingsley et al., Nano Lett. 2020 (20, 3, 1578-1589), McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. Nanoparticles incorporating lipids selected from those described in FIGS. 1-18 are also used. Nanoparticle-incorporated mRNA are transfected into T cells under conditions described in manufacturer's protocols for Lipofectamine, Fugene, or Polyplus, or in Billingsley et al., Nano Lett. 2020 (20, 3, 1578-1589), McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. In each replicate in each condition, 50 nanograms to 10 micrograms of mRNA are used per 1 million cells. Viral transduction is performed according to methods known in the art, for example as described in Egusa et al. (PLoS One. 2010 Sep. 14; 5(9):e12743). After nanoparticle transfection or viral transduction, cells are returned to culture conditions. Via transfection or transduction, cells are exposed to mRNA for 0-10 days and then cultured for 0-18 days. Cells are then collected for analysis.

For analysis of T-cell identity, T-cell markers CD8 and CD3 are assessed using flow cytometry. Other markers of T-cell identity or lineage known in the art may also be used.

For analysis of rejuvenation, T-cell stemness markers CCR7, CD62L, TCF7 are assessed using flow cytometry. Other markers of T-cell sternness or T-cell rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, 2B4, and TIM-3 (CD366) proteins is assessed using flow cytometry. Other markers of T-cell exhaustion or T-cell senescence known in the art may also be used.

For analysis of proliferation, cell numbers are counted by Invitrogen Countess Automated Cell Counter. For analysis of tumor cell killing efficacy, cytokine release (IFNγ, TNF-α, GM-CSF, IL-2, IL-6, IL-10, IL-13, IL12p70, GzmB, Perforin, and IL-8) assay using ELISA or Luminex is performed. Short-term (20 h-3 days) cytotoxicity assays are performed using a ONE-Glo™ Luciferase Assay System Kit (Promega, Cat: E6110) following manufacturer's protocol. Briefly, eGFP-FLuc expressing CD19+ Daudi target cells are incubated with anti-CD19 CAR-T cells for 20 h-72 h at various effector target ratios and placed into 96-well plates. The amount of oxyfluoroluciferin that is converted into 5′-fluoroluciferin by live Daudi target cells expressing FLuc is measured on a luminometer. Measurements in the long-term killing assay are identical to those in the short-term killing assay. However, the starting effector:target ratio is 1:2, 1:1, 2:1, or 4:1. Every 3 days, half of the culture is transferred to a new plate and analyzed for percent killing, and the rest of the culture is re-challenged with fresh Daudi target cells. Additionally, short-term and long-term killing assays are performed according to methods well known to those skilled in the art, for example as described in Sommer et al. (Molecular Therapy, 2019 Jun. 5; 27(6):1126-38), in Davenport et al. (Cancer Immunology Research, 2015 May 1; 3(5):483-94), Zolov et al. (Cytotherapy, 2018 Oct 1; 20(10):1259-66), Lee et al. (Molecular Therapy, 2022 Feb. 2; 30(2):579-92), Wei et al. (Journal for Immunotherapy of Cancer. 2019 December; 7(1):1-5), or Konduri et al. (Science Translational Medicine. 2021 May 5; 13(592):eabc3196).

Data from Example 6 is analyzed to demonstrate that delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to viral gene delivery of reprogramming factors. In some experiments, delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to viral gene delivery of reprogramming factors.

Data from Example 6 experiments demonstrate reprogramming factors which provide advantageous T cell features and characteristics. Reprogramming factors, and combinations thereof, that decrease or reverse T cell exhaustion, increase T cell rejuvenation, provide retention of T cell identity, enhance T cell proliferation and provide increased efficacy, such as increased tumor killing, may provide attractive candidates for immune cell rejuvenation compositions, methods, kits and associated therapies.

### Example 7

### Rejuvenation, Exhaustion, Proliferation, and Efficacy of Tumor Infiltrating Lymphocytes

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for tumor-infiltrating lymphocytes using similar protocols, conditions, and parameters used for T cells in Examples 1-6 above, except for the specific protocols, conditions, and parameters for tumor-infiltrating lymphocytes set forth below. Tumor infiltrating lymphocytes are obtained and expanded from commercial sources or according to methods known to those skilled in the art, for example as described by Wu et al. (Nanomedicine. 2019 April, 14(8):955-67), Friedman et al. (Journal of immunotherapy (Hagerstown, Md.: 1997). 2012 June; 35(5):400), Belldegrun et al. (Cancer research. 1988 Jan. 1; 48(1):206-14), or Rosenberg et al. (New England Journal of Medicine. 1990 Aug. 30; 323(9):570-8), incorporated herein by reference. Tumor infiltrating lymphocytes are stimulated with IL-2, for example as described by Rosenberg et al. (New England Journal of Medicine. 1990 Aug. 30; 323(9):570-8) or Wu et al. (Nanomedicine. 2019 April, 14(8):955-67), or left unstimulated. In additional conditions, stimulation is performed for different intervals, for example for 8 hours to 21 days are performed. In some experiments, delivery of mRNA molecules with lipid nanoparticles and/or polymer nanoparticles results in enhanced tumor infiltrating lymphocyte rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of tumor infiltrating lymphocyte identity, tumor infiltrating lymphocyte markers CD8, CD3, CD45, CD4, and FOXP3 are assessed using flow cytometry. Other markers of tumor infiltrating lymphocyte identity or lineage known in the art may also be used.

For analysis of rejuvenation, stemness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, and TIM-3 (CD366) proteins is assessed using flow cytometry. Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, and IL-8) assay using ELISA or Luminex are performed. Additionally, tumor killing assays are performed according to methods well known to those skilled in the art, for example as described in Ritthipichai et al. (Clinical Cancer Research. 2017 Oct. 15; 23(20):6151-64), Inozume et al. (Journal of Investigative Dermatology 136.1 (2016): 255-263), Ortegel et al. (Lung cancer. 2002 Apr. 1; 36(1):17-25), or Nielsen et al. (Cancer Immunology, Immunotherapy 69, no. 11(2020): 2179-219), incorporated herein by reference.

Data from Example 7 experiments demonstrate reprogramming factors which provide advantageous tumor infiltrating lymphocyte features and characteristics. Reprogramming factors, and combinations, mass or molar ratios, reprogramming durations and intervals thereof, that decrease or reverse tumor infiltrating lymphocyte exhaustion, increase tumor infiltrating lymphocyte rejuvenation, provide retention of tumor infiltrating lymphocyte identity, enhance tumor infiltrating lymphocyte proliferation and provide increased efficacy, such as increased tumor killing, may provide attractive candidates for immune cell rejuvenation compositions, methods, kits and associated therapies.

### Example 8

### Rejuvenation, Exhaustion, Proliferation, and Efficacy of Regulatory T Cells (TREGS)

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for Tregs using similar protocols, conditions, and parameters used for T cells in Examples 1-6 above, except for the specific protocols, conditions, and parameters for tumor-infiltrating lymphocytes set forth below. Tregs are obtained and expanded from commercial sources or according to methods known to those skilled in the art, for example as described by Collison et al. (Regulatory T cells 2011 (pp. 21-37). Humana Press, Totowa, NJ), incorporated herein by reference. Tregs are activated using anti-CD3+anti-CD28 coated beads as described in Example 1 above, or using irradiated splenocytes or purified dendritic cells combined with soluble anti-CD3 or peptide, as described in Collison et al. (Regulatory T cells 2011 (pp. 21-37). Humana Press, Totowa, NJ), or left un-activated. In some experiments, delivery of mRNA molecules with lipid nanoparticles and/or polymer nanoparticles results in enhanced Treg rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of Treg identity, Treg markers CD3, CD4, CD25, CD127, IL-2R alpha, and FoxP3 are assessed using flow cytometry. Other markers of Treg identity or lineage known in the art may also be used.

For analysis of rejuvenation, the markers CCR7, CD62L, TCF7, and GARP are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, and TIM-3 (CD366) proteins, or of CD4, CD25, and CD279, is assessed using flow cytometry. Other markers of exhaustion or senescence known in the art may also be used.

For analysis of proliferation, cell numbers are counted by Invitrogen Countess Automated Cell Counter.

For analysis of anti-inflammatory or immunomodulatory efficacy, assays known to those skilled in the art are used, for example inhibition of effector T cell proliferation as described by Pedersen et al. (Immunopharmacology and Immunotoxicology, Volume 37, 2015—Issue 1) or Court et al. (EMBO Reports (2020)21:e48052).

Data from Example 8 experiments demonstrate reprogramming factors which provide advantageous Treg features and characteristics. Reprogramming factors, and combinations, ratios, reprograming durations and intervals thereof, that decrease or reverse Treg exhaustion, increase Treg rejuvenation, provide retention of Treg identity, enhance Treg proliferation and provide increased efficacy, such as immunotolerance or decreased inflammation, may provide attractive candidates for immune cell rejuvenation compositions, methods, kits and associated therapies.

### Example 9

### Rejuvenation, Exhaustion, Proliferation, and Efficacy of Gamma Delta T Cells

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for gamma delta T cells using similar protocols, conditions, and parameters used for T cells in Examples 1-6 above, except for the specific protocols, conditions, and parameters for gamma delta T cells set forth below. Gamma delta T cells are obtained and expanded from commercial sources or according to methods known to those skilled in the art, for example as described by Pauza et al. (Frontiers in immunology. 2018 Jun. 8; 9:1305) or Deniger et al. (Clin Cancer Res (2014) 20 (22): 5708-5719), incorporated herein by reference. Gamma delta T cells are stimulated with IL-2 and/or IL-21, for example as described by Pauza et al. (Frontiers in immunology. 2018 Jun. 8; 9:1305) or Deniger et al. (Clin Cancer Res (2014) 20 (22): 5708-5719), or left unstimulated. In additional conditions, stimulation is performed for different intervals, for example for 8 hours to 21 days. In some experiments, delivery of mRNA molecules with lipid nanoparticles and/or polymer nanoparticles results in enhanced gamma delta T cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of gamma delta T cell identity, CD8 and CD3 are assessed using flow cytometry. Other markers of gamma delta T cell identity or lineage known in the art may also be used.

For analysis of rejuvenation, stemness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, and TIM-3 (CD366) proteins is assessed using flow cytometry. Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, IL-8, and IL-17) assay using ELISA or Luminex are performed. Additionally, tumor killing assays are performed according to methods well known to those skilled in the art, for example against solid tumor models of colorectal, bladder, prostate, and breast cancer cells as described in Paiza et al. (Frontiers in immunology. 2018 Jun. 8; 9:1305), or against various solid and hematological tumor cell lines as described by Deniger et al. (Clin Cancer Res (2014) 20 (22): 5708-5719), incorporated herein by reference.

Data from Example 9 experiments demonstrate reprogramming factors which provide advantageous gamma delta T cell features and characteristics. Reprogramming factors, and combinations, mass or molar ratios, reprogramming durations and intervals thereof, that decrease or reverse gamma delta T cell exhaustion, increase gamma delta T cell rejuvenation, provide retention of gamma delta T cell identity, enhance gamma delta T cell proliferation and provide increased efficacy, such as increased tumor killing, may provide attractive candidates for immune cell rejuvenation compositions, methods, kits and associated therapies.

### Example 10

### Rejuvenation, Exhaustion, Proliferation, and Efficacy of Natural Killer T Cells

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for natural killer T (NKT) cells using similar protocols, conditions, and parameters used for T cells in Examples 1-6 above, except for the specific protocols, conditions, and parameters for NKT cells set forth below. NKT cells are obtained and expanded from commercial sources or according to methods known to those skilled in the art, for example as described by Pule et al. (Frontiers in immunology. 2018 Jun. 8; 9:1305) or Metelitsa et al. (J Exp Med, 2004, vol. 199 9 (pg. 1213-1221), incorporated herein by reference. NKT cells are transduced with CAR, activated with αGalCer-pulsed irradiated PBMCs, and/or stimulated with IL-2, for example as described by Heczey et al. (Blood, 2014 Oct. 30; 124(18):2824-33), or left untransduced and/or unstimulated. In additional conditions, stimulation is performed for different intervals, for example for 8 hours to 21 days. In some experiments, delivery of mRNA molecules with lipid nanoparticles and/or polymer nanoparticles results in enhanced NKT cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of NKT cell identity, CD161 and CD94 are assessed using flow cytometry. Other markers of NKT cell identity or lineage known in the art may also be used.

For analysis of rejuvenation, stemness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, and TIM-3 (CD366) proteins is assessed using flow cytometry. Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, IL-8, and IL-17) assay using ELISA or Luminex are performed. Additionally, tumor killing assays are performed according to methods well known to those skilled in the art, for example using NB cells and M2 macrophages as effector cells as described in Rooney et al. (Int J Cancer, 1984, vol. 34 3 (pg. 339-348) and Heczey et al. (Blood, 2014 Oct. 30; 124(18):2824-33), incorporated herein by reference.

Data from Example 10 experiments demonstrate reprogramming factors which provide advantageous NKT cell features and characteristics. Reprogramming factors, and combinations, mass or molar ratios, reprogramming durations and intervals thereof, that decrease or reverse NKT cell exhaustion, increase NKT cell rejuvenation, provide retention of NKT cell identity, enhance NKT cell proliferation and provide increased efficacy, such as increased tumor killing, may provide attractive candidates for immune cell rejuvenation compositions, methods, kits and associated therapies.

### Example 11

### Rejuvenation, Exhaustion, Proliferation, and Efficacy of Natural Killer Cells

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for natural killer (NK) cells using similar protocols, conditions, and parameters used for T cells in Examples 1-6 above, except for the specific protocols, conditions, and parameters for NK cells set forth below. NK cells are obtained and expanded from commercial sources or according to methods known to those skilled in the art, for example as described by Fujisaki et al. (Cancer Res (2009) 69 (9): 4010-4017), Shook et al. (Tissue antigens. 2011 December; 78(6):409-15), or Liu et al. (Clin Cancer Res (2013) 19 (8): 2132-2143), incorporated herein by reference. NK cells are transduced with CAR, activated with αGalCer-pulsed irradiated PBMCs, and/or stimulated with IL-2, IL-12, IL-15, and/or IL-21, for example as described by Fujisaki et al. (Cancer Res (2009) 69 (9): 4010-4017), Shook et al. (Tissue antigens. 2011 Dec; 78(6):409-15), or Liu et al. (Clin Cancer Res (2013) 19 (8): 2132-2143), or left untransduced and/or unstimulated. In additional conditions, stimulation is performed for different intervals, for example for 8 hours to 21 days. In some experiments, delivery of mRNA molecules with lipid nanoparticles and/or polymer nanoparticles results in enhanced NK cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of NK cell identity, CD161, CD94, CD56, CD3, CD16 are assessed using flow cytometry. Other markers of NK cell identity or lineage known in the art may also be used.

For analysis of rejuvenation, sternness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, and TIM-3 (CD366) proteins is assessed using flow cytometry. Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, IL-8, IL-17, sCD40L, CCL2/MCP-1, CXCL9/MIG, and CXCL11/I-TAC) assay using ELISA or Luminex are performed. Additionally, tumor killing assays are performed according to methods well known to those skilled in the art, for example as described by Fujisaki et al. (Cancer Res (2009) 69 (9): 4010-4017), Shook et al. (Tissue antigens. 2011 December; 78(6):409-15), and Liu et al. (Clin Cancer Res (2013) 19 (8): 2132-2143), incorporated herein by reference.

Data from Example 11 experiments demonstrate reprogramming factors which provide advantageous NK cell features and characteristics. Reprogramming factors, and combinations, mass or molar ratios, reprogramming durations and intervals thereof, that decrease or reverse NK cell exhaustion, increase NK cell rejuvenation, provide retention of NK cell identity, enhance NK cell proliferation and provide increased efficacy, such as increased tumor killing, may provide attractive candidates for immune cell rejuvenation compositions, methods, kits and associated therapies.

### Example 12

### Rejuvenation, Exhaustion, Proliferation, and Efficacy of B Cells

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for B cells using similar protocols, conditions, and parameters used for T cells in Examples 1-6 above, except for the specific protocols, conditions, and parameters for B cells set forth below. B cells are obtained and expanded from commercial sources or according to methods known to those skilled in the art, for example as described by Kardava et al. (The Journal of Clinical Investigation, 2011 Jul. 1; 121(7):2614-24), incorporated herein by reference. Exhaustion is induced according to methods known in the art, for example as described by Kardava et al. (The Journal of Clinical Investigation, 2011 Jul. 1; 121(7):2614-24). In some experiments, delivery of mRNA molecules with lipid nanoparticles and/or polymer nanoparticles results in enhanced B cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of B cell identity, CD27 and CD21 are assessed using flow cytometry. Other markers of B cell identity or lineage known in the art may also be used.

For analysis of rejuvenation, stemness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, TIM-3 (CD366), FCRL4, CD32b, CD22, CD85j, and CD85d proteins is assessed using flow cytometry. Other markers of exhaustion or senescence known in the art may also be used.

For analysis of proliferation, cell numbers are counted by Invitrogen Countess Automated Cell Counter.

For analysis of efficacy, effector function and response to stimulation are evaluated according to methods well known to those skilled in the art, for example as described by Kardava et al. (The Journal of Clinical Investigation, 2011 Jul. 1; 121(7):2614-24), incorporated herein by reference.

Data from Example 12 experiments demonstrate reprogramming factors which provide advantageous B cell features and characteristics. Reprogramming factors, and combinations, mass or molar ratios, and reprogramming durations and intervals thereof, that decrease or reverse B cell exhaustion, increase B cell rejuvenation, provide retention of B cell identity, enhance B cell proliferation and provide increased efficacy, such as increased tumor killing, may provide attractive candidates for immune cell rejuvenation compositions, methods, kits and associated therapies.

### Example 13

### Rejuvenation, Exhaustion, Proliferation, and Efficacy of Dendritic Cells

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for dendritic cells using similar protocols, conditions, and parameters used for T cells in Examples 1-6 above, except for the specific protocols, conditions, and parameters for dendritic cells set forth below. Dendritic cells are obtained and expanded from commercial sources or according to methods known to those skilled in the art, for example as described by Hokey et al. (Cancer Research. 2005 Nov. 1; 65(21):10059-67), incorporated herein by reference. Dendritic cells are stimulated with OVA, CpGs, and/or poly(I:C), for example as described by Hokey et al. (Cancer Research. 2005 Nov. 1; 65(21):10059-67). In some experiments, delivery of mRNA molecules with lipid nanoparticles and/or polymer nanoparticles results in enhanced dendritic cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of dendritic cell identity, BDCA-1, CD8, CD11b, CD11c, CD8-α, CD103, CD205, CD40, CD80, CD86, and MHC Class I and Class II are assessed for classical dendritic cells and BDCA-2, BDCA-4, CD11c, CD45RA, CD123, ILT-7, MHC Class II, TLR7, and TLR9 are assessed for plasmacytoid dendritic cells using flow cytometry. Other markers of dendritic cell identity or lineage known in the art may also be used.

For analysis of rejuvenation, sternness markers CCR7, CD62L, TCF7 are assessed using flow cytometry. Other markers of sternness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, TIM-3 (CD366), FCRL4, CD32b, CD22, CD85j, and CD85d proteins is assessed using flow cytometry. Other markers of exhaustion or senescence known in the art may also be used.

For analysis of proliferation, cell numbers are counted by Invitrogen Countess Automated Cell Counter.

For analysis of efficacy, cytokine release (IL-2, IL-12p70) assay using ELISA or Luminex are performed. Effector function, antigen presentation, and response to stimulation are evaluated according to methods well known to those skilled in the art, for example as described by Hokey et al. (Cancer Research. 2005 Nov. 1; 65(21):10059-67), incorporated herein by reference.

Data from Example 13 experiments demonstrate reprogramming factors which provide advantageous dendritic cell features and characteristics. Reprogramming factors, and combinations, mass or molar ratios, reprogramming duration and intervals thereof, that decrease or reverse dendritic cell exhaustion, increase dendritic cell rejuvenation, provide retention of dendritic cell identity, enhance dendritic cell proliferation and provide increased efficacy, such as increased tumor killing, may provide attractive candidates for immune cell rejuvenation compositions, methods, kits and associated therapies.

### Example 14

### Rejuvenation, Exhaustion, Proliferation, and Efficacy of Monocytes/Macrophages

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for monocytes/macrophages using similar protocols, conditions, and parameters used for T cells in Examples 1-6 above, except for the specific protocols, conditions, and parameters for monocytes/macrophages set forth below. Monocytes/macrophages are obtained and expanded from commercial sources or according to methods known to those skilled in the art, for example as described by Zhang et al. (Journal of hematology & oncology. 2020 December; 13(1):1-5), Pasch et al. (Cells 2022, 11(6), 994), Suzuki et al. (Nature volume 514, pages 450-454 (2014), or Mao et al. (Journal of cellular and molecular medicine. 2020 March, 24(6):3314-27), incorporated herein by reference. Monocytes/macrophages are transduced with CAR, polarized with LPS/IFN-7, and/or polarized with IL-4/IL-10, for example as described by Zhang et al. (Journal of hematology & oncology. 2020 December; 13(1):1-5) or Pasch et al. (Cells 2022, 11(6), 994), or left untransduced and/or unpolaized. In some experiments, delivery of mRNA molecules with lipid nanoparticles and/or polymer nanoparticles results in enhanced monocyte/macrophage cell rejuvenation, proliferation, recovery from or prevention of exhaustion, anti-pathogenic effects, anti-cancer effects, or anti-inflammatory effects in the exposed immune cell compared to delivery of mRNA molecules using electroporation. For analysis of monocyte/macrophage cell identity, CD11b and CD14 are assessed using flow cytometry. Other markers of monocyte/macrophage cell identity or lineage known in the art may also be used.

For analysis of rejuvenation, stemness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, and TIM-3 (CD366) proteins is assessed using flow cytometry. Other markers of exhaustion or senescence known in the art may also be used.

For analysis of proliferation, cell numbers are counted by Invitrogen Countess Automated Cell Counter.

For analysis of tumor cell killing efficacy, cytokine release (IFNγ, TNF-α, GM-CSF, IL-2, IL-6, IL-10, IL-13, IL12p70, IL-8, and IL-17) assay using ELISA or Luminex are performed. Additionally, phagocytic tumor killing assays are performed according to methods well known to those skilled in the art, for example as described in Rooney et al. Zhang et al. (Journal of hematology & oncology. 2020 December; 13(1):1-5), Pasch et al. (Cells 2022, 11(6), 994), Suzuki et al. (Nature volume 514, pages 450-454(2014), or Mao et al. (Journal of cellular and molecular medicine. 2020 March; 24(6):3314-27), incorporated herein by reference.

Data from Example 14 experiments demonstrate reprogramming factors which provide advantageous monocyte/macrophage features and characteristics. Reprogramming factors, and combinations, mass or molar ratios, reprogramming durations and intervals thereof, that decrease or reverse monocyte/macrophage exhaustion, increase monocyte/macrophage cell rejuvenation, provide retention of monocyte/macrophage cell identity, enhance monocyte/macrophage cell proliferation and provide increased efficacy, such as increased tumor killing, may provide attractive candidates for immune cell rejuvenation compositions, methods, kits and associated therapies.

### Example 15

### Rejuvenation, Exhaustion, Proliferation, and Efficacy of Neutrophils

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for neutrophils using similar protocols, conditions, and parameters used for T cells in Examples 1-6 above, except for the specific protocols, conditions, and parameters for neutrophils set forth below. Neutrophils are obtained and expanded from commercial sources or according to methods known to those skilled in the art, for example as described by Lin et al. (Scientific reports. 2020 Sep. 1; 10(1):1-2), incorporated herein by reference. Neutrophils are stimulated with LPS, for example as described by Lin et al. (Scientific reports. 2020 Sep. 1; 10(1):1-2), or left unstimulated. In some experiments, delivery of mRNA molecules with lipid nanoparticles and/or polymer nanoparticles results in enhanced neutrophil cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of neutrophil cell identity, CD11b, CD14, CD15, CD16, CD32, CD33, CD44, CD45, CEACAM-8, HLA-DR, integrin alpha 4, integrin beta 2, and L-selectin are assessed using flow cytometry. Other markers of neutrophil cell identity or lineage known in the art may also be used.

For analysis of rejuvenation, sternness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, TIM-3 (CD366), and TICAM-2 proteins is assessed using flow cytometry. Other markers of exhaustion or senescence known in the art may also be used.

For analysis of proliferation, cell numbers are counted by Invitrogen Countess Automated Cell Counter.

For analysis of efficacy, assays known to skilled in the art are performed, such as the swarming assay as described in Lin et al. (Scientific reports. 2020 Sep. 1; 10(1):1-2), incorporated herein by reference.

Data from Example 15 experiments demonstrate reprogramming factors which provide advantageous neutrophil cell features and characteristics. Reprogramming factors, and combinations, mass or molar ratios, reprogramming durations and intervals thereof, that decrease or reverse neutrophil exhaustion, increase neutrophil rejuvenation, provide retention of neutrophil identity, enhance neutrophil proliferation and provide increased efficacy, such as increased tumor killing and/or effector activity, may provide attractive candidates for immune cell rejuvenation compositions, methods, kits and associated therapies.

### Example 16

### REJUVENATION, EXHAUSTION, PROLIFERATION, AND EFFICACY OF Eosinophils

Effects of dosing duration, dosing interval, reprogramming factor combination, reprogramming factor mass or molar ratio, persistent reprogramming factor exposure, nanoparticle-mediated delivery vs. electroporation-mediated delivery, and mRNA transfection vs. viral transduction are observed for eosinophils using similar protocols, conditions, and parameters used for T cells in Examples 1-6 above, except for the specific protocols, conditions, and parameters for neutrophils set forth below. Eosinophils are obtained and expanded from commercial sources or according to methods known to those skilled in the art, for example as described by Ebisawa et al. (J Immunol May 1, 1994, 152 (9) 4590-4596), incorporated herein by reference. In some experiments, delivery of mRNA molecules with lipid nanoparticles and/or polymer nanoparticles results in enhanced eosinophil rejuvenation, proliferation, recovery from or prevention of exhaustion, anti-pathogenic effects, anti-cancer effects, or anti-inflammatory effects in the exposed immune cell compared to delivery of mRNA molecules using electroporation.

For analysis of eosinophil cell identity, CCR3, CD11b, CD14, CD15, CD16, CD45, CEACAM-8, EMR1, HLA-DR, IL-5 receptor alpha, integrin alpha 4, and siglec-8 are assessed using flow cytometry. Other markers of eosinophil cell identity or lineage known in the art may also be used.

For analysis of rejuvenation, sternness markers CCR7, CD62L, TCF7 are assessed using flow cytometry. Other markers of stemness or rejuvenation known in the art may also be used.

For analysis of prevention and reversal of exhaustion and senescence, surface expression of CD39, CD57, PD-1, LAG-3 (CD223), KLRG-1 (MAFA), TIGIT, CTLA-4, CD56, CD27, CD28, TIM-3 (CD366), and TICAM-2 proteins is assessed using flow cytometry. Other markers of exhaustion or senescence known in the art may also be used.

For analysis of proliferation, cell numbers are counted by Invitrogen Countess Automated Cell Counter.

For analysis of efficacy, assays known to skilled in the art are performed, for example co-culture assays as described in Reichman et al. (Cancer Immunol Res (2019) 7 (3): 388-400), incorporated herein by reference.

Data from Example 16 experiments demonstrate reprogramming factors which provide advantageous eosinophil features and characteristics. Reprogramming factors, and combinations, mass or molar ratios, reprogramming durations and intervals thereof, that decrease or reverse eosinophil exhaustion, increase eosinophil rejuvenation, provide retention of eosinophil identity, enhance eosinophil proliferation and provide increased efficacy, such as increased tumor killing, may provide attractive candidates for immune cell rejuvenation compositions, methods, kits and associated therapies.

### Example 17

### Transplantation of Rejuvenated T Cells in a Tumor Model

mRNA molecules encoding OCT4 (0), SOX2 (S), KLF4 (K), c-MYC (M), LIN28 (L), and NANOG (N)(i.e., one factor per molecule) are synthesized in vitro from plasmid DNA and purified. Pan T-cells isolated by using MACSxpress® Buffy Coat Pan T Cell Isolation Kit, human (Cat: 130-120-001) from Buffy Coat from blood samples of young (age 20-25 years), middle-aged (age 40-50 years), and aged (age >65 years) individuals, or from commercially available human buffy coat, human buffy coat leukocytes, or human blood samples. Pan T-cells are transduced with lentivirus containing Anti-CD19-ScFv-CD28 (no CD3ζ, Anti-CD19-ScFv-CD28-CD3ζ, and Anti-CD19-ScFv-4-1BB-CD3ζ obtained from GenTarget Inc. and activated by Dynabeads™ Human T-Activator CD3/CD28 for T Cell Expansion and Activation kit (ThermoFisher Cat: 11161D) in X-VIVO-15 (Lonza Cat:BEBP02-054Q) medium, or used in a naïve, non-transduced, non-activated state. In additional conditions, multiple activations are performed, for example 2, 3, or 4 sequential activations.

mRNA molecules in the combination OSKMLN are prepared as naked mRNA in nuclease free H₂O and transfected into T cells using electroporation with the following parameters and conditions: 3-days post activation with Dynabeads, Pan T-cells are electroporated with Neon Transfection System at 1350V, 10 ms, 3 pulse or 1600V, 10 ms, 3 pulse. In each replicate in each condition, 50 nanograms to 10 micrograms of mRNA are used per 1 million cells. An electroporation-only and non-treated control are also included. In additional conditions, transfection is performed at the same time intervals but using lipid nanoparticles and polymer nanoparticles for transfection rather than electroporation; such nanoparticles are as in Example 3. In some experiments, delivery of mRNA molecules with lipid nanoparticles and/or polymer nanoparticles results in enhanced T cell rejuvenation, proliferation, recovery from or prevention of exhaustion, anti-pathogenic effects, anti-cancer effects, or anti-inflammatory effects in the exposed immune cell compared to delivery of mRNA molecules using electroporation.

An in vivo functional ‘CAR stress test’ is performed using limited numbers of CAR T cells in the Nalm6 leukemia model. Total rejuvenated and non-rejuvenated T cells (2×10⁶, 7×10⁵ or 2×10⁵) prepared as above are adoptively transferred into NSG mice bearing pre-established Nalm6 xenografts. Briefly, 6-10-week-old NOD-SCID γc−/− (NSG) mice, which lack an adaptive immune system, are used. In all experiments, the animals are assigned to treatment/control groups using a randomized approach. The animals are injected via the tail vein with 2×10⁶ Nalm6 or 1×10⁶ MOLM14 cells expressing click beetle green luciferase and enhanced green fluorescent protein (eGFP) in 0.1 ml of sterile PBS. CAR T cells or NTD human T cells are injected via the tail vein at the indicated dose in a volume of 100 μl 4 d after the injection with leukemic cells. The mice are given an intraperitoneal injection of 150 mg kg−1 d-luciferin (Caliper Life Sciences). Anesthetized mice are imaged using a Xenogen IVIS Spectrum system (Caliper Life Science).The total flux is quantified using Living Image 4.4 (PerkinElmer). T-cell engraftment is defined as >1% human CD45+ cells in the peripheral blood by flow cytometry. Treatment response is measured according to luciferin chemiluminescence or GFP fluorescence according to methods known in the art.

Data from Example 17 experiments to demonstrate reprogramming factors which provide advantageous T cell features and characteristics in vivo. Reprogramming factors, and combinations thereof, that decrease or reverse T cell exhaustion, increase T cell rejuvenation, provide retention of T cell identity, enhance T cell proliferation and provide increased efficacy, such as increased tumor killing, may provide attractive candidates for immune cell rejuvenation compositions, methods, kits and associated therapies.

### Example 18

### In Vivo Immune Regeneration

mRNA molecules encoding OCT4 (0), SOX2 (S), KLF4 (K), c-MYC (M), LIN28 (L), and NANOG (N) (i.e., one factor per molecule) are synthesized in vitro from plasmid DNA, purified, and incorporated into nanoparticles as described in manufacturer's protocols for Fugene or Polyplus, or in Billingsley et al., Nano Lett. 2020 (20, 3, 1578-1589), McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. Nanoparticles incorporating lipids selected from those described in FIGS. 1-18 are also used.

Naïve mice or aged mice such as C57B/6 aged mice from Charles River are obtained. Nanoparticle-encapsulated mRNA is prepared for injection, for example as described in McKinlay et al. 2018 (PNAS Jun. 26, 2018 115 (26) E5859-E5866) or Rurik et al. (Science 375.6576 (2022): 91-96), incorporated herein by reference.

Nanoparticle-encapsulated mRNA in the combination OSKMLN is injected via the tail vein into mice at a dose of 10-1000 pg. Injections are performed every day for a dosing interval 4-10 days. In some experiments, the dosing interval is repeated up to four times, with 7-60 days between intervals. At 2-14 days after the last injection, immune cells including T cells, NKT cells, gamma delta T cells, NK cells, monocytes/macrophages, dendritic cells, B cells, neutrophils, and eosinophils are isolated from the blood, spleen, lymph nodes, thymus, and liver of mice according to standard methods known to those skilled in the art. Rejuvenation, prevention and reversal of senescence and exhaustion, proliferation, and efficacy of the isolated cells are performed as described in the example above.

Data from Example 18 experiments demonstrate reprogramming factors which provide advantageous immune cell features and characteristics in vivo. Reprogramming factors, and combinations, mass or molar ratios, reprogramming durations and intervals thereof, that decrease or reverse immune cell exhaustion, increase immune cell rejuvenation, provide retention of immune cell identity, enhance immune cell proliferation and provide increased efficacy may provide attractive candidates for immune cell rejuvenation compositions, methods, kits and associated therapies.

### Example 19

### Polycistronic RNA Vector for Expression of Codon-Optimized Oct4, Sox2, and KLF4

A pMK expression vector (Life Technologies), containing a polynucleotide sequence of SEQ ID NOs: 1, a polynucleotide sequence of SEQ ID NO: 2, a polynucleotide sequence of SEQ ID NO: 4, an additionally added internal ribosome entry site (IRES)-GFP, 5′ and 3′ UTRs, and linker regions, is amplified in E. coli and plasmids are isolated using QIAPrep (Qiagen, Hilden, Germany). After the linearization, 10 pg template DNA is transcribed in vitro using RiboMAX large-scale production system T7 Kit (Promega, Madison, WI, USA) according to the manufacturer's instructions. Afterwards, 2 U TURBO DNase is added for 15 min at 37° C. For 5′-end capping, ScriptCap Cap1 Capping System is used followed by 30-end polyadenylation with A-Plus Poly(A) Polymerase Tailing Kit (both from Cellscript, Madison, WI, USA) according to the manufacturer's instructions. Following each reaction step, RNA is purified using RNeasy Kit (Qiagen). The specific lengths of the generated DNA and RNA products are analyzed using 1% agarose gel electrophoresis.

### Example 20

### Self-Replicating RNA (srRNA)

A T7-VEE-OKS-iM plasmid, as described in PCT/US2013/041980, containing sequences encoding the non-structural proteins (nsP1 to nsP4) for self-replication, the reprogramming factors Oct4, Klf4, Sox2, and cMyc and an additionally added internal ribosome entry site (IRES)-GFP is amplified in E. coli and plasmids are isolated using QIAPrep (Qiagen, Hilden, Germany). After the linearization with MluI restriction enzyme (Thermo Fisher Scientific), 10 pg template DNA is transcribed in vitro using RiboMAX large-scale production system T7 Kit (Promega, Madison, WI, USA) according to the manufacturer's instructions. Afterwards, 2 U TURBO DNase is added for 15 min at 37° C. For 5′-end capping, ScriptCap Cap1 Capping System is used followed by 30-end polyadenylation with A-Plus Poly(A) Polymerase Tailing Kit (both from Cellscript, Madison, WI, USA) according to the manufacturer's instructions. Following each reaction step, srRNA is purified using Rneasy Kit (Qiagen). The specific lengths of the generated DNA and srRNA products are analyzed using 1% agarose gel electrophoresis.

### Example 21

### In Vitro Production of Circular RNA

Unmodified linear RNA is synthesized by in vitro transcription using T7 RNA polymerase from a DNA segment having 5′- and 3′-ZKSCAN1 introns and an open reading frame (ORF) encoding green fluorescent protein (GFP) linked to stagger element sequences. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), and the circular RNA is isolated following enrichment with Rnase R treatment. RNA quality is assessed by agarose gel or through automated electrophoresis (Agilent).

### Example 22

### T Cell Rejuvenation

T cells are collected from patients via apheresis, obtained as “off the shelf” allogeneic T cell products, or purchased from a commercial source (e.g., Precision For Medicine). The T cells are maintained in culture under conditions and for a duration that promotes T cell exhaustion. T cells are monitored for exhaustion by analysis of the levels of interleukin-2 (IL-2) and tumor necrosis factor-α (TNF-α), whereby a decreased level of IL-2 and TNF-α indicates onset of exhaustion. T cell activity in cell killing assays is also monitored whereby decreased killing activity in the cell killing assay is also indicative of the onset of T cell exhaustion. The exhausted T cells are maintained and expanded in culture for additional experimentation.

The mRNA vector, self-replicating RNA, or circular RNA from Examples 3 or 7-9 is used to transfect the cultured, exhausted T cells. T cell transfection is performed as described in Examples 3 or 7-9 or as described in Sambrook et al. (Molecular Cloning, a laboratory manual, 3rd edition, Cold Spring Harbor Laboratories, 2001).

Next, transfected T cells are cultured under conditions to promote and allow expression of the reprogramming factors (OCT4, SOX2, and KLF4, and cMYC) from the transfected mRNA constructs. Analysis of the T cells for expression of the reprogramming factors confirms sufficient transfection and expression of OCT4, SOX2, and KLF4, and cMYC.

Transfected, exhausted T cells confirmed to be expressing the reprogramming factors are then cultured to allow ample time for reprogramming factor expression and then assayed for T cell rejuvenation. T cells are again analyzed for levels of IL-2 and TNF-α, whereby an increased level of IL-2 and TNF-α, compared to the levels for exhausted T cells, is indicative of T cell rejuvenation. T cell activity in cell killing assays is also monitored whereby increased killing activity, compared to the killing activity level of exhausted T cells, is also indicative of T cell rejuvenation.

### Example 23

### Rejuvenated T Cell Manufacture, Cyotoxicity, Proliferation and Marker Expression

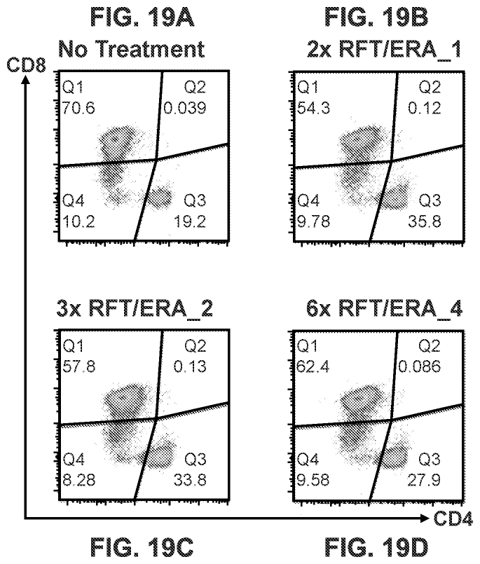

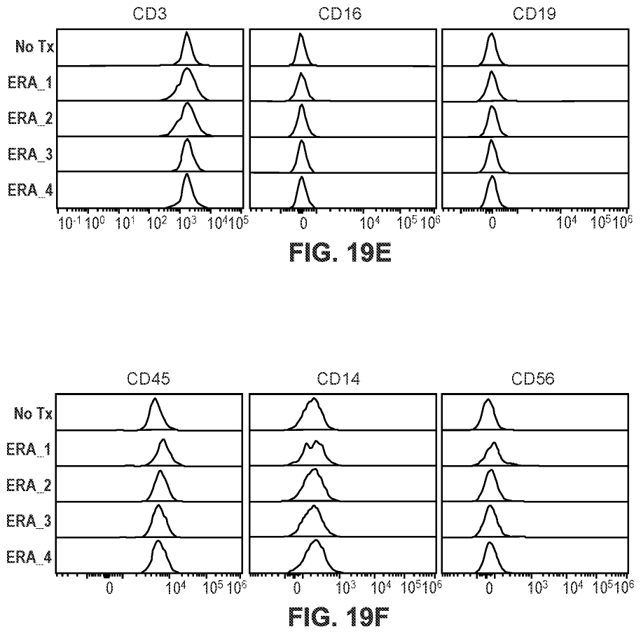

Rejuvenated T cells expressing reprogramming factors were produced as described herein in the manufacturing of a cellular therapy product. Marker expression (CD3, CD4, CD8, CD14, CD16, CD19, CD45, and CD56) was analyzed using flow cytometry at day 15 of manufacture. Marker expression indicates T cell identity is preserved at day 15 of manufacture after 1×, 2×, 3× and/or 6× reprogramming factor treatments (RFT) as shown in FIGS. 19(A) through 19(F).

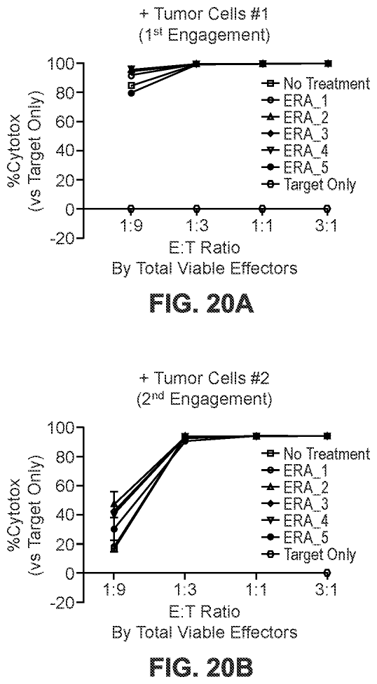

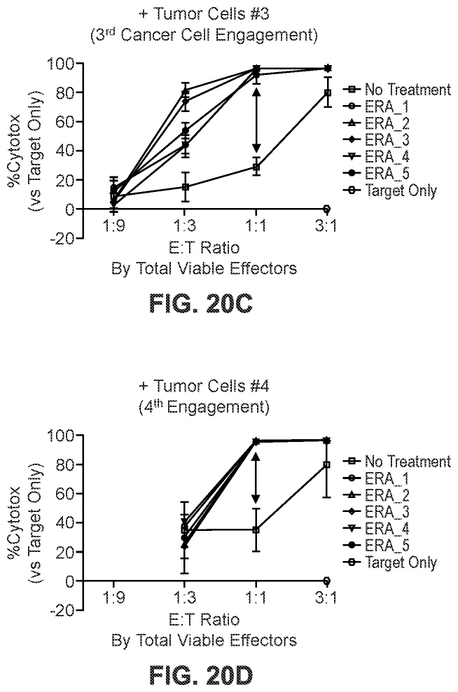

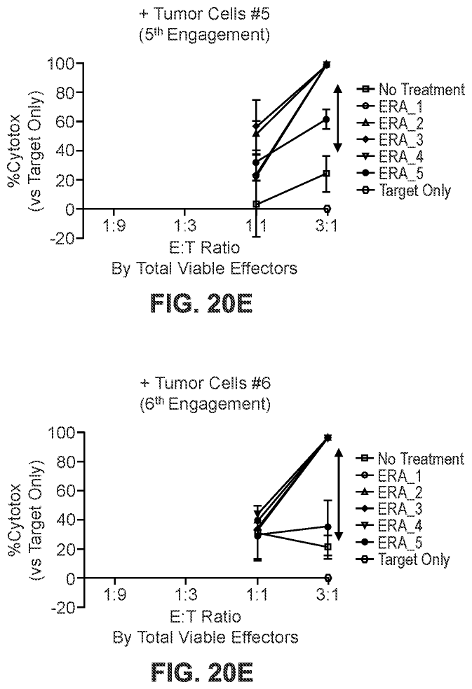

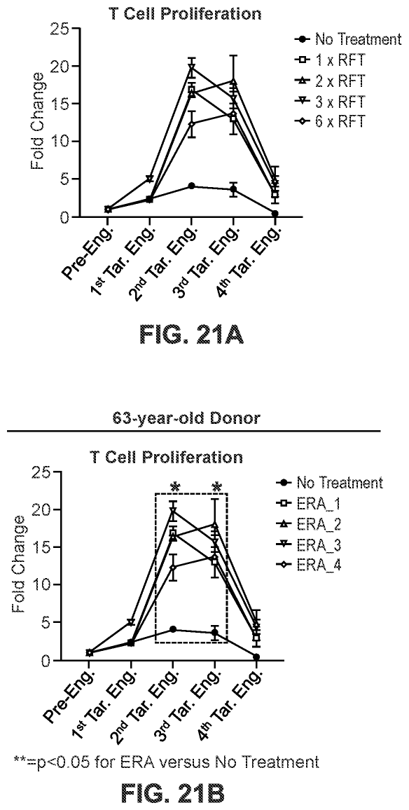

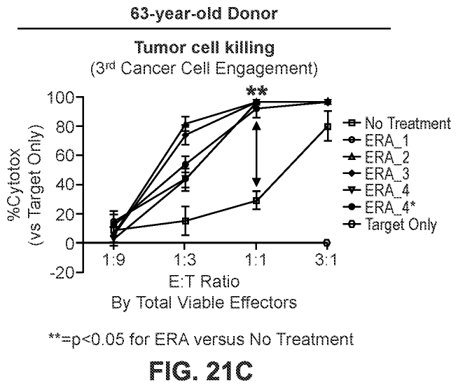

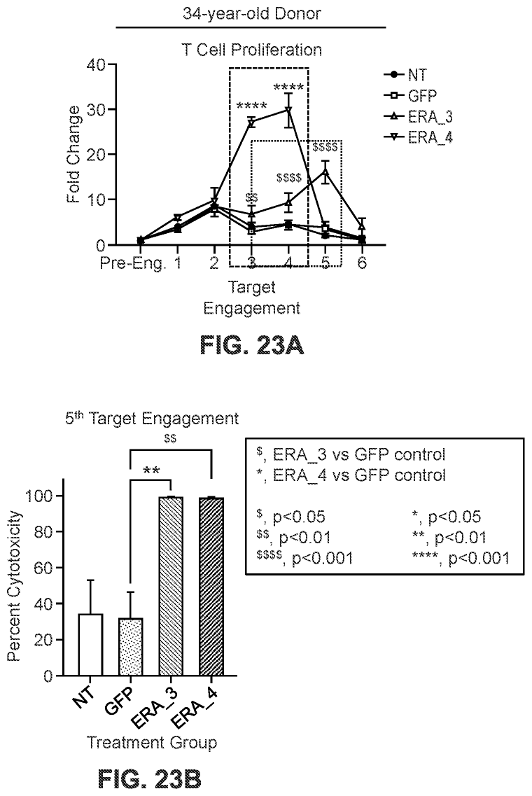

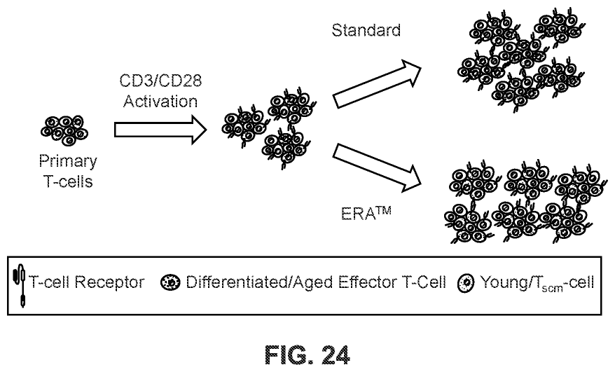

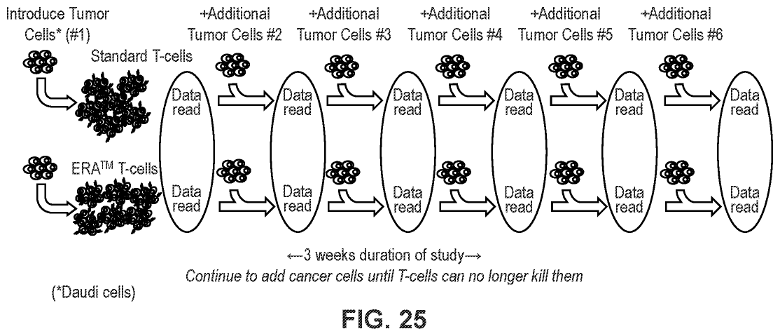

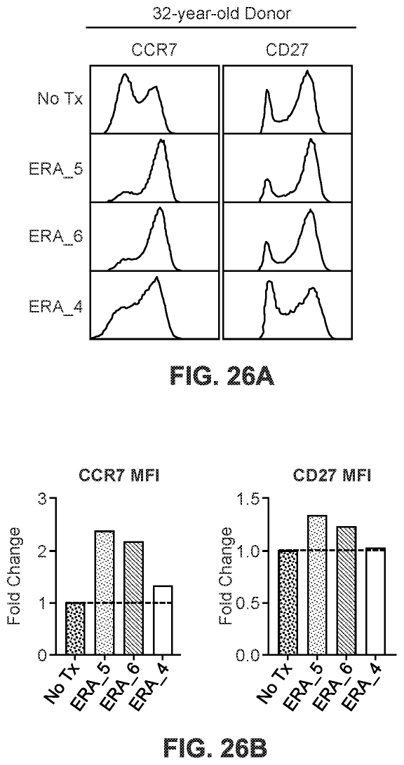

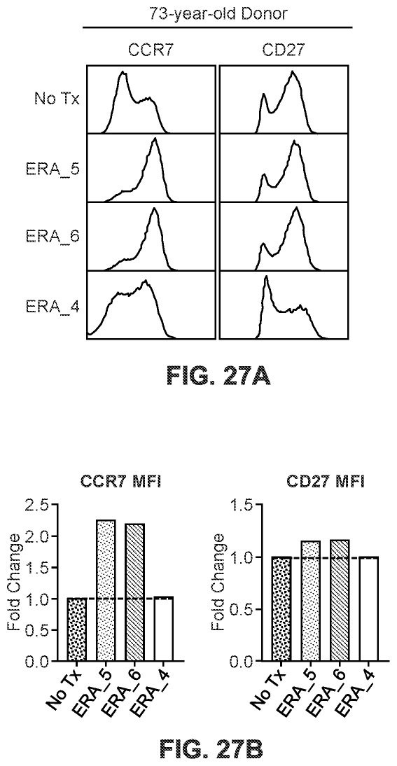

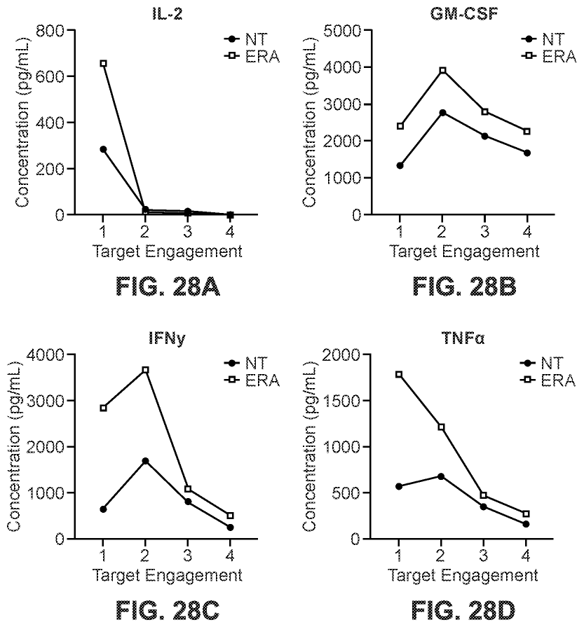

A rejuvenated T cell cellular therapy product, comprising T cells expressing reprogramming factors as described herein, was tested for T cell mediated cytotoxicity. T cell mediated cytotoxicity was tested in a cell killing assay with Daudi target cells and T cells expressing reprogramming factors as described herein compared with un-rejuvenated control T cells. Reprogrammed and control T cells were tested after 1×, 2×, 3×and/or 6× reprogramming factor treatments (RFT). At the third, and subsequent, addition of target tumor cells, rejuvenated T cells expressing reprogramming factors, as provided herein, exhibited significantly increased target cell cytotoxicity (4×-5× more cytotoxicity) compared to un-rejuvenated T cell controls, as shown in (FIG. 20) and FIG. 23(A). This increased efficiency in killing tumor cells remained high, even after several additions of tumor cells, as shown in FIGS. 20(C)-20(F) and FIGS. 23(C)-23(F). A rejuvenated T cell cellular therapy product, comprising T cells expressing reprogramming factors as described herein, was tested for proliferation and compared with un-rejuvenated control T cells. Reprogrammed T cells were tested with T cell proliferation assays after 1×, 2×, 3× and/or 6× reprogramming factor treatments (RFT). Rejuvenated T cells expressing reprogramming factors, as provided herein, exhibit increased proliferation (3×-5× more proliferation) compared to un-rejuvenated T cell controls (FIG. 21).

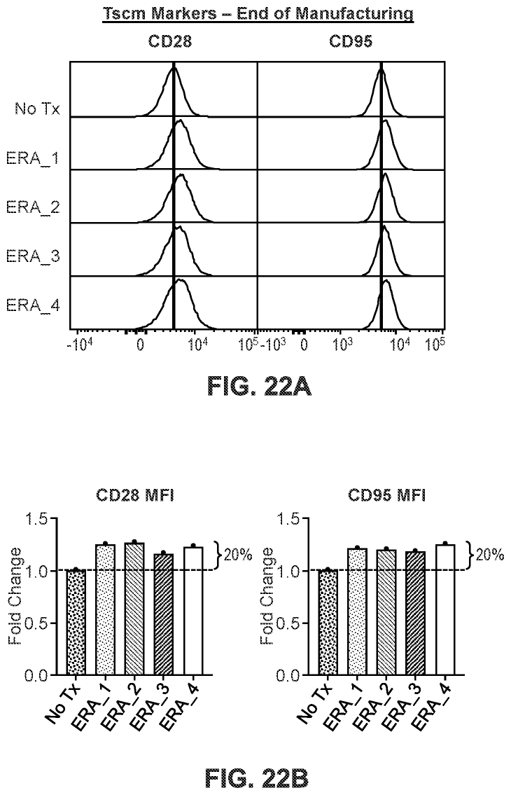

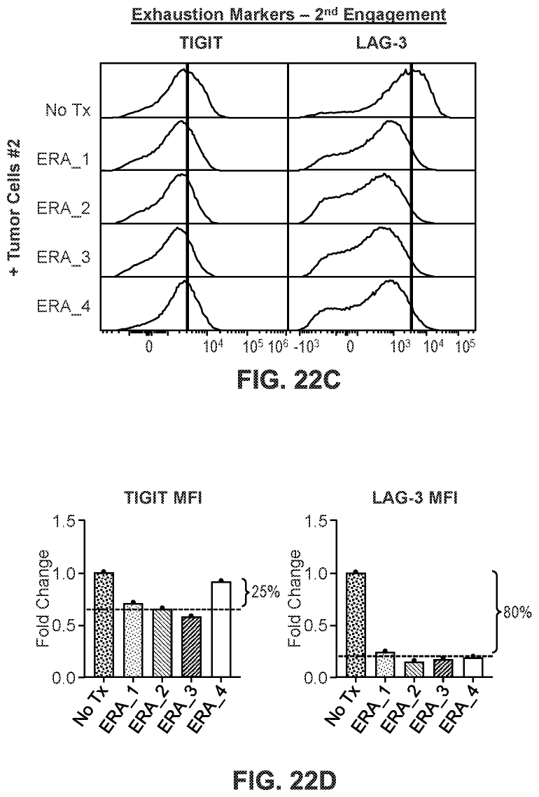

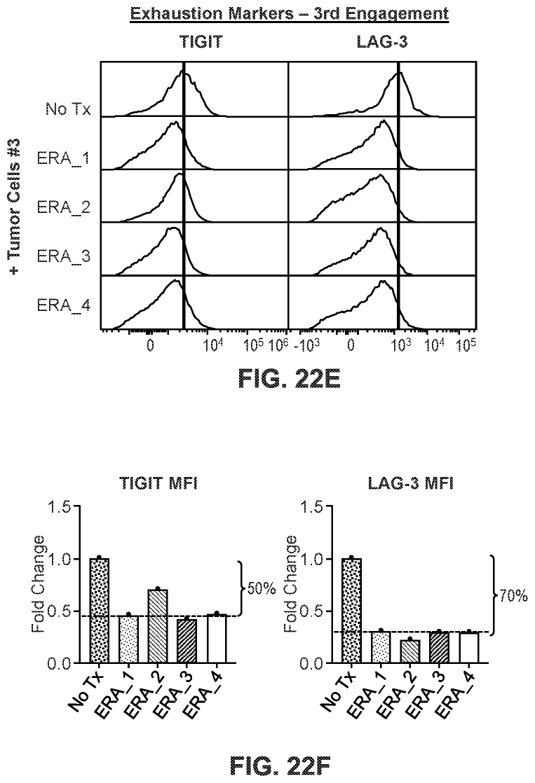

A rejuvenated T cell cellular therapy product, comprising T cells expressing reprogramming factors as described herein, was tested for marker expression at the end of manufacture and upon Daudi target cell engagement. Reprogrammed T cells were analyzed using flow cytometry after 1X, 2X, 3X and/or 6X reprogramming factor treatments (RFT). Marker expression analysis indicates T cells expressing reprogramming factors, as provided herein, express higher levels of T cell markers (CD28 and CD95) at the end of manufacture and lower levels of T cell exhaustion markers (TIGIT and LAG3) after Daudi cell target engagement (FIG. 22).

### Example 24

### Rejuvenated T Cell Manufacture, Cytotoxicity, Proliferation and Marker Expression

mRNA molecules encoding six reprogramming factors (OCT4, SOX2, c-MYC, KLF4, LIN28 and NANOG, collectively, “OSKMLN”) and three reprogramming factors (OMN) were prepared as naked mRNA in nuclease free H₂O and applied to T cells in a mass ratio of (3:1:1:1:1:1) (OCT4: SOX2: c-MYC: KLF4: LIN28: NANOG, where the mass of OCT4 that was used was three times the mass used for any one of the other five factors) and a mass ratio of 1:1:1 (OCT4: c-MYC: NANOG). The mRNA molecules were transfected into the T cells using electroporation with the following parameters and conditions: 3-days post activation with Dynabeads, Pan T-cells were electroporated with Neon Transfection System at 1350V, 10 ms, 3 pulse or 1600V, 10 ms, 3 pulse. In each replicate in each condition, 10⁶ (one million) cells were transfected. An electroporation-only and non-treated control were also included. After electroporation, cells were returned to culture conditions. Cells were cultured for total of 10 days (5 days rested post ERA) or 18 days (13 days rested post ERA), with electroporation delivery of mRNA performed twice per day for three consecutive days. The first of the two daily doses was between 100 ng to 10 ug of mRNA encoding OSKMLN per million cells, and the second of the two daily doses was between 100 ng to 10 ug of mRNA encoding OMN per million cells.

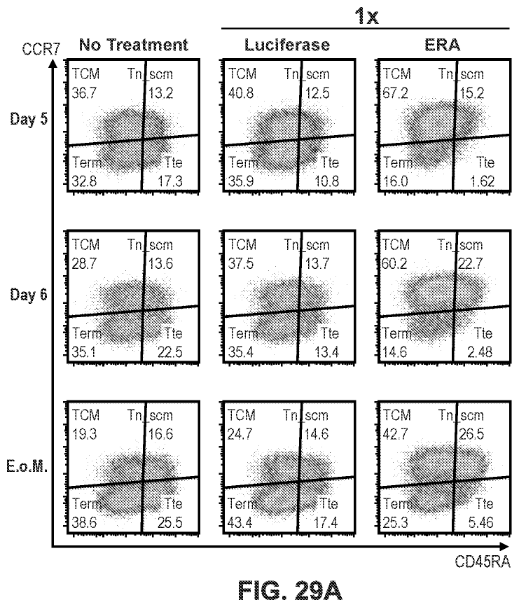

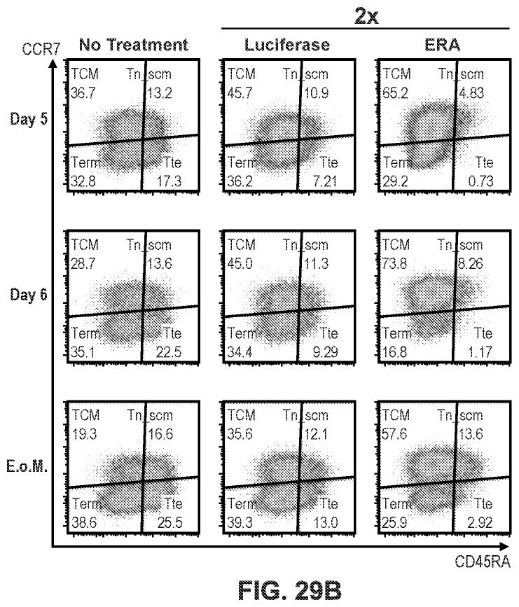

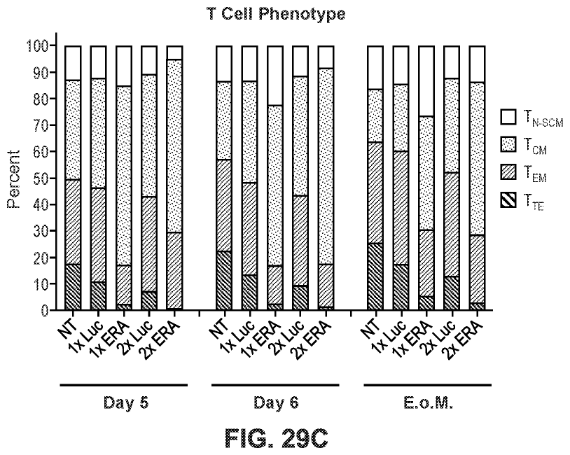

For analysis of prevention and reversal of exhaustion CD45RA and CCR7 were assessed using flow cytometry, and the results at the End of Manufacture (E.o.M.) and on Day 5 and Day 6 are shown in FIG. 29A and FIG. 29B, respectively. In this figure, “Day 5” and “Day 6” indicate the day after the first ERA treatment. 1×ERA and 2×ERA indicate different doses of ERA treatment. 1×Luc (Luciferase) and 2×Luc (Luciferase) are the corresponding control samples, which received similar mock mRNA doses. Increased CCR7 expression indicates this ERA treatment regimen, with mRNA encoding reprogramming factors transfected twice a day over three consecutive days at the specified doses for mRNA molecules encoding OSKMLN and OMN reprogramming factors, enhances central memory (Tcm) and stem memory T cell (Tscm)-like phenotype. The percentages of four specific T cell phenotypes are shown in FIG. 29C (bar graphs are generated from the percentages from the CCR7/CD45RA quadrants shown in FIG. 29A and FIG. 29B). Increased percentages of Tcm and Tscm cells within the T cell population are expected to lead to higher proliferation and longer persistence in patients. (EoM: End of Manufacturing. T_{NSCM} (T Naïve-Stem Cell Memory), TCM (T central memory), TEM (T Effector Memory), TE (T Effector)).

### Example 25

### Rejuvenated T Cell Manufacture, Cytotoxicity, Proliferation and Marker Expression at Lower and Higher RNA Doses

mRNA molecules encoding six reprogramming factors (OCT4, SOX2, c-MYC, KLF4, LIN28 and NANOG, collectively, “OSKMLN”) and three reprogramming factors (OMN) are prepared as naked mRNA in nuclease free H₂O and applied to T cells in mass ratios from (1-3:1-3:1-3:1-3:1-3:1-3) (OCT4: SOX2: c-MYC: KLF4: LIN28: NANOG) and in mass ratios from (1-3:1-3:1-3) (OCT4: c-MYC: NANOG). The mRNA molecules are transfected into T cells using electroporation with the following parameters and conditions: 3-days post activation with Dynabeads, Pan T-cells are electroporated with Neon Transfection System at 1350V, 10 ms, 3 pulse or 1600V, 10 ms, 3 pulse. To allow for comparison, parallel experiments are conducted where mRNA molecules are transfected into T cells using lipid nanoparticles as described in Example 18. In each replicate in each condition, 10⁶ (one million) cells are transfected. Electroporation-only and non-treated controls are also included. After electroporation or transfection using lipid nanoparticles, cells are returned to culture conditions. Cells are cultured for total of 10 days (5 days rested post ERA) or 18 days (13 days rested post ERA), with electroporation or lipid nanoparticle delivery of mRNA performed twice per day for three consecutive days. The first of the two daily doses is between 1 ng to 100 ug of mRNA encoding OSKMLN per million cells, and the second of the two daily doses is between 1 ng to 100 ug of mRNA encoding OMN per million cells.

For analysis of prevention and reversal of exhaustion CD45RA and CCR7 are assessed using flow cytometry, and the results at the End of Manufacture (E.o.M.) and on Day 5 and Day 6. Increases in CCR7 expression indicate this ERA treatment regimen, where mRNA encoding reprogramming factors is transfected twice a day over three consecutive days at the specified doses for mRNA molecules encoding OSKMLN and OMN reprogramming factors, enhances central memory (Tcm) and stem memory T cell (Tscm)-like phenotype. The percentages of four specific T cell phenotypes are generated based on the percentages CCR7/CD45RA quadrants using flow cytometry as described in the Examples above. Increased percentages of Tcm and Tscm cells within the T cell population are expected to lead to higher proliferation and longer persistence in patients.

Data from the experiments in Examples 1-25 describe reprogramming factors which provide advantageous immune cell features and characteristics. Reprogramming factors, and combinations thereof, that decrease or reverse immune cell exhaustion, increase immune cell rejuvenation, provide retention of immune cell identity, enhance immune cell proliferation and provide increased efficacy, such as increased tumor killing, may provide attractive candidates for immune cell rejuvenation compositions, methods, kits and associated therapies. For example, rejuvenated immune cells, such as rejuvenated T cells, may then be used in cellular therapies for immune cell related diseases and disorders.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope.

1-200. (canceled)

201. An ionizable lipid of Formula (I) 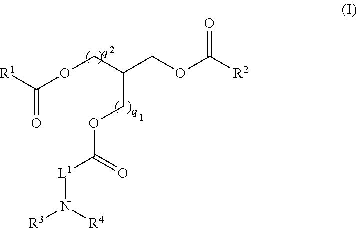
or a pharmaceutically acceptable salt or a stereoisomer thereof, wherein:
L¹ is C₁-C₆ alkylene; R¹ is C₆-C₂₀ alkenyl; R² is C₆-C₂₀ alkyl; R³ and R⁴ are each independently H or C₁-C₃ alkyl; q₁ is absent or 1; and q₂ is absent or 1.

202. The ionizable lipid of claim 201, wherein the ionizable lipid has one of the following structures:

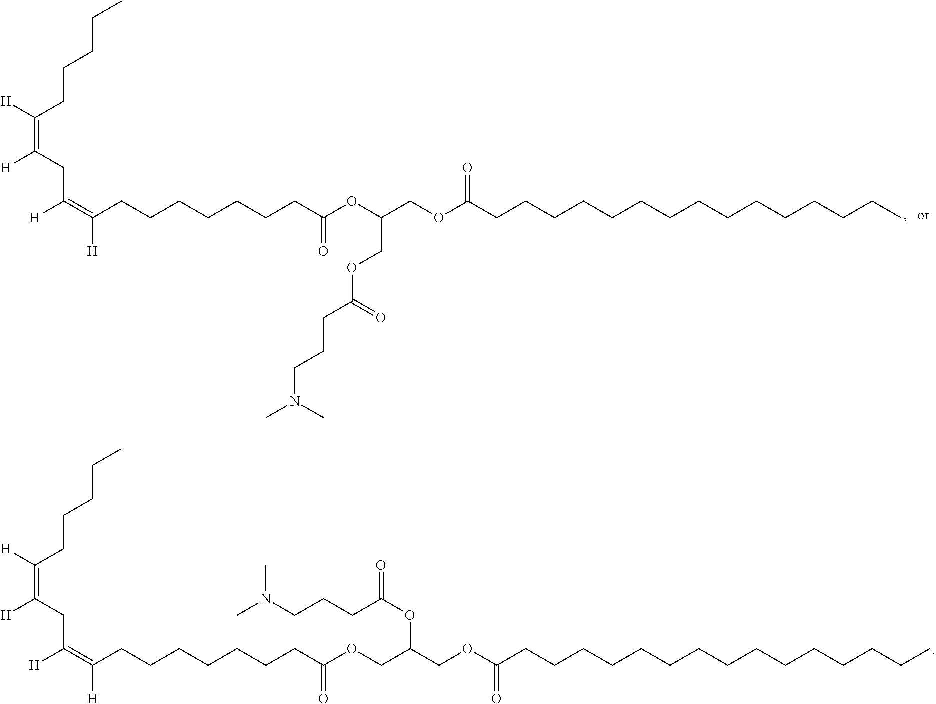 [SMILES: CCCCC/C=C\C/C=C\CCCCCCCC(=O)OC(COC(=O)CCCCCCCCCCCCCCC)COC(=O)CCCN(C)C ; CCCCC/C=C\C/C=C\CCCCCCCC(=O)OCC(COC(=O)CCCCCCCCCCCCCCC)OC(=O)CCCN(C)C]

203. An ionizable lipid of Formula (I-B) 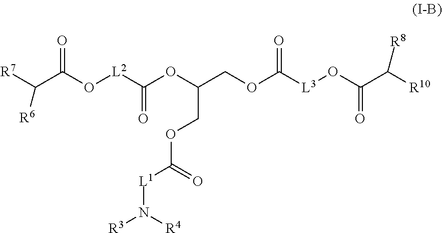
or a pharmaceutically acceptable salt or a stereoisomer thereof, wherein:
L¹ is C₁-C₆ alkylene; L² is C₁-C₈ alkylene; L³ is C₁-C₈ alkylene; R³ and R⁴ are each independently H or C₁-C₃ alkyl; R⁶ is C₄-C₂₀ alkyl; R⁷ is C₄-C₂₀ alkyl; R⁸ is C₄-C₂₀ alkyl; and R¹⁰ is C₄-C₂₀ alkyl.

204. The ionizable lipid of claim 203, wherein the ionizable lipid has the following structure:

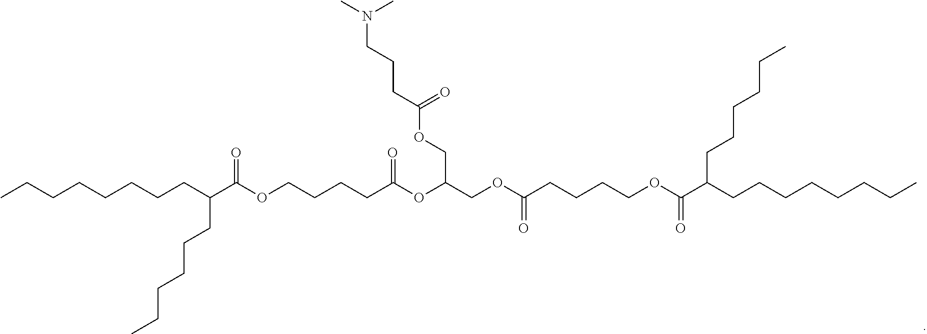 [SMILES: CCCCCCCCC(CCCCCC)C(=O)OCCCCC(=O)OCC(COC(=O)CCCN(C)C)OC(=O)CCCCOC(=O)C(CCCCCC)CCCCCCCC]

205. A lipid-nanoparticle composition, comprising an ionizable lipid of claim 201.

206. The lipid-nanoparticle composition of claim 205, wherein the lipid-nanoparticle composition further comprises an uncharged lipid, a lipid conjugated to a moiety, and/or a biodegradable lipid.

207. The lipid-nanoparticle composition of claim 205, wherein the lipid-nanoparticle composition comprises a helper lipid selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

208. The lipid-nanoparticle composition of claim 205, wherein the lipid-nanoparticle composition comprises a structural lipid selected from the group consisting of cholesterol, cholesterol derivatives, fecosterol, sitosterol, ergosterol, campesterol, stigmasterol, brassicasterol, tomatidine, ursolic acid, alpha tocopherol, and mixtures thereof.

209. The lipid-nanoparticle composition of claim 206, wherein the uncharged lipid is a phospholipid or cholesterol.

210. The lipid-nanoparticle composition of claim 205, wherein the ionizable lipid has the following structure:

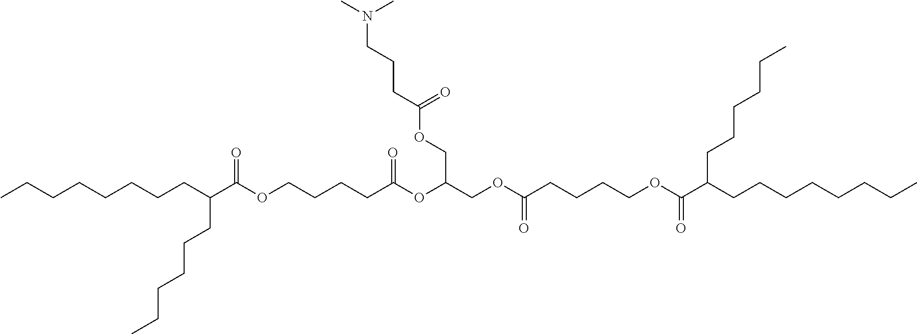 [SMILES: CCCCCCCCC(CCCCCC)C(=O)OCCCCC(=O)OCC(COC(=O)CCCN(C)C)OC(=O)CCCCOC(=O)C(CCCCCC)CCCCCCCC]

211. A lipid-nanoparticle composition, comprising an ionizable lipid of claim 203.

212. The lipid-nanoparticle composition of claim 211, wherein the lipid-nanoparticle composition further comprises an uncharged lipid, a lipid conjugated to a moiety, and/or a biodegradable lipid.

213. The lipid-nanoparticle composition of claim 211, wherein the lipid-nanoparticle composition comprises a helper lipid selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

214. The lipid-nanoparticle composition of claim 211, wherein the lipid-nanoparticle composition comprises a structural lipid selected from the group consisting of cholesterol, cholesterol derivatives, fecosterol, sitosterol, ergosterol, campesterol, stigmasterol, brassicasterol, tomatidine, ursolic acid, alpha tocopherol, and mixtures thereof.

215. The lipid-nanoparticle composition of claim 212, wherein the uncharged lipid is a phospholipids or cholesterol.

216. The lipid-nanoparticle composition of claim 211, wherein the ionizable lipid has the following structure: 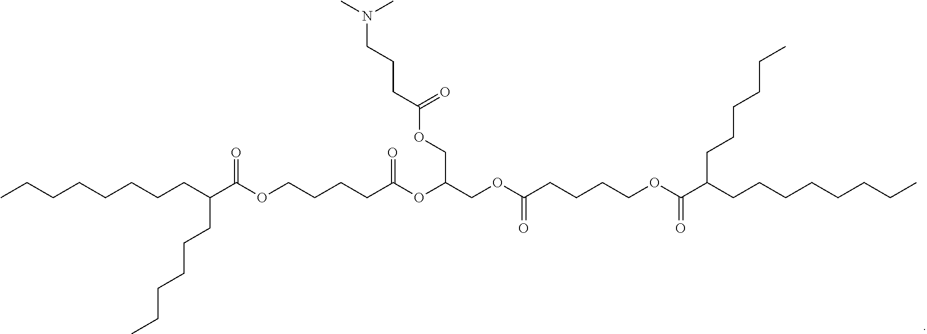 [SMILES: CCCCCCCCC(CCCCCC)C(=O)OCCCCC(=O)OCC(COC(=O)CCCN(C)C)OC(=O)CCCCOC(=O)C(CCCCCC)CCCCCCCC]
